(19) United States
(12) Patent Application Publication
(10) Pub. No.: US 2026/0306667 A1
(43) Pub. Date: Oct. 8, 2026

(54) RECOMBINANT ADENO-ASSOCIATED VIRAL VECTORS FOR MULTIPARTITE GENE DELIVERY AND STARGARDT DISEASE TREATMENT

(71) Applicant: Abeona Therapeutics Inc., Cleveland, OH (US)

(72) Inventors: Linas PADEGIMAS, Mayfield, OH (US); Brianna BARRETT, Cleveland, OH (US)

(21) Appl. No.: 19/480,957

(22) PCT Filed: May 3, 2024
(86) PCT No.: PCT/US2024/027593

§ 371 Date: Nov. 3, 2025

Related U.S. Application Data
(60) Provisional application No. 63/500,134, filed on May 4, 2023.

(51) Int. Cl. A61K 48/00; A61K 38/17; A61P 27/02; C12N 15/86

(52) U.S. Cl. CPC A61K 48/005; A61K 38/1709; A61P 27/02; C12N 15/86; C12N 2750/14143; C12N 2830/50

(57) ABSTRACT

Provided herein are recombinant AAV vectors, AAV vector genomes, AAV viral vectors, and capsid proteins for improved gene therapy, and methods for their manufacture and use in a multipartite (e.g., bipartite) delivery system. The vectors provide portions of transgene and direct their recombination in a cell to provide complete transgenes. Also provided are regulatory sequences (e.g., enhancer, promoter) that results in high level expression of the transgene (e.g., ABCA4), which are useful for gene therapy (e.g., Stargardt disease treatment).

### CROSS-REFERENCE TO RELATED APPLICATIONS

This application claims the benefit of U.S. Provisional Application No. 63/500,134, filed May 4, 2023, which is herein incorporated by reference in its entirety.

### REFERENCE TO AN ELECTRONIC SEQUENCE LISTING

The content of the electronic sequence listing (ABEO_010_01WO_SeqList_ST26.xml; Size: 574,947 bytes; and Date of Creation: Apr. 29, 2024) is herein incorporated by reference in its entirety.

### BACKGROUND

Adeno-associated viral (AAV) vectors are promising delivery vectors for gene therapy. However, their therapeutic efficacy is undermined by limitations regarding delivery of genes above a certain size. For example, Stargardt disease is an inherited retinal disease that causes juvenile macular degeneration. Autosomal recessive Stargardt disease is caused by mutations in the ABCA4 gene. The ORF of the human ABCA4 gene is about 6.8 kb in length, which exceeds the packaging capacity of about 4.7 kb typical of AAV vectors. Therefore, there is an urgent need for AAV viral vector approaches for delivering large genes with regulatory sequences that allow optimal protein expression.

### SUMMARY

The present disclosure relates generally to the field of gene therapy and, in particular, to recombinant adeno-associated viral (AAV) particles and their use to deliver transgenes in a multipartite (e.g., bipartite) approach to treat or prevent a disease or disorder, such as Stargardt disease.

In one aspect, the disclosure provides AAV vector genomes comprising, in 5′ to 3′ orientation: (a) a 5′ AAV inverted terminal repeat; (b) an interphotoreceptor retinoid-binding protein (IRBP) enhancer; (c) an eye-specific promoter; (d) a 5′ portion of a transgene; (e) a splice donor (SD) site; (f) a recombination site; (g) a polynucleotide encoding a recombinase; (h) a poly-A site; and (i) a 3′ AAV inverted terminal repeat. In embodiments, the IRBP enhancer comprises or consists of a sequence having at least 90%, at least 95%, at least 96%, at least 97%, at least 98%, at least 99%, or 100% identity to SEQ ID NO: 208.

In embodiments, the eye-specific promoter is a human rhodopsin kinase (hGRK1) promoter, a human rhodopsin (hRho) promoter, a human rod cGMP phosphodiesterase β-subunit (βPDE) promoter, a human interphotoreceptor retinoid-binding protein (IRBP) promoter, a human red/green opsin promoter, a human blue opsin (HB) promoter, a human vitelliform macular dystrophy-2 (VMD2), or a human RPE65 promoter. In embodiments, the eye-specific promoter is a human rod cGMP phosphodiesterase β-subunit (βPDE) promoter comprising or consisting of a sequence having at least 90%, at least 95%, at least 96%, at least 97%, at least 98%, at least 99%, or 100% identity to SEQ ID NO: 200. In embodiments, the eye-specific promoter is a human rhodopsin (hRho) promoter comprising or consisting of a sequence having at least 90%, at least 95%, at least 96%, at least 97%, at least 98%, at least 99%, or 100% identity to SEQ ID NO: 209. In embodiments, the eye-specific promoter is a human rhodopsin kinase (hGRK1) promoter comprising or consisting of a sequence having at least 90%, at least 95%, at least 96%, at least 97%, at least 98%, at least 99%, or 100% identity to SEQ ID NO: 188.

In one aspect, the disclosure provides AAV vector genomes, comprising, in 5′ to 3′ orientation: (a) a 5′ AAV inverted terminal repeat; (b) a human rhodopsin (hRho) promoter; (c) a 5′ portion of a transgene; (d) a splice donor (SD) site; (e) a recombination site; (f) a polynucleotide encoding a recombinase; (g) a poly-A site; and (h) a 3′ AAV inverted terminal repeat. In embodiments, the human rhodopsin (hRho) promoter comprises or consists of a sequence having at least 90%, at least 95%, at least 96%, at least 97%, at least 98%, at least 99%, or 100% identity to SEQ ID NO: 209. In embodiments, the AAV vector genome comprises an enhancer between the 5′ AAV inverted terminal repeat and the human rhodopsin (hRho) promoter.

In embodiments, the AAV vector genome comprises a CBA-MVM hybrid sequence between the eye-specific promoter and the 5′ portion of the transgene. In embodiments, the CBA-MVM hybrid sequence comprises or consists of a sequence having at least 90%, at least 95%, at least 96%, at least 97%, at least 98%, at least 99%, or 100% identity to SEQ ID NO: 210.

In one aspect, the disclosure provides AAV vector genomes comprising, in 5′ to 3′ orientation: (a) a 5′ AAV inverted terminal repeat; (b) a CMV enhancer; (c) a chicken beta-actin (CBA) promoter; (d) a 5′ portion of a transgene; (e) a splice donor (SD) site; (f) a recombination site; (g) a polynucleotide encoding a recombinase; (h) a poly-A site; and (i) a 3′ AAV inverted terminal repeat. In embodiments, the CMV enhancer comprises or consists of a sequence having at least 90%, at least 95%, at least 96%, at least 97%, at least 98%, at least 99%, or 100% identity to SEQ ID NO: 218. In embodiments, the CBA promoter comprises or consists of a sequence having at least 90%, at least 95%, at least 96%, at least 97%, at least 98%, at least 99%, or 100% identity to SEQ ID NO: 219. In embodiments, the AAV vector genome comprises a sequence having at least 90%, at least 95%, at least 96%, at least 97%, at least 98%, at least 99%, or 100% identity to SEQ ID NO: 211 between the 5′ AAV inverted terminal repeat and the 5′ portion of the transgene.

In embodiments, the 5′ portion of the transgene is at least 0.5 kb, at least 1 kb, at least 1.5 kb, at least 2 kb, or at least 2.5 kb in length. In embodiments, the transgene encodes an ATP-binding cassette, sub-family A, member 4 (ABCA4) protein. In embodiments, the ABCA4 protein comprises or consists of a sequence having at least 90%, at least 95%, at least 96%, at least 97%, at least 98%, at least 99%, or 100% identity to SEQ ID NO: 190. In embodiments, the 5′ portion of the transgene comprises or consists of a sequence having at least 90%, at least 95%, at least 96%, at least 97%, at least 98%, at least 99%, or 100% identity to SEQ ID NO: 212. In embodiments, the 5′ portion of the transgene encodes a polypeptide sequence comprising amino acids 1-884 of human ABCA4 protein (SEQ ID NO: 190). In embodiments, the 5′ portion of the transgene comprises one or more of the following nucleotides: (i) a nucleotide “G” at the position corresponding to nucleotide 813 of SEQ ID NO: 212; (ii) a nucleotide “A” at the position corresponding to nucleotide 1311 of SEQ ID NO: 212; (iii) a nucleotide “A” at the position corresponding to nucleotide 2274 of SEQ ID NO: 212; (iv) a nucleotide “C” at the position corresponding to nucleotide 2277 of SEQ ID NO: 212; (v) a nucleotide “T” at the position corresponding to nucleotide 2278 of SEQ ID NO: 212; and (vi) a nucleotide “T” at the position corresponding to nucleotide 2568 of SEQ ID NO: 212. In embodiments, the 5′ portion of the transgene comprises all of the nucleotides (i)-(vi).

In embodiments, the splice donor (SD) site comprises or consists of a sequence having at least 90%, at least 95%, at least 96%, at least 97%, at least 98%, at least 99%, or 100% identity to SEQ ID NO: 186.

In embodiments, the recombination site is a Lox71 site comprising or consisting of SEQ ID NO: 178, or a sequence having at most 1, at most 2, or at most 3 nucleotides mutations (deletion, insertion, and/or substitution) thereto.

In embodiments, expression of the recombinase is operably linked to the promoter.

In embodiments, the recombinase is a Cre recombinase. In embodiments, the Cre recombinase comprises a sequence having at least 90%, at least 95%, at least 96%, at least 97%, at least 98%, at least 99%, or 100% identity to SEQ ID NO: 164. In embodiments, the polynucleotide encoding the recombinase comprises an intron comprising or consisting of a sequence having at least 90%, at least 95%, at least 96%, at least 97%, at least 98%, at least 99%, or 100% identity to SEQ ID NO: 214. In embodiments, the polynucleotide encoding the recombinase comprises a sequence having at least 90%, at least 95%, at least 96%, at least 97%, at least 98%, at least 99%, or 100% identity to SEQ ID NO: 213.

In embodiments, the recombinase comprises a N-terminal nuclear localization sequence (NLS) comprising or consisting of SEQ ID NO: 165.

In embodiments, the AAV vector genome comprises a polynucleotide encoding an internal cleavage polypeptide located between the recombination site and the polynucleotide encoding the recombinase, and wherein the 5′ portion of the transgene, the polynucleotide encoding the internal cleavage polypeptide, and the polynucleotide encoding the recombinase are in the same reading frame. In embodiments, the internal cleavage polypeptide is a self-cleaving peptide selected from the group consisting of T2A, P2A, E2A and F2A. In embodiments, the self-cleaving peptide is a P2A peptide comprising or consisting of SEQ ID NO: 181.

In embodiments, the poly-A site comprises or consists of SEQ ID NO: 189, or a sequence having at most 1, at most 2, at most 3, at most 4, at most 5, or at most 6 nucleotides mutations (deletion, insertion, and/or substitution) thereto.

In embodiments, the AAV vector genome comprises an internal ribosome entry site (IRES) located between the recombination site and the polynucleotide encoding the recombinase, and wherein the IRES is operably linked to the polynucleotide encoding the recombinase.

In one aspect, the disclosure provides AAV vector genomes comprising, in 5′ to 3′ orientation: (a) a 5′ AAV inverted terminal repeat; (b) (b-1) a Lox JTZ17 recombination site comprising or consisting of SEQ ID NO: 217, or a sequence having at most 1, at most 2, or at most 3 nucleotides mutations (deletion, insertion, and/or substitution) thereto; or (b-2) a Lox 66 recombination site comprising or consisting of SEQ ID NO: 179, or a sequence having at most 1, at most 2, or at most 3 nucleotides mutations (deletion, insertion, and/or substitution) thereto; (c) a splice acceptor (SA) site; (d) a 3′ portion of a transgene; (e) a poly-A site; and (f) a 3′ AAV inverted terminal repeat. In embodiments, the splice acceptor (SA) site comprises or consists of a sequence having at least 90%, at least 95%, at least 96%, at least 97%, at least 98%, at least 99%, or 100% identity to SEQ ID NO: 187. In embodiments, the transgene encodes an ABCA4 protein. In embodiments, the 3′ portion of the transgene comprises or consists of a sequence having at least 90%, at least 95%, at least 96%, at least 97%, at least 98%, at least 99%, or 100% identity to SEQ ID NO: 193. In embodiments, the poly-A site comprises or consists of SEQ ID NO: 189, or a sequence having at most 1, at most 2, at most 3, at most 4, at most 5, or at most 6 nucleotides mutations (deletion, insertion, and/or substitution) thereto.

In embodiments, the 5′ AAV inverted terminal repeat comprises or consists of a sequence having at least 90%, at least 95%, at least 96%, at least 97%, at least 98%, at least 99%, or 100% identity to SEQ ID NO: 215. In embodiments, the 3′ AAV inverted terminal repeat comprises or consists of a sequence having at least 90%, at least 95%, at least 96%, at least 97%, at least 98%, at least 99%, or 100% identity to SEQ ID NO: 216.

In one aspect, the disclosure provides AAV viral particles comprising (i) an AAV capsid, wherein the AAV capsid comprises an AAV capsid protein; and (ii) the AAV vector genome of the disclosure. In embodiments, the AAV capsid protein comprises or consists of a sequence having at least 90%, at least 95%, at least 96%, at least 97%, at least 98%, at least 99%, or 100% identity to a sequence selected from SEQ ID NOs: 1-3, 67, 71, 196, 205 and 206. In embodiments, the AAV capsid protein comprises or consists of a sequence having at least 90%, at least 95%, at least 96%, at least 97%, at least 98%, at least 99%, or 100% identity to SEQ ID NO: 3. In embodiments, the AAV capsid protein comprises or consists of a sequence having at least 90%, at least 95%, at least 96%, at least 97%, at least 98%, at least 99%, or 100% identity to SEQ ID NO: 67.

In one aspect, the disclosure provides pharmaceutical compositions comprising the AAV vector genome of the disclosure or the AAV viral particle of the disclosure.

In one aspect, the disclosure provides pharmaceutical compositions comprising (i) a first AAV viral particle comprising the first AAV vector genome of the disclosure; and (ii) a second AAV viral particle comprising the second AAV vector genome of the disclosure.

In one aspect, the disclosure provides pharmaceutical compositions comprising (i) a first AAV viral particle comprising the first AAV vector genome of the disclosure; and (ii) a second AAV viral particle comprising an AAV vector genome comprising, in 5′ to 3′ orientation: (a) a 5′ AAV inverted terminal repeat; (b) a recombination site; (c) a splice acceptor (SA) site; (d) a 3′ portion of the transgene; (e) a poly-A site; and (f) a 3′ AAV inverted terminal repeat.

In one aspect, the disclosure provides methods of expressing a protein encoded by a transgene in a cell, comprising: (1) transducing the cell with a first AAV viral particle comprising the first AAV vector genome of the disclosure; and (2) transducing the cell with a second AAV viral particle comprising the second AAV vector genome of the disclosure.

In one aspect, the disclosure provides methods of expressing a protein encoded by a transgene in a cell, comprising: (1) transducing the cell with a first AAV viral particle comprising the first AAV vector genome of the disclosure; and (2) transducing the cell with a second AAV viral particle comprising an AAV vector genome comprising, in 5′ to 3′ orientation: (a) a 5′ AAV inverted terminal repeat; (b) a recombination site; (c) a splice acceptor (SA) site; (d) a 3′ portion of the transgene; (e) a poly-A site; and (f) a 3′ AAV inverted terminal repeat.

In one aspect, the disclosure provides methods of treating a subject having a disease or disorder, comprising: (1) administering to the subject a first AAV viral particle comprising the first AAV vector genome of the disclosure; and (2) administering to the subject a second AAV viral particle comprising the second AAV vector genome of the disclosure.

In one aspect, the disclosure provides methods of treating a subject having a disease or disorder, comprising: (1) administering to the subject a first AAV viral particle comprising the first AAV vector genome of the disclosure; and (2) administering to the subject a second AAV viral particle comprising an AAV vector genome comprising, in 5′ to 3′ orientation: (a) a 5′ AAV inverted terminal repeat; (b) a recombination site; (c) a splice acceptor (SA) site; (d) a 3′ portion of the transgene; (e) a poly-A site; and (f) a 3′ AAV inverted terminal repeat.

In embodiments, the recombination site in the first AAV vector genome of the first AAV viral particle and the recombination site in the AAV vector genome of the second AAV viral particle prevent backwards recombination.

In embodiments, the AAV viral particle comprises an AAV capsid protein comprising or consisting of a sequence having at least 90%, at least 95%, at least 96%, at least 97%, at least 98%, at least 99%, or 100% identity to a sequence selected from SEQ ID NOs: 1-3, 67, 71, 196, 205 and 206. In embodiments, the AAV capsid protein comprises or consists of a sequence having at least 90%, at least 95%, at least 96%, at least 97%, at least 98%, at least 99%, or 100% identity to SEQ ID NO: 3. In embodiments, the AAV capsid protein comprises or consists of a sequence having at least 90%, at least 95%, at least 96%, at least 97%, at least 98%, at least 99%, or 100% identity to SEQ ID NO: 67.

In embodiments, administration of the first AAV viral particle and the second AAV viral particle leads to recombination of the first AAV vector genome and the second AAV vector genome via the recombination site in the AAV vector genome of the first AAV viral particle and the recombination site in the second AAV vector genome of the second AAV viral particle. In embodiments, administration of the first AAV viral particle and the second AAV viral particle leads to expression of the full transgene.

In embodiments, the protein encoded by the transgene is an ABCA4 protein. In embodiments, the disease or disorder is Stargardt disease. In embodiments, the disease or disorder is caused by an ABCA4 gene defect. In embodiments, the ABCA4 gene defect results in one or more conditions selected from the group consisting of reduced expression of ABCA4 protein, eliminated expression of ABCA4 protein, expression of a mutated ABCA4 protein, and reduced function of ABCA4 protein.

In embodiments, the first AAV viral particle and the second AAV viral particle are co-administered. In embodiments, the first AAV viral particle is administered before, or after, the second AAV viral particle. In embodiments, the viral particles are administered by intravitreal, para-retinal, or subretinal injection. In embodiments, the viral particles are administered by subretinal injection.

In embodiments, no stable expression of the recombinase is detected in the transduced cell or in the subject.

In one aspect, the disclosure provides transduced cells comprising: (i) a defective genomic copy of an endogenous gene; (ii) a recombinant nucleic acid comprising, in 5′ to 3′ orientation: (a) a 5′ AAV inverted terminal repeat; (b) an interphotoreceptor retinoid-binding protein (IRBP) enhancer; (c) an eye-specific promoter; (d) a 5′ portion of a transgene, wherein the transgene is a working copy of the defective endogenous gene; (e) a splice donor (SD) site; (f) a recombination site; (g) a splice acceptor (SA) site; (h) a 3′ portion of the transgene; (i) a poly-A site; and (j) a 3′ AAV inverted terminal repeat.

In one aspect, the disclosure provides transduced cells comprising (i) a defective genomic copy of an endogenous gene; (ii) a recombinant nucleic acid comprising, in 5′ to 3′ orientation: (a) a 5′ AAV inverted terminal repeat; (b) optionally, an enhancer; (c) a human rhodopsin (hRho) promoter; (d) a 5′ portion of a transgene, wherein the transgene is a working copy of the defective endogenous gene; (e) a splice donor (SD) site; (f) a recombination site; (g) a splice acceptor (SA) site; (h) a 3′ portion of the transgene; (i) a poly-A site; and (j) a 3′ AAV inverted terminal repeat.

In one aspect, the disclosure provides transduced cells comprising (i) a defective genomic copy of an endogenous gene; (ii) a recombinant nucleic acid comprising, in 5′ to 3′ orientation: (a) a 5′ AAV inverted terminal repeat; (b) a CMV enhancer; (c) a chicken beta-actin (CBA) promoter; (d) a 5′ portion of a transgene, wherein the transgene is a working copy of the defective endogenous gene; (e) a splice donor (SD) site; (f) a recombination site; (g) a splice acceptor (SA) site; (h) a 3′ portion of the transgene; (i) a poly-A site; and (j) a 3′ AAV inverted terminal repeat.

In embodiments, the defective endogenous gene is an ABCA4 gene and the transgene encodes an ABCA4 protein.

In embodiments, the transduced cell or the recombinant nucleic acid comprises a nucleic acid sequence comprising, in 5′ to 3′ orientation: a 5′ AAV inverted terminal repeat, a recombination site, a polynucleotide encoding a recombinase, and a 3′ AAV inverted terminal repeat; wherein the second recombinant nucleic acid sequence lacks a promoter. In embodiments, the nucleic acid sequence is located downstream of the 3′ end of the (ii)(j) 3′ AAV inverted terminal repeat.

In embodiments, the cell is an ex vivo cell. In embodiments, the cell is a photoreceptor cell.

In one aspect, the disclosure provides nucleic acids comprising a 5′ portion of an ABCA4 gene, wherein the 5′ portion of the ABCA4 gene comprises one or more of: (i) a nucleotide “G” at the position corresponding to nucleotide 813 of SEQ ID NO: 212; (ii) a nucleotide “A” at the position corresponding to nucleotide 1311 of SEQ ID NO: 212; (iii) a nucleotide “A” at the position corresponding to nucleotide 2274 of SEQ ID NO: 212; (iv) a nucleotide “C” at the position corresponding to nucleotide 2277 of SEQ ID NO: 212; (v) a nucleotide “T” at the position corresponding to nucleotide 2278 of SEQ ID NO: 212; and (vi) a nucleotide “T” at the position corresponding to nucleotide 2568 of SEQ ID NO: 212. In embodiments, the nucleic acid comprises 2 or more, 3 or more, 4 or more, 5 or more, or all 6 of, the nucleotides (i)-(vi). In embodiments, the 5′ portion of the ABCA4 gene is at least 1 kb, at least 1.5 kb, at least 2 kb, or at least 2.5 kb in length. In embodiments, the 5′ portion of the ABCA4 gene comprises or consists of a sequence having at least 90%, at least 95%, at least 96%, at least 97%, at least 98%, at least 99%, or 100% identity to SEQ ID NO: 212. In embodiments, the 5′ portion of the transgene encodes a polypeptide sequence comprising amino acids 1-884 of human ABCA4 protein (SEQ ID NO: 190).

In one aspect, the disclosure provides AAV vector genomes comprising the nucleic acid of the disclosure.

In one aspect, the disclosure provides AAV viral particles comprising (i) an AAV capsid, wherein the AAV capsid comprises an AAV capsid protein; and (ii) the AAV vector genome of the disclosure.

In one aspect, the disclosure provides pharmaceutical compositions comprising the AAV vector genome of the disclosure or the AAV viral particle of the disclosure.

In one aspect, the disclosure provides methods of treating a subject having a disease or disorder, comprising administering to the subject the AAV viral particle of the disclosure. In embodiments, the disease or disorder is Stargardt disease.

In one aspect, the disclosure provides transduced cells comprising the nucleic acid of the disclosure.

### BRIEF DESCRIPTION OF THE DRAWINGS

FIGS. 1A-1B depict illustrative AAV vector genomes for multipartite gene delivery. FIG. 1A depicts illustrative AAV vector genomes for bipartite gene delivery. “ITR” stands for “inverted terminal repeat”; “SD” stands for “splice donor”; “RS” stands for “recombination site”; “IC” stands for “internal cleavage polypeptide”; “Rec” stands for “recombinase”; “NLS” stands for “nuclear localization sequence”; “pA” stands for “poly-A site”; and “SA” stands for “splice acceptor”. FIG. 1B depicts illustrative AAV vector genomes for multipartite gene delivery of a transgene in three portions. “ITR” stands for “inverted terminal repeat”; “SD” stands for “splice donor”; “RS” stands for “recombination site”; “IC” stands for “internal cleavage polypeptide”; “Rec” stands for “recombinase”; “NLS” stands for “nuclear localization sequence”; “pA” stands for “poly-A site”; and “SA” stands for “splice acceptor”.

FIG. 2 depicts ABCA4 constructs used in tissue culture studies.

FIG. 3A shows the necessity of Cre recombinase for expression of full-length ABCA4 protein mediated by recombination of partial ABCA4 constructs transfected into HEK293 cells. FIG. 3B shows the expression level of recombined full-length ABCA4 protein in HEK293 cells transfected with various ABCA4 constructs.

FIG. 4 shows significant decrease of Cre recombinase expression in HEK293 cells after recombination of partial ABCA4 constructs.

FIG. 5 shows the expression level of full-length ABCA4 mRNA in HEK293 cells transfected with various ABCA4 constructs.

FIG. 6 shows the expression level of full-length ABCA4 mRNA overtime in cells transduced with indicated AAV9 viral particles.

FIGS. 7A-7B depict representative diagrams of AAV vector genomes bipartite delivery of ABCA4 gene. FIG. 7A depicts the construction of a first AAV vector genome (5′ ABCA4+Cre) comprising a human GRK1 promoter, a 5′ portion of ABCA4 gene, a Cre recombinase gene and other genomic elements. FIG. 7B depicts the construction of a second AAV vector genome (3′ ABCA4) comprising a 3′ portion of ABCA4 gene and other genomic elements.

FIG. 8 contains charts showing the gene expression levels of mouse ABCA4 (mABCA4), human ABCA4 (hABCA4), and Cre recombinase.

FIG. 9 shows Western analysis of the protein expression levels of mABCA4, hABCA4, and Cre recombinase.

FIG. 10 shows the in vivo animal study design for the new bipartite AAV systems.

FIG. 11A shows schematics of vector designs for the new bipartite AAV systems.

FIG. 11B depicts Cre-mediated recombination of the first and the second AAV vector genomes.

FIG. 12A and FIG. 12B show Western analysis results of hABCA4 protein expression in mouse retina samples.

FIG. 13A shows Western analysis results of Cre recombinase, hABCA4 and Brn3a protein expression levels. FIG. 13B shows a correlation analysis of Cre recombinase versus hABCA4 protein expression levels.

### DETAILED DESCRIPTION

Some embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein.

Unless the context indicates otherwise, it is specifically intended that the various features of the invention described herein can be used in any combination. Moreover, the disclosure also contemplates that in embodiments, any feature or combination of features set forth herein can be excluded or omitted.

Unless explicitly indicated otherwise, all specified embodiments, features, and terms intend to include both the recited embodiment, feature, or term and biological equivalents thereof.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

### Definitions

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989); Current Protocols in Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (RI. Freshney, ed. (1987)).

As used in the description of the invention and the appended claims, the singular forms “a,” “an” and “the” are intended to include the plural forms as well, unless the context clearly indicates otherwise.

As used herein, the term “comprising” is intended to mean that the compositions and methods include the recited elements (e.g., materials or steps), but do not exclude others. As used herein, “comprising” is synonymous with “including,” “containing,” or “characterized by.” As used herein, the transitional phrase “consisting essentially of” (and grammatical variants) is to be interpreted as encompassing the recited elements (e.g., materials or steps) and those that do not materially affect the basic and novel characteristic(s) of the recited embodiment. Thus, the term “consisting essentially of” as used herein should not be interpreted as equivalent to “comprising.” As used herein, the transitional phrase “consisting of” excludes any elements (e.g., materials or steps) not specified in the compositions or methods. Any recitation herein of the term “comprising,” or its equivalents, is understood to encompass those compositions and methods consisting essentially of and consisting of the recited elements (e.g., materials or steps).

The term “about” as used herein when referring to a measurable value such as an amount or concentration and the like, is meant to encompass variations of +/−10% of the specified amount, unless otherwise apparent from context that it is impossible to extend the boundary beyond certain points (e.g., below 0%).

The terms “acceptable,” “effective,” or “sufficient” when used to describe the selection of any components, ranges, dose forms, etc. disclosed herein intend that said component, range, dose form, etc. is suitable for the disclosed purpose.

Also, as used herein, “and/or” refers to and encompasses any and all possible combinations of one or more of the associated listed items, as well as the lack of combinations when interpreted in the alternative (“or”).

Unless specifically recited, the term “host cell” includes a eukaryotic host cell, including, for example, fungal cells, yeast cells, higher plant cells, insect cells and mammalian cells. Non-limiting examples of eukaryotic host cells include simian, bovine, porcine, murine, rat, avian, reptilian and human, e.g., HEK293 cells and 293T cells.

The term “isolated” as used herein refers to molecules or biologicals or cellular materials being substantially free from other materials.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising, consisting essentially of, or consisting of purine and pyrimidine bases or other natural, chemically or biochemically modified, non-natural, or derivatized nucleotide bases.

A “gene” refers to a polynucleotide containing at least one open reading frame (ORF) that is capable of encoding a particular polypeptide or protein. A “gene product” or, alternatively, a “gene expression product” refers to the amino acid sequence (e.g., peptide or polypeptide) generated when a gene is transcribed and translated.

As used herein, “expression” refers to the two-step process by which polynucleotides are transcribed into mRNA and/or the process by which the transcribed mRNA is subsequently translated into peptides, polypeptides, or proteins. If the polynucleotide is derived from genomic DNA, expression may include splicing of the mRNA in a eukaryotic cell.

“Under transcriptional control” is a term well understood in the art and indicates that transcription of a polynucleotide sequence, usually a DNA sequence, depends on its being operably linked to an element that contributes to the initiation of, or promotes, transcription. “Operably linked” intends that the polynucleotides are arranged in a manner that allows them to function in a cell. In aspects, this disclosure provides promoters operably linked to the downstream sequences.

The term “encode” as it is applied to polynucleotides refers to a polynucleotide which is said to “encode” a polypeptide if, in its native state or when manipulated by methods well known to those skilled in the art, it can be transcribed to produce the mRNA for the polypeptide and/or a fragment thereof. The antisense strand is the complement of such a nucleic acid, and the encoding sequence can be deduced therefrom.

The term “promoter” as used herein means a control sequence that is a region of a polynucleotide sequence at which the initiation and rate of transcription of a coding sequence, such as a gene or a transgene, are controlled. Promoters may be constitutive, inducible, repressible, or tissue-specific, for example. Promoters may contain genetic elements at which regulatory proteins and molecules such as RNA polymerase and transcription factors may bind. Non-limiting exemplary promoters include a human rhodopsin kinase (hGRK1) promoter, a human interphotoreceptor retinoid-binding protein promoter (IRBP), a human red/green opsin promoter (pR2.1), a human blue opsin promoter (HB), a mouse opsin promoter (mOP), a mouse short wave opsin promoter (mBP) (short wave opsin promoters may also be referred to as S-opsin or blue opsin promoters), a human rod cGMP phosphodiesterase β-subunit promoter (DPDE), a Rous sarcoma virus (RSV) LTR promoter (optionally with the RSV enhancer), a cytomegalovirus (CMV) promoter, an SV40 promoter, a dihydrofolate reductase promoter, a β-actin promoter, a phosphoglycerol kinase (PGK) promoter, a U6 promoter, an H1 promoter, a ubiquitous chicken β-actin hybrid (CBh) promoter, a small nuclear RNA (U1a or U1b) promoter, an MeCP2 promoter, an MeP418 promoter, an MeP426 promoter, a minimal MeCP2 promoter, a VMD2 promoter, an mRho promoter, or an elongation factor-1 alpha (EF1 alpha) promoter.

Additional non-limiting exemplary promoters provided herein include, but are not limited to, EF1a, Ube, human β-actin, CAG, TRE, Ac5, polyhedrin, CaMKIIa, Gal1, TEF1, GDS, ADH1, Ubi, and α-1-antitrypsin (hAAT). It is known in the art that the nucleotide sequences of such promoters may be modified in order to increase or decrease the efficiency of mRNA transcription. See, e.g., Gao et al. (2018) Mol. Ther.: Nucleic Acids 12:135-145 (modifying TATA box of 7SK, U6 and H1 promoters to abolish RNA polymerase III transcription and stimulate RNA polymerase II-dependent mRNA transcription). Synthetically derived promoters may be used for ubiquitous or tissue specific expression. Further, virus-derived promoters, some of which are noted above, may be useful in the methods disclosed herein, e.g., CMV, HIV, adenovirus, and AAV promoters. In embodiments, the promoter is used together with an enhancer to increase the transcription efficiency. Non-limiting examples of enhancers include an interphotoreceptor retinoid-binding protein (IRBP) enhancer, an RSV enhancer or a CMV enhancer.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter. Non-limiting examples of linked enhancer/promoter for use in the methods, compositions and constructs provided herein include a PDE promoter plus IRBP enhancer or a CMV enhancer plus U1a promoter. It is understood in the art that enhancers can operate from a distance and irrespective of their orientation relative to the location of an endogenous or heterologous promoter. It is thus further understood that an enhancer operating at a distance from a promoter is thus “operably linked” to that promoter irrespective of its location in the vector or its orientation relative to the location of the promoter.

The term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise, consist essentially of, or consist of a protein's or peptide's sequence. As used herein the term “amino acid” refers to either natural and/or unnatural or synthetic amino acids, including glycine and both the D and L optical isomers, amino acid analogs and peptidomimetics.

As used herein, the term “signal peptide” or “signal polypeptide” intends an amino acid sequence usually present at the N-terminal end of newly synthesized secretory or membrane polypeptides or proteins. It acts to direct the polypeptide to a specific cellular location, e.g. across a cell membrane, into a cell membrane, or into the nucleus. In embodiments, the signal peptide is removed following localization. Examples of signal peptides are well known in the art. Non-limiting examples are those described in U.S. Pat. Nos. 8,853,381, 5,958,736, and 8,795,965. In embodiments, the signal peptide can be an IDUA signal peptide.

The terms “equivalent” or “biological equivalent” are used interchangeably when referring to a particular molecule, biological material, or cellular material and intend those having minimal homology while still maintaining desired structure or functionality. Non-limiting examples of equivalent polypeptides include a polypeptide having at least about 60%, at least about 65%, at least about 70%, at least about 75%, at least about 80%, at least about 85%, at least about 90%, at least about 95% identity or at least about 99% identity to a reference polypeptide (for instance, a wild-type polypeptide); or a polypeptide which is encoded by a polynucleotide having at least about 70%, at least about 75%, at least about 80%, at least about 85%, at least about 90%, at least about 95% identity, at least about 97% sequence identity or at least about 99% sequence identity to the reference polynucleotide (for instance, a wild-type polynucleotide).

“Homology” or “identity” or “similarity” refers to sequence similarity between two peptides or between two nucleic acid molecules. Percent identity can be determined by comparing a position in each sequence that may be aligned for purposes of comparison. When a position in the compared sequence is occupied by the same base or amino acid, then the molecules are identical at that position. A degree of identity between sequences is a function of the number of matching positions shared by the sequences. “Unrelated” or “non-homologous” sequences share less than 40% identity, less than 25% identity, with one of the sequences of the present disclosure. Alignment and percent sequence identity may be determined for the nucleic acid or amino acid sequences provided herein by importing said nucleic acid or amino acid sequences into and using Basic Local Alignment Search Tool (BLAST). Unless noted otherwise, the sequence alignment and identity calculation are performed by BLAST version 2.11.0 using default parameters.

The terms “corresponding to” or “correspond to”, as used herein in relation to the amino acid or nucleic acid position(s), refer to the position(s) in a first polypeptide/polynucleotide sequence that aligns with a given amino acid/nucleic acid in a reference polypeptide/polynucleotide sequence when the first and the reference polypeptide/polynucleotide sequences are aligned.

As used herein, amino acid modifications may be substitutions, deletions or insertions. Amino acid substitutions may be conservative amino acid substitutions or non-conservative amino acid substitutions. A conservative replacement (also called a conservative mutation, a conservative substitution or a conservative variation) is an amino acid replacement in a protein that changes a given amino acid to a different amino acid with similar biochemical properties (e.g., charge, hydrophobicity or size). As used herein, “conservative variations” refer to the replacement of an amino acid residue by another, biologically similar residue. Examples of conservative variations include the substitution of one hydrophobic residue such as isoleucine, valine, leucine or methionine for another; or the substitution of one charged or polar residue for another, such as the substitution of arginine for lysine, glutamic acid for aspartic acid, glutamine for asparagine, and the like. Other illustrative examples of conservative substitutions include the changes of: alanine to serine; asparagine to glutamine or histidine; aspartate to glutamate; cysteine to serine; glycine to proline; histidine to asparagine or glutamine; lysine to arginine, glutamine, or glutamate; phenylalanine to tyrosine, serine to threonine; threonine to serine; tryptophan to tyrosine; tyrosine to tryptophan or phenylalanine; and the like.

As used herein, the term “vector” refers to a nucleic acid comprising, consisting essentially of, or consisting of an intact replicon such that the vector may be replicated when placed within a cell, for example by a process of transfection, infection, or transformation. It is understood in the art that once inside a cell, a vector may replicate as an extrachromosomal (episomal) element or may be integrated into a host cell chromosome. Vectors may include nucleic acids derived from retroviruses, adenoviruses, herpesvirus, baculoviruses, modified baculoviruses, papovaviruses, AAV viral vectors, lentiviral vectors, adenovirus vectors, alphavirus vectors, or otherwise modified or naturally-occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising, consisting essentially of, or consisting of DNA condensed with cationic polymers such as heterogeneous polylysine, defined-length oligopeptides, and polyethyleneimine, in some cases contained in liposomes; and the use of ternary complexes comprising, consisting essentially of, or consisting of a virus and polylysine-DNA.

With respect to general recombinant techniques, vectors that contain both a promoter and a cloning site into which a polynucleotide can be operably linked are well known in the art. Such vectors are capable of transcribing RNA in vitro or in vivo, and are commercially available from sources such as Agilent Technologies (Santa Clara, Calif) and Promega Biotech (Madison, Wis.). In order to optimize expression and/or in vitro transcription, it may be necessary to remove, add or alter 5′ and/or 3′ untranslated portions of cloned transgenes to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

A “viral vector” is defined as a recombinantly produced virus or viral particle that contains a polynucleotide to be delivered into a host cell, either in vivo, ex vivo or in vitro. Examples of viral vectors include retroviral vectors, AAV viral vectors, lentiviral vectors, adenovirus vectors, alphavirus vectors and the like.

As used herein, the term “recombinant expression system” or “recombinant vector” refers to a genetic construct or constructs for the expression of certain genetic material formed by recombination.

A “gene delivery vehicle” is defined as any molecule that can carry inserted polynucleotides into a host cell. Examples of gene delivery vehicles are liposomes, micelles biocompatible polymers, including natural polymers and synthetic polymers; lipoproteins; polypeptides; polysaccharides; lipopolysaccharides; artificial viral envelopes; metal particles; bacteria; viruses, such as baculoviruses, adeno-associated viruses, adenoviruses and retroviruses; bacteriophage, cosmid, plasmid, and fungal vectors; and other recombination vehicles typically used in the art which have been described for expression in a variety of eukaryotic and prokaryotic hosts, and may be used for gene therapy as well as for simple protein expression. Liposomes that also comprise, consist essentially of, or consist of a targeting antibody or fragment thereof can be used in the methods disclosed herein. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins disclosed herein are other non-limiting techniques.

A polynucleotide disclosed herein can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “plasmid” is a DNA molecule that is typically separate from and capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or, alternatively, the proteins produced may act as toxins under similar circumstances. It is known in the art that while plasmid vectors often exist as extrachromosomal circular DNA molecules, plasmid vectors may also be designed to be stably integrated into a host chromosome either randomly or in a targeted manner, and such integration may be accomplished using either a circular plasmid or a plasmid that has been linearized prior to introduction into the host cell.

“Plasmids” used in genetic engineering are called “plasmid vectors”. Many plasmids are commercially available for such uses. The gene to be replicated is inserted into copies of a plasmid containing genes that make cells resistant to particular antibiotics, and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria or eukaryotic cells containing a plasmid harboring the gene of interest, which can be induced to produce large amounts of proteins from the inserted gene.

The term “adeno-associated virus” or “AAV” as used herein refers to a member of the class of viruses associated with this name and belonging to the genus Dependoparvovirus, family Parvoviridae. Adeno-associated virus is a single-stranded DNA virus that grows only in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3: 1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control. Multiple serotypes of this virus are known to be suitable for gene delivery; all known serotypes can infect cells from various tissue types. At least 11 sequentially numbered AAV serotypes are known in the art. Non-limiting exemplary serotypes useful in the methods disclosed herein include any of the 11 serotypes, e.g., AAV2, AAV8, AAV9, or variant serotypes, e.g., AAV-DJ and AAV PHP.B. The AAV particle usually comprises three major viral proteins: VP1, VP2 and VP3. In embodiments, the AAV refers to the serotype AAV1, AAV2, AAV4, AAV5, AAV6, AAV7, AAV8, AAV9, AAV10, AAV11, AAV12, AAV13, AAVPHP.B, AAVrh74, AAV 110, AAV 204, AAV 214, AAV 214A, AAV 214e, AAV 214e8, AAV 214e9, AAV 214e10, AAV ITB102_45, or AAV 214AB.

An “AAV vector” as used herein refers to a vector comprising one or more heterologous nucleic acid (HNA) sequences and one or more AAV inverted terminal repeat sequences (ITRs). Such AAV vectors can be replicated when present in a host cell that provides the functionality of rep and cap gene products, and allow the ITRs and the nucleic acid between the ITRs to be packaged into infectious viral particles. In embodiments, AAV vectors comprise a promoter, at least one nucleic acid that may encode at least one protein or RNA, and/or an enhancer and/or a terminator within the flanking ITRs that is packaged into the infectious AAV particle. The ITRs and the nucleic acid between the ITRs can be encapsidated into the AAV capsid, and this encapsidated portion of the nucleic acid may be referred to as the “AAV vector genome.” AAV vectors may contain elements in addition to the encapsidated portion, for example, antibiotic resistance genes or other elements known in the art included in the plasmid for manufacturing purposes but not packaged into the AAV particle.

As used herein, the term “viral capsid” or “capsid” refers to the proteinaceous shell or coat of a viral particle. Capsids function to encapsidate, protect, transport, and/or release into the host cell a viral genome. Capsids are generally comprised of oligomeric structural subunits of protein (“capsid proteins”). As used herein, the term “encapsidated” means enclosed within a viral capsid. The viral capsid of AAV is composed of a mixture of three viral capsid proteins: VP1, VP2, and VP3. The mixture of VP1, VP2 and VP3 contains 60 monomers that are arranged in a T=1 icosahedral symmetry in a ratio of 1:1:10 (VP1:VP2:VP3) or 1:1:20 (VP1:VP2:VP3) as described in Sonntag F et al., (June 2010). “A viral assembly factor promotes AAV2 capsid formation in the nucleolus”. Proceedings of the National Academy of Sciences of the United States of America. 107 (22): 10220-5, and Rabinowitz J E, Samulski R J (December 2000). “Building a better vector: the manipulation of AAV virions”. Virology. 278 (2): 301-8, each of which is incorporated herein by reference in its entirety.

An “AAV virion” or “AAV viral particle” or “AAV viral vector” or “AAV vector particle” or “AAV particle” refers to a viral particle composed of at least one AAV capsid protein and an encapsidated AAV vector genome.

As used herein, the term “helper” in reference to a virus or plasmid refers to a virus or plasmid used to provide the additional components necessary for replication and packaging of any one of the AAV vector genomes disclosed herein. The components encoded by a helper virus or plasmid may include any genes required for virion assembly, encapsidation, genome replication, and/or packaging. For example, the helper virus or plasmid may encode enzymes or other factors necessary for replication of the viral genome in the host cell. Non-limiting examples of helper viruses and plasmids suitable for use with AAV constructs include pHELP (plasmid), adenovirus (virus), or herpesvirus (virus).

As used herein, a packaging cell (or a helper cell) is a cell used to produce viral vectors. Producing recombinant AAV viral vectors requires Rep and Cap proteins provided in trans as well as gene sequences from adenovirus that help AAV replicate. In aspects, packaging/helper cells contain a plasmid that is stably incorporated into the genome of the cell. In other aspects, the packaging cell may be transiently transfected. Typically, a packaging cell is a eukaryotic cell, such as a mammalian cell or an insect cell.

As used herein, a reporter protein is a detectable protein that is operably linked to a promoter to assay the expression (for example, tissue specificity and/or strength) of the promoter. In aspects, a reporter protein may be operably linked to a polypeptide. In aspects, reporter proteins may be used in monitoring DNA delivery methods, functional identification and characterization of promoter and enhancer elements, translation and transcription regulation, mRNA processing and protein:protein interactions. Non-limiting examples of reporter proteins are β-galactosidase; fluorescent proteins such as Green Fluorescent Protein (GFP) or Red Fluorescent Protein (RFP); luciferase; glutathione S-transferase (GST); and maltose binding protein (MBP).

A “pharmaceutical composition” is intended to include the combination of an active ingredient such as a polypeptide, a polynucleotide, an antibody, or a viral vector, with a carrier, inert or inactive such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

A “subject” of diagnosis or treatment is a cell or an animal such as a mammal, or a human. A subject is not limited to a specific species and includes non-human animals subject to diagnosis or treatment and those subject to infections or animal models, including, without limitation, simian, murine, rat, canine, or leporid species, as well as other livestock, sport animals, or pets. In embodiments, the subject is a human.

The term “tissue” is used herein to refer to tissue of a living or deceased organism or any tissue derived from or designed to mimic a living or deceased organism. The tissue may be healthy, diseased, and/or have genetic mutations. The biological tissue may include any single tissue (e.g., a collection of cells that may be interconnected), or a group of tissues making up an organ or part or region of the body of an organism. The tissue may comprise, consist essentially of, or consist of a homogeneous cellular material or it may be a composite structure such as that found in regions of the body including the thorax which for instance can include lung tissue, skeletal tissue, and/or muscle tissue. Exemplary tissues include, but are not limited to those derived from eye, liver, lung, thyroid, skin, pancreas, blood vessels, bladder, kidneys, brain, biliary tree, duodenum, abdominal aorta, iliac vein, heart and intestines, including any combination thereof.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease) progression, amelioration or palliation of the condition (including disease) states and remission (whether partial or total), whether detectable or undetectable.

As used herein the term “effective amount” intends to mean a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of gene therapy, in embodiments the effective amount is the amount sufficient to result in regaining part or full function of a gene that is deficient in a subject. In embodiments, the effective amount of an AAV viral particle is the amount sufficient to result in expression of a gene in a subject. The skilled artisan will be able to determine appropriate amounts depending on these and other factors.

In embodiments, the effective amount will depend on the size and nature of the application in question. It will also depend on the nature and sensitivity of the target subject and the methods in use. The skilled artisan will be able to determine the effective amount based on these and other considerations. The effective amount may comprise, consist essentially of, or consist of one or more administrations of a composition depending on the embodiment.

As used herein, the term “administer” or “administration” intends to mean delivery of a substance to a subject such as an animal or human. Administration can be effected in one dose, continuously or intermittently throughout the course of treatment. Methods of determining the most effective means and dosage of administration will vary with the composition used for therapy, the purpose of the therapy, as well as the age, health or gender of the subject being treated. Single or multiple administrations can be carried out with the dose level and pattern being selected by the treating physician or in the case of pets and other animals, treating veterinarian.

As used herein, the term “substantially retain” in reference to the function of a biological sequence (e.g., the promoter function of a promoter sequence) refers to retaining at least 70% of the function of a reference sequence, such as at least 80%, at least 90%, at least 95%, or at least 100% of the function of the reference sequence. For example, a promoter sequence may contain a number of mutations compared to a reference promoter sequence yet substantially retain (i.e., retaining at least 70% of) the promoter function of the reference promoter sequence. Unless otherwise indicated, the disclosure encompasses sequences having a specified identity level (e.g., at least 90% identical) to a reference sequence (e.g., an enhancer sequence or a promoter sequence) disclosed herein and substantially retaining the function of that reference sequence. Unless otherwise indicated, the function of the biological sequences can be measured using assays known in the art—for example, the function of the enhancer or promoter sequence can be determined based on the RNA transcription level as measured by quantitative RT-PCR.

### AAV Structure and Function

AAV is a replication-deficient parvovirus, the single-stranded DNA genome of which is about 4.7 kb in length, including two about 145-nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_001729; the complete genome of AAV-4 is provided in GenBank Accession No. NC_001829; the AAV-5 genome is provided in GenBank Accession No. NC_006152; the complete genome of AAV-6 is provided in GenBank Accession No. NC_001862; the complete genome of AAV-7 is provided in GenBank Accession No. NC_006260; the complete genome of AAV-8 is provided in GenBank Accession No. NC_006261; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). The sequence of the AAV rh.74 genome is provided in U.S. Pat. No. 9,434,928, which is hereby incorporated herein by reference in its entirety. U.S. Pat. No. 9,434,928 also provide the sequences of the capsid proteins and a self-complementary genome. In one aspect, the genome is a self-complementary genome. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome.

The cap gene is expressed from the p40 promoter and encodes the three capsid proteins, VP1, VP2, and VP3. Alternative splicing and non-consensus translational start sites are responsible for the production of the three related capsid proteins. More specifically, after the single mRNA from which each of the VP1, VP2 and VP3 proteins are translated is transcribed, it can be spliced in two different manners: either a longer or shorter intron can be excised, resulting in the formation of two pools of mRNAs: a 2.3 kb- and a 2.6 kb-long mRNA pool. The longer intron is often preferred and thus the 2.3-kb-long mRNA can be called the major splice variant. This form lacks the first AUG codon, from which the synthesis of VP1 protein starts, resulting in a reduced overall level of VP1 protein synthesis. The first AUG codon that remains in the major splice variant is the initiation codon for the VP3 protein. However, upstream of that codon in the same open reading frame lies an ACG sequence (encoding threonine) which is surrounded by an optimal Kozak (translation initiation) context. This contributes to a low level of synthesis of the VP2 protein, which is actually the VP3 protein with additional N terminal residues, as is VP1, as described in Becerra S P et al., (December 1985), “Direct mapping of adeno-associated virus capsid proteins B and C: a possible ACG initiation codon”, Proceedings of the National Academy of Sciences of the United States of America, 82 (23): 7919-23; Cassinotti P et al., (November 1988), “Organization of the adeno-associated virus (AAV) capsid gene: mapping of a minor spliced mRNA coding for virus capsid protein 1”, Virology, 167 (1): 176-84; Muralidhar S et al., (January 1994), “Site-directed mutagenesis of adeno-associated virus type 2 structural protein initiation codons: effects on regulation of synthesis and biological activity”, Journal of Virology, 68 (1): 170-6; and Trempe J P, Carter B J (September 1988), “Alternate mRNA splicing is required for synthesis of adeno-associated virus VP1 capsid protein”, Journal of Virology, 62 (9): 3356-63, each of which is herein incorporated by reference. A single consensus polyadenylation signal (poly-A site) is located at map position 95 of the AAV genome. The life cycle and genetics of AAV are reviewed in Muzyczka, Current Topics in Microbiology and Immunology, 158: 97-129 (1992). Non-limiting exemplary capsid proteins include an AAV 110 capsid protein, an AAV 204 capsid protein, an AAV 214 capsid protein, an AAV 214A capsid protein, an AAV 214e capsid protein, an AAV 214e8 capsid protein, an AAV 214e9 capsid protein, an AAV 214e10 capsid protein, an AAV ITB102_45 capsid protein, an AAV 214AB capsid protein, an AAV6 capsid protein, an AAV8 capsid protein, an AAV9 capsid protein and an AAV2 capsid protein according to Tables 1-3 of the disclosure. Additional non-limiting exemplary capsid proteins include those disclosed in WO2016081811, which is hereby incorporated herein by reference in its entirety and, in particular, for AAV capsid protein sequences.

Each VP1 protein contains a VP1 portion, a VP2 portion and a VP3 portion. The VP1 portion is the N-terminal portion of the VP1 protein that is unique to the VP1 protein. The VP2 portion is the amino acid sequence present within the VP1 protein that is also found in the N-terminal portion of the VP2 protein. The VP3 portion and the VP3 protein have the same sequence. The VP3 portion is the C-terminal portion of the VP1 protein that is shared with the VP1 and VP2 proteins.

The VP3 protein can be further divided into discrete variable surface regions I-IX (VR-I-IX). Each of the variable surface regions (VRs) can comprise or contain specific amino acid sequences that either alone or in combination with the specific amino acid sequences of each of the other VRs can confer unique infection phenotypes (e.g., decreased antigenicity, improved transduction and/or tissue-specific tropism relative to other AAV serotypes) to a particular serotype as described in DiMatta et al., “Structural Insight into the Unique Properties of Adeno-Associated Virus Serotype 9” J. Virol., Vol. 86 (12): 6947-6958, June 2012, the contents of which are hereby incorporated herein by reference in their entirety.

AAV possesses unique features that make it attractive as a viral vector for delivering foreign DNA to cells, for example, in gene therapy. AAV infection of cells in culture is noncytopathic, and natural infection of humans and other animals is silent and asymptomatic. Moreover, AAV infects many mammalian cell types, allowing the possibility of targeting many different tissues in vivo. Moreover, AAV transduces slowly dividing and non-dividing cells, and can persist essentially for the lifetime of those cells as a transcriptionally active nuclear episome (extrachromosomal element). Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA to generate AAV vector genomes. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8: 659-669 (1997); Kessler et al., Proc Nat. Acad Sc. USA, 93: 14082-14087 (1996); and Xiao et al., J Virol, 70: 8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94: 5804-5809 (1997) and Murphy et al., Proc Natl Acad Sci USA, 94: 13921-13926 (1997). Moreover, Lewis et al., J Virol, 76: 8769-8775 (2002) demonstrated that skeletal myofibers possess the necessary cellular factors for correct antibody glycosylation, folding, and secretion, indicating that muscle is capable of stable expression of secreted protein therapeutics. Recombinant AAV (rAAV) genomes of the invention comprise, consist essentially of, or consist of a nucleic acid molecule encoding a therapeutic protein or portion thereof (e.g., the ABCA4) and one or more AAV ITRs flanking the nucleic acid molecule. AAV DNA in the rAAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV PHP.B and AAV rh74. Production of pseudotyped rAAV is disclosed in, for example, WO2001083692. Other types of rAAV variants, for example rAAV with capsid mutations, are also contemplated. See, e.g., Marsic et al., Molecular Therapy, 22(11): 1900-1909 (2014). The nucleotide sequences of the genomes of various AAV serotypes are known in the art.

### AAV Vector Particles, Capsid Proteins, and AAV Vectors

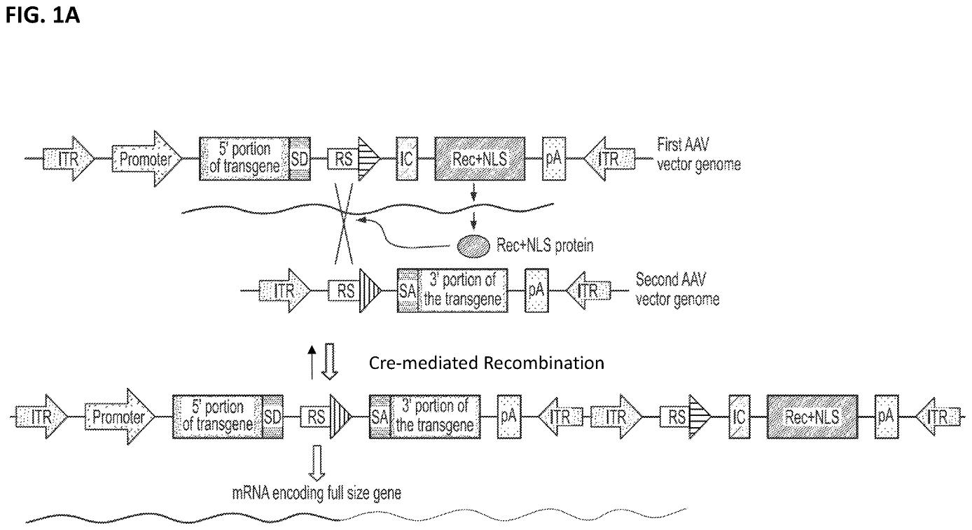

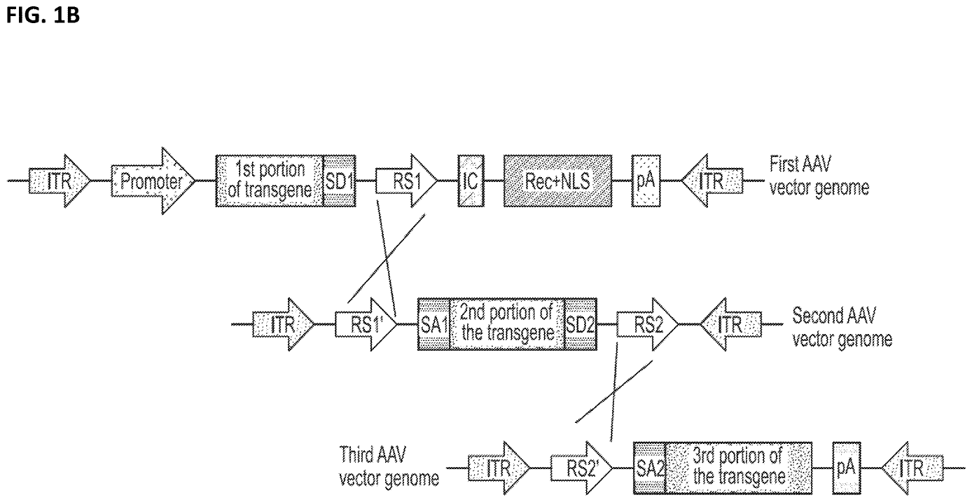

Provided herein are AAV vector particles, AAV vectors, and capsid proteins that find use in delivering a variety of therapeutic payloads, including nucleic acids and proteins useful in the treatment of disease. The methods and compositions disclosed herein may be used to deliver to a subject transgenes that are larger than the packaging capacity of about 4.7 kb typical of single AAV vector genome. Delivery of the larger transgenes may be achieved by delivering portions of the transgene in two or more AAV vector particles and recombining the portions in a cell to provide a completed transgene (for example, as illustrated in FIG. 1A and FIG. 1B). In a novel preferred approach described herein, portions of transgenes are joined by Cre-lox mediated recombination. Advantageously, the design of AAV vector genomes ensures that the 5′ portion of the transgene is located upstream of the 3′ portion of the transgene after recombination. Recombination sites may also dictate the directionality of the recombination event (e.g., to reduce or eliminate reverse recombination). In a further advantage, the recombination event may also remove the promoter operably linked to the expression of Cre recombinase protein, leaving a promoter-less vector region encoding the Cre recombinase, thereby lowering, or preventing, further expression of the Cre recombinase in the host cell.

Additional description of multipartite (e.g., bipartite) AAV delivery systems can be found, for example, in international publication no. WO 2022/015788, the content of which is incorporated by reference in its entirety.

### AAV Capsid Proteins

The disclosure provides AAV particles and AAV vector genomes for multipartite (e.g., bipartite) delivery of large genes. In embodiments, two AAV particles are used for bipartite delivery: a first AAV particle comprises a 5′ portion of the gene, and a second AAV particle comprises a 3′ portion of the gene. The first and second AAV particles may have the same capsid protein or a different capsid protein.

In embodiments, the VP1 capsid protein comprises, or consists of, any one of the amino acid sequences listed in Table 1, or a sequence having up to 1, 2, 3, 4, 5, 6, 7, 8, 9, or 10 amino acids mutated, deleted or added as compared to, any one of the amino acid sequences listed in Table 1. In embodiments, up to 15 amino acids, up to 20 amino acids, up to 30 amino acids, or up to 40 amino acids may be mutated, deleted or added compared to these sequences. In embodiments, the VP1 capsid protein is encoded by any one of the nucleic acid sequences listed in Table 1, or a sequence having up to 5, up to 10, up to 30, or up to 60 nucleotide changes to any one of the nucleic acid sequences listed in Table 1.

TABLE 1
VP1 Capsid Proteins

| Amino Acid SEQ | Nucleic Acid SEQ | AAV Capsid Protein |
|---|---|---|
| ID NO: | ID NO: | Name |
| 1 | 98 | AAV 110 |
| 2 | 15 | AAV 204 |
| 3 | 18 | AAV 214 |
| 30 | 19 | AAV 214A |
| 31 | 20 | AAV 214e |
| 32 | 21 | AAV 214e8 |
| 33 | 22 | AAV 214e9 |
| 34 | 23 | AAV 214e10 |
| 49 | 47 | AAV ITB102_45 |
| 84 | 82 | AAV 214AB |
| 63 | — | AAV6 |
| 67 | 66 | AAV8 |
| 71 | 70 | AAV9 |
| 196 | 197 | AAV2 |
| 205 | — | AAV5 |
| 206 | — | AAV7 |

In embodiments, the AAV VP1 protein comprises, consists essentially of, or consists of an amino acid sequence of SEQ ID NO: 1-3, 30-34, 49, 63, 67, 71, 84, or 196, or a sequence having up to 1, 2, 3, 4, 5, 6, 7, 8, 9, or 10 amino acids different from SEQ ID NO: 1-3, 30-34, 49, 63, 67, 71, 84, or 196. Also provided are polynucleotides encoding these VP1 proteins. In embodiments, the polynucleotides encoding the VP1 proteins comprise, consist essentially of, or consist of the sequence of SEQ ID NO: 15, 18-23, 47, 66, 70, 82, 98, or 197 or a sequence having up to 5, up to 10, or up to 30 nucleotide changes to SEQ ID NO: 15, 18-23, 47, 66, 70, 82, 98, or 197.

In embodiments, the AAV capsid protein is an AAV-110 capsid protein (SEQ ID NO: 1), AAV204 capsid protein (SEQ ID NO: 2), AAV214 capsid protein (SEQ ID NO: 3) or AAV ITB102_45 capsid protein (SEQ ID NO: 49). In embodiments, the AAV capsid protein is a variant of the AAV214 capsid protein. In embodiments, the AAV capsid protein is AAV214A (SEQ ID NO: 30), AAV-214-AB (SEQ ID NO: 84), AAV214e (SEQ ID NO: 31), AAV214e8 (SEQ ID NO: 32), AAV214e9 (SEQ ID NO: 33) or AAV214e10 (SEQ ID NO: 34). In embodiments, the AAV capsid protein is an AAV9 capsid protein (SEQ ID NO: 71.

Sequences for exemplary VP2 and VP3 proteins are provided in Table 2 and Table 3. Given the VP2 and VP3 sequences, the VP1 portions may be determined by alignment with the full, VP1 protein sequence.

TABLE 2
VP2 Capsid Proteins

| Amino Acid SEQ ID NO: | Name |
|---|---|
| 35 | 214 |
| 36 | 214A |
| 37 | 214e |
| 38 | 214e8 |
| 39 | 214e9 |
| 40 | 214e10 |
| 85 | 214AB |
| 50 | ITB102_45 |

Exemplary nucleic acids for the other capsid VP2 portions may be derived from the corresponding portions of the VP1 capsid protein nucleic acids.

TABLE 3
VP3 Capsid proteins

| Amino Acid SEQ ID NO: | NA SEQ ID NO: | AAV Capsid Name |
|---|---|---|
| 17 | 16 | 204 |
| 41 | 24 | 214 |
| 42 | 25 | 214A |
| 43 | 26 | 214e |
| 44 | 27 | 214e8 |
| 45 | 28 | 214e9 |
| 46 | 29 | 214e10 |
| 86 | 83 | 214AB |
| 51 | 48 | ITB102_45 |

The VP3 proteins of AAV214, AAV214e, AAV214e8, AAV214e9, AAV214e10 have the same amino acid (SEQ ID NO:41) and nucleic acid (SEQ ID NO: 24) sequences.

In embodiments, the AAV VP2 proteins comprise, consist essentially of, or consist of an amino acid sequence of any one of SEQ ID NOs: 35-40, 50 or 85, or a sequence having up to 1, 2, 3, 4, 5, 6, 7, 8, 9, or 10 amino acids different from SEQ ID NO: 35-40, 50 or 85. Also provided are polynucleotides encoding these VP2 proteins. In embodiments, the polynucleotide encoding the VP2 protein comprises, consists essentially of, or consists of the sequence of SEQ ID NO: 47, or a sequence having up to 5, up to 10, or up to 30 nucleotide changes to SEQ ID NO: 47.

In embodiments, the AAV VP3 proteins comprise, consist essentially of, or consist of an amino acid sequence of SEQ ID NO: 17, 41-46, 51, or 86, or a sequence having up to 1, 2, 3, 4, 5, 6, 7, 8, 9, or 10 amino acids different from SEQ ID NO: 17, 41-46, 51, or 86. Also provided are polynucleotides encoding these VP3 proteins. In embodiments, the polynucleotide encoding the VP3 protein comprise, consist essentially of, or consist of one of SEQ ID NOs: 16, 24-29, 48 and 83, or a sequence having up to 5, up to 10, or up to 30 nucleotide changes to one of SEQ ID NOs: 16, 24-29, 48 and 83.

In embodiments, the AAV capsid protein is a chimeric protein. In embodiments, a VP1, VP2, or VP3 portion of the AAV capsid protein disclosed herein may be replaced with a VP1, VP2, or VP3 portion from a different AAV capsid protein disclosed herein.

In embodiments, provided herein is an AAV capsid protein comprising a leucine residue at amino acid position 129, an asparagine residue at amino acid position 586 and a glutamic acid residue at amino acid position 723, wherein the amino acid positions in the AAV capsid protein are numbered with respect to amino acid positions in the amino acid sequence of SEQ ID NO: 2. In some cases, the protein comprises the amino acid sequence of SEQ ID NO: 2. In other cases, these amino acids may be introduced into other capsid proteins.

In embodiments, provided herein is an AAV VP1 capsid protein comprising a VP1 portion, a VP2 portion and a VP3 portion, wherein the VP1 portion comprises a leucine (L) residue at amino acid 129, wherein the VP2 portion comprises a threonine (T) or asparagine (N) residue at amino acid 157 and a lysine (K) or serine (S) residue at amino acid 162, and wherein the VP3 portion comprises asparagine (N) residue at amino acid 223, an alanine (A) residue at amino acid 224, a histidine (H) residue at amino acid 272, a threonine (T) residue at amino acid 410, a histidine (H) residue at amino acid 724 and a proline (P) residue at amino acid 734, wherein amino acid positions in the AAV capsid protein are numbered with respect to amino acid positions in the amino acid sequence of SEQ ID NO: 3 (i.e., VP1 capsid subunit numbering).

In embodiments, the VP1 portion further comprises an aspartic acid (D) or alanine (A) residue at amino acid position 24, wherein amino acid positions in the AAV capsid protein are numbered with respect to amino acid positions in the amino acid sequence of SEQ ID NO: 3. In embodiments, the VP2 portion further comprises one or more of (i) a proline (P) residue at amino acid 148; (ii) an arginine (R) residue inserted at amino acid 152; (iii) an arginine (R) residue at amino acid 168; (iv) an isoleucine (I) residue at amino acid 189; and (v) a seine (S) residue at amino acid 200, wherein amino acid positions in the AAV capsid protein are numbered with respect to amino acid positions in the amino acid sequence of SEQ ID NO: 3.

In embodiments, one or more variable regions I through IX in the disclosed VP3 portion capsid proteins may be removed and replaced with alternative regions. Suitable alternatives are identified in Table 4 below. The location for these, as well as the identity of additional alternatives may be identified by alignment to SEQ ID NO:41. In embodiments, one or more VRs may have an insertion of 1, 2 or 3 amino acids. In embodiments, one or more VRs may have a deletion of 1, 2 or 3 amino acids.

TABLE 4
VR Alternatives

| VR | Sequence |
|---|---|
| I | SASTGAS (SEQ ID NO. 52); NSTSGGSS (SEQ ID |
| | NO. 53); SSTSGGSS (SEQ ID NO: 87) |
| II | DNNGVK (SEQ ID NO. 54) |
| III | NDGS (SEQ ID NO. 55) |
| IV | INGSGQNQQT (SEQ ID NO. 56) |
| VI | RVSTTTGQNNNSNFAWTA (SEQ ID NO. 57) |
| VII | HKEGEDRFFPLSG (SEQ ID NO. 58); |
| VIII | ADNLQQONTAPQI (SEQ ID NO. 60); |
| IX | NYYKSTSVDF (SEQ ID NO. 61). |

The disclosure provides nucleic acids encoding any one of the AAV capsid proteins disclosed herein. The disclosure also provides vectors comprising any one of the nucleic acids disclosed herein. The AAV may be an AAV2, an AAV8, or an AAV9 serotype.

### AAV Vectors

AAV vectors supply the nucleic acid that becomes encapsidated into the AAV vector particle including elements involved in controlling expression of the nucleic acids in the subject, as well as ITRs to facilitate encapsidation. In embodiments, the AAV vector comprises an AAV vector genome of the disclosure. In embodiments, the AAV vectors disclosed herein comprise at least one heterologous nucleic acid (HNA) sequence or a portion thereof, which, when expressed in a cell of a subject and recombined with the remaining portion of an HNA sequence, encodes a transgene that treats a disease or disorder. Thus, the HNA sequence comprises a transgene, or a portion thereof. In embodiments, the AAV vectors comprise at least one ITR sequence and at least one transgene or portion thereof. In embodiments, the AAV vectors comprise at least one ITR sequence and a portion of one transgene. In embodiments, the transgene encodes a therapeutic protein or a therapeutic RNA.

In embodiments, control of transgene expression in the host cell may be regulated by regulatory elements contained within the AAV vector, including promoter sequences, and polyadenylation signals. In embodiments, the AAV vector also encodes a signal peptide. In embodiments, the AAV vectors have 5′ and 3′ inverted terminal repeats (ITRs). In embodiments, the 5′ and 3′ ITR have the same sequence. In embodiments, they have different sequences.

In embodiments, the 5′ AAV inverted terminal repeat comprises or consists of a sequence having at least 70%, at least 75%, at least 80%, at least 85%, at least 90%, at least 95%, at least 96%, at least 97%, at least 98%, at least 99%, or 100% identity to SEQ ID NO: 215.

In embodiments, the 3′ AAV inverted terminal repeat comprises or consists of a sequence having at least 70%, at least 75%, at least 80%, at least 85%, at least 90%, at least 95%, at least 96%, at least 97%, at least 98%, at least 99%, or 100% identity to SEQ ID NO: 216.

The present disclosure provides methods of expressing transgenes encoded by genes above a size limit. The size limit is determined by the packaging capacity of the viral particles carrying the heterologous nucleic acid sequence and, for most of AAV viral vectors, the size limit of the encapsidated vector genome is typically about 4.7 kb. The present disclosure provides approaches for delivering to host cells and expressing transgenes that are up to about 8 kb, up to about 7.9 kb, up to about 7.8 kb, up to about 7.7 kb, up to about 7.6 kb, up to about 7.5 kb, or up to about 7.4 kb, using AAV viral particles. The length of the transgene suitable for the bipartite gene delivery approach is between about 4.0 kb to about 8.0 kb; for example between about 4.1 kb to about 7.9 kb, between about 4.2 kb to about 7.8 kb, between about 4.3 kb to about 7.7 kb, between about 4.4 kb to about 7.7 kb, between about 4.5 kb to about 7.7 kb, or between about 4.6 kb to about 7.7 kb. In embodiments, the present disclosure provides multipartite gene delivery approaches for delivering to host cells and expressing transgenes that are up to about 8 kb, about 9 kb, about 10 kb, about 11 kb, about 12 kb, about 13 kb, about 14 kb, about 15 kb, about 16 kb, about 17 kb, about 18 kb, about 19 kb, about 20 kb, or any values in between.

Previous approaches for delivering a transgene above the size limit of the AAV system include using homologous recombination mediated by the host cell's own cellular machinery. However, the efficiency of homologous recombination varies widely among different organisms and cell types, and endogenous homologous recombination process is error-prone.

The novel methods described herein employ exogenous recombinases to mediate efficient and precise recombination between the AAV vector genomes encoding different parts of the transgene, which overcomes the problems associated with endogenous homologous recombination. In embodiments, the exogenous recombinase is a Cre recombinase. Cre recombinase belongs to the integrase family of site-specific recombinases. It catalyzes recombination between two recognition sites called Lox sites. A Lox site is typically a 34 bp polynucleotide sequence consisting of a core spacer region of 8 bp and two 13 bp flanking sequences (the recognition regions). The asymmetric core sequence defines an orientation to the Lox site. By using Cre recombination approach methods as disclosed herein, we obtained highly efficient recombination, regardless of the cellular environment and in the absences of additional co-factors. Other exogenous recombinases such as yeast Flippase (Flp) may be used.

The multipartite viral delivery systems of the disclosure use exogenous recombinase (e.g., Cre recombinase) to mediate the recombination and integration of different portions of the transgene of interest via specific recombination sites within the AAV vector genomes that are delivered to the host cell inside the viral vector. In embodiments, three or more portions of a transgene of interest can be delivered using multipartite viral delivery system and form the complete transgene in the host cell using the same strategy.

An illustrative approach for delivering to host cells and expressing a transgene above the size limit of a single AAV vector genome is depicted in FIG. 1A. The transgene is split into two parts: a 5′ portion of the transgene, and a 3′ portion of the transgene. The 5′ portion of the transgene is incorporated into the first AAV vector genome, and the 3′ portion of the transgene is incorporated into the second AAV vector genome. The first AAV vector genome also comprises an HNA sequence encoding a recombinase (Rec); for example a Cre recombinase. Once expressed in cells, the recombinase mediates recombination between the first AAV vector genome and the second AAV vector genome through the recombination sites (RS) in the first and second AAV vector genomes. After recombination, the recombined nucleic acid comprises both the 5′ and the 3′ portions of the transgene which, once transcribed, can be joined together during RNA splicing due to the presence of splice donor (SD) and splice acceptor (SA) sites, resulting in the expression of full-length transgene.

A first AAV vector genome may comprise, in 5′ to 3′ orientation:
a. a 5′ AAV inverted terminal repeat (ITR);
b. a promoter;
c. the 5′ portion of the transgene;
d. a splice donor (SD) site;
e. a recombination site (RS);
f. optionally, a nucleic acid sequence encoding an internal cleavage (IC) polypeptide or an internal ribosome entry site (IRES);
g. a nucleic acid sequence encoding a recombinase, optionally with a nuclear localization sequence (NLS);
h. a poly-A site (pA); and
i. a 3′ AAV inverted terminal repeat.

Typically, there is no stop codon between the 5′ portion of the transgene and the recombinase nucleic acid, meaning that the HNA region comprising the 5′ portion of the transgene and the gene encoding the recombinase (optionally with a nuclear localization sequence) forms a continuous ORF (i.e., are in the same reading frame).

In embodiments, the first AAV vector genome comprises, in 5′ to 3′ orientation,
(a) a 5′ AAV inverted terminal repeat;
(b) an enhancer (e.g., an interphotoreceptor retinoid-binding protein (IRBP) promoter);
(c) a promoter (e.g., an eye-specific promoter);
(d) a 5′ portion of a transgene (e.g., a transgene encoding an ABCA4 protein);
(e) a splice donor (SD) site;
(f) a recombination site;
(g) a polynucleotide encoding a recombinase;
(h) a poly-A site; and
(i) a 3′ AAV inverted terminal repeat.

In embodiments, the first AAV vector genome comprises, in 5′ to 3′ orientation,
(a) a 5′ AAV inverted terminal repeat;
(b) a human rhodopsin (hRho) promoter;
(c) a 5′ portion of a transgene (e.g., a transgene encoding an ABCA4 protein);
(d) a splice donor (SD) site;
(e) a recombination site;
(f) a polynucleotide encoding a recombinase;
(g) a poly-A site; and
(h) a 3′ AAV inverted terminal repeat.
   In embodiments, the first AAV vector genome comprises an enhancer (e.g., an interphotoreceptor retinoid-binding protein (IRBP) promoter) between the 5′ AAV inverted terminal repeat and the human rhodopsin (hRho) promoter.

In embodiments, the first AAV vector genome comprises, in 5′ to 3′ orientation,
(a) a 5′ AAV inverted terminal repeat;
(b) a CMV enhancer;
(c) a promoter (e.g., a chicken beta-actin (CBA) promoter);
(d) a 5′ portion of a transgene (e.g., a transgene encoding an ABCA4 protein);
(e) a splice donor (SD) site;
(f) a recombination site;
(g) a polynucleotide encoding a recombinase;
(h) a poly-A site; and
(i) a 3′ AAV inverted terminal repeat.

A second AAV vector genome may comprise, in 5′ to 3′ orientation:
a. a 5′ AAV inverted terminal repeat;
b. a recombination site;
c. a splice acceptor (SA) site;
d. the 3′ portion of the transgene ORF;
e. a poly-A site;
f. and a 3′ AAV inverted terminal repeat.
   In embodiments, the recombination site of the second AAV vector genome is a Lox 66 recombination site. In embodiments, the Lox 66 recombination site comprises or consists of SEQ ID NO: 179, or a sequence having at most 1, at most 2, at most 3, at most 4, at most 5, or at most 6 nucleotides mutations (deletion, insertion, and/or substitution) thereto. In embodiments, the recombination site of the second AAV vector genome is a Lox JTZ17 recombination site. In embodiments, the Lox JTZ17 recombination site comprises or consists of SEQ ID NO: 217, or a sequence having at most 1, at most 2, at most 3, at most 4, at most 5, or at most 6 nucleotides mutations (deletion, insertion, and/or substitution) thereto. In embodiments, the mutated recombination site sequence(s) substantially retain the recombination function of the reference sequence (e.g., SEQ ID NO: 179 or 217). In embodiments, the mutated recombination site sequence(s) retain at least 80%, at least 90%, at least 95%, or at least 100% of the recombination function of the reference sequence (e.g., SEQ ID NO: 179 or 217).

Elements of the first and second AAV vector genomes facilitate production of the full-length transgene. When both the first AAV vector genome and the second AAV vector genome are present in the same cell, a recombinase is expressed under the control of a promoter in the first AAV vector genome. The recombinase mediates recombination of the first AAV vector genome and the second AAV vector genome via the recombination site in the AAV vector genome and the recombination site in the second AAV vector genome, resulting in the formation of new recombined nucleic acids. The recombined nucleic acid comprises both the 5′ and 3′ portions of the transgene, separated by splice donor and acceptor sites. Following transcription and RNA maturation, full-size mRNA is produced from the first recombined nucleic acid.

Three or more portions of the transgene can be delivered to a host cell using a multipartite viral delivery system, and recombined to allow expression of a complete transgene. An illustrative approach for delivering three portions of the transgene is depicted in FIG. 1B, and the same strategy can be used to deliver a transgene using more than three portions. Briefly, in FIG. 1B, a 1st portion of the transgene is incorporated into a first AAV vector genome, a 2nd portion of the transgene is incorporated into a second AAV vector genome, and a 3rd portion of the transgene is incorporated into a third AAV vector genome. The AAV vector genomes are then encapsidated to produce AAV vector particles which are delivered to the host cells and introduce the AAV vector genomes. Similar to the design in FIG. 1A, the first AAV vector genome in FIG. 1B comprises a promoter, a first splice donor (SD1) site, a recombination site RS1, optionally a nucleic acid sequence encoding an internal cleavage (IC) polypeptide or an internal ribosome entry site (IRES), a nucleic acid sequence encoding a recombinase (Rec) optionally with a nuclear localization sequence (NLS), and a poly-A site (pA). The second AAV vector genome also comprises a recombination site RS1′, a first splice acceptor (SA1) site, a second splice donor (SD2) site, and a recombination site RS2. The third AAV vector genome comprises a recombination site RS2′, a second splice acceptor (SA2) site and a poly-A site.

Once the three AAV vector genomes are delivered in the same host cell, the expression of recombinase (e.g., Cre recombinase) mediates the recombination between RS1 and RS1′ sites and the recombination between RS2 and RS2′ sites. In embodiments, the RS1 and RS1′ sites are orthogonal to the RS2 and RS2′ sites (i.e., minimal to no recombination between RS1/RS1′ and RS2/RS2′). As a non-limiting example, when the recombinase is a Cre recombinase, the RS1 and RS1′ can be LoxP sites and the RS2 and RS2′ can be LoxN sites. After the recombination, an HNA comprising all three portions of the transgene is formed, and its precursor mRNA transcript can undergo RNA splicing mediated by the SD1/SA1 splicing pair and the SD2/SA2 splicing pair to form a mature mRNA transcript that comprises the integrated transgene without any of the recombination sites. In embodiments, the SD1/SA1 splicing pair and the SD2/SA2 splicing pair are orthogonal to each other (i.e., no splicing occurs between SD1/SA2 pair or SD2/SA1 pair), thereby ensuring that all portions of the transgene are retained in their correct positions within the mature mRNA transcript.

In embodiments, the recombinase is cleaved from the N-terminal portion of the protein via an internal cleavage polypeptide. In embodiments, expression of the recombinase may be driven by an internal ribosome entry site (IRES).

The recombined nucleic acid may comprise, in 5′ to 3′ orientation: a 5′ AAV inverted terminal repeat (ITR); a promoter; a 5′ portion of the transgene, a splice donor (SD) site; a recombination site (RS) formed by the recombination process; a splice acceptor (SA) site; a 3′ portion of the transgene; a poly-A site (pA); and a 3′ AAV inverted terminal repeat. The promoter drives efficient transcription of the HNA sequence comprising the transgene, and the 5′ portion and 3′ portion of the transgene are joined together after splicing of the RNA transcript via the splice donor and acceptor sites, which leads to formation of mRNA transcript of a full-length transgene.

In embodiments, the gene encoding the recombinase located in the recombined nucleic acids cannot be efficiently transcribed due to lack of promoter. As a result, in embodiments, little or no stable expression of the recombinase can be detected in cells transduced with both the first and the second AAV vector genomes.

The recombinase may be a Cre recombinase. The Cre recombinase may comprise, consist essentially of, or consist of, the amino acid sequence of SEQ ID NO: 164 or may have at least about 70%, at least about 75%, at least about 80%, at least about 85%, at least about 90%, at least about 95% identity or at least about 99% identity to SEQ ID NO: 164. In embodiments, the Cre recombinase may have 1, 2, 3, 4, 5, 6, 7, 8, 9, or up to 10 amino acids different than SEQ ID NO: 164. In embodiments, the Cre recombinase substantially retains the recombinase function of SEQ ID NO: 164. In embodiments, the Cre recombinase retains at least 80%, at least 90%, at least 95%, or at least 100% of the recombinase function of SEQ ID NO: 164.

The Cre recombinase may be fused to a nuclear localization sequence (NLS). The location of the NLS may be at the N-terminus, the C-terminus, or in the middle of the Cre recombinase. The NLS may have the amino acid sequence of PKKKRKV (SEQ ID NO: 165), or up to 1, 2, or 3 amino acids variation from SEQ ID NO: 165.

The Cre recombinase with NLS may comprise, consist essentially of, or consist of the amino acid sequence of SEQ ID NO: 166 or may have at least about 70%, at least about 75%, at least about 80%, at least about 85%, at least about 90%, at least about 95% identity or at least about 99% identity to SEQ ID NO: 166. In embodiments, the polynucleotide encoding the Cre recombinase with NLS comprises, consists essentially of, or consists of, a sequence at least about 70%, at least about 75%, at least about 80%, at least about 85%, at least about 90%, at least about 95% identity, or 100% identity to SEQ ID NO: 167.

In embodiments, the polynucleotide encoding the recombinase (e.g., Cre-recombinase) comprises an intron. In embodiments, the intron comprises or consists of a sequence having at least 70%, at least 75%, at least 80%, at least 85%, at least 90%, at least 95%, at least 96%, at least 97%, at least 98%, at least 99%, or 100% identity to SEQ ID NO: 214.

In embodiments, the polynucleotide encoding the Cre recombinase (and associated intron) comprises or consists of a sequence having at least 70%, at least 75%, at least 80%, at least 85%, at least 90%, at least 95%, at least 96%, at least 97%, at least 98%, at least 99%, or 100% identity to SEQ ID NO: 213.

The recombination sites recognized by the Cre recombinase may comprise one or more LoxP (locus of X-over P1) sequences. Examples of LoxP sequences are listed in Table 5 below. A representative LoxP sequence is 34 bp in length and comprises an asymmetric 8 bp spacer region in between two sets of 13 bp recognition regions. In embodiments, the LoxP sequence comprises a spacer region having the nucleotide sequence of ATGTATGC. The pairing of recombination sites (one in the first AAV vector genome and the other in the second AAV vector genome) may promote unidirectional recombination and/or prevent backwards recombination.

The recombination site recognized by the Cre recombinase may comprise, consist essentially of, or consist of one or more variant Lox site comprising a spacer region sequence that is different from the canonical sequence (ATGTATGC). In embodiments, the variant Lox site is orthogonal to the LoxP site (i.e., no cross-recombination between the variant Lox site and the LoxP site). In embodiments, the Lox site is a loxN site comprising a spacer region sequence of GTATACCT. In embodiments, the Lox site is a lox2272 site comprising a spacer region sequence of AAGTATCC. In embodiments, the Lox site is a lox511 site comprising a spacer region sequence of ATGTATAC. In embodiments, two or more Lox sites comprising different spacer region sequences are used and these Lox sites are orthogonal to each other. In embodiments, the Lox sites undergo recombination with Lox sites comprising the same spacer region sequence, but are not cross-compatible. More discussions about orthogonal Lox sites can be found in Livet et al. Nature. 2007 Nov. 1, 450(7166):56-62 and Missirlis et al. BMC Genomics, 2006 Apr. 4, 7:73, each of which is incorporated herein by reference in its entirety. Exemplary variant Lox site sequences are provided in Table 5 below.

TABLE 5
Exemplary LoxP Sequences and Variant Lox Site Sequences

| | 13 bp | 8 bp | 13 bp | |
|---|---|---|---|---|
| | Recognition | Spacer | Recognition | |
| Name | Region | Region | Region | SEQ ID NO |
| Wild- | ATAACTTCGTATA | ATGTATGC | TATACGAAGTTAT | SEQ ID NO: 168 |
| Type | | | | |
| Lox 511 | ATAACTTCGTATA | ATGTATaC | TATACGAAGTTAT | SEQ ID NO: 169 |
| Lox 5171 | ATAACTTCGTATA | ATGTgTaC | TATACGAAGTTAT | SEQ ID NO: 170 |
| Lox 2272 | ATAACTTCGTATA | AaGTATcC | TATACGAAGTTAT | SEQ ID NO: 171 |
| M2 | ATAACTTCGTATA | AgaaAcca | TATACGAAGTTAT | SEQ ID NO: 172 |
| M3 | ATAACTTCGTATA | taaTACCA | TATACGAAGTTAT | SEQ ID NO: 173 |
| M7 | ATAACTTCGTATA | AgaTAGAA | TATACGAAGTTAT | SEQ ID NO: 174 |
| M11 | ATAACTTCGTATA | cgaTAcca | TATACGAAGTTAT | SEQ ID NO: 175 |
| Lox 71 | TACCGTTCGTATA | NNNTANNN | TATACGAAGTTAT | SEQ ID NO: 176 |
| Lox 66 | ATAACTTCGTATA | NNNTANNN | TATACGAACGGTA | SEQ ID NO: 177 |
| Lox 71 | TACCGTTCGTATA | ATGTATGC | TATACGAAGTTAT | SEQ ID NO: 178 |
| Lox 66 | ATAACTTCGTATA | ATGTATGC | TATACGAACGGTA | SEQ ID NO: 179 |
| LoxN | ATAACTTCGTATA | GTATACCT | TATACGAAGTTAT | SEQ ID NO: 207 |
| Lox | ATAACTT CGTATA | ATGTATGC | TATAGCAATTTAT | SEQ ID NO: 217 |
| JTZ17 | | | | |

In embodiments, the recombination site in the first AAV vector genome may comprise, consist essentially of, or consist of a Lox 71 sequence; for example, SEQ ID NO: 178, or a sequence having at most 1, at most 2, at most 3, at most 4, at most 5, or at most 6 nucleotides mutations (deletion, insertion, and/or substitution) thereto. In embodiments, the mutated Lox 71 sequence substantially retain the recombination function of SEQ ID NO: 178. In embodiments, the mutated Lox 71 sequence retain at least 80%, at least 90%, at least 95%, or at least 100% of the recombination function of SEQ ID NO: 178.

In embodiments, the recombination site in the second AAV vector genome may comprise, consist essentially of, or consist of a Lox JTZ17 sequence; for example, SEQ ID NO: 217, or a sequence having at most 1, at most 2, at most 3, at most 4, at most 5, or at most 6 nucleotides mutations (deletion, insertion, and/or substitution) thereto. In embodiments, the mutated Lox JTZ17 sequence substantially retain the recombination function of SEQ ID NO: 217. In embodiments, the mutated Lox JTZ17 sequence retain at least 80%, at least 90%, at least 95%, or at least 100% of the recombination function of SEQ ID NO: 217.

In embodiments, the recombination site in the second AAV vector genome may comprise, consist essentially of, or consist of a Lox 66 sequence; for example, SEQ ID NO: 179, or a sequence having at most 1, at most 2, at most 3, at most 4, at most 5, or at most 6 nucleotides mutations (deletion, insertion, and/or substitution) thereto. In embodiments, the mutated Lox 66 sequence substantially retain the recombination function of SEQ ID NO: 179. In embodiments, the mutated Lox 66 sequence retain at least 80%, at least 90%, at least 95%, or at least 100% of the recombination function of SEQ ID NO: 179.

The first AAV vector genome may encode an internal cleavage polypeptide to facilitates the cleavage of the recombinase from the rest of the polypeptide. The nucleic acid encoding the internal cleavage polypeptide may be located about 1-1000 bp upstream of the 5′ end of the nucleic acid portion encoding the recombinase; for example, about 1-500 bp, about 1-300 bp, about 1-200 bp, about 1-150 bp, about 1-100 bp, about 1-50 bp, about 1-30 bp, about 1-20 bp, or about 1-10 bp upstream of the 5′ end of the nucleic acid portion encoding the recombinase.

The internal cleavage polypeptide may be a 2A self-cleaving peptide. Suitable examples of 2A self-cleaving peptide are listed in Table 6 below. In embodiments, the 2A self-cleaving peptide comprises or consists of P2A (SEQ ID NO: 181). In embodiments, the 2A self-cleaving peptide further comprises three amino acids “GSG” attached immediately adjacent to the N-term of one of SEQ ID NO: 180-183. Suitable examples include, an internal cleavage peptide having an amino acid sequence of SEQ ID NO: 184, which is encoded by a nucleic acid having the sequence of SEQ ID NO: 185. The C-terminal residues of the internal cleavage peptide may overlap with the N-terminal residues of the Cre recombinase with NLS.

TABLE 6
Exemplary 2A Self-cleaving Peptides

| Name | Sequence | SEQ ID NO |
|---|---|---|
| T2A | EGRGSLLTCGDVEENPGP | SEQ ID NO: 180 |
| P2A | ATNFSLLKQAGDVEENPGP | SEQ ID NO: 181 |
| E2A | QCTNYALLKLAGDVESNPGP | SEQ ID NO: 182 |
| F2A | VKQTLNFDLLKLAGDVESNPGP | SEQ ID NO: 183 |

In embodiments, little or no stable expression of the recombinase can be detected in cells transduced with both the first and the second AAV vector genomes. In embodiments, no expression of the recombinase can be detected by western blot in cells after about 1 hour, about 3 hours, about 6 hours, about 12 hours, about 24 hours, about 48 hours, about 3 days, about 4 days, about 5 days, about 6 days, about 1 week, about 2 weeks, about 3 weeks, or about 1 month, post transduction with both AAV vector genomes. In embodiments, no expression of the recombinase can be detected by western blot in cells after about 48 hours post transduction with both AAV vector genomes. Western blot analysis of the recombinase can be performed by a skilled artisan using commercially available antibodies. For example, the expression level of Cre recombinase can be analyzed by Western blot using rabbit anti-Cre mAb (Cell signaling technology #15036) at 1:10,000 dilution following the protocol described in Example 3.

In embodiments, little or no stable expression of the mRNA encoding the recombinase can be detected in cells transduced with both the first and the second AAV vector genomes. In embodiments, no expression of the mRNA encoding the recombinase can be detected by quantitative PCR (qPCR) in cells after about 1 hour, about 3 hours, about 6 hours, about 12 hours, about 24 hours, about 48 hours, about 3 days, about 4 days, about 5 days, about 6 days, about 1 week, about 2 weeks, about 3 weeks, or about 1 month, post transduction with both AAV vector genomes. In embodiments, no expression of the mRNA encoding the recombinase can be detected by qPCR in cells after about 1 week post transduction with both AAV vector genomes.

The first AAV vector genome may comprise an internal ribosome entry site (IRES) to facilitate expression of a downstream recombinase.

Splice donor (SD) and splice acceptor (SA) sites can be used to remove portions of an mRNA transcript. The splice donor (SD) site may be located downstream of the 5′ portion of the transgene in an AAV vector genome. For example, the splice donor (SD) site may be about 1-500 bp, about 1-300 bp, about 1-200 bp, about 1-150 bp, about 1-100 bp, about 1-50 bp, about 1-30 bp, about 1-20 bp, or about 1-10 bp downstream of the 5′ portion of the transgene.

The splice acceptor (SA) site may be located upstream of the 3′ portion of the transgene in an AAV vector genome. For example, about 1-500 bp, about 1-300 bp, about 1-200 bp, about 1-150 bp, about 1-100 bp, about 1-50 bp, about 1-30 bp, about 1-20 bp, or about 1-10 bp upstream of the 3′ portion of the transgene.

The splice donor (SD) site and the splice acceptor (SA) sites may be located within different AAV vector genomes, respectively, such that, after the recombination between the first and the second AAV vector genomes, the mRNA transcript comprising the 5′ portion of the transgene and the 3′ portion of the transgene can be processed to form a mature mRNA transcript comprising a continuous full-length transgene. See FIG. 1A.

The splice donor (SD) site may comprise, consist essentially of, or consist of the nucleotide sequence of SEQ ID NO: 186, or a sequence having up to 1, up to 3, up to 5, or up to 10 nucleotide changes to SEQ ID NO: 186. The splice acceptor (SA) site may comprise, consist essentially of, or consist of the nucleotide sequence of SEQ ID NO: 187, or a sequence having up to 1, up to 3, up to 5, or up to 10 nucleotide changes to SEQ ID NO: 187. In embodiments, the mutated splice donor and/or splice acceptor site substantially retains the splicing function of SEQ ID NO: 186 or 187. In embodiments, the mutated splice donor and/or splice acceptor site retains at least 80%, at least 90%, at least 95%, or at least 100% of the splicing function of SEQ ID NO: 186 or 187.

Control of transgene expression in the host cell may be regulated by regulatory elements contained within the AAV vector genome, including promoter sequences, and poly-A sites. The AAV vector may also encode a signal peptide. In embodiments, the AAV vector genomes have 5′ and 3′ inverted terminal repeats (ITRs). The 5′ ITR is located upstream of a promoter, which in turn is upstream of the transgene. In embodiments, the 5′ and 3′ ITR have the same sequence. In embodiments, they have a different sequence. In embodiments, an AAV vector genome of the disclosure may comprise, in 5′ to 3′ orientation, a first (5′) ITR, a promoter, a transgene, a poly-A site, and a second (3′) ITR.

The HNA (for example, an HNA comprising a transgene or portion thereof) may be operably linked to one or more regulatory element(s) (e.g., a promoter). In embodiments, the HNA is operably linked to a promoter.

In embodiments, the HNA is operably linked to a constitutive promoter. The constitutive promoter can be any constitutive promoter known in the art and/or provided herein. In embodiments, the constitutive promoter comprises, consists essentially of, or consists of a Rous sarcoma virus (RSV) LTR promoter (optionally with the RSV enhancer), a cytomegalovirus (CMV) promoter, an SV40 promoter, a dihydrofolate reductase promoter, a beta-actin promoter, a phosphoglycerol kinase (PGK) promoter, a U6 promoter, an H1 promoter, a hybrid chicken beta actin promoter, a MeCP2 promotor, an H1 promoter, a U1a promoter, a mMeP418 promoter, a mMeP426 promoter, a minimal MeCP2 promoter, a CAG promoter, or an EF1 promoter. It is known in the art that the nucleotide sequences of such promoters may be modified in order to increase or decrease the efficiency of mRNA transcription. See, e.g., Gao et al. (2018) Mol. Ther.: Nucleic Acids 12:135-145 (modifying TATA box of 7SK, U6 and H1 promoters to abolish RNA polymerase III transcription and stimulate RNA polymerase II-dependent mRNA transcription).

In embodiments, the promoter used herein comprises, consists essentially of, or consists of a polynucleotide having the sequence of SEQ ID NO: 96 (mouse U1 promoter) or a SEQ ID NO: 97 (a H1 promoter). In embodiments, the promoter may be a U1a or U1b promoter, an EF1 promoter, or a chicken beta-actin (CBA) promoter.

In embodiments, the promoter is a chicken beta-actin (CBA) promoter. In embodiments, the CBA promoter comprises, consists essentially of, or consists of a polynucleotide sequence having at least 70%, at least 75%, at least 80%, at least 85%, at least 90%, at least 95%, at least 97%, at least 98%, at least 99%, or 100% identity to SEQ ID NO: 219. In embodiments, the CBA promoter substantially retains the promoter function of SEQ ID NO: 219. In embodiments, the CBA promoter retains at least 80%, at least 90%, at least 95%, or at least 100% of the promoter function of SEQ ID NO: 219.

In embodiments, the HNA sequence is operably linked to a tissue-specific promoter, or an inducible promoter. In embodiments, the tissue-specific promoter is a central nervous system (CNS) cell-specific promoter, a lung-specific promoter, a skin-specific promoter, a muscle-specific promoter, a liver-specific promoter, or an eye-specific promoter.

In embodiments, the HNA sequence is operably linked to an eye-specific promoter. In embodiments, the eye-specific control promoter is selected from a human rhodopsin kinase (hGRK1) promoter, a human rhodopsin (hRho) promoter, a human rod cGMP phosphodiesterase β-subunit (βPDE) promoter, a human interphotoreceptor retinoid-binding protein (IRBP) promoter, a human red/green opsin (pR2.1) promoter, a human blue opsin (HB) promoter, a human vitelliform macular dystrophy-2 (VMD2) promoter, a human RPE65 promoter, a mouse opsin (mOP) promoter, a mouse short wave opsin promoter (mBP) (short wave opsin promoters may also be referred to as S-opsin or blue opsin promoters), and a mouse rhodopsin (mRho) promoter.

In embodiments, the promoter is a human rod cGMP phosphodiesterase β-subunit (βPDE) promoter. In embodiments, the βPDE promoter comprises, consists essentially of, or consists of a nucleic acid sequence having at least 70%, at least 75%, at least 80%, at least 85%, at least 90%, at least 95%, at least 97%, at least 98%, at least 99%, or 100% identity to SEQ ID NO: 200. In embodiments, the βPDE promoter substantially retains the promoter function of SEQ ID NO: 200. In embodiments, the βPDE promoter retains at least 80%, at least 90%, at least 95%, or at least 100% of the promoter function of SEQ ID NO: 200.

In embodiments, the promoter is a human rhodopsin (hRho) promoter. In embodiments, the hRho promoter comprises, consists essentially of, or consists of a nucleic acid sequence having at least 70%, at least 75%, at least 80%, at least 85%, at least 90%, at least 95%, at least 97%, at least 98%, at least 99%, or 100% identity to SEQ ID NO: 209. In embodiments, the hRho promoter substantially retains the promoter function of SEQ ID NO: 209. In embodiments, the hRho promoter retains at least 80%, at least 90%, at least 95%, or at least 100% of the promoter function of SEQ ID NO: 209.

In embodiments, the promoter is a human rhodopsin kinase (hGRK1) promoter. In embodiments, the hGRK1 promoter comprises, consists essentially of, or consists of a nucleic acid sequence having at least 70%, at least 75%, at least 80%, at least 85%, at least 90%, at least 95%, at least 97%, at least 98%, at least 99%, or 100% identity to SEQ ID NO: 188. In embodiments, the hGRK1 promoter substantially retains the promoter function of SEQ ID NO: 188. In embodiments, the hGRK1 promoter retains at least 80%, at least 90%, at least 95%, or at least 100% of the promoter function of SEQ ID NO: 188.

In embodiments, the promoter comprises, consists essentially of, or consists of any one of the nucleic acid sequences listed in Table 7, or a sequence having up to 5, up to 10, or up to 30 nucleotide changes to any one of the nucleic acid sequences listed in Table 7. In embodiments, the promoter comprises, consists essentially of, or consists of a nucleic acid sequence having at least 70%, at least 75, at least 80%, at least 85, at least 90%, at least 95%, at least 97%, at least 98%, at least 99%, or 100% identity to any one of the nucleic acid sequences listed in Table 7. In embodiments, the promoter substantially retains the promoter function ofany one of the sequences listed in Table 7. In embodiments, the promoter retains at least 80%, at least 90%, at least 95%, or at least 100% of the promoter function of any one of the sequences listed in Table 7.

TABLE 7
Non-limiting Examples of Promoters

| | Nucleic acid |
|---|---|
| Promoter name | SEQ ID No. |
| Mouse U1a promoter | 152 |
| Polymerase III H1 mutant promoter | 153 |
| Chicken β-actin hybrid promoter CBh (CBh promoter | 154 |
| consists of CMV enhancer, CBA promoter, first CBA | |
| exon and partial intron) | |
| MeCP2 min promoter sequence | 155 |
| MeCP2 promoter sequence | 156 |
| MeCP418 promoter sequence | 157 |
| MeCP426 promoter sequence | 158 |
| VMD2 promoter | 159 |
| PDE6b promoter | 160 |
| mRho promoter | 161 |
| CMV promoter | 162 |
| UbC promoter | 163 |
| Human Rhodopsin Kinase (hGRK1) promoter | 188 |
| Human Interphotoreceptor Retinoid-binding Protein | 198 |
| (IRBP) promoter | |
| Human Red/Green Opsin promoter (pR2.1) | 199 |
| Human Rod cGMP Phosphodiesterase β-subunit | 200 |
| promoter (βPDE) | |
| Human rhodopsin (hRho) promoter | 209 |
| Chicken beta-actin (CBA) promoter | 219 |

In embodiments, the AAV vector comprises a CBA-MVM hybrid sequence between the promoter and the 5′ portion of the transgene. In embodiments, the CBA-MVM hybrid sequence comprises or consists of a sequence having at least 70%, at least 75%, at least 80%, at least 85%, at least 90%, at least 95%, at least 96%, at least 97%, at least 98%, at least 99%, or 100% identity to SEQ ID NO: 210.

In embodiments, the AAV vector comprises an enhancer. In embodiments, the HNA sequence is operably linked to the enhancer. In embodiments, the enhancer is located upstream of the promoter. In embodiments, the enhancer is located immediately upstream of the promoter, without any additional nucleotide in between.

In embodiments, the enhancer is an interphotoreceptor retinoid-binding protein (IRBP) enhancer. In embodiments, the IRBP enhancer comprises or consists of a sequence having at least 70%, at least 75%, at least 80%, at least 85%, at least 90%, at least 95%, at least 96%, at least 97%, at least 98%, at least 99%, or 100% identity to SEQ ID NO: 208. In embodiments, the IRBP enhancer substantially retains the enhancer function of SEQ ID NO: 208. In embodiments, the IRBP enhancer retains at least 80%, at least 90%, at least 95%, or at least 100% of the enhancer function of SEQ ID NO: 208.

In embodiments, the enhancer is a CMV enhancer. In embodiments, the CMV enhancer comprises or consists of a sequence having at least 70%, at least 75%, at least 80%, at least 85%, at least 90%, at least 95%, at least 96%, at least 97%, at least 98%, at least 99%, or 100% identity to SEQ ID NO: 218. In embodiments, the CMV enhancer substantially retains the enhancer function of SEQ ID NO: 218. In embodiments, the CMV enhancer retains at least 80%, at least 90%, at least 95%, or at least 100% of the enhancer function of SEQ ID NO: 218.

In embodiments, the AAV vector genome comprises a sequence comprising a CMV enhancer and a CBA promoter, wherein the sequence has at least 70%, at least 75%, at least 80%, at least 85%, at least 90%, at least 95%, at least 96%, at least 97%, at least 98%, at least 99%, or 100% identity to SEQ ID NO: 211. In embodiments, the CMV enhancer/CBA promoter sequence is located between the 5′ AAV inverted terminal repeat and the 5′ portion of the transgene. In embodiments, the CMV enhancer/CBA promoter sequence substantially retains the enhancer and promoter functions of SEQ ID NO: 211. In embodiments, the CMV enhancer/CBA promoter sequence retains at least 80%, at least 90%, at least 95%, or at least 100% of the enhancer and promoter functions of SEQ ID NO: 211.

In embodiments, the HNA sequence is operably linked to an additional regulatory element. The additional regulatory element can be a woodchuck hepatitis virus post-transcriptional regulatory element (“WPRE”). In embodiments, the AAV vectors may comprise regulatory components suitable for growth and culture of the vector in a bacterial host for vector production purposes. For example, the vector may comprise genes for antibiotic resistance, and maintenance of the plasmid in bacteria, as well as associated regulatory elements to control protein expression in bacteria.

In embodiments, the HNA sequence is operably linked to a polyadenylation (poly-A) signal, also know as a poly-A site. The poly-A site comprises, consists essentially of or consists of an MeCP2 poly-A site, a retinol dehydrogenase 1 (RDH1) poly-A site, a bovine growth hormone (BGH) poly-A site, an SV40 poly-A site, a SPA49 poly-A site (SEQ ID NO: 189), a sNRP-TK65 poly-A site, a sNRP poly-A site, or a TK65 poly-A site. An exemplary SPA49 poly-A sequence is described in Ostedgaard et al., Proc. Nat'l Acad. Sci. USA (Feb. 22, 2005) 102:2952-2957, incorporated herein by reference.

In embodiments, the polyadenylation signal (poly-A site) comprises, consists essentially of, or consists of a nucleic acid sequence having at least 70%, at least 75%, at least 80%, at least 85%, at least 90%, at least 95%, at least 97%, at least 98%, at least 99%, or 100% identity to SEQ ID NO: 189. In embodiments, the polyadenylation signal (poly-A site) comprises, consists essentially of, or consists of SEQ ID NO: 189, or a sequence having at most 1, at most 2, at most 3, at most 4, at most 5, at most 6, at most 7, at most 8, at most 9, or at most 10 nucleotides mutations (deletion, insertion, and/or substitution) thereto. In embodiments, the poly-A site substantially retains the mRNA transport and/or stabilization function of SEQ ID NO: 189. In embodiments, the poly-A site retains at least 80%, at least 90%, at least 95%, or at least 100% of the mRNA transport and/or stabilization function of SEQ ID NO: 189.

### Heterologous Nucleic Acids (HNA)

The AAV viral vectors disclosed herein infect and deliver one or more heterologous nucleic acids (HNA) to target tissues. The HNA sequences of the first and the second AAV vector genomes are recombined within cells to form new HNA sequences. The HNA sequences are transcribed and, optionally, translated in the cells of the target tissue. In embodiments, the new recombinant HNA sequences comprise a transgene encoding a protein. In embodiments, the HNA is operably linked to a promoter.

In embodiments, the cell of the disclosure comprises a defective genomic copy of an endogenous gene, and the transgene is a working copy of the defective endogenous gene, thereby compensating for the function of the defective endogenous gene. In embodiments, both alleles of the endogenous gene are defective in the cells. In embodiments, the “working copy” of the gene substantially retains the function of a wildtype version of the endogenous gene. In embodiments, the transgene retains at least 80%, at least 90%, at least 95%, or at least 100% of the function of the wildtype gene. In embodiments, the transgene encodes a protein, and the protein substantially retains the function of a wildtype protein encoded by a wildtype endogenous gene. In embodiments, the transgene encodes a protein that retains at least 80%, at least 90%, at least 95%, or at least 100% of the function of the wildtype protein encoded by the wildtype endogenous gene. In embodiments, the transgene encodes an intact, wildtype protein. In embodiments, the transgene encodes a fragment of the wildtype protein that substantially retains the function of the wildtype protein.

For transgenes that encode proteins, a transgene encoding the full-length protein is split into two portions, the 5′ portion and the 3′ portion. The length of each portion is less than the length of the full transgene. The 5′ portion of the transgene encodes the N-terminal portion of the protein, and the 3′ portion of the transgene encodes the C-terminal portion of the protein. Those skilled in the art will understand that the 5′ portion and/or the 3′ portion of the transgene does not need to have a nucleic acid length that is the multiple of 3 (i.e., the splitting point of the transgene does not have to be in between two codons), because after the recombination of the first and the second AAV vector genomes, these two portions can be rejoined via mRNA splicing to form the full length transgene. As such, the portion of the transgene and the corresponding amino acid sequence it encodes may be a perfect match or have a small mismatch of 1 or 2 nucleotides at the splitting point. Regarding size limits, the 5′ portion of the transgene in a first AAV vector genome may have a size limit of up to about 3.5 kb, about 3.4 kb, about 3.3 kb or about 3.2 kb. The 3′ portion of the transgene, in a second AAV vector genome, may have a size limit of up to about 4.6 kb, about 4.5 kb, about 4.4 kb, or about 4.3 kb.

In embodiments, the 5′ portion of the transgene in the first AAV vector genome is at least 0.5 kb, at least 1 kb, at least 1.5 kb, at least 2 kb, at least 2.5 kb, at least 3 kb, at least 3.2, or at least 3.5 kb, in length. In embodiments, the 5′ portion of the transgene in the first AAV vector genome is between 0.5-1 kb, 1-1.5 kb, 1.5-2 kb, 2-2.5 kb, 2.5-3 kb, 3-3.2 kb, 3.2-3.5 kb, 0.5-1.5 kb, 1-2 kb, 1.5-2.5 kb, 2-3 kb, 2.5-3.2 kb, 3-3.5 kb, 0.5-2 kb, 1-2.5 kb, 1.5-3 kb, 2-3.2 kb, 2.5-3.5 kb, 0.5-2.5 kb, 1-3 kb, 1.5-3.2 kb, 2-3.5 kb, 0.5-3 kb, 1-3.2 kb, 1.5-3.5 kb, 0.5-3.2 kb, 1-3.5 kb, or 0.5-3.5 kb, in length (inclusive of end points).

In embodiments, the 3′ portion of the transgene in the second AAV vector genome is at least 0.5 kb, at least 1 kb, at least 1.5 kb, at least 2 kb, at least 2.5 kb, at least 3 kb, at least 3.5 kb, at least 4 kb, or at least 4.5 kb, in length. In embodiments, the 3′ portion of the transgene in the second AAV vector genome is between 0.5-1 kb, 1-1.5 kb, 1.5-2 kb, 2-2.5 kb, 2.5-3 kb, 3-3.5 kb, 3.5-4 kb, 4-4.7 kb, 0.5-1.5 kb, 1-2 kb, 1.5-2.5 kb, 2-3 kb, 2.5-3.5 kb, 3-4 kb, 3.5-4.7 kb, 0.5-2 kb, 1-2.5 kb, 1.5-3 kb, 2-3.5 kb, 2.5-4 kb, 3-4.7 kb, 0.5-2.5 kb, 1-3 kb, 1.5-3.5 kb, 2-4 kb, 2.5-4.7 kb, 0.5-3 kb, 1-3.5 kb, 1.5-4 kb, 2-4.7 kb, 0.5-3.5 kb, 1-4 kb, 1.5-4.7 kb, 0.5-4 kb, 1-4.7 kb, or 0.5-4.7 kb, in length (inclusive of end points).

A representative example of a protein encoded by a transgene is ABCA4 (ATP-binding cassette, sub-family A, member 4). Mutations of ABCA4 protein can cause Stargardt disease. In humans, wild-type ABCA4 protein is encoded by the ABCA4 gene and has an amino acid sequence according to Uniprot accession number P78363 (SEQ ID NO: 190). An exemplary nucleic acid sequence encoding the ABCA4 protein is SEQ ID NO: 191. The function of ABCA4 protein includes removing potentially toxic retinoid compounds from photoreceptors, actively flipping N-retinylidene-phosphatidylethanolamine (N-Ret-PE) and phosphatidylethanolamine from the lumen to the cytoplasmic leaflet of disc membranes. Various mutations decrease the transport activity of ABCA4 and cause Stargardt disease. Additional description of the function of ABCA4 protein, and the assay for measuring ABCA4 transport function, can be found, for example, in Quazi et al., Nat Commun. 2012 Jun. 26:3:925, the content of which is incorporated by reference in its entirety for all purposes.

In embodiments, the ABCA4 protein comprises or consists of a sequence having at least 70%, at least 75%, at least 80%, at least 85%, at least 90%, at least 95%, at least 98%, or at least 99% identity to SEQ ID NO: 190. In embodiments, the ABCA4 protein is identical to SEQ ID NO: 190, or up to 1, 2, 3, 4, 5, 6, 7, 8, 9, or 10 amino acids different from SEQ ID NO: 190. In embodiments, the ABCA4 protein substantially retains the N-Ret-PE transport function of SEQ ID NO: 190. In embodiments, the ABCA4 protein retains at least 80%, at least 90%, at least 95%, or at least 100% of the N-Ret-PE transport function of SEQ ID NO: 190.

In embodiments, the ABCA4 transgene is split into a 5′ portion and a 3′ portions that are put into separate AAV vector genomes.

In embodiments, the 5′ portion of the ABCA4 transgene comprises or consists of SEQ ID NO: 192, or a nucleotide sequence having at least 70%, at least 75%, at about 80%, at least 85%, at least 90%, at least 95% identity or at least 99% identity to SEQ ID NO: 192. In embodiments, the 5′ portion of the ABCA4 transgene comprises or consists of SEQ ID NO: 212, or a nucleotide sequence having at least 70%, at least 75%, at least 80%, at least 85%, at least 90%, at least 95% identity or at least 99% identity to SEQ ID NO: 212. In embodiments, the 5′ portion of the ABCA4 transgene comprises one or more of the following nucleotides:
(i) a nucleotide “G” at the position corresponding to nucleotide 813 of SEQ ID NO: 212;
(ii) a nucleotide “A” at the position corresponding to nucleotide 1311 of SEQ ID NO: 212;
(iii) a nucleotide “A” at the position corresponding to nucleotide 2274 of SEQ ID NO: 212;
(iv) a nucleotide “C” at the position corresponding to nucleotide 2277 of SEQ ID NO: 212;
(v) a nucleotide “T” at the position corresponding to nucleotide 2278 of SEQ ID NO: 212;
(vi) a nucleotide “T” at the position corresponding to nucleotide 2568 of SEQ ID NO: 212.
   In embodiments, the 5′ portion of the ABCA4 transgene comprises 2 or more, 3 or more, 4 or more, 5 or more, or all 6 of these nucleotides. In embodiments, the 5′ portion of the ABCA4 transgene does not comprise any BglII restriction sites. In embodiments, the 5′ portion of the ABCA4 transgene does not comprise any BamHI restriction sites. In embodiments, the 5′ portion of the ABCA4 transgene does not comprise any KpnI restriction sites. In embodiments, the 5′ portion of the ABCA4 transgene does not comprise any BglII or KpnI restriction sites. In embodiments, the 5′ portion of the ABCA4 transgene comprises a HindIII restriction site (5′-AAGCTT-3′) (e.g., at a position corresponding to nucleotides 2274-2279 of SEQ ID NO: 212).

In embodiments, the 3′ portion of the ABCA4 transgene comprises or consists of SEQ ID NO: 193, or a nucleotide sequence having at least about 70%, at least about 75%, at least about 80%, at least about 85%, at least about 90%, at least about 95% identity or at least about 99% identity to SEQ ID NO: 193.

Additional examples of transgenes that are suitable for the bipartite gene delivery method include MYO7A and CEP290.

MYO7A encodes myosin VIIa protein and mutations in MYO7A have been implicated in Usher syndrome. In humans, myosin VIIa protein has an amino acid sequence according to SEQ ID NO: 201. An exemplary nucleic acid sequence of MYO7A gene is SEQ ID NO: 202. Suitable myosin VIIa proteins may have at least about 70%, at least about 75%, at least about 80%, at least about 85%, at least about 90%, at least about 95% identity, or at least about 99% identity to SEQ ID NO: 201. The myosin VIIa protein may be identical to SEQ ID NO: 201, or may have up to 1, 2, 3, 4, 5, 6, 7, 8, 9, or 10 amino acids different from SEQ ID NO: 201.

CEP290 gene encodes a centrosomal protein that is localized to the connecting cilium of the photoreceptors and is involved in both ciliogenesis and ciliary trafficking; mutations in CEP290 have been implicated in Leber congenital amaurosis (LCA). In humans, the CEP290 protein (also known as centrosomal protein of 290 kDa) has an amino acid sequence according to SEQ ID NO: 203. An exemplary nucleic acid sequence of CEP290 gene is SEQ ID NO: 204. Suitable CEP290 proteins may have at least about 70%, at least about 75%, at least about 80%, at least about 85%, at least about 90%, at least about 95% identity, or at least about 99% identity to SEQ ID NO: 203. The CEP290 protein may be identical to SEQ ID NO: 203, or may have up to 1, 2, 3, 4, 5, 6, 7, 8, 9, or 10 amino acids different from SEQ ID NO: 203.

### Methods of Producing AAV Viral Vectors

A variety of approaches may be used to produce AAV viral vectors. In embodiments, packaging is achieved by using a helper virus or helper plasmid and a cell line. The helper virus or helper plasmid contains elements and sequences that facilitate viral vector production. In another aspect, the helper plasmid is stably incorporated into the genome of a packaging cell line, such that the packaging cell line does not require additional transfection with a helper plasmid.

In embodiments, the cell is a packaging or helper cell line. In embodiments In aspects, the helper cell line is eukaryotic cell; for example, an HEK 293 cell or 293T cell. In embodiments, the helper cell is a yeast cell or an insect cell.

A helper plasmid may comprise, for example, at least one viral helper DNA sequence derived from a replication-incompetent viral genome encoding in trans all virion proteins required to package a replication incompetent AAV, and for producing virion proteins capable of packaging the replication-incompetent AAV at high titer, without the production of replication-competent AAV.

Helper plasmids for packaging AAV are known in the art, see, e.g., U.S. Patent Pub. No. 2004/0235174 A1, which is hereby incorporated herein by reference in its entirety. As stated therein, an AAV helper plasmid may contain as helper virus DNA sequences, by way of non-limiting example, the Ad5 genes E2A, E4 and VA, controlled by their respective original promoters or by heterologous promoters. AAV helper plasmids may additionally contain an expression cassette for the expression of a marker protein such as a fluorescent protein to permit the simple detection of transfection of a desired target cell.

The disclosure provides methods of producing AAV particles comprising transfecting a packaging cell line with any one of the AAV helper plasmids disclosed herein; and any one of the AAV vectors disclosed herein. In embodiments, the AAV helper plasmid and the AAV vector are co-transfected into the packaging cell line. In embodiments, the cell line is a mammalian cell line, for example, human embryonic kidney (HEK) 293 cell line. The disclosure provides cells comprising any one of the AAV vectors, AAV vector genomes, and/or AAV particles disclosed herein.

### Pharmaceutical Compositions

The disclosure provides pharmaceutical compositions comprising any one of the AAV vectors, AAV vector genomes, AAV capsids and/or AAV particles described herein. Typically, the AAV particles are administered for therapy.

The pharmaceutical composition, as described herein, may be formulated by any methods known or developed in the art of pharmaceutical drug development, which methods include but are not limited to contacting the active ingredients (e.g., viral particles or recombinant vectors) with an excipient or other accessory ingredient, dividing or packaging the product to a dose unit. In embodiments, the pharmaceutical composition is suitable for ocular injection. The viral particles of this disclosure may be formulated with desirable features, e.g., increased stability, increased cell transfection, sustained or delayed release, biodistributions or tropisms, modulated or enhanced translation of encoded protein in vivo, and the release profile of encoded protein in vivo.

In embodiments, the pharmaceutical composition may further comprise saline, lipidoids, liposomes, lipid nanoparticles, polymers, lipoplexes, core-shell nanoparticles, peptides, proteins, cells transduced with viral vectors (e.g., for transplantation into a subject), nanoparticle mimics or combinations thereof. In embodiments, the pharmaceutical composition is formulated as a nanoparticle. In embodiments, the pharmaceutical composition is formulated as sterile and substantially isotonic solution.

A pharmaceutical composition in accordance with the present disclosure may be prepared, packaged, and/or sold in bulk, as a single unit dose, and/or as a plurality of single unit doses. The amount of the active ingredient is generally equal to the dosage of the active ingredient which would be administered to a subject and/or a convenient fraction of such a dosage such as, for example, one-half or one-third of such a dosage. The formulations of the invention can include one or more excipients, each in an amount that together increases the stability of the viral vector, increases cell transfection or transduction by the viral vector, increases the expression of vector genome encoded protein, and/or alters the release profile of vector genome encoded proteins. In embodiments, the pharmaceutical composition comprises an excipient. Non limiting examples of excipients include solvents, dispersion media, diluents, or other liquid vehicles, dispersion or suspension aids, surface active agents, isotonic agents, thickening or emulsifying agents, preservatives, or combination thereof.

In embodiments, the pharmaceutical composition comprises a cryoprotectant. The term “cryoprotectant” refers to an agent capable of reducing or eliminating damage to a substance during freezing. Non-limiting examples of cryoprotectants include sucrose, trehalose, lactose, glycerol, dextrose, raffinose and/or mannitol.

### Therapeutic Methods

This disclosure provides methods of preventing or treating a genetic disorder, comprising, consisting essentially of, or consisting of administering to a subject a therapeutically effective amount of the pharmaceutical compositions disclosed herein.

In embodiments, the genetic disorder is an eye disorder, a CNS disorder, a skin disorder, a lung disorder, a muscle disorder, a liver disorder, a digestive disorder, a blood or lymphatic disorder, an inflammatory disorder, or a cancer. In embodiments, the disorder is Stargardt disease.

In embodiments, the disorder is hypophosphatasia, amyotrophic lateral sclerosis (ALS), spinal muscular atrophy (SMA), recessive dystrophic epidermolysis bullosa (RDEB), lysosomal storage disorder (including Duchenne's Muscular Dystrophy, and Becker muscular dystrophy), juvenile Batten disease, infantile Batten disease, autosomal dominant disorders, muscular dystrophy, Bietti's Crystalline Dystrophy, retinoschisis (e.g., degenerative, hereditary, tractional, exudative), hemophilia A, hemophilia B, multiple sclerosis, diabetes mellitus, Fabry disease, Pompe disease, neuronal ceroid lipofuscinosis 1 (CLN1), CLN3 disease (or juvenile neuronal ceroid lipofuscinosis), Gaucher disease, cancer, arthritis, muscle wasting, heart disease, intimal hyperplasia, Rett syndrome, epilepsy, Huntington's disease, Parkinson's disease, Alzheimer's disease, an autoimmune disease, cystic fibrosis, thalassemia, Hurler's Syndrome (MPS IH), Sly syndrome, Scheie Syndrome, Hurler-Scheie Syndrome, Hunter's Syndrome, Sanfilippo Syndrome A (mucopolysaccharidosis IIIA or MPS IIIA), Sanfilippo Syndrome B (mucopolysaccharidosis IIIB or MPS IIIB), Sanfilippo Syndrome C, Sanfilippo Syndrome D, Morquio Syndrome, Maroteaux-Lamy Syndrome, Krabbe's disease, phenylketonuria, spinal cerebral ataxia, LDL receptor deficiency, hyperammonemia, anemia, arthritis, or adenosine deaminase deficiency.

In addition to specific transgenes disclosed herein, known active enzymes, structural proteins, or RNA sequences may be used as transgenes to deliver functional activity.

The disorder may be an ophthalmic disease. The eye is immune privileged tissue and a low dose of viruses may provide a therapeutic benefit. The ophthalmic disease may affect photoreceptor and/or RPE cells. For example, the ophthalmic disease may be retinitis pigmentosa (e.g., autosomal recessive (SPATA7 gene; LRAT gene; TULP1 gene), autosomal dominant (AIPL1 gene), and X-linked (RPGR gene)), an eye disorder related to mutations in the bestrophin-1 (BEST-1) gene (e.g., vitelliform macular dystrophy, age-related macular degeneration, autosomal dominant vitreoretinochoroidopathy, glaucoma, cataracts), Leber congenital amaurosis (LCA; aryl-hydrocarbon interacting protein-like 1 (AIPL1) gene), cone-rod dystrophy (CRD; ABCA4 gene), Stargardt's (ABCA4 gene), choroideremia (CHM gene), Usher Syndrome (MYO7A gene; CDH23 gene; USH2A gene; CLRN1 gene), retinoschisis (RS1 gene), Bietti's Crystalline Dystrophy (CYP4V2 gene) or Achromatopsia (CNGA3 gene, CNGB3 gene, GNAT2 gene, PDE6C gene, or PDE6H gene).

Suitable disorders to treat using the methods and compositions of the disclosure include Usher syndrome (USH) and Leber congenital amaurosis (LCA). Usher syndrome is an autosomal recessive disorder characterized by hearing impairment associated with retinitis pigmentosa and in some cases vestibular dysfunction. Usher syndrome type I (USH1) is the most serious type, characterized by severe to profound congenital sensorineural hearing loss, balance deficiency and prepubertal onset of retinitis pigmentosa leading to blindness. Six loci for USH1 (USH1B-USH1G) have been mapped. Mutations in MYO7A gene were found to be responsible for USH1B which is the most common subtype of USH1. MYO7A gene encodes myosin VIIA protein. In the human retina, myosin VIIA displays an active function in the migration of RPE melanosomes, phagocytosis of photoreceptor cell outer segment tips and opsin transport through the cilium of these photoreceptors. The coding sequence of MYO7A has a size of ~6.6 kb and the encoded myosin VIIA has an amino acid sequence according to Uniprot accession number Q13402-1 (www.uniprot.org/uniprot/Q13402#Q13402-1). Leber congenital amaurosis (LCA) refers to a group of severe inherited retinal disorders characterized by poor vision within the first year of life, nystagmus, and a non-recordable electroretinogram. Mutations in the CEP290 are one of the most common contributors to LCA. CEP290 gene encodes a centrosomal protein that is localized to the connecting cilium of the photoreceptors and is involved in both ciliogenesis and ciliary trafficking. CEP290 plays a critical role in primary cilium formation and ciliary protein transport. Patients with CEP290-associated LCA retain some cone nuclei in the central cone-rich foveal region. However, these cone photoreceptors have abnormal inner and outer segments resulting in severe vision loss in most patients. The coding sequence of CEP290 gene has a size of ~7.4 kb and encodes a protein sequence according to Uniprot accession number O15078-1 (www.uniprot.org/uniprot/O15078#O15078-1).

In embodiments, the cancer is a solid tumor; for example, bladder, breast, cervical, colon, rectal, endometrial, kidney, lip, oral, liver, melanoma, mesothelioma, non-small cell lung, non-melanoma skin, ovarian, pancreatic, prostate, sarcoma, small cell lung, or thyroid, tumor.

In embodiments, the subject is a mammal; for example, a human. In embodiments, the human is an infant; for example, under 3 years old, 2 years old, or under 1 year old.

The methods of treatment and prevention disclosed herein may be combined with appropriate diagnostic techniques to identify and select patients for the therapy or prevention. For example, the method of treating or preventing a disorder, for example, Stargardt disease, disclosed herein may further comprise steps of performing a genetic test to identify a gene mutation or deletion related to the disorder in the subject. In embodiments, the method of treating or preventing a disorder, for example, Stargardt disease, comprises administering to a subject who has been previously identified as carrying a mutation related to the disorder, or as being at high risk for developing the disorder (for example, based on hereditary factors).

The disclosure provides methods of increasing the level of a protein in a host cell, comprising contacting the host cell with two AAV viral particles as disclosed herein, wherein the first AAV viral particle comprises a first AAV vector genome, and the second AAV viral particle comprises a second AAV vector genome. Cre-lox mediated recombination of the first AAV vector genome and the second AAV vector genome results in an HNA sequence encoding the protein. In embodiments, the protein is a therapeutic protein. In embodiments, the host cell is in vitro, in vivo, or ex vivo. In embodiments, the host cell is derived from a subject. In embodiments, the subject suffers from a disorder, which results in a reduced level and/or functionality of the protein, as compared to the level and/or functionality of the protein in a normal subject.

The disclosure provides methods of preventing or treating a disorder of a subject by introducing a transgene into the subject. In embodiments, the method comprises co-administration of effective amounts of two AAV viral particles—the first AAV viral particle comprises a first AAV vector genome comprising a 5′ portion of the transgene, and the second AAV viral particle comprises a second AAV vector genome comprising a 3′ portion of the transgene. For example, the disclosure provides methods of treating Stargardt disease in a subject. In embodiments, the methods comprise co-administration of two AAV viral particles—the first AAV viral particle comprises the first AAV vector genome comprising a 5′ portion of a transgene (e.g., the ABCA4 gene), and the second AAV viral particle comprises the second AAV vector genome comprising a 3′ portion of the transgene (e.g., the ABCA4 gene).

### Dosage and Administration

Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. It is noted that dosage may be impacted by the route of administration. Suitable dosage formulations and methods of administering the agents are known in the art. Non-limiting examples of such suitable dosages may be as low as 10⁹ vector genomes to as much as 10¹⁷ vector genomes per administration.

In embodiments of the methods described herein, the number of viral particles (e.g., AAV) administered to the subject ranges from about 10⁹ to about 10¹⁷. In embodiments, about 10¹⁰ to about 10¹², about 10¹¹ to about 10¹³, about 10¹¹ to about 10¹², about 10¹¹ to about 10¹⁴, about 5×10¹¹ to about 5×10¹², or about 10¹² to about 10¹³ viral particles are administered to the subject. For administration to a human eye, a total dose of about 1×10¹⁰ vg/eye may be used, and a total dose of 5×10⁹ vg/eye may be used for a mouse eye. Non-invasive, in vivo imaging techniques can be used to monitor efficacy/safety in animals, which include but are not limited to scanning laser ophthalmoscopy (SLO), optical coherence tomography (OCT), multi-photon microscopy, fluorescein angiography.

In embodiments, the viral particle is introduced to the subject intravenously, intrathecally, intracerebrally, intraventricularly, intranasally, intratracheally, intra-aurally, intra-ocularly, peri-ocularly, orally, rectally, transmucosally, inhalationally, transdermally, parenterally, subcutaneously, intradermally, intramuscularly, intrapleurally, topically, intralymphatically, or intracisternally. In embodiments, such introduction may also be intra-arterial, intracardiac, subventricular, epidural, intracerebral, intracerebroventricular, sub-retinal, intravitreal, intraarticular, intraperitoneal, intrauterine, or any combination thereof. In embodiments, the viral particles are delivered to a desired target tissue, e.g., to the lung, eye, or CNS. In embodiments, delivery of viral particles is systemic. The intracisternal route of administration involves administration of a drug directly into the cerebrospinal fluid of the brain ventricles. It could be performed by direct injection into the cisterna magna or via a permanently positioned tube.

For treating an ophthalmic disease (an eye disorder) intraocularly, there are multiple modes of administration known to those skilled in the art, including but not limited to: lacrimal gland (LG) administration, topical eye drop, intra-stromal administration to the cornea, intra-cameral administration (anterior chamber), intravitreal administration, sub-retinal administration, systemic administration, or a combination thereof. About 80% of genetic eye disorders occur in the photoreceptors. Intravitreal delivery of small volume gene therapies can occur in an out-patient clinic.

Some AAV particles show tropism for brain and cervical spine and may also cross the blood-brain-barrier (BBB). Some AAV particles show high retinal tropism by subretinal, para-retinal and/or intravitreal injections. AAV particles of may target multiple eye cell types, for example, cones, rods, and retinal pigment epithelium (RPE). Advantageously, AAV particles may escape neutralizing antibodies against natural serotypes, and thus enable potential redosing. In a further aspect, AAV particles and compositions may be administered in combination with other known treatments for the disorder being treated.

The AAV particles may be administered via subretinal injection. Routes of subretinal injection comprises: (1) a transcorneal route through the pupil, passing the lens, vitreous, and retina; (2) a transscleral route entering the pars plana or limbus areas, crossing through the vitreous and the opposite side of retina into the subretinal space; and (3) a transscleral route through the choroid and Bruch's membrane without penetrating the retina. Subretinal injection is described in Peng et al. Ophthalmic Res. 2017; 58(4):217-226, which is incorporated herein by reference in its entirety.

The AAV particles may be administered via para-retinal injection. In embodiments, para-retinal administration comprises injecting at a distance of between 0-13 millimeters (mm), between 0-10 mm, between 0-5 mm, or between 0-3 mm, from the surface of the retina in the posterior vitreous cavity of the eye. In embodiments, para-retinal administration comprises injecting at a distance of between 0-13 mm from the surface of the retina in the posterior vitreous cavity. In embodiments, para-retinal administration comprises injecting at a distance of between 0-10 mm from the surface of the retina in the posterior vitreous cavity. In embodiments, para-retinal administration comprises injecting at a distance of between 0-5 mm from the surface of the retina in the posterior vitreous cavity. In embodiments, para-retinal administration comprises injecting at a distance of between 0-3 mm from the surface of the retina in the posterior vitreous cavity.

As used herein, the term “para-retinal administration” refers to a form of intravitreal administration that injects the therapeutic agent (e.g., an AAV particle) into the vitreous cavity in close proximity to the desired region of the retina (i.e., targeted delivery). In embodiments, the desired region of the retina is near the fovea area of the retina. In contrast to routine intravitreal administrations, which are done using short needles designed to deposit product in the mid-vitreous cavity, and do not require direct visualization, para-retinal injection is typically done under direct visualization of a longer needle capable of delivering product in the posterior vitreous cavity close to the retina. In embodiments, the therapeutic agent is deposited in the vitreous cavity at a distance of 0 mm to 13 mm from the surface of the retina, at a distance of 0 mm to 10 mm from the surface of the retina, at a distance of 0 mm to 5 mm from the surface of the retina, or at a distance of 0 mm to 3 mm from the surface of the retina. In embodiments, the therapeutic agent is deposited in the vitreous cavity at a distance of between 0-13 mm, between 0-12 mm, between 0-11 mm, between 0-10 mm, between 0-9 mm, between 0-8 mm, between 0-7 mm, between 0-6 mm, between 0-5 mm, between 0-4 mm, between 0-3 mm, between 0-2 mm, or between 0-1 mm, from the surface of the retina.

In embodiments, para-retinal administration is used in situations in which subretinal injection is not appropriate. In embodiments, para-retinal administration is used for targeted transduction of optic nerves. In embodiments, para-retinal administration is used for treating diseases or disorders that are related to dysfunction of the optic nerves. In embodiments, para-retinal administration is used for treating Dominant Optic Atrophy or Retinoschisis.

In embodiments, para-retinal administration involves the use of a small gauge needle (30 gauge or similar) with length sufficient to reach the posterior pole of the human eye (25 mm or similar), exo- or endo-illumination and visualization using a microscope, and use of a corneal contact lens to allow focus on the posterior vitreous cavity and retina. This is typically conducted after adequate analgesia and antisepsis, at which time the corneal contact lens is coupled to the eye and the microscope is positioned to view the posterior retina. The needle is inserted through the eye wall in the pars plana region and its tip is visualized. Under direct visualization, the needle tip is advanced to the desired location close to the retinal surface. The syringe plunger is advanced to slowly deposit the viral vector (which may be contained in any suitable composition or formulation). The needle is withdrawn and the eye is inspected. The port may be closed with a suture, but for a sufficiently small-caliber needle (such as 30 gauge), no suture is needed to close the needle tract. Ointment and an eye shield may be applied and, if desired, the subject can be kept in a supine position for a period post-operatively to further facilitate a high para-retinal concentration of the product. Variations on delivery instrumentation can include creation of a sclerotomy with or without the use of a vitrectomy port to allow use of a blunt cannula, and/or a cannula design with a tapered and/or flexible extendable tip or side ports to optimize proximity to the retinal surface and safety, and/or use of a pneumatic system in lieu of a simple syringe plunger. Additional descriptions of para-retinal administration are disclosed, for example, in WO 2020/018766, WO 2022/245919, and Zeng et al., Mol Ther Methods Clin Dev. 2020 Sep. 11; 18: 422-427, the contents of each of which are incorporated herein by reference in their entireties for all purposes.

Administration of each of the AAV vectors, AAV viral particles or compositions of this disclosure can be effected in one dose, continuously or intermittently throughout the course of treatment. In embodiments, the AAV vectors, AAV particles or compositions of this disclosure are parenterally administered by injection, infusion or implantation.

An AAV viral particle comprising the first AAV vector genome and an AAV viral particle comprising the second AAV vector genome may be co-administered or administered sequentially. “Co-administration” refers to administration of a first and a second AAV viral particle together (i.e., both AAV viral particles may be administered in the same composition). “Sequential administration” means that two AAV viral particles are administered separately but within a time interval such that they can act together on the target cells and achieve a physiological effect (i.e., transduce the same cell in a subject and recombine the transgene portions to produce of full-length transgene). For example, administration of one viral particle can precede administration of the other viral particle by up to about 10 minutes, up to about 20 minutes, up to about 30 minutes, up to about 60 minutes, up to about 2 hours, up to about 3 hours, up to about 6 hours, up to about 12 hours, up to about 24 hours, up to about 2 days, up to about 4 days, up to about 1 week, up to about 2 weeks, or up to about 4 weeks.

The AAV viral particle comprising the first AAV vector genome and the AAV viral particle comprising the second AAV vector genome may be administered at a specific ratio or within a specific range of ratios. In embodiments, the ratio of the AAV viral particle comprising the first AAV vector genome to the AAV viral particle comprising the second AAV vector genome is about 0.1:1, about 0.2:1, about 0.3:1, about 0.4:1, about 0.5:1, about 0.6:1, about 0.7:1, about 0.8:1, about 0.9:1, about 1:1, about 1.1:1, about 1.2:1, about 1.3:1, about 1.5:1, about 1.7:1, about 2:1, about 3:1, about 4:1, about 5:1, about 6:1, about 7:1, about 8:1, about 9:1, or about 10:1. In embodiments, the ratio of the AAV viral particle comprising the first AAV vector genome to the AAV viral particle comprising the second AAV vector genome is within the range of 0.1:1 to 10:1, 0.2:1 to 5:1, 0.3:1 to 3:1, 0.4:1 to 2.5:1, 0.5:1 to 2:1, 0.7:1 to 1.5:1, 0.8:1 to 1.2:1, or 0.9:1 to 1.1:1. In embodiments, the ratio of the AAV viral particle comprising the first AAV vector genome to the AAV viral particle comprising the second AAV vector genome is less than 0.1:1, less than 0.2:1, less than 0.3:1, less than 0.4:1, less than 0.5:1, less than 0.6:1, less than 0.7:1, less than 0.8:1, less than 0.9:1, or less than 1:1. In embodiments, the ratio of the AAV viral particle comprising the first AAV vector genome to the AAV viral particle comprising the second AAV vector genome is about 1:1. In embodiments, the ratio is calculated by comparing the total number of the AAV viral particle comprising the first AAV vector genome to the total number of the AAV viral particle comprising the second AAV vector genome during the course of administration (e.g., co-administration or sequential administration).

### Kits

The agents, vectors, or compositions described herein may, in embodiments, be assembled into pharmaceutical or diagnostic or research kits to facilitate their use in therapeutic, diagnostic or research applications. In embodiments, the kits of the present disclosure include any one of the modified AAV capsid proteins, AAV vectors, AAV vector genomes, AAV particles, host cells, isolated tissues, compositions, or pharmaceutical compositions as described herein.

A kit may further comprise instructions for use. Specifically, such kits may include one or more agents described herein, along with instructions describing the intended application and the proper use of these agents. In embodiments, the kit may include instructions for mixing one or more components of the kit and/or isolating and mixing a sample and applying to a subject. In embodiments, agents in a kit are in a pharmaceutical formulation and dosage suitable for a particular application and for a method of administration of the agents. Kits for research purposes may contain the components in appropriate concentrations or quantities for running various experiments.

The kit may be designed to facilitate use of the methods described herein and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit.

In embodiments, the kit contains any one or more of the components described herein in one or more containers. Thus, in embodiments, the kit may include a container housing agents described herein. The agents may be in the form of a liquid, gel or solid (powder). The agents may be prepared sterilely, packaged in a syringe and shipped refrigerated. Alternatively, they may be housed in a vial or other container for storage. A second container may have other agents prepared sterilely. Alternatively, the kit may include the active agents premixed and shipped in a syringe, vial, tube, or other container. The kit may have one or more or all of the components required to administer the agents to a subject, such as a syringe, topical application devices, or IV needle tubing and bag.

It is to be understood that while the invention has been described in conjunction with the above embodiments, the foregoing description and examples are intended to illustrate and not limit the scope of the invention. Other aspects, advantages and modifications within the scope of the invention will be apparent to those skilled in the art to which the invention pertains.

### FURTHER NUMBERED EMBODIMENTS

Further numbered embodiments of the present disclosure are provided as follows:

Embodiment 1. An AAV vector genome comprising, in 5′ to 3′ orientation:
(a) a 5′ AAV inverted terminal repeat;
(b) an interphotoreceptor retinoid-binding protein (IRBP) enhancer;
(c) an eye-specific promoter;
(d) a 5′ portion of a transgene;
(e) a splice donor (SD) site;
(f) a recombination site;
(g) a polynucleotide encoding a recombinase;
(h) a poly-A site; and
(i) a 3′ AAV inverted terminal repeat.

Embodiment 2. The AAV vector genome of Embodiment 1, wherein the IRBP enhancer comprises or consists of a sequence having at least 90%, at least 95%, at least 96%, at least 97%, at least 98%, at least 99%, or 100% identity to SEQ ID NO: 208.

Embodiment 3. The AAV vector genome of Embodiment 1 or 2, wherein the eye-specific promoter is a human rod cGMP phosphodiesterase β-subunit (βPDE) promoter comprising or consisting of a sequence having at least 90%, at least 95%, at least 96%, at least 97%, at least 98%, at least 99%, or 100% identity to SEQ ID NO: 200.

Embodiment 4. The AAV vector genome of Embodiment 1 or 2, wherein the eye-specific promoter is a human rhodopsin (hRho) promoter comprising or consisting of a sequence having at least 90%, at least 95%, at least 96%, at least 97%, at least 98%, at least 99%, or 100% identity to SEQ ID NO: 209.

Embodiment 5. The AAV vector genome of Embodiment 1 or 2, wherein the eye-specific promoter is a human rhodopsin kinase (hGRK1) promoter comprising or consisting of a sequence having at least 90%, at least 95%, at least 96%, at least 97%, at least 98%, at least 99%, or 100% identity to SEQ ID NO: 188.

Embodiment 6. The AAV vector genome of Embodiment 1 or 2, wherein the eye-specific promoter is a human rhodopsin kinase (hGRK1) promoter, a human rhodopsin (hRho) promoter, a human rod cGMP phosphodiesterase β-subunit (βPDE) promoter, a human interphotoreceptor retinoid-binding protein (IRBP) promoter, a human red/green opsin promoter, a human blue opsin (HB) promoter, a human vitelliform macular dystrophy-2 (VMD2), or a human RPE65 promoter.

Embodiment 7. An AAV vector genome comprising, in 5′ to 3′ orientation:
(a) a 5′ AAV inverted terminal repeat;
(b) a human rhodopsin (hRho) promoter;
(c) a 5′ portion of a transgene;
(d) a splice donor (SD) site;
(e) a recombination site;
(f) a polynucleotide encoding a recombinase;
(g) a poly-A site; and
(h) a 3′ AAV inverted terminal repeat.

Embodiment 8. The AAV vector genome of Embodiment 7, where the human rhodopsin (hRho) promoter comprises or consists of a sequence having at least 90%, at least 95%, at least 96%, at least 97%, at least 98%, at least 99%, or 100% identity to SEQ ID NO: 209.

Embodiment 9. The AAV vector genome of Embodiment 7 or 8, comprising an enhancer between the 5′ AAV inverted terminal repeat and the human rhodopsin (hRho) promoter.

Embodiment 10. The AAV vector genome of any one of Embodiments 1-9, comprising a CBA-MVM hybrid sequence between the eye-specific promoter and the 5′ portion of the transgene.

Embodiment 11. The AAV vector genome of Embodiment 10, wherein the CBA-MVM hybrid sequence comprises or consists of a sequence having at least 90%, at least 95%, at least 96%, at least 97%, at least 98%, at least 99%, or 100% identity to SEQ ID NO: 210.

Embodiment 12. An AAV vector genome comprising, in 5′ to 3′ orientation:
(a) a 5′ AAV inverted terminal repeat;
(b) a CMV enhancer;
(c) a chicken beta-actin (CBA) promoter;
(d) a 5′ portion of a transgene;
(e) a splice donor (SD) site;
(f) a recombination site;
(g) a polynucleotide encoding a recombinase;
(h) a poly-A site; and
(i) a 3′ AAV inverted terminal repeat.

Embodiment 13. The AAV vector genome of Embodiment 12, wherein the CMV enhancer comprises or consists of a sequence having at least 90%, at least 95%, at least 96%, at least 97%, at least 98%, at least 99%, or 100% identity to SEQ ID NO: 218.

Embodiment 14. The AAV vector genome of Embodiment 12 or 13, wherein the CBA promoter comprises or consists of a sequence having at least 90%, at least 95%, at least 96%, at least 97%, at least 98%, at least 99%, or 100% identity to SEQ ID NO: 219.

Embodiment 15. The AAV vector genome of any one of Embodiments 12-14, comprising a sequence having at least 90%, at least 95%, at least 96%, at least 97%, at least 98%, at least 99%, or 100% identity to SEQ ID NO: 211 between the 5′ AAV inverted terminal repeat and the 5′ portion of the transgene.

Embodiment 16. The AAV vector genome of any one of Embodiments 1-15, wherein the 5′ portion of the transgene is at least 0.5 kb, at least 1 kb, at least 1.5 kb, at least 2 kb, or at least 2.5 kb in length.

Embodiment 17. The AAV vector genome of any one of Embodiments 1-16, wherein the transgene encodes an ATP-binding cassette, sub-family A, member 4 (ABCA4) protein.

Embodiment 18. The AAV vector genome of Embodiment 17, wherein the ABCA4 protein comprises or consists of a sequence having at least 90%, at least 95%, at least 96%, at least 97%, at least 98%, at least 99%, or 100% identity to SEQ ID NO: 190.

Embodiment 19. The AAV vector genome of Embodiments 17 or 18, wherein the 5′ portion of the transgene comprises or consists of a sequence having at least 90%, at least 95%, at least 96%, at least 97%, at least 98%, at least 99%, or 100% identity to SEQ ID NO: 212.

Embodiment 20. The AAV vector genome of Embodiments 19, wherein the 5′ portion of the transgene comprises one or more of the following nucleotides: (i) a nucleotide “G” at the position corresponding to nucleotide 813 of SEQ ID NO: 212; (ii) a nucleotide “A” at the position corresponding to nucleotide 1311 of SEQ ID NO: 212; (iii) a nucleotide “A” at the position corresponding to nucleotide 2274 of SEQ ID NO: 212; (iv) a nucleotide “C” at the position corresponding to nucleotide 2277 of SEQ ID NO: 212; (v) a nucleotide “T” at the position corresponding to nucleotide 2278 of SEQ ID NO: 212; and (vi) a nucleotide “T” at the position corresponding to nucleotide 2568 of SEQ ID NO: 212.

Embodiment 21. The AAV vector genome of Embodiment 19 or 20, wherein the 5′ portion of the transgene comprises all of the nucleotides (i)-(vi).

Embodiment 22. The AAV vector genome of any one of Embodiments 19-21, wherein the 5′ portion of the transgene encodes a polypeptide sequence comprising amino acids 1-884 of human ABCA4 protein (SEQ ID NO: 190).

Embodiment 23. The AAV vector genome of any one of Embodiments 1-22, wherein the splice donor (SD) site comprises or consists of a sequence having at least 90%, at least 95%, at least 96%, at least 97%, at least 98%, at least 99%, or 100% identity to SEQ ID NO: 186.

Embodiment 24. The AAV vector genome of any one of Embodiments 1-23, wherein the recombination site is a Lox71 site comprising or consisting of SEQ ID NO: 178, or a sequence having at most 1, at most 2, or at most 3 nucleotides mutations (deletion, insertion, and/or substitution) thereto.

Embodiment 25. The AAV vector genome of any one of Embodiments 1-24, wherein expression of the recombinase is operably linked to the promoter.

Embodiment 26. The AAV vector genome of any one of Embodiments 1-25, wherein the recombinase is a Cre recombinase.

Embodiment 27. The AAV vector genome of Embodiment 26, wherein the Cre recombinase comprises a sequence having at least 90%, at least 95%, at least 96%, at least 97%, at least 98%, at least 99%, or 100% identity to SEQ ID NO: 164.

Embodiment 28. The AAV vector genome of Embodiment 26 or 27, wherein the polynucleotide encoding the recombinase comprises an intron comprising or consisting of a sequence having at least 90%, at least 95%, at least 96%, at least 97%, at least 98%, at least 99%, or 100% identity to SEQ ID NO: 214.

Embodiment 29. The AAV vector genome of any one of Embodiments 26-28, wherein the polynucleotide encoding the recombinase comprises a sequence having at least 90%, at least 95%, at least 96%, at least 97%, at least 98%, at least 99%, or 100% identity to SEQ ID NO: 213.

Embodiment 30. The AAV vector genome of any one of Embodiments 1-29, wherein the recombinase comprises a N-terminal nuclear localization sequence (NLS) comprising or consisting of SEQ ID NO: 165.

Embodiment 31. The AAV vector genome of any one of Embodiments 1-30, wherein the AAV vector genome comprises a polynucleotide encoding an internal cleavage polypeptide located between the recombination site and the polynucleotide encoding the recombinase, and wherein the 5′ portion of the transgene, the polynucleotide encoding the internal cleavage polypeptide, and the polynucleotide encoding the recombinase are in the same reading frame.

Embodiment 32. The AAV vector genome of Embodiment 31, wherein the internal cleavage polypeptide is a self-cleaving peptide selected from the group consisting of T2A, P2A, E2A and F2A, optionally wherein the self-cleaving peptide is a P2A peptide comprising or consisting of SEQ ID NO: 181.

Embodiment 33. The AAV vector genome of any one of Embodiments 1-32, wherein the poly-A site comprises or consists of SEQ ID NO: 189, or a sequence having at most 1, at most 2, at most 3, at most 4, at most 5, or at most 6 nucleotides mutations (deletion, insertion, and/or substitution) thereto.

Embodiment 34. The AAV vector genome of any one of Embodiments 1-33, wherein the AAV vector genome comprises an internal ribosome entry site (IRES) located between the recombination site and the polynucleotide encoding the recombinase, and wherein the IRES is operably linked to the polynucleotide encoding the recombinase.

Embodiment 35. An AAV vector genome comprising, in 5′ to 3′ orientation:
(a) a 5′ AAV inverted terminal repeat;
(b) (b-1) a Lox JTZ17 recombination site comprising or consisting of SEQ ID NO: 217, or a sequence having at most 1, at most 2, or at most 3 nucleotides mutations (deletion, insertion, and/or substitution) thereto; or (b-2) a Lox 66 recombination site comprising or consisting of SEQ ID NO: 179, or a sequence having at most 1, at most 2, or at most 3 nucleotides mutations (deletion, insertion, and/or substitution) thereto;
(c) a splice acceptor (SA) site;
(d) a 3′ portion of a transgene;
(e) a poly-A site; and
(f) a 3′ AAV inverted terminal repeat.

Embodiment 36. The AAV vector genome of Embodiment 35, wherein the splice acceptor (SA) site comprises or consists of a sequence having at least 90%, at least 95%, at least 96%, at least 97%, at least 98%, at least 99%, or 100% identity to SEQ ID NO: 187.

Embodiment 37. The AAV vector genome of Embodiment 35 or 36, wherein the transgene encodes an ABCA4 protein.

Embodiment 38. The AAV vector genome of Embodiment 37, wherein the 3′ portion of the transgene comprises or consists of a sequence having at least 90%, at least 95%, at least 96%, at least 97%, at least 98%, at least 99%, or 100% identity to SEQ ID NO: 193.

Embodiment 39. The AAV vector genome of any one of Embodiments 35-38, wherein the poly-A site comprises or consists of SEQ ID NO: 189, or a sequence having at most 1, at most 2, at most 3, at most 4, at most 5, or at most 6 nucleotides mutations (deletion, insertion, and/or substitution) thereto.

Embodiment 40. The AAV vector genome of any one of Embodiments 1-39, wherein the 5′ AAV inverted terminal repeat comprises or consists of a sequence having at least 90%, at least 95%, at least 96%, at least 97%, at least 98%, at least 99%, or 100% identity to SEQ ID NO: 215.

Embodiment 41. The AAV vector genome of any one of Embodiments 1-40, wherein the 3′ AAV inverted terminal repeat comprises or consists of a sequence having at least 90%, at least 95%, at least 96%, at least 97%, at least 98%, at least 99%, or 100% identity to SEQ ID NO: 216.

Embodiment 42. An AAV viral particle, comprising
(i) an AAV capsid, wherein the AAV capsid comprises an AAV capsid protein; and
(ii) the AAV vector genome of any one of Embodiments 1-41.

Embodiment 43. The AAV viral particle of Embodiment 42, wherein the AAV capsid protein comprises or consists of a sequence having at least 90%, at least 95%, at least 96%, at least 97%, at least 98%, at least 99%, or 100% identity to a sequence selected from SEQ ID NOs: 1-3, 67, 71, 196, 205 and 206.

Embodiment 44. The AAV viral particle of Embodiment 42, wherein the AAV capsid protein comprises or consists of a sequence having at least 90%, at least 95%, at least 96%, at least 97%, at least 98%, at least 99%, or 100% identity to SEQ ID NO: 3.

Embodiment 45. The AAV viral particle of Embodiment 42, wherein the AAV capsid protein comprises or consists of a sequence having at least 90%, at least 95%, at least 96%, at least 97%, at least 98%, at least 99%, or 100% identity to SEQ ID NO: 67.

Embodiment 46. A pharmaceutical composition comprising the AAV vector genome of any one of Embodiments 1-41 or the AAV viral particle of any one of Embodiments 42-45.

Embodiment 47. A pharmaceutical composition comprising
(i) a first AAV viral particle comprising the AAV vector genome of any one of Embodiments 1-34 and 40-41; and
(ii) a second AAV viral particle comprising the AAV vector genome of any one of Embodiments 35-41.

Embodiment 48. A pharmaceutical composition comprising
(i) a first AAV viral particle comprising the AAV vector genome of any one of Embodiments 1-34 and 40-41; and
(ii) a second AAV viral particle comprising an AAV vector genome comprising, in 5′ to 3′ orientation:
   (a) a 5′ AAV inverted terminal repeat;
   (b) a recombination site;
   (c) a splice acceptor (SA) site;
   (d) a 3′ portion of the transgene;
   (e) a poly-A site; and
   (f) a 3′ AAV inverted terminal repeat.

Embodiment 49. A method of expressing a protein encoded by a transgene in a cell, comprising:
(1) transducing the cell with a first AAV viral particle comprising the AAV vector genome of any one of Embodiments 1-34 and 40-41; and
(2) transducing the cell with a second AAV viral particle comprising the AAV vector genome of any one of Embodiments 35-41.

Embodiment 50. A method of expressing a protein encoded by a transgene in a cell, comprising:
(1) transducing the cell with a first AAV viral particle comprising the AAV vector genome of any one of Embodiments 1-34 and 40-41; and
(2) transducing the cell with a second AAV viral particle comprising an AAV vector genome comprising, in 5′ to 3′ orientation:
   (a) a 5′ AAV inverted terminal repeat;
   (b) a recombination site;
   (c) a splice acceptor (SA) site;
   (d) a 3′ portion of the transgene;
   (e) a poly-A site; and
   (f) a 3′ AAV inverted terminal repeat.

Embodiment 51. A method of treating a subject having a disease or disorder, comprising:
(1) administering to the subject a first AAV viral particle comprising the AAV vector genome of any one of Embodiments 1-34 and 40-41;
(2) administering to the subject a second AAV viral particle comprising the AAV vector genome of any one of Embodiments 35-41.

Embodiment 52. A method of treating a subject having a disease or disorder, comprising:
(1) administering to the subject a first AAV viral particle comprising the AAV vector genome of any one of Embodiments 1-34 and 40-41;
(2) administering to the subject a second AAV viral particle comprising an AAV vector genome comprising, in 5′ to 3′ orientation:
   (a) a 5′ AAV inverted terminal repeat;
   (b) a recombination site;
   (c) a splice acceptor (SA) site;
   (d) a 3′ portion of the transgene;
   (e) a poly-A site; and
   (f) a 3′ AAV inverted terminal repeat.

Embodiment 53. The pharmaceutical composition of Embodiment 47 or 48 or the method of any one of Embodiments 49-52, wherein the recombination site in the AAV vector genome of the first AAV viral particle and the recombination site in the AAV vector genome of the second AAV viral particle prevent backwards recombination.

Embodiment 54. The pharmaceutical composition of any of Embodiments 47-48 and 53 or the method of any one of Embodiments 49-53, wherein the AAV viral particle comprises an AAV capsid protein comprising or consisting of a sequence having at least 90%, at least 95%, at least 96%, at least 97%, at least 98%, at least 99%, or 100% identity to a sequence selected from SEQ ID NOs: 1-3, 67, 71, 196, 205 and 206.

Embodiment 55. The pharmaceutical composition or the method of Embodiment 54, wherein the AAV capsid protein comprises or consists of a sequence having at least 90%, at least 95%, at least 96%, at least 97%, at least 98%, at least 99%, or 100% identity to SEQ ID NO: 3.

Embodiment 56. The pharmaceutical composition or the method of Embodiment 54, wherein the AAV capsid protein comprises or consists of a sequence having at least 90%, at least 95%, at least 96%, at least 97%, at least 98%, at least 99%, or 100% identity to SEQ ID NO: 67.

Embodiment 57. The method of any one of Embodiments 49-56, wherein administration of the first AAV viral particle and the second AAV viral particle leads to recombination of the first AAV vector genome and the second AAV vector genome via the recombination site in the AAV vector genome of the first AAV viral particle and the recombination site in the second AAV vector genome of the second AAV viral particle.

Embodiment 58. The method of any one of Embodiments 49-57, wherein administration of the first AAV viral particle and the second AAV viral particle leads to expression of the full transgene.

Embodiment 59. The method of any one of Embodiments 51-58, wherein the disease or disorder is Stargardt disease.

Embodiment 60. The method of any one of Embodiments 49-59, wherein the protein is an ABCA4 protein, or wherein the disease or disorder is caused by an ABCA4 gene defect.

Embodiment 61. The method of Embodiment 60, wherein the ABCA4 gene defect results in one or more conditions selected from the group consisting of reduced expression of ABCA4 protein, eliminated expression of ABCA4 protein, expression of a mutated ABCA4 protein, and reduced function of ABCA4 protein.

Embodiment 62. The method of any one of Embodiments 49-61, wherein the first AAV viral particle and the second AAV viral particle are co-administered.

Embodiment 63. The method of any one of Embodiments 49-61, wherein the first AAV viral particle is administered before, or after, the second AAV viral particle.

Embodiment 64. The method of any one of Embodiments 51-63, wherein the viral particles are administered by intravitreal, para-retinal, or subretinal injection; optionally by subretinal injection.

Embodiment 65. The method of any one of Embodiments 49-64, wherein no stable expression of the recombinase is detected in the transduced cell or in the subject.

Embodiment 66. A transduced cell comprising
(i) a defective genomic copy of an endogenous gene;
(ii) a recombinant nucleic acid comprising, in 5′ to 3′ orientation:
   (a) a 5′ AAV inverted terminal repeat;
   (b) an interphotoreceptor retinoid-binding protein (IRBP) enhancer;
   (c) an eye-specific promoter;
   (d) a 5′ portion of a transgene, wherein the transgene is a working copy of the defective endogenous gene;
   (e) a splice donor (SD) site;
   (f) a recombination site;
   (g) a splice acceptor (SA) site;
   (h) a 3′ portion of the transgene;
   (i) a poly-A site; and
   (j) a 3′ AAV inverted terminal repeat.

Embodiment 67. A transduced cell comprising
(i) a defective genomic copy of an endogenous gene;
(ii) a recombinant nucleic acid comprising, in 5′ to 3′ orientation:
   (a) a 5′ AAV inverted terminal repeat;
   (b) optionally, an enhancer;
   (c) a human rhodopsin (hRho) promoter;
   (d) a 5′ portion of a transgene, wherein the transgene is a working copy of the defective endogenous gene;
   (e) a splice donor (SD) site;
   (f) a recombination site;
   (g) a splice acceptor (SA) site;
   (h) a 3′ portion of the transgene;
   (i) a poly-A site; and
   (j) a 3′ AAV inverted terminal repeat.

Embodiment 68. A transduced cell comprising
(i) a defective genomic copy of an endogenous gene;
(ii) a recombinant nucleic acid comprising, in 5′ to 3′ orientation:
   (a) a 5′ AAV inverted terminal repeat;
   (b) a CMV enhancer;
   (c) a chicken beta-actin (CBA) promoter;
   (d) a 5′ portion of a transgene, wherein the transgene is a working copy of the defective endogenous gene;
   (e) a splice donor (SD) site;
   (f) a recombination site;
   (g) a splice acceptor (SA) site;
   (h) a 3′ portion of the transgene;
   (i) a poly-A site; and
   (j) a 3′ AAV inverted terminal repeat.

Embodiment 69. The transduced cell of any one of Embodiments 66-68, wherein the defective endogenous gene is an ABCA4 gene and the transgene encodes an ABCA4 protein.

Embodiment 70. The transduced cell of any one of Embodiments 66-69, wherein the transduced cell or the recombinant nucleic acid comprises a nucleic acid sequence comprising, in 5′ to 3′ orientation: a 5′ AAV inverted terminal repeat, a recombination site, a polynucleotide encoding a recombinase, and a 3′ AAV inverted terminal repeat; wherein the nucleic acid sequence lacks a promoter.

Embodiment 71. The transduced cell of Embodiment 70, wherein the nucleic acid sequence is located downstream of the 3′ end of the (ii)(j) 3′ AAV inverted terminal repeat.

Embodiment 72. The transduced cell of any one of Embodiments 66-71, wherein the cell is an ex vivo cell.

Embodiment 73. The transduced cell of any one of Embodiments 66-72, wherein the cell is a photoreceptor cell.

Embodiment 74. A nucleic acid comprising a 5′ portion of an ABCA4 gene, wherein the 5′ portion of the ABCA4 gene comprises one or more of: (i) a nucleotide “G” at the position corresponding to nucleotide 813 of SEQ ID NO: 212; (ii) a nucleotide “A” at the position corresponding to nucleotide 1311 of SEQ ID NO: 212; (iii) a nucleotide “A” at the position corresponding to nucleotide 2274 of SEQ ID NO: 212; (iv) a nucleotide “C” at the position corresponding to nucleotide 2277 of SEQ ID NO: 212; (v) a nucleotide “T” at the position corresponding to nucleotide 2278 of SEQ ID NO: 212; and (vi) a nucleotide “T” at the position corresponding to nucleotide 2568 of SEQ ID NO: 212.

Embodiment 75. The nucleic acid of Embodiment 74, comprising 2 or more, 3 or more, 4 or more, 5 or more, or all 6 of, the nucleotides (i)-(vi).

Embodiment 76. The nucleic acid of Embodiment 74 or 75, wherein the 5′ portion of the ABCA4 gene is at least 1 kb, at least 1.5 kb, at least 2 kb, or at least 2.5 kb in length.

Embodiment 77. The nucleic acid of any one of Embodiments 74-76, wherein the 5′ portion of the ABCA4 gene comprises or consists of a sequence having at least 90%, at least 95%, at least 96%, at least 97%, at least 98%, at least 99%, or 100% identity to SEQ ID NO: 212.

Embodiment 78. The nucleic acid of Embodiment 77, wherein the 5′ portion of the transgene encodes a polypeptide sequence comprising amino acids 1-884 of human ABCA4 protein (SEQ ID NO: 190).

Embodiment 79. An AAV vector genome comprising the nucleic acid of any one of Embodiments 74-78.

Embodiment 80. An AAV viral particle comprising
(i) an AAV capsid, wherein the AAV capsid comprises an AAV capsid protein; and (ii) the AAV vector genome of Embodiment 79.

Embodiment 81. A pharmaceutical composition comprising the AAV vector genome of Embodiment 79 or the AAV viral particle of Embodiment 80.

Embodiment 82. A method of treating a subject having a disease or disorder, comprising administering to the subject the AAV viral particle of Embodiment 80, optionally wherein the disease or disorder is Stargardt disease.

Embodiment 83. A transduced cell comprising the nucleic acid of any one of Embodiments 74-78.

### EXAMPLES

### Example 1: Construction of Plasmids with Bipartite Design

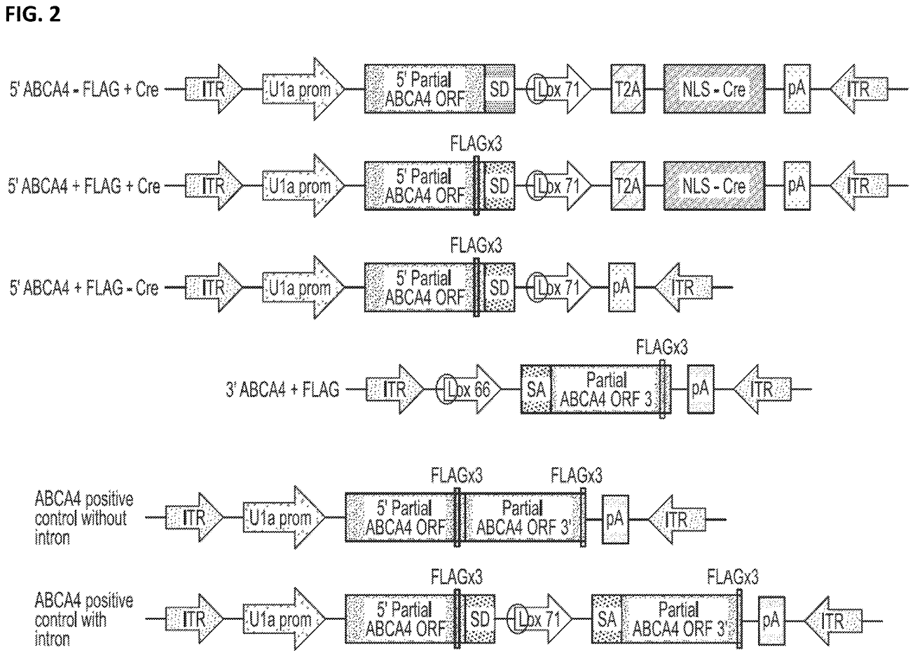

Several plasmids were generated to test the expression of full length ABCA4 gene in cells using the bipartite vector system (FIG. 2).

A “5′ ABCA4 −FLAG+Cre” plasmid was constructed, which comprises, from 5′ to 3′: 5′ AAV inverted terminal repeat, a U1a promoter, a 5′ portion of the ABCA4 gene ORF, a splice donor (SD) site, a Lox71 site, the nucleic acid sequence encoding T2A peptide, the nucleic acid sequence encoding Cre recombinase with nuclear localization sequence (NLS), a poly-A site, and 3′ AAV inverted terminal repeat.

A “5′ ABCA4+FLAG+Cre” plasmid was generated by incorporating a 3×FLAG tag coding sequence into the 5′ portion of the ABCA4 gene in the “5′ ABCA4 −FLAG+Cre” plasmid.

A “5′ ABCA4+FLAG−Cre” plasmid was generated by removing the Cre recombinase coding sequence in the “5′ ABCA4+FLAG+Cre” plasmid.

A “3′ ABCA4+FLAG” plasmid was constructed, which comprises, from 5′ to 3′: 5′ AAV inverted terminal repeat, a Lox66 site, a splice acceptor (SA) site, 3′ portion of the ABCA4 gene ORF incorporated with a 3×FLAG tag coding sequence, a poly-A site, and a 3′ AAV inverted terminal repeat.

Two positive control plasmid constructs comprising the full length ABCA4 gene were also generated (FIG. 2). One positive control construct has no intron region, whereas the other positive control comprises an intron region which comprises a splice donor (SD) site, a Lox71 site, and a splice acceptor (SA) site, mimicking the construct generated by the recombination between “5′ ABCA4+FLAG+Cre” and “3′ ABCA4+FLAG” plasmid. Both positive control constructs comprise 3×FLAG tag coding sequences in both the 5′ portion of the ABCA4 gene and the 3′ portion of the ABCA4 gene.

### Example 2: Expression of Full-Length Protein Using a Bipartite Vector System

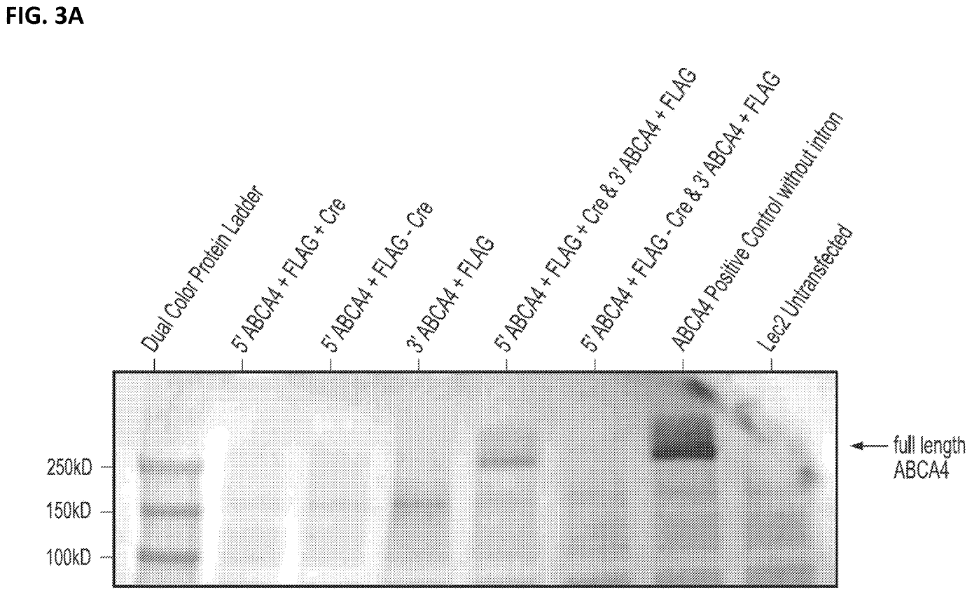

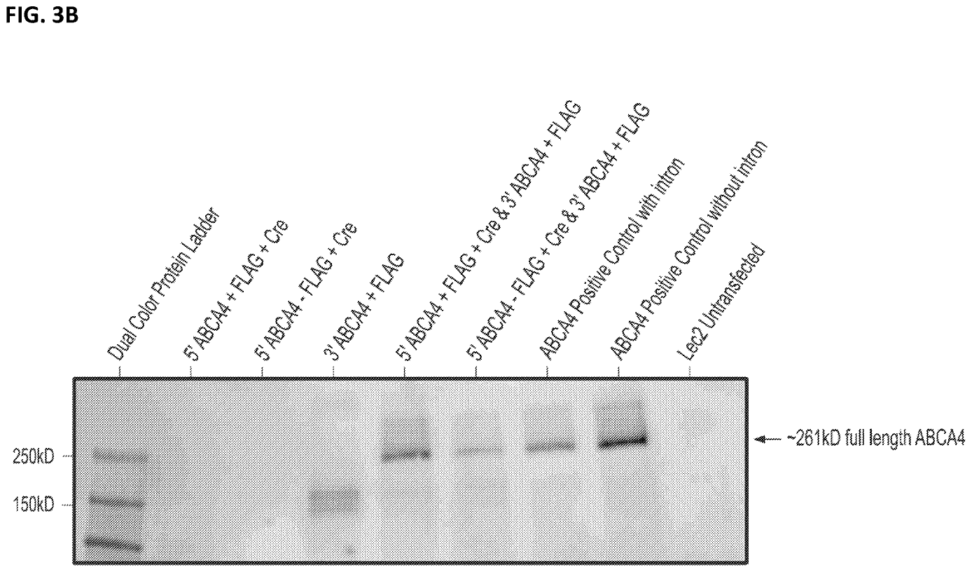

Cell studies were performed using HEK293 cells transfected with the indicated plasmids described in Example 1 and FIG. 2, and the expression of full length ABCA4 protein was examined using Western blotting. Full length ABCA4 protein (with attached FLAG tags) has an estimated molecular weight of ~261 kD. As shown in FIG. 3A, among the cells that were transfected with the plasmids other than the positive control, expression of full length ABCA4 protein was only observed when both “5′ ABCA4+FLAG+Cre” and “3′ ABCA4+FLAG” plasmids were applied to the cells. On the other hand, there was no expression of full length ABCA4 protein in cells transfected with the dual vector system without Cre recombinase (i.e., the “5′ ABCA4+FLAG−Cre” and “3′ ABCA4+FLAG” pair). Similarly, in a repeat experiment (FIG. 3B), among the cell groups that were transfected with the indicated plasmids other than the positive controls, only two experimental groups showed expression of full length ABCA4. One group was transfected with “5′ ABCA4+FLAG+Cre” and “3′ ABCA4+FLAG” plasmids, and the other group was transfected with “5′ ABCA4 −FLAG+Cre” and “3′ ABCA4+FLAG” plasmids. These results indicate that the dual vector system can carry out efficient Cre-lox mediated recombination between the two plasmids which in turn results in expression of the full-length ABCA4 protein.

### Example 3: Expression of Cre Recombinase Expression is Significantly Decreased after Recombination of AAV Vector Genomes

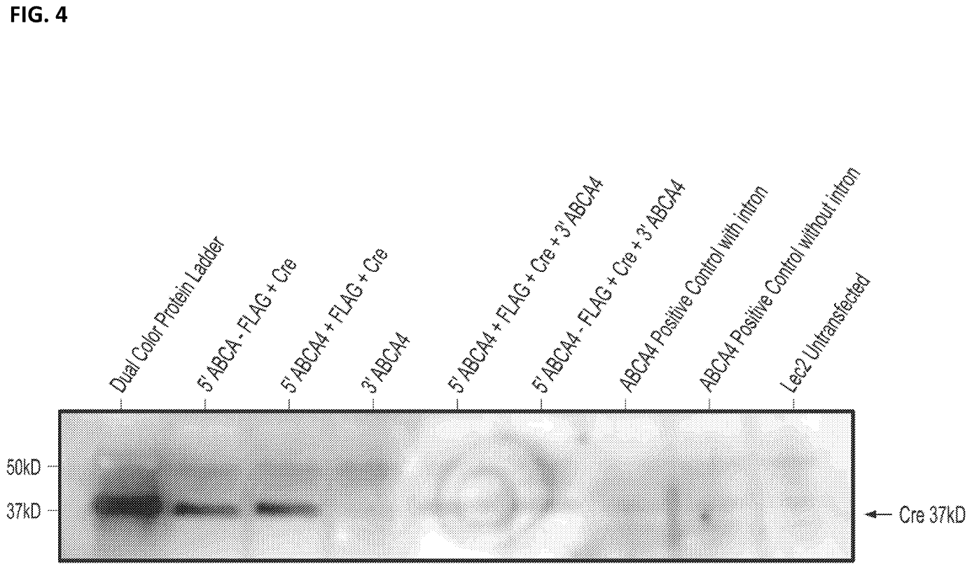

The expression level of Cre recombinase in cells containing the plasmids was analyzed (FIG. 4). HEK293 cells were transfected with various plasmids and controls as described in Example 2, and 48 hours after transfection, cell samples were processed using sample buffer containing 4M urea, and stable expression of Cre recombinase was quantified by Western blotting using 3% milk as blocking agent, rabbit anti-Cre mAb (Cell signaling technology #15036) at 1:10,000 dilution as primary antibody, and goat anti-rabbit-HRP (azure) at 1:10,000 dilution as secondary antibody. Auto-cleaved Cre recombinase has a molecular weight of ~37 kD. As expected, transfection of either “5′ABCA4 −FLAG+Cre” or “5′ABCA4+FLAG+Cre” plasmid resulted in clear expression of Cre recombinase in HEK293 cells. Remarkably, there was much lower expression of Cre recombinase in cells co-transfected with the second plasmid “3′ABCA4+FLAG”, suggesting that the dual vector system allows efficient recombination between the first and second plasmids, which in turn leads to generation of a recombinant nucleic acid containing the Cre recombinase ORF without a promoter, and therefore the expression of Cre recombinase was significantly decreased.

### Example 4: Cre-Mediated Recombination of the Bipartite Vector System Achieves Expression of Full-length ABCA4 mRNA

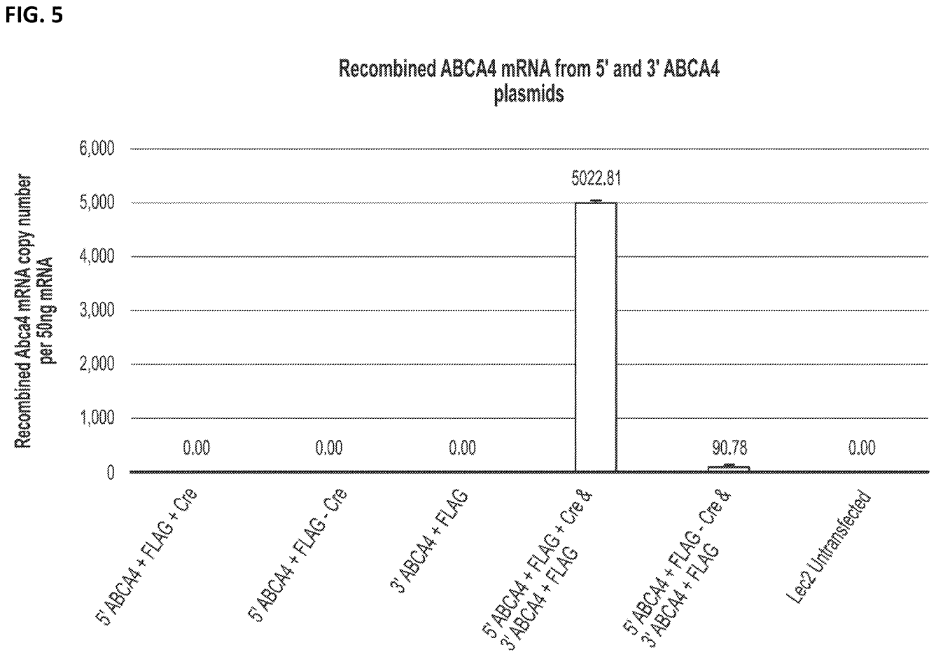

We quantified the expression levels of full-length ABCA4 mRNA in cells transfected with various plasmids. As shown in FIG. 5, cells transfected with a dual vector system encoding Cre recombinase (“5′ABCA4+FLAG+Cre” and “3′ABCA4+FLAG”) showed about 55 times higher expression level of full-length ABCA4 mRNA compared to cells transfected with a corresponding dual vector system not encoding Cre recombinase (“5′ABCA4+FLAG−Cre” and “3′ABCA4+FLAG”). These results show that Cre recombinase effectively mediates the recombination between the two starting vectors shortly after they are transfected into the cells and drives expression of the full-length mRNA.

### Example 5: Delivery of Bipartite Vector System Via Dual AAV Viral Particles Results in Expression of Full-length ABCA4 mRNA

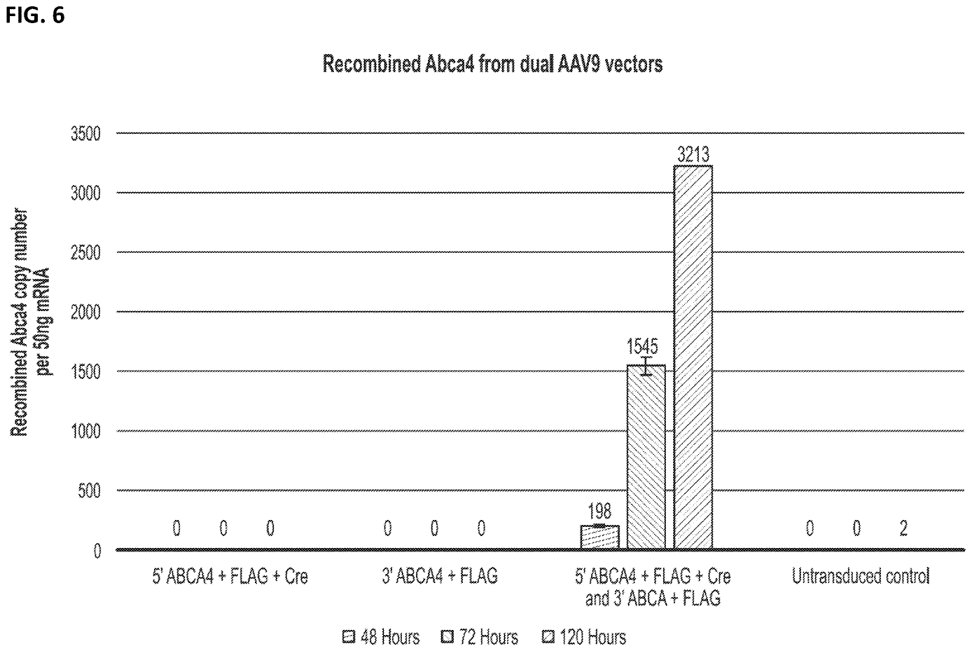

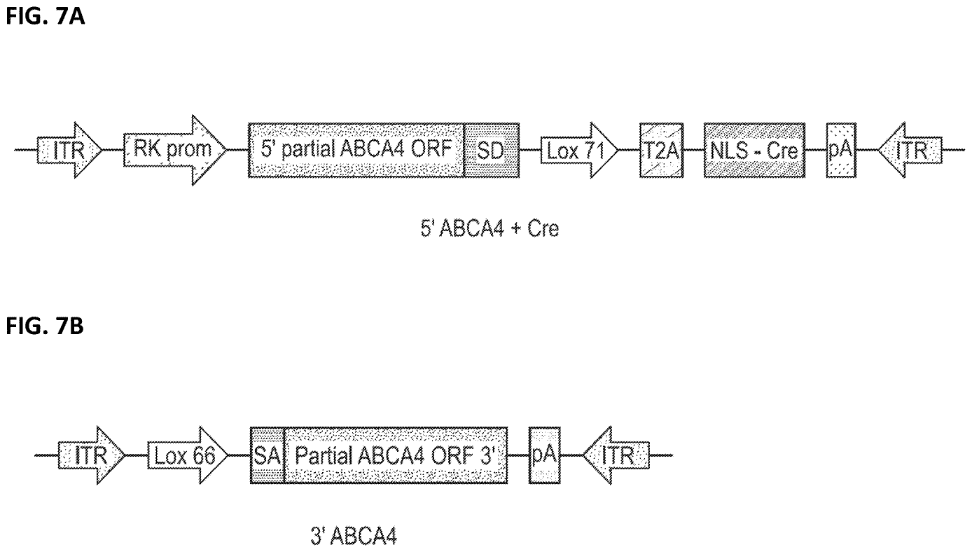

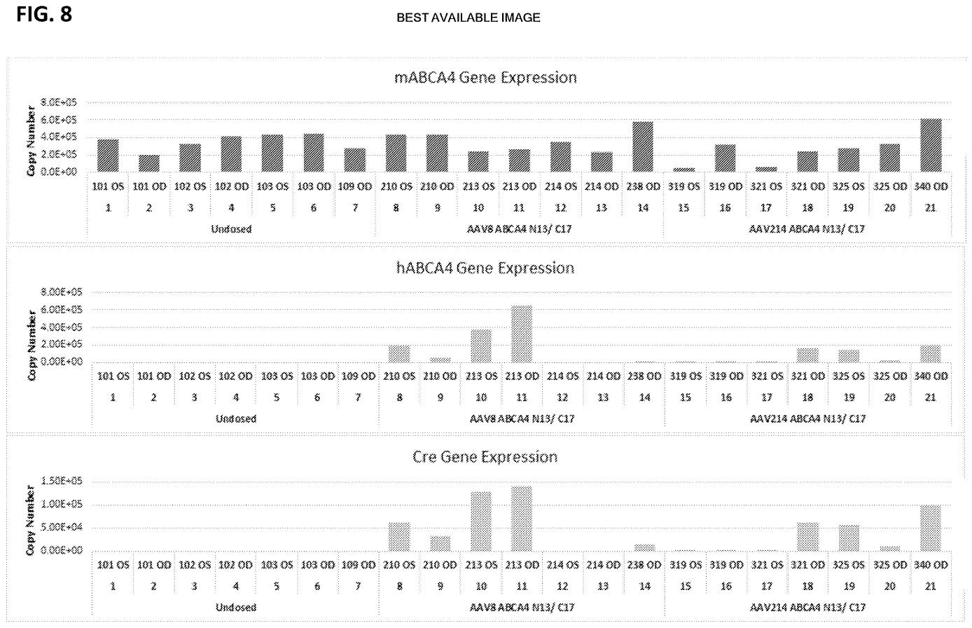

We tested whether the bipartite vector system could be delivered by AAV viral particles to express full-length ABCA4 mRNA within the cells. Two types of AAV9 viral particles were prepared using the expression cassettes described in Example 1. One type of AAV9 viral particles comprise an AAV vector genome encoding “5′ABCA4+FLAG+Cre”, and the other type of AAV9 viral particles comprise an AAV vector genome encoding “3′ABCA4+FLAG”. As shown in FIG. 6, ABCA4 negative cells were transduced with the indicated AAV9 viral particles, and the expression level of the full-length ABCA4 mRNA were quantified in each group. Only cells transduced with both types of AAV9 viral particles displayed high expression level of full-length ABCA4 mRNA, and the expression level continued to increase for over 120 hours post-transduction. This result demonstrated that AAV viral particles can effectively deliver the bipartite vector system in the same cell and recombine so that the mRNA payload is properly reconstituted by the system.

### Example 6: The hABCA4 Dual AAV Vector System Achieved Robust In Vivo Expression of Full-length Recombinant RNA Encoding hABCA4 in Wild-Type Mice

We evaluated AAV dual vector system designed for full size human ABCA4 (hABCA4) protein expression in photoreceptors, delivered by subretinal injection into 4-week-old B6129SF1/J mice. Two AAV viral particles, AAV8 and AAV214, were tested, according to the study design of Table 8 below. The animals were administered once on Day 1 of the study.

TABLE 8
Study Design

| | | | Dose | | Dose | | |
|---|---|---|---|---|---|---|---|
| | | Administration | Levels | Titer | Volume | Dosing | Number of |
| Group | Treatment* | Route | (vg/eye) | (vg/mL) | (μL) | Regimen | Animals |
| 1 | Untreated | N/A | N/A | N/A | N/A | Both | 9 |
| | | | | | | eyes | |
| 2 | AAV8.ABCA4N13/C17 | Subretinal | 3.0E+9 | 3.0E+12 | 1.0 | Both | 9 |
| | | | | | | eyes | |
| 3 | AAV214.ABCA4N13/C17 | Subretinal | 3.0E+9 | 3.0E+12 | 1.0 | Both | 9 |
| | | | | | | eyes | |

*N13 depicts hABCA4 N′-part encoding virus based on pA-ABCA13 plasmid; C17 depicts hABCA4 C′-part encoding virus based on pA-ABCA17 plasmid. Vector ratio was 1:1.

On Day 28 (+/−1 day), all animals were euthanized by CO2 asphyxiation followed by cervical dislocation. Both eyes of each animal were harvested, and lens dissection was performed. Each eye sample was placed in RNase/DNase free tube and snap frozen in liquid nitrogen.

Total mouse RNA was isolated with RNeasy Mini Kit (QIAGEN). RNA quantitation was performed on Nanodrop spectrophotometer (THERMOFISHER). Cre-dependent ABCA4 gene expression was assessed by qPCR assay. Seven mouse eyes from each test group (un-dosed, dosed with AAV8 ABCA4 N13/C17 and dosed with AAV214 ABCA4 N13/C17 vectors) were utilized in protein extraction. Mouse eyes were suspended in RIPA lysis buffer supplemented with protease inhibitors and disrupted via FastPrep Bead Beating Grinder and Lysis System (MP BIOMEDICALS) as recommended by manufacturer.

### ABCA4 and Cre Gene Expression Analysis:

As shown in FIG. 8, mouse ABCA4 gene expression was successfully detected in all prepared test samples and varied from 5.5E+4 to 6.2E+5 copies per qPCR reaction, and human ABCA4 gene expression was successfully detected in all dosed mice and varied from 117 copies (0.18% of mABCA4 RNA) to 6.5E+5 copies (245% of mABCA) for 10 ng of total RNA input. Less than 100 copies of hABCA4 were detected in two out of 14 dosed animals (214 OS and 214 OD).

These results confirmed Cre-dependent hABCA4 recombination in dosed mice. No statistically significant difference (p=0.29) in ABCA4 gene expression between AAV8 or AAV214 capsids was observed.

No human Cre gene expression was detected in un-dosed animals. Cre gene expression in dosed mice varied from 593 copies to 1.4E+5 copies for 10 ng of total RNA input. Less than 100 copies of Cre were detected in two out of 14 dosed animals tested (214 OS and 214 OD). Cre gene expression correlated well with hABCA4 gene expression in this study.

### ABCA4 and Cre Protein Expression Analysis:

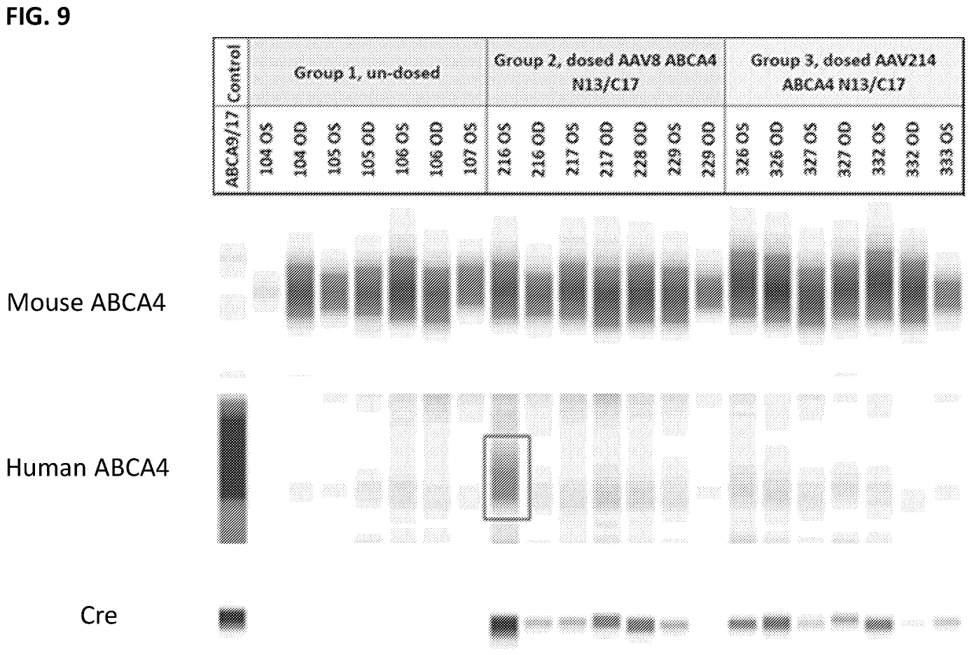

Protein lysates were probed on an automated Western platform and FIG. 9 shows the signals of mouse endogenous ABCA4, human recombinant ABCA4 (FLAG tagged), and Cre recombinase. Endogenous mouse ABCA4 protein was detected in all mouse eye protein lysates. Molecular weight of ABCA4 protein band was 226 kDa on average. Amount of mouse ABCA4 protein varied from 2.8E+4 to 1.9E+5 chemiluminescent units (measured as protein peaks).

Human recombinant ABCA4 signals were detected using rabbit monoclonal anti-FLAG antibodies from NOVUS BIOLOGICALS, MAB8529. Corresponding human ABCA4 protein signal was observed only in the 216 OS mouse eye lysate. This signal was confirmed using manual Western Blot assay.

Cre recombinase protein was not detected in un-dosed mouse eyes. Transgenic Cre recombinase protein was detected in all dosed mouse eye as well as in positive control sample. Molecular weight of detected Cre protein band was 38 kDa on average. The 216 OS mouse eye lysate (which produced detectable transgenic human ABCA4 protein) also had the highest level of detectable transgenic Cre recombinase.

### Conclusion:

Overall, the results in this Proof-of-Concept in vivo study demonstrated that Cre-recombinase is capable of inducing full-size human ABCA4 gene (mRNA) expression after subretinal dosing of partial hABCA4 expression cassettes encapsidated into AAV8 or AAV214 virions. It would be desirable to further optimize regulatory sequences (e.g., in the promoter region) to achieve higher hABCA4 protein expression.

### Example 7: Optimized Regulatory Sequences in Bipartite AAV Systems Provided Increased hABCA4 Protein Expression

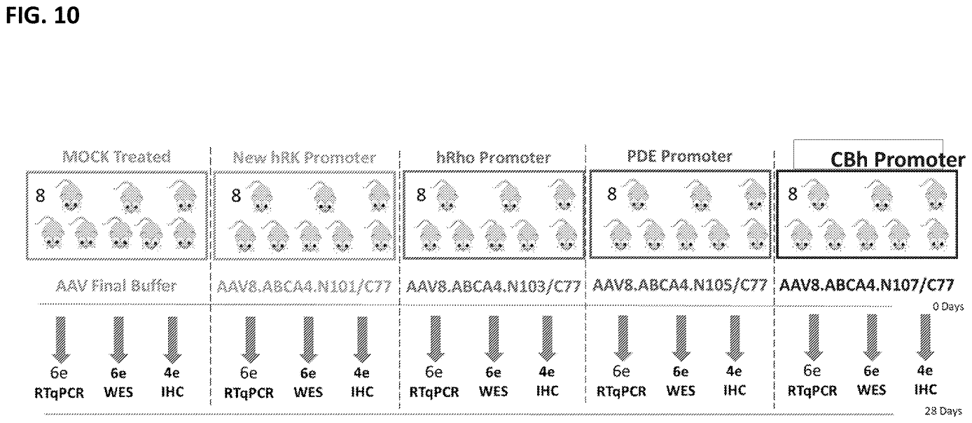

We evaluated different regulatory sequences for expressing hABCA4 protein using the bipartite AAV system. As shown in FIG. 10, four new bipartite AAV systems with new regulatory sequences were tested in ABCA4 (−/−) mice. Each group of bipartite AAV viral vectors were encapsidated in AAV8 and 1 μL of the AAV viral particles were administered subretinally to deliver 3.0E+9 vg to the eyes of eight mice. For each bipartite AAV system, the ratio of the first AAV vector genome to the second AAV vector genome was kept at 1:1. Four weeks after administration, all sixteen eye samples were taken in each group, of which six eyes were subject to RTqPCR, six eyes were subject to Western analysis, and the other four eyes were subject to IHC.

For Western analysis, mouse retinas were suspended in RIPA lysis buffer supplemented with protease inhibitors and disrupted via FASTPREP bead beating grinder and lysis system. Protein lysates were processed and then probed on automated Western platform with anti-HA tag antibodies to confirm recombination event and estimate the amount of transgenic human ABCA4 protein accumulated in mouse eye one-month post-injection.

### Design of Study Groups and Bipartite AAV Systems

Mice in Group 1 serve as the control and were mock treated.

In Group 2 (new hGRK1 promoter), the first AAV vector genome pA-ABCA4.N101 comprises, from 5′ to 3′:
a 5′ AAV ITR according to SEQ ID NO: 215;
an IRBP enhancer according to SEQ ID NO: 208;
an hGRK1 promoter according to SEQ ID NO: 188;
a CBA-MVM hybrid sequence according to SEQ ID NO: 210;
a 5′ portion of hABCA4 gene according to SEQ ID NO: 212;
a splice donor (SD) site according to SEQ ID NO: 186;
a Lox 71 recombination site according to SEQ ID NO: 178;
a P2A (SEQ ID NO: 181) encoding sequence;
a polynucleotide encoding a Cre recombinase (SEQ ID NO: 164) with a nuclear localization signal (SEQ ID NO: 165) and an intron (SEQ ID NO: 214);
an SPA49 poly-A site according to SEQ ID NO: 189; and
a 3′ AAV ITR according to SEQ ID NO: 216.
   pA-ABCA4.N101 was encoded by the vector comprising SEQ ID NO: 220.

In Group 3 (hRho promoter), the first AAV vector genome pA-ABCA4.N103 comprises the same sequence as the pA-ABCA4.N101, except for a hRho promoter (SEQ ID NO: 209) that replaces the hGRK1 promoter sequence in the latter. pA-ABCA4.N103 was encoded by the vector comprising SEQ ID NO: 221.

In Group 4 (βPDE promoter), the first AAV vector genome pA-ABCA4.N105 comprises the same sequence as the pA-ABCA4.N101, except for a βPDE promoter (SEQ ID NO: 200) that replaces the hGRK1 promoter sequence in the latter. pA-ABCA4.N105 was encoded by the vector comprising SEQ ID NO: 222.

In Group 5 (CBh promoter), the first AAV vector genome pA-ABCA4.N107 comprises the same sequence as the pA-ABCA4.N101, except for a CBh sequence (SEQ ID NO: 211; comprising a CMV enhancer, a chicken β-actin promoter, and a CVA exon) that replaces the IRBP enhancer to CBA-MVM hybrid sequence region in the latter. pA-ABCA4.N107 was encoded by the vector comprising SEQ ID NO: 223.

For each of Groups 2-5, the second AAV vector genome pA-ABCA4.C77 comprises, from 5′ to 3′:
a 5′ AAV ITR according to SEQ ID NO: 215;
a Lox JTZ17 recombination site according to SEQ ID NO: 217;
a splice acceptor (SA) site according to SEQ ID NO: 187;
a 3′ portion of hABCA4 gene according to SEQ ID NO: 193 with a C-terminal HA-tag encoding sequence;
an SPA49 poly-A site according to SEQ ID NO: 189; and
a 3′ AAV ITR according to SEQ ID NO: 216.
   pA-ABCA4.C77 was encoded by the vector comprising SEQ ID NO: 224.

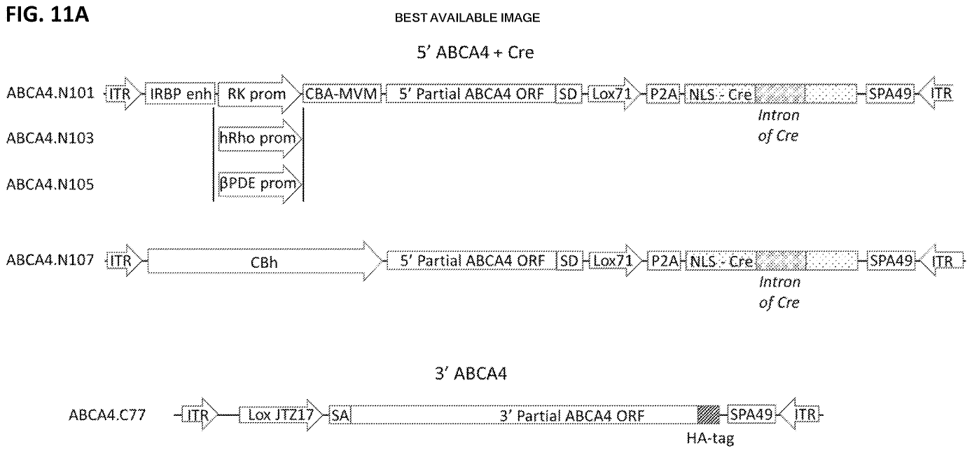

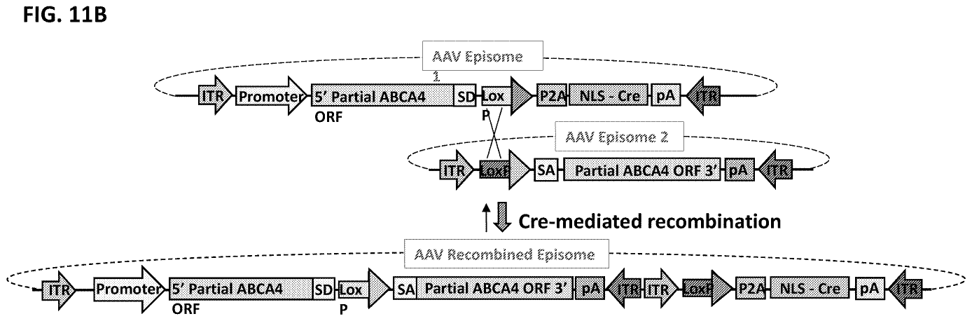

Schematics of all these vectors are shown in FIG. 11A. And Cre-mediated recombination of the first and the second AAV vector genomes is depicted in FIG. 11B.

### Results of the Study

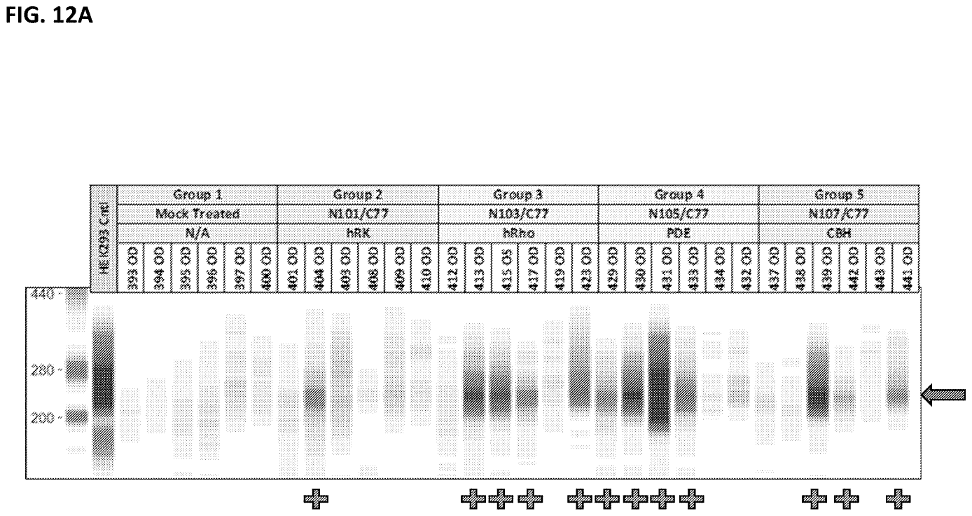

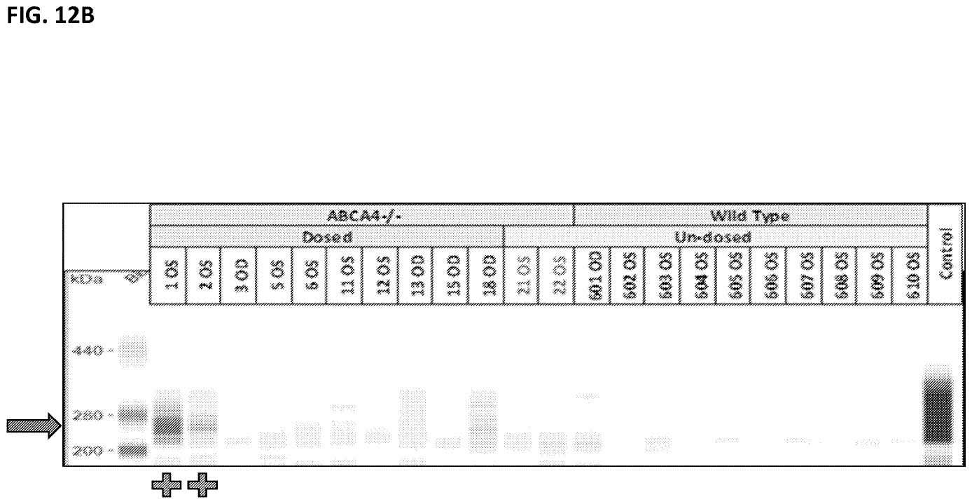

As shown in FIG. 12A, robust expression of full-length hABCA4 protein was detected in most of the mice dosed with the new bipartite AAV systems in Groups 3 & 4 (with a photoreceptor-specific hRho or βPDE promoter) and Group 5 (with a universal CBh promoter). In contrast, the previous promoter design in Example 6 (as shown in FIG. 12B) and Group 2 (both with an hGRK1 promoter) resulted in less protein expression.

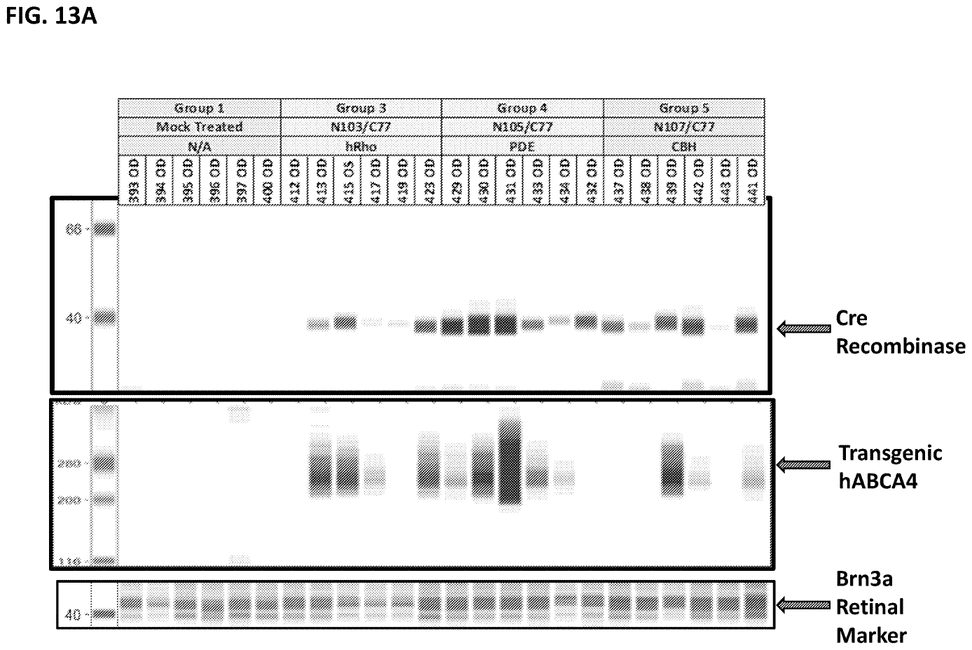

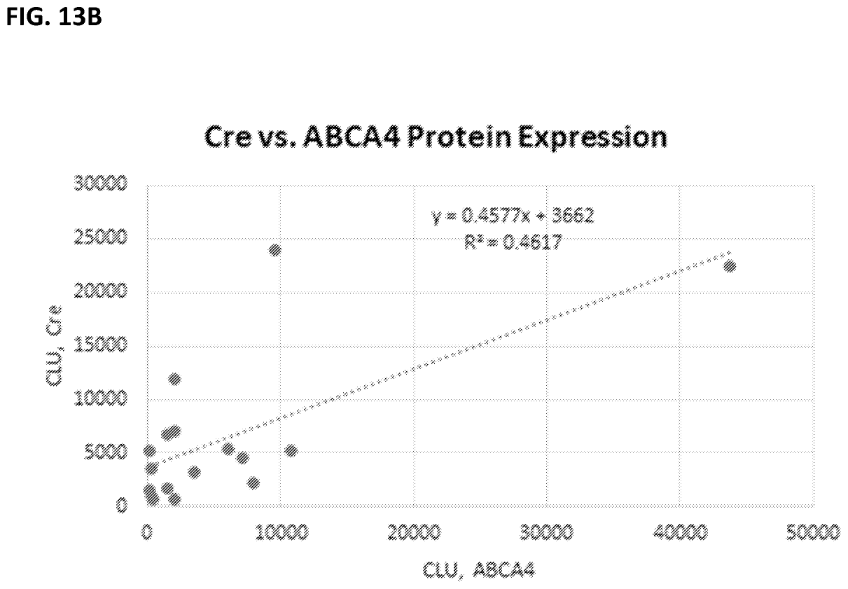

The expression of Cre recombinase was then evaluated in Groups 1 and 3-5. Protein lysate samples were probed with anti-Cre antibodies, anti-Brn3a antibodies and re-tested with anti-HA antibodies. As shown in FIG. 13A, expression of Cre recombinase was detected in many eye samples that showed hABCA4 protein expression. However, there was no strong correlation between Cre and hABCA4 protein expression in this animal study (FIG. 13B), contrary to a strong correlation detected in a prior in-vitro study using HEK293 cells.

In summary, the bipartite AAV systems with new promoters provided more robust expression of hABCA4 proteins in vivo, without strong correlation with Cre recombinase expression. Thus, these bipartite AAV systems have the potential to achieve selective, high expression of target proteins and at the same time lower the expression of the recombinase.

While preferred embodiments of the present disclosure are shown and described herein, it will be obvious to those skilled in the art that such embodiments are provided by way of example only. Numerous variations, changes, and substitutions can be implemented by those skilled in the art without departing from the disclosure. It should be understood that various alternatives to the embodiments of the disclosure described herein may be employed in practicing the disclosure.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not, be taken as an acknowledgment or any form of suggestion that they constitute valid prior art or form part of the common general knowledge in any country in the world.

1.-34. (canceled)

35. An AAV vector genome comprising, in 5′ to 3′ orientation:
(a) a 5′ AAV inverted terminal repeat;
(b) (b-1) a Lox JTZ17 recombination site comprising or consisting of SEQ ID NO: 217, or a sequence having at most 1, at most 2, or at most 3 nucleotides mutations (deletion, insertion, and/or substitution) thereto; or (b-2) a Lox 66 recombination site comprising or consisting of SEQ ID NO: 179, or a sequence having at most 1, at most 2, or at most 3 nucleotides mutations (deletion, insertion, and/or substitution) thereto;
(c) a splice acceptor (SA) site;
(d) a 3′ portion of a transgene;
(e) a poly-A site; and
(f) a 3′ AAV inverted terminal repeat.

36. The AAV vector genome of claim 35, wherein the splice acceptor (SA) site comprises or consists of a sequence having at least 90%, at least 95%, at least 96%, at least 97%, at least 98%, at least 99%, or 100% identity to SEQ ID NO: 187.

37. The AAV vector genome of claim 35, wherein the transgene encodes an ABCA4 protein.

38. The AAV vector genome of claim 37, wherein the 3′ portion of the transgene comprises or consists of a sequence having at least 90%, at least 95%, at least 96%, at least 97%, at least 98%, at least 99%, or 100% identity to SEQ ID NO: 193.

39. The AAV vector genome of claim 35, wherein the poly-A site comprises or consists of SEQ ID NO: 189, or a sequence having at most 1, at most 2, at most 3, at most 4, at most 5, or at most 6 nucleotides mutations (deletion, insertion, and/or substitution) thereto.

40. The AAV vector genome of claim 35, wherein the 5′ AAV inverted terminal repeat comprises or consists of a sequence having at least 90%, at least 95%, at least 96%, at least 97%, at least 98%, at least 99%, or 100% identity to SEQ ID NO: 215.

41. The AAV vector genome of claim 35, wherein the 3′ AAV inverted terminal repeat comprises or consists of a sequence having at least 90%, at least 95%, at least 96%, at least 97%, at least 98%, at least 99%, or 100% identity to SEQ ID NO: 216.

42. An AAV viral particle, comprising
(i) an AAV capsid, wherein the AAV capsid comprises an AAV capsid protein; and (ii) the AAV vector genome of claim 35.

43. The AAV viral particle of claim 42, wherein the AAV capsid protein comprises or consists of a sequence having at least 90%, at least 95%, at least 96%, at least 97%, at least 98%, at least 99%, or 100% identity to a sequence selected from SEQ ID NOs: 1-3, 67, 71, 196, 205 and 206.

44. The AAV viral particle of claim 42, wherein the AAV capsid protein comprises or consists of a sequence having at least 90%, at least 95%, at least 96%, at least 97%, at least 98%, at least 99%, or 100% identity to SEQ ID NO: 3.

45. The AAV viral particle of claim 42, wherein the AAV capsid protein comprises or consists of a sequence having at least 90%, at least 95%, at least 96%, at least 97%, at least 98%, at least 99%, or 100% identity to SEQ ID NO: 67.

46. A pharmaceutical composition comprising the AAV viral particle of claim 42.

47.-48. (canceled)

49. A method of expressing a protein encoded by a transgene in a cell, comprising:
transducing the cell with an AAV viral particle comprising the AAV vector genome of claim 35.

50. (canceled)

51. A method of treating a subject having a disease or disorder, comprising:
administering to the subject an AAV viral particle comprising the AAV vector genome of claim 35.

52.-58. (canceled)

59. The method of claim 51, wherein the disease or disorder is Stargardt disease.

60.-73. (canceled)

74. A nucleic acid comprising a 5′ portion of an ABCA4 gene, wherein the 5′ portion of the ABCA4 gene comprises one or more of: (i) a nucleotide “G” at the position corresponding to nucleotide 813 of SEQ ID NO: 212; (ii) a nucleotide “A” at the position corresponding to nucleotide 1311 of SEQ ID NO: 212; (iii) a nucleotide “A” at the position corresponding to nucleotide 2274 of SEQ ID NO: 212; (iv) a nucleotide “C” at the position corresponding to nucleotide 2277 of SEQ ID NO: 212; (v) a nucleotide “T” at the position corresponding to nucleotide 2278 of SEQ ID NO: 212; and (vi) a nucleotide “T” at the position corresponding to nucleotide 2568 of SEQ ID NO: 212.

75.-78. (canceled)

79. An AAV vector genome comprising the nucleic acid of claim 74.

80. An AAV viral particle comprising
(i) an AAV capsid, wherein the AAV capsid comprises an AAV capsid protein; and (ii) the AAV vector genome of claim 79.

81. (canceled)

82. A method of treating a subject having a disease or disorder, comprising administering to the subject the AAV viral particle of claim 80, optionally wherein the disease or disorder is Stargardt disease.

83. A transduced cell comprising the nucleic acid of claim 74.


SEQUENCE LISTING (224 sequences)

SEQ ID NO: 1 - protein, 737 aa, synthetic construct
  REGION 1..737: note = AAV110 VPl
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - protein, 736 aa, synthetic construct
  REGION 1..736: note = AAV204 VP1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQNSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 3 - protein, 735 aa, synthetic construct
  REGION 1..735: note = AAV214 VPl
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 4 - DNA, 4287 bp, synthetic construct
  misc_feature 1..4287: note = CFTRdeltaR
  source 1..4287: mol_type = other DNA
atgcagagaagccccctggagaaggcctctgtggtgagcaagctgttcttcagctggaccagacccatcctgagaaagggctacagacagagactggagctgtctgacatctaccagatcccctctgtggactctgctgacaacctgtctgagaagctggagagagagtgggacagagagctggccagcaagaagaaccccaagctgatcaatgccctgagaagatgcttcttctggagattcatgttctatggcatcttcctgtacctgggggaggtgaccaaggctgtgcagcccctgctgctgggcagaatcattgccagctatgaccctgacaacaaggaggagagaagcattgccatctacctgggcattggcctgtgcctgctgttcattgtgagaaccctgctgctgcaccctgccatctttggcctgcaccacattggcatgcagatgagaattgccatgttcagcctgatctacaagaagaccctgaagctgagcagcagagtgctggacaagatcagcattggccagctggtgagcctgctgagcaacaacctgaacaagtttgatgagggcctggccctggcccactttgtgtggattgcccccctgcaggtggccctgctgatgggcctgatctgggagctgctgcaggcctctgccttctgtggcctgggcttcctgattgtgctggccctgttccaggctggcctgggcagaatgatgatgaagtacagagaccagagagctggcaagatctctgagagactggtgatcacctctgagatgattgagaacatccagtctgtgaaggcctactgctgggaggaggccatggagaagatgattgagaacctgagacagacagagctgaagctgaccagaaaggctgcctatgtgagatacttcaacagctctgccttcttcttctctggcttctttgtggtgttcctgtctgtgctgccctatgccctgatcaagggcatcatcctgagaaagatcttcaccaccatcagcttctgcattgtgctgagaatggctgtgaccagacagttcccctgggctgtgcagacctggtatgacagcctgggggccatcaacaagatccaggacttcctgcagaagcaggagtacaagaccctggagtacaacctgaccaccacagaggtggtgatggagaatgtgacagccttctgggaggagggctttggggagctgtttgagaaggccaagcagaacaacaacaacagaaagaccagcaatggggatgacagcctgttcttcagcaacttcagcctgctgggcacccctgtgctgaaggacatcaacttcaagattgagagaggccagctgctggctgtggctggcagcacaggggctggcaagaccagcctgctgatgatgatcatgggggagctggagccctctgagggcaagatcaagcactctggcagaatcagcttctgcagccagttcagctggatcatgcctggcaccatcaaggagaacatcatctttggggtgagctatgatgagtacagatacagatctgtgatcaaggcctgccagctggaggaggacatcagcaagtttgctgagaaggacaacattgtgctgggggaggggggcatcaccctgtctgggggccagagagccagaatcagcctggccagagctgtgtacaaggatgctgacctgtacctgctggacagcccctttggctacctggatgtgctgacagagaaggagatctttgagagctgtgtgtgcaagctgatggccaacaagaccagaatcctggtgaccagcaagatggagcacctgaagaaggctgacaagatcctgatcctgcatgagggcagcagctacttctatggcaccttctctgagctgcagaacctgcagcctgacttcagcagcaagctgatgggctgtgacagctttgaccagttctctgctgagagaagaaacagcatcctgacagagaccctgcacagattcagcctggagggggatgcccctgtgagctggacagagaccaagaagcagagcttcaagcagacaggggagtttggggagaagagaaagaacagcatcctgaaccccatcaacagcaccctgcaggccagaagaagacagtctgtgctgaacctgatgacccactctgtgaaccagggccagaacatccacagaaagaccacagccagcaccagaaaggtgagcctggccccccaggccaacctgacagagctggacatctacagcagaagactgagccaggagacaggcctggagatctctgaggagatcaatgaggaggacctgaaggagtgcttctttgatgacatggagagcatccctgctgtgaccacctggaacacctacctgagatacatcacagtgcacaagagcctgatctttgtgctgatctggtgcctggtgatcttcctggctgaggtggctgccagcctggtggtgctgtggctgctgggcaacacccccctgcaggacaagggcaacagcacccacagcagaaacaacagctatgctgtgatcatcaccagcaccagcagctactatgtgttctacatctatgtgggggtggctgacaccctgctggccatgggcttcttcagaggcctgcccctggtgcacaccctgatcacagtgagcaagatcctgcaccacaagatgctgcactctgtgctgcaggcccccatgagcaccctgaacaccctgaaggctgggggcatcctgaacagattcagcaaggacattgccatcctggatgacctgctgcccctgaccatctttgacttcatccagctgctgctgattgtgattggggccattgctgtggtggctgtgctgcagccctacatctttgtggccacagtgcctgtgattgtggccttcatcatgctgagagcctacttcctgcagaccagccagcagctgaagcagctggagtctgagggcagaagccccatcttcacccacctggtgaccagcctgaagggcctgtggaccctgagagcctttggcagacagccctactttgagaccctgttccacaaggccctgaacctgcacacagccaactggttcctgtacctgagcaccctgagatggttccagatgagaattgagatgatctttgtgatcttcttcattgctgtgaccttcatcagcatcctgaccacaggggagggggagggcagagtgggcatcatcctgaccctggccatgaacatcatgagcaccctgcagtgggctgtgaacagcagcattgatgtggacagcctgatgagatctgtgagcagagtgttcaagttcattgacatgcccacagagggcaagcccaccaagagcaccaagccctacaagaatggccagctgagcaaggtgatgatcattgagaacagccatgtgaagaaggatgacatctggccctctgggggccagatgacagtgaaggacctgacagccaagtacacagaggggggcaatgccatcctggagaacatcagcttcagcatcagccctggccagagagtgggcctgctgggcagaacaggctctggcaagagcaccctgctgtctgccttcctgagactgctgaacacagagggggagatccagattgatggggtgagctgggacagcatcaccctgcagcagtggagaaaggcctttggggtgatcccccagaaggtgttcatcttctctggcaccttcagaaagaacctggacccctatgagcagtggtctgaccaggagatctggaaggtggctgatgaggtgggcctgagatctgtgattgagcagttccctggcaagctggactttgtgctggtggatgggggctgtgtgctgagccatggccacaagcagctgatgtgcctggccagatctgtgctgagcaaggccaagatcctgctgctggatgagccctctgcccacctggaccctgtgacctaccagatcatcagaagaaccctgaagcaggcctttgctgactgcacagtgatcctgtgtgagcacagaattgaggccatgctggagtgccagcagttcctggtgattgaggagaacaaggtgagacagtatgacagcatccagaagctgctgaatgagagaagcctgttcagacaggccatcagcccctctgacagagtgaagctgttcccccacagaaacagcagcaagtgcaagagcaagccccagattgctgccctgaaggaggagaccgaggaggaggtgcaggacaccagactgtaa

SEQ ID NO: 5 - DNA, 2859 bp, synthetic construct
  misc_feature 1..2859: note = GAA
  source 1..2859: mol_type = other DNA
atgggagtccgccacccgccctgctcacatcgcctgcttgctgtctgtgccctcgtgtcacttgctaccgccgcgctgcttggtcacattctgctgcacgactttttactagttccgagggaactgtcgggatccagccccgtgctcgaggaaactcaccccgcgcaccaacagggggcgtccaggccgggaccgcgcgacgcccaggcccacccgggccggcctcgggccgtgccaactcagtgcgatgtgccgccgaactcccgcttcgactgtgcgcctgacaaggccataacccaggaacagtgcgaagcacgcggctgctgctatattccggcgaagcagggcttgcagggtgcccaaatgggtcagccttggtgcttctttcccccgtcgtacccctcgtacaagctggagaacctgagcagcagcgaaatggggtacaccgccactctgacccggacgaccccgaccttcttcccgaaagacatcctgaccctgcggctggatgtgatgatggaaactgagaacagactgcacttcactatcaaggaccccgcgaaccgcagatatgaggtgccactggaaacccctcatgtgcattcccgggccccatcccctctgtactcggtggaattctccgaagaacccttcggggtcattgtgcgccggcagcttgatggccgggtcctgctcaacaccaccgtggcaccccttttcttcgctgaccagttcctccagctgagcacctcgctgccgagccagtacatcaccggactggccgagcacctctcccctctgatgctgtccactagctggactaggatcactctgtggaaccgggatctggcccctaccccgggcgcgaacctgtacggatcgcaccccttctacctggccctcgaggacggaggctccgcccacggagtgttcctgctgaactccaacgctatggacgtggtgctccagccgtcccctgcactgtcctggcggagcacagggggtattctggatgtctacatcttcctcggcccggagccaaagtccgtggtgcaacagtatctggatgtcgtgggttacccattcatgccgccatactggggccttggcttccacctgtgccgctggggatacagctccaccgccatcactagacaggtcgtggaaaacatgactagagcccacttccccctcgatgtccagtggaatgacctggactacatggattccagacgcgacttcactttcaacaaggatggattcagagatttccccgctatggtccaagaactgcaccagggtggccggcggtacatgatgattgtggaccccgccatttcaagctccggaccagcgggctcgtaccggccctacgacgaaggtttgcgccgcggcgtgttcatcactaacgaaaccggccagccactgattgggaaggtctggcctggaagcaccgcgttcccggacttcactaacccaacggccttggcgtggtgggaggacatggtggccgaattccacgaccaagtcccattcgacggaatgtggatcgacatgaacgagcccagcaacttcatccgaggctccgaggacggctgccctaacaacgaacttgagaaccctccgtacgtgcctggcgtcgtcggcggaacactgcaggccgctacgatctgtgcctcatcgcatcagttcctgtcaacccactacaacctccataatctgtacggcctcaccgaagccatcgcctcccaccgggccctggtcaaggcccgggggactaggcccttcgtgattagccggagcactttcgccggacacggaagatacgccggacattggaccggcgacgtgtggtcatcgtgggagcagctcgcctcctccgtccccgaaatcctgcagttcaatctcctgggagtccccctcgtgggcgcggacgtgtgcggattcctgggcaatacctctgaggagctgtgcgtgagatggacccagctgggggcgttctaccccttcatgcggaaccacaactcactgctgtccctgcctcaagagccgtactcattctccgagccggcacaacaggccatgcgaaaggctctgaccctccgctatgcgctcttgccccacctctacactctgtttcaccaagcccatgtcgcgggcgaaacagtggccagaccactctttctggaattcccaaaggactcctcaacctggactgtggatcatcagctgctctggggagaggcactgctgatcaccccggtgctccaagccggaaaggcggaagtgaccggatacttccctctcggtacttggtacgacctccaaaccgtgccggtcgaggccctgggcagcttgcctccgccgccggctgccccgcgggagcctgcaatccactccgaggggcaatgggtgaccctccctgcaccactggacaccatcaacgtgcacctccgggccggctacatcatcccgctgcaaggaccgggtctgactaccaccgaatcccggcagcagcccatggcactggccgtggccctgaccaagggaggggaagcacggggagaactcttttgggacgatggagaatccctggaagtgctcgagcggggagcctacactcaagtcatctttcttgcccgcaacaacaccatcgtgaacgaattggtccgcgtgacctccgagggggccggactccagctgcaaaaagtgaccgtgctgggggtggcaaccgccccgcaacaagtgttgtctaacggagtgccggtgtccaacttcacctactcccctgataccaaagttctagatatttgcgtgagcctgctgatgggagaacagttcctggtgtcctggtgctga

SEQ ID NO: 6 - DNA, 2859 bp, synthetic construct; identical to SEQ ID NO: 5
  misc_feature 1..2859: note = GAA codon-optimized nucleotide sequence 1 (GAA 15)
  source 1..2859: mol_type = other DNA

SEQ ID NO: 7 - DNA, 2859 bp, synthetic construct
  misc_feature 1..2859: note = GAA Codon-optimized 2 (GAA21)
  source 1..2859: mol_type = other DNA
atgggagttagacaccctccatgtagccacagactgctggccgtgtgtgctctggtgtctctggctacagctgccctgctgggacatatcctgctgcacgacttcttactagttcccagagagctgtccggcagcagccctgtgctggaagaaacacaccctgcacatcagcagggcgcctctagacctggacctagagatgctcaggcccatcctggcagacctagagctgtgcccacacagtgtgacgtgccacctaacagcagattcgactgcgcccctgacaaggccatcacacaagagcagtgtgaagccagaggctgctgctacatccctgccaaacaaggactgcagggcgctcagatgggacagccctggtgcttcttcccaccatcttaccccagctacaagctggaaaacctgagcagcagcgagatgggctacaccgccacactgaccagaaccacacctacattcttcccgaaggacatcctgacactgcggctggacgtgatgatggaaaccgagaaccggctgcacttcaccatcaaggaccccgccaatcggagatacgaggtgccactggaaacccctcacgtgcactctagagccccatctccactgtacagcgtggaattcagcgaggaacccttcggcgtgatcgtgcggagacagctggatggaagagtgctgctgaacaccacagtggcccctctgttcttcgccgaccagtttctgcagctgtccaccagcctgcctagccagtatatcacaggcctggccgagcacctgtctccactgatgctgtctaccagctggacccggatcaccctgtggaacagggatcttgctcctacacctggcgccaacctgtacggctctcaccctttttatctggccctggaagatggcggatctgcccacggtgtctttctgctgaactccaacgccatggacgtggtgctgcagccatctcctgctctgtcttggagaagcacaggcggcatcctggacgtgtacatctttctgggccccgagcctaagagcgtggtgcagcagtatctggacgtcgtgggctaccccttcatgcctccttattggggcctgggcttccacctgtgcagatggggatacagcagcaccgccatcaccagacaggtggtggaaaacatgacccgggctcacttcccactggatgtgcagtggaacgacctggactacatggacagcagacgggacttcaccttcaacaaggacggcttcagagacttccccgccatggtgcaagaactgcaccaaggcggcagacggtacatgatgatcgtggatccagccatcagctctagcggccctgccggctcttacagaccttacgatgagggcctgagaagaggcgtgttcatcaccaacgagacaggccagcctctgatcggcaaagtgtggcctggcagcacagcctttccagacttcacaaaccccaccgctctggcttggtgggaagatatggtggccgagtttcacgatcaggtgcccttcgacggcatgtggatcgacatgaacgagcccagcaacttcatccggggcagcgaggatggctgccccaacaacgaactggaaaatcctccttacgtgcccggcgttgtcggcggaacacttcaggccgctacaatctgtgccagcagccaccagttcctcagcacccactacaacctgcacaatctgtatggcctgaccgaggccattgccagccatagagccctggttaaggccaggggcaccagacctttcgtgatcagcagaagcaccttcgccggccacggcagatatgccggacattggacaggcgacgtgtggtctagttgggagcagctggctagcagcgtgccagagatcctgcagttcaatctgctgggcgtgccactcgtgggagccgatgtttgtggcttcctgggcaacacctccgaggaactgtgtgtgcgttggacacagctgggcgccttctatcccttcatgagaaaccacaacagccttctcagcctgccacaagagccctacagcttctctgagcctgcacagcaggccatgagaaaggccctgactctgagatacgctctgctgccccacctgtacaccctgtttcaccaggctcatgtggccggggagacagtggctagacctctgttcctggaattccccaaggacagctccacctggaccgtggatcatcagctgctgtggggagaagccctgctcatcacacctgttctgcaggccggaaaggccgaagtgaccggctattttcctctcggcacttggtacgacctgcagaccgtgcctgttgaggctctgggatctcttcctccacctcctgccgctcctagagagcctgccattcactctgaaggccagtgggttaccctgcctgctcctctggacaccatcaacgtgcacctgagagctggctacatcatccctctgcaaggccctggcctgacaaccaccgaatctagacagcagcccatggctctggccgtggctttgacaaaaggcggagaggctagaggcgagctgttctgggatgatggcgagagcctggaagtgctggaacggggcgcttatacccaagtgatcttcctggccagaaacaacaccatcgtgaacgaactcgtgcgcgtgaccagtgaaggtgctggactgcaactgcagaaagtgaccgtgctcggagtggccacagcacctcagcaggttctgtctaatggcgtgcccgtgtccaacttcacatacagccccgacaccaaggtcctggacatctgtgtgtcactgctgatgggcgagcagttcctggtgtcctggtgttga

SEQ ID NO: 8 - protein, 952 aa, Homo sapiens
  REGION 1..952: note = MISC_FEATURE - Acid Alpha-Glucosidase (GAA)
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 9 - DNA, 1290 bp, synthetic construct
  misc_feature 1..1290: note = GLA
  source 1..1290: mol_type = other DNA
atgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 10 - DNA, 1290 bp, synthetic construct
  misc_feature 1..1290: note = GLA codon-optimized
  source 1..1290: mol_type = other DNA
atgcagctgagaaatcctgaactgcacctgggctgtgccctggctctgagatttctggctctggtgtcctgggacattcctggcgctagagccctggataatggcctggccagaacacctacaatgggctggctgcactgggagagattcatgtgcaacctggactgccaagaggaacccgacagctgcatcagcgagaagctgttcatggaaatggccgagctgatggtgtccgaaggctggaaggatgccggctacgagtacctgtgcatcgacgattgctggatggcccctcagagagattctgagggcagactgcaggccgatcctcagagatttcctcacggaatccggcagctggccaactacgtgcactctaagggactgaagctgggcatctacgccgacgtgggcaacaagacatgtgccggctttccaggcagcttcggctactacgatatcgacgcccagacctttgccgattggggcgtcgacctgctgaagttcgatggctgctactgcgacagcctggaaaacctggccgacggctacaaacacatgtctctggccctgaaccggaccggcagatctatcgtgtactcttgcgagtggcccctgtacatgtggcccttccagaagcctaactacaccgagatcagacagtactgcaaccactggcggaacttcgccgacatcgatgacagctggaagtccatcaagagcatcctggactggaccagcttcaatcaagagcggatcgtggatgtggctggcccaggcggatggaacgatcctgatatgctggtcatcggcaacttcggcctgagctggaatcagcaagtgacccagatggccctgtgggccattatggccgctcctctgttcatgagcaacgacctgagacacatcagccctcaggccaaggctctgctgcaggataaggacgtgatcgccatcaaccaggatcctctgggcaagcagggctatcagctgagacagggcgacaatttcgaagtgtgggaaagacctctgagcggcctggcttgggccgtcgccatgatcaatagacaagagatcggcggaccccggtcctatacaattgccgtggcttctctcggaaaaggcgtggcctgcaatcctgcctgctttatcacacagctgctccccgtgaagagaaagctgggcttttacgagtggaccagcagactgagatcccacatcaaccccacaggcactgttctgctgcaactggaaaacacaatgcagatgagcctgaaggacctgctgtag

SEQ ID NO: 11 - protein, 429 aa, synthetic construct
  REGION 1..429: note = GLA
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 12 - DNA, 1317 bp, synthetic construct
  misc_feature 1..1317: note = CLN3
  source 1..1317: mol_type = other DNA
atgggaggctgtgcaggctcgcggcggcgcttttcggattccgagggggaggagaccgtcccggagccccggctccctctgttggaccatcagggcgcgcattggaagaacgcggtgggcttctggctgctgggcctttgcaacaacttctcttatgtggtgatgctgagtgccgcccacgacatccttagccacaagaggacatcgggaaaccagagccatgtggacccaggcccaacgccgatcccccacaacagctcatcacgatttgactgcaactctgtctctacggctgctgtgctcctggcggacatcctccccacactcgtcatcaaattgttggctcctcttggccttcacctgctgccctacagcccccgggttctcgtcagtgggatttgtgctgctggaagcttcgtcctggttgccttttctcattctgtggggaccagcctgtgtggtgtggtcttcgctagcatctcatcaggccttggggaggtcaccttcctctccctcactgccttctaccccagggccgtgatctcctggtggtcctcagggactgggggagctgggctgctgggggccctgtcctacctgggcctcacccaggccggcctctcccctcagcagaccctgctgtccatgctgggtatccctgccctgctgctggccagctatttcttgttgctcacatctcctgaggcccaggaccctggaggggaagaagaagcagagagcgcagcccggcagcccctcataagaaccgaggccccggagtcgaagccaggctccagctccagcctctcccttcgggaaaggtggacagtgttcaagggtctgctgtggtacattgttcccttggtcgtagtttactttgccgagtatttcattaaccagggactttttgaactcctctttttctggaacacttccctgagtcacgctcagcaataccgctggtaccagatgctgtaccaggctggcgtctttgcctcccgctcttctctccgctgctgtcgcatccgtttcacctgggccctggccctgctgcagtgcctcaacctggtgttcctgctggcagacgtgtggttcggctttctgccaagcatctacctcgtcttcctgatcattctgtatgaggggctcctgggaggcgcagcctacgtgaacaccttccacaacatcgccctggagaccagtgatgagcaccgggagtttgcaatggcggccacctgcatctctgacacactggggatctccctgtcggggctcctggctttgcctctgcatgacttcctctgccagctctcctga

SEQ ID NO: 13 - DNA, 1317 bp, synthetic construct
  misc_feature 1..1317: note = CLN3 codon-optimized
  source 1..1317: mol_type = other DNA
atgggaggatgtgctgggtcaagaagacggtttagcgattccgaaggagaggagactgtgcctgagccaagactgcccctgctggatcaccagggagcacactggaagaacgcagtgggattctggctgctgggcctgtgcaacaacttcagctacgtggtcatgctgtccgccgcccacgacatcctgtcccacaagcggacctccggcaatcagtctcacgtggaccccggccctacaccaatcccccacaacagcagcagccggttcgactgtaattccgtgtctaccgcagccgtgctgctggcagacatcctgcccaccctggtcatcaagctgctggcaccactgggcctgcacctgctgccttattctccaagggtgctggtgagcggcatctgcgcagcaggcagcttcgtgctggtggcctttagccactccgtgggcacctctctgtgcggagtggtgtttgcaagcatcagctccggcctgggagaggtgaccttcctgagcctgacagccttttaccctcgcgccgtgatctcctggtggtctagcggcacaggaggagcaggcctgctgggcgccctgtcctatctgggcctgacccaggcaggcctgtccccacagcagacactgctgtctatgctgggcatccctgccctgctgctggcaagctacttcctgctgctgacctccccagaggcacaggaccccggaggagaggaggaggccgagagcgccgcaaggcagccactgatcaggaccgaggcaccagagtccaagcctggctcctctagctccctgtctctgcgggagagatggacagtgttcaagggcctgctgtggtacatcgtgcccctggtggtggtgtacttcgccgagtacttcatcaaccagggcctgtttgagctgctgttcttttggaatacctctctgagccacgcccagcagtaccggtggtatcagatgctgtatcaggcaggcgtgttcgcctcccggtctagcctgagatgctgtcggatcagattcacctgggcactggccctgctgcagtgcctgaacctggtgttcctgctggccgacgtgtggttcggctttctgccctctatctacctggtgtttctgatcatcctgtatgagggcctgctgggaggagcagcctatgtgaacaccttccacaatatcgccctggagacatctgacgagcacagagagtttgctatggccgccacctgtatcagcgatacactgggcatctctctgagcggactgctggctctgcctctgcatgactttctgtgccagctgagttaa

SEQ ID NO: 14 - protein, 438 aa, Homo sapiens
  REGION 1..438: note = MISC_FEATURE - Ceroid Lipofuscinosis, Neuronal 3 (CLN3)
MGGCAGSRRRFSDSEGEETVPEPRLPLLDHQGAHWKNAVGFWLLGLCNNFSYVVMLSAAHDILSHKRTSGNQSHVDPGPTPIPHNSSSRFDCNSVSTAAVLLADILPTLVIKLLAPLGLHLLPYSPRVLVSGICAAGSFVLVAFSHSVGTSLCGVVFASISSGLGEVTFLSLTAFYPRAVISWWSSGTGGAGLLGALSYLGLTQAGLSPQQTLLSMLGIPALLLASYFLLLTSPEAQDPGGEEEAESAARQPLIRTEAPESKPGSSSSLSLRERWTVFKGLLWYIVPLVVVYFAEYFINQGLFELLFFWNTSLSHAQQYRWYQMLYQAGVFASRSSLRCCRIRFTWALALLQCLNLVFLLADVWFGFLPSIYLVFLIILYEGLLGGAAYVNTFHNIALETSDEHREFAMAATCISDTLGISLSGLLALPLHDFLCQLS

SEQ ID NO: 15 - DNA, 2211 bp, synthetic construct
  misc_feature 1..2211: note = AAV204 VP1
  source 1..2211: mol_type = other DNA
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcaccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcggacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggacaggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagaacagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtagacaacaatggactttatactgagcctcgccccattggcacccgttacctcacccgtcccctgtaa

SEQ ID NO: 16 - DNA, 1605 bp, synthetic construct
  misc_feature 1..1605: note = AAV204 VP3
  source 1..1605: mol_type = other DNA
atggcttcaggcggtggcgcaccaatggcggacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggacaggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagaacagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtagacaacaatggactttatactgagcctcgccccattggcacccgttacctcacccgtcccctgtaa

SEQ ID NO: 17 - protein, 534 aa, synthetic construct
  REGION 1..534: note = AAV204 VP3
MASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQNSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 18 - DNA, 2208 bp, synthetic construct
  misc_feature 1..2208: note = ITB102 214 (AAV214) VP1
  source 1..2208: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaaccttttggtctggttgaggaaggtgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcggacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtcctcggctctgcgcaccagggctgcctccctccgttcccggcggacgtgttcatgattccgcagtacggctacctaacgctcaacgacggcagccaggcagtgggacggtcatccttttactgcctggaatatttcccatcgcagatgctgagaacgggcaacaactttaccttcagctacacctttgaggacgttcctttccacagcagctacgctcacagccagagtctggaccgtctcatgaatcctctgattgaccagtacctgtactacttgtctaagactatcaacggatccggccagaatcagcagactctgaagttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 19 - DNA, 2211 bp, synthetic construct
  misc_feature 1..2211: note = AAV214-A VP1
  source 1..2211: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaaccttttggtctggttgaggaaggtgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcggacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccaacagcacatctggaggatcttcaaatgacaacgcctacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtcctcggctctgcgcaccagggctgcctccctccgttcccggcggacgtgttcatgattccgcagtacggctacctaacgctcaacgacggcagccaggcagtgggacggtcatccttttactgcctggaatatttcccatcgcagatgctgagaacgggcaacaactttaccttcagctacacctttgaggacgttcctttccacagcagctacgctcacagccagagtctggaccgtctcatgaatcctctgattgaccagtacctgtactacttgtctaagactatcaacggatccggccagaatcagcagactctgaagttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 20 - DNA, 2211 bp, synthetic construct
  misc_feature 1..2211: note = AAV214e VP1
  source 1..2211: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtcctcggctctgcgcaccagggctgcctccctccgttcccggcggacgtgttcatgattccgcagtacggctacctaacgctcaacgacggcagccaggcagtgggacggtcatccttttactgcctggaatatttcccatcgcagatgctgagaacgggcaacaactttaccttcagctacacctttgaggacgttcctttccacagcagctacgctcacagccagagtctggaccgtctcatgaatcctctgattgaccagtacctgtactacttgtctaagactatcaacggatccggccagaatcagcagactctgaagttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 21 - DNA, 2211 bp, synthetic construct
  misc_feature 1..2211: note = AAV214e8 VP1
  source 1..2211: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggcttcaggcggtggcgcaccaatggcggacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtcctcggctctgcgcaccagggctgcctccctccgttcccggcggacgtgttcatgattccgcagtacggctacctaacgctcaacgacggcagccaggcagtgggacggtcatccttttactgcctggaatatttcccatcgcagatgctgagaacgggcaacaactttaccttcagctacacctttgaggacgttcctttccacagcagctacgctcacagccagagtctggaccgtctcatgaatcctctgattgaccagtacctgtactacttgtctaagactatcaacggatccggccagaatcagcagactctgaagttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 22 - DNA, 2208 bp, synthetic construct
  misc_feature 1..2208: note = AAV214e9 VP1
  source 1..2208: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctccccaggaaccggactcctccgcgggtattggcaaatcgggtgcacagcccgctaaaaagagactcaatttcggtcagactggcgacacagagtcagtcccagaccctcaaccaatcggagaacctcccgcagccccctctggtgtgggatctcttacaatggcttcaggcggtggcgcaccaatggcggacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtcctcggctctgcgcaccagggctgcctccctccgttcccggcggacgtgttcatgattccgcagtacggctacctaacgctcaacgacggcagccaggcagtgggacggtcatccttttactgcctggaatatttcccatcgcagatgctgagaacgggcaacaactttaccttcagctacacctttgaggacgttcctttccacagcagctacgctcacagccagagtctggaccgtctcatgaatcctctgattgaccagtacctgtactacttgtctaagactatcaacggatccggccagaatcagcagactctgaagttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 23 - DNA, 2211 bp, synthetic construct
  misc_feature 1..2211: note = AAV214e10 VP1
  source 1..2211: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggcttcaggcggtggcgcaccaatggcggacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtcctcggctctgcgcaccagggctgcctccctccgttcccggcggacgtgttcatgattccgcagtacggctacctaacgctcaacgacggcagccaggcagtgggacggtcatccttttactgcctggaatatttcccatcgcagatgctgagaacgggcaacaactttaccttcagctacacctttgaggacgttcctttccacagcagctacgctcacagccagagtctggaccgtctcatgaatcctctgattgaccagtacctgtactacttgtctaagactatcaacggatccggccagaatcagcagactctgaagttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 24 - DNA, 1602 bp, synthetic construct
  misc_feature 1..1602: note = ITB102 214 (AAV214) VP3
  source 1..1602: mol_type = other DNA
atggcttcaggcggtggcgcaccaatggcggacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtcctcggctctgcgcaccagggctgcctccctccgttcccggcggacgtgttcatgattccgcagtacggctacctaacgctcaacgacggcagccaggcagtgggacggtcatccttttactgcctggaatatttcccatcgcagatgctgagaacgggcaacaactttaccttcagctacacctttgaggacgttcctttccacagcagctacgctcacagccagagtctggaccgtctcatgaatcctctgattgaccagtacctgtactacttgtctaagactatcaacggatccggccagaatcagcagactctgaagttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 25 - DNA, 1605 bp, synthetic construct
  misc_feature 1..1605: note = AAV214-A VP3
  source 1..1605: mol_type = other DNA
atggcttcaggcggtggcgcaccaatggcggacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccaacagcacatctggaggatcttcaaatgacaacgcctacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtcctcggctctgcgcaccagggctgcctccctccgttcccggcggacgtgttcatgattccgcagtacggctacctaacgctcaacgacggcagccaggcagtgggacggtcatccttttactgcctggaatatttcccatcgcagatgctgagaacgggcaacaactttaccttcagctacacctttgaggacgttcctttccacagcagctacgctcacagccagagtctggaccgtctcatgaatcctctgattgaccagtacctgtactacttgtctaagactatcaacggatccggccagaatcagcagactctgaagttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 26 - DNA, 1602 bp, synthetic construct
  misc_feature 1..1602: note = AAV214e VP3
  source 1..1602: mol_type = other DNA
atggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtcctcggctctgcgcaccagggctgcctccctccgttcccggcggacgtgttcatgattccgcagtacggctacctaacgctcaacgacggcagccaggcagtgggacggtcatccttttactgcctggaatatttcccatcgcagatgctgagaacgggcaacaactttaccttcagctacacctttgaggacgttcctttccacagcagctacgctcacagccagagtctggaccgtctcatgaatcctctgattgaccagtacctgtactacttgtctaagactatcaacggatccggccagaatcagcagactctgaagttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 27 - DNA, 1602 bp, synthetic construct; identical to SEQ ID NO: 24
  misc_feature 1..1602: note = AAV214e8 VP3
  source 1..1602: mol_type = other DNA

SEQ ID NO: 28 - DNA, 1602 bp, synthetic construct; identical to SEQ ID NO: 24
  misc_feature 1..1602: note = AAV214e9 VP3
  source 1..1602: mol_type = other DNA

SEQ ID NO: 29 - DNA, 1602 bp, synthetic construct; identical to SEQ ID NO: 24
  misc_feature 1..1602: note = AAV214e10 VP3
  source 1..1602: mol_type = other DNA

SEQ ID NO: 30 - protein, 736 aa, synthetic construct
  REGION 1..736: note = AAV214A VP1
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 31 - protein, 736 aa, synthetic construct
  REGION 1..736: note = AAV214e VP1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 32 - protein, 736 aa, synthetic construct
  REGION 1..736: note = AAV214e8 VP1
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 33 - protein, 735 aa, synthetic construct
  REGION 1..735: note = AAV214e9 VP1
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 34 - protein, 736 aa, synthetic construct
  REGION 1..736: note = AAV214e10 VP1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 35 - protein, 598 aa, synthetic construct
  REGION 1..598: note = AAV214 VP2
MAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 36 - protein, 599 aa, synthetic construct
  REGION 1..599: note = AAV214A VP2
MAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 37 - protein, 599 aa, synthetic construct
  REGION 1..599: note = AAV214e VP2
MAPGKKRPVEPSPQRSPDSSTGIGKKGQQPARKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 38 - protein, 599 aa, synthetic construct
  REGION 1..599: note = AAV214e8 VP2
MAPGKKRPVEPSPQRSPDSSTGIGKKGQQPARKRLNFGQTGDSESVPDPQPLGEPPAAPSGVGPNTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 39 - protein, 598 aa, synthetic construct
  REGION 1..598: note = AAV214e9 VP2
MAPGKKRPVEQSPQEPDSSAGIGKSGAQPAKKRLNFGQTGDTESVPDPQPIGEPPAAPSGVGSLTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 40 - protein, 599 aa, synthetic construct
  REGION 1..599: note = AAV214e10 VP2
MAPGKKRPVEPSPQRSPDSSTGIGKKGQQPAKKRLNFGQTGDSESVPDPQPIGEPPAGPSGLGSGTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 41 - protein, 533 aa, synthetic construct
  REGION 1..533: note = AAV214 VP3
MASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 42 - protein, 534 aa, synthetic construct
  REGION 1..534: note = AAV214A VP3
MASGGGAPMADNNEGADGVGNASGNWHCDSTWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 43 - protein, 533 aa, synthetic construct; identical to SEQ ID NO: 41
  REGION 1..533: note = AAV214E VP3

SEQ ID NO: 44 - protein, 533 aa, synthetic construct; identical to SEQ ID NO: 41
  REGION 1..533: note = AAV214E8 VP3

SEQ ID NO: 45 - protein, 533 aa, synthetic construct; identical to SEQ ID NO: 41
  REGION 1..533: note = AAV214E9 VP3

SEQ ID NO: 46 - protein, 533 aa, synthetic construct; identical to SEQ ID NO: 41
  REGION 1..533: note = AAV214E10 VP3

SEQ ID NO: 47 - DNA, 2211 bp, synthetic construct
  misc_feature 1..2211: note = ITB102 45 VP1
  source 1..2211: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaaccttttggtctggttgaggaaggtgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcggacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacggctacctaacgctcaacaatggcagccaggcagtgggacggtcatccttttactgcctggaatatttcccatcgcagatgctgagaacgggcaacaactttaccttcagctacacctttgaggacgttcctttccacagcagctacgctcacagccagagtctggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaggcactggcagagacaatgtggatgccgacaaagtcatgatcaccaacgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 48 - DNA, 1605 bp, synthetic construct
  misc_feature 1..1605: note = ITB102 45 VP3
  source 1..1605: mol_type = other DNA
atggcttcaggcggtggcgcaccaatggcggacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacggctacctaacgctcaacaatggcagccaggcagtgggacggtcatccttttactgcctggaatatttcccatcgcagatgctgagaacgggcaacaactttaccttcagctacacctttgaggacgttcctttccacagcagctacgctcacagccagagtctggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaggcactggcagagacaatgtggatgccgacaaagtcatgatcaccaacgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 49 - protein, 736 aa, synthetic construct
  REGION 1..736: note = ITB102 45 VP1
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNNGSQAVGRSSFYCLEYFPSQMLRTGNNFTFSYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLMNPGPAMASHKEGEDRFFPLSGSLIFGKQGTGRDNVDADKVMITNEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 50 - protein, 599 aa, synthetic construct
  REGION 1..599: note = ITB102 45 VP2
MAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNNGSQAVGRSSFYCLEYFPSQMLRTGNNFTFSYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLMNPGPAMASHKEGEDRFFPLSGSLIFGKQGTGRDNVDADKVMITNEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 51 - protein, 534 aa, synthetic construct
  REGION 1..534: note = ITB102 45 VP3
MASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNNGSQAVGRSSFYCLEYFPSQMLRTGNNFTFSYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLMNPGPAMASHKEGEDRFFPLSGSLIFGKQGTGRDNVDADKVMITNEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 52 - protein, 7 aa, synthetic construct
  REGION 1..7: note = VR-I
SASTGAS

SEQ ID NO: 53 - protein, 8 aa, synthetic construct
  REGION 1..8: note = VR-I
NSTSGGSS

SEQ ID NO: 54 - protein, 6 aa, synthetic construct
  REGION 1..6: note = VR-II
DNNGVK

SEQ ID NO: 55 - protein, 4 aa, synthetic construct
  REGION 1..4: note = VR-III
NDGS

SEQ ID NO: 56 - protein, 10 aa, synthetic construct
  REGION 1..10: note = VR-IV
INGSGQNQQT

SEQ ID NO: 57 - protein, 18 aa, synthetic construct
  REGION 1..18: note = VR-V
RVSTTTGQNNNSNFAWTA

SEQ ID NO: 58 - protein, 13 aa, synthetic construct
  REGION 1..13: note = VR-VI
HKEGEDRFFPLSG

SEQ ID NO: 59 - protein, 14 aa, synthetic construct
  REGION 1..14: note = VR-VII
KQNAARDNADYSDV

SEQ ID NO: 60 - protein, 13 aa, synthetic construct
  REGION 1..13: note = VR-VIII
ADNLQQQNTAPQI

SEQ ID NO: 61 - protein, 10 aa, synthetic construct
  REGION 1..10: note = VR-IX
NYYKSTSVDF

SEQ ID NO: 62 - DNA, 2211 bp, synthetic construct
  misc_feature 1..2211: note = AAV6 VP1
  source 1..2211: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 63 - protein, 736 aa, synthetic construct
  REGION 1..736: note = AAV6 VP1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 64 - protein, 599 aa, synthetic construct
  REGION 1..599: note = AAV6 VP2
TAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 65 - protein, 534 aa, synthetic construct
  REGION 1..534: note = AAV6 VP3
MASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 66 - DNA, 2217 bp, synthetic construct
  misc_feature 1..2217: note = AAV8 VP1
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 67 - protein, 738 aa, synthetic construct
  REGION 1..738: note = AAV8 VP1
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 68 - protein, 601 aa, synthetic construct
  REGION 1..601: note = AAV8 VP2
MAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 69 - protein, 535 aa, synthetic construct
  REGION 1..535: note = AAV8 VP3
MAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 70 - DNA, 2214 bp, synthetic construct
  misc_feature 1..2214: note = AAV9 VP1
  source 1..2214: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacggcaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtaattcctcgggaaattggcattgcgattccacatggctgggggacagagtcatcaccaccagcacccgaacctgggcattgcccacctacaacaaccacctctacaagcaaatctccaatggaacatcgggaggaagcaccaacgacaacacctactttggctacagcaccccctgggggtattttgacttcaacagattccactgccacttctcaccacgtgactggcagcgactcatcaacaacaactggggattccggccaaagagactcaacttcaagctgttcaacatccaggtcaaggaggttacgacgaacgaaggcaccaagaccatcgccaataaccttaccagcaccgtccaggtctttacggactcggagtaccagctaccgtacgtcctaggctctgcccaccaaggatgcctgccaccgtttcctgcagacgtcttcatggttcctcagtacggctacctgacgctcaacaatggaagtcaagcgttaggacgttcttctttctactgtctggaatacttcccttctcagatgctgagaaccggcaacaactttcagttcagctacactttcgaggacgtgcctttccacagcagctacgcacacagccagagtctagatcgactgatgaaccccctcatcgaccagtacctatactacctggtcagaacacagacaactggaactgggggaactcaaactttggcattcagccaagcaggccctagctcaatggccaatcaggctagaaactgggtacccgggccttgctaccgtcagcagcgcgtctccacaaccaccaaccaaaataacaacagcaactttgcgtggacgggagctgctaaattcaagctgaacgggagagactcgctaatgaatcctggcgtggctatggcatcgcacaaagacgacgaggaccgcttctttccatcaagtggcgttctcatatttggcaagcaaggagccgggaacgatggagtcgactacagccaggtgctgattacagatgaggaagaaattaaagccaccaaccctgtagccacagaggaatacggagcagtggccatcaacaaccaggccgctaacacgcaggcgcaaactggacttgtgcataaccagggagttattcctggtatggtctggcagaaccgggacgtgtacctgcagggccctatttgggctaaaatacctcacacagatggcaactttcacccgtctcctctgatgggtggatttggactgaaacacccacctccacagattctaattaaaaatacaccagtgccggcagatcctcctcttaccttcaatcaagccaagctgaactctttcatcacgcagtacagcacgggacaagtcagcgtggaaatcgagtgggagctgcagaaagaaaacagcaagcgctggaatccagagatccagtatacttcaaactactacaaatctacaaatgtggactttgctgtcaataccaaaggtgtttactctgagcctcgccccattggtactcgttacctcacccgtaatttgtaa

SEQ ID NO: 71 - protein, 736 aa, synthetic construct
  REGION 1..736: note = AAV9 VP1
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 72 - protein, 599 aa, synthetic construct
  REGION 1..599: note = AAV9 VP2
MAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 73 - protein, 534 aa, synthetic construct
  REGION 1..534: note = AAV9 VP3
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 74 - protein, 6 aa, synthetic construct
  REGION 1..6: note = VRII-204/AAV6
TNDGVK

SEQ ID NO: 75 - protein, 4 aa, synthetic construct
  REGION 1..4: note = VRIII-204/AAV6
NNGS

SEQ ID NO: 76 - protein, 11 aa, synthetic construct
  REGION 1..11: note = VRIV-204/AAV6
QNQSGSAQNKD

SEQ ID NO: 77 - protein, 18 aa, synthetic construct
  REGION 1..18: note = VRV-204/AAV6
RVSKTKTDNNNSNFTWTG

SEQ ID NO: 78 - protein, 13 aa, synthetic construct
  REGION 1..13: note = VRVI-204/AAV6
HKDDKDKFFPMSG

SEQ ID NO: 79 - protein, 14 aa, synthetic construct
  REGION 1..14: note = VRVII-204/AAV6
KESAGASNTALDNV

SEQ ID NO: 80 - protein, 13 aa, synthetic construct
  REGION 1..13: note = VRVIII-204/AAV6
AVNLQNSSTDPAT

SEQ ID NO: 81 - protein, 10 aa, synthetic construct
  REGION 1..10: note = VRIX-204/AAV6
NYAKSANVDF

SEQ ID NO: 82 - DNA, 2211 bp, synthetic construct
  misc_feature 1..2211: note = AAV214-AB VP1
  source 1..2211: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaaccttttggtctggttgaggaaggtgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcggacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagcagcacatctggaggatcttcaaatgacaacgcctacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtcctcggctctgcgcaccagggctgcctccctccgttcccggcggacgtgttcatgattccgcagtacggctacctaacgctcaacgacggcagccaggcagtgggacggtcatccttttactgcctggaatatttcccatcgcagatgctgagaacgggcaacaactttaccttcagctacacctttgaggacgttcctttccacagcagctacgctcacagccagagtctggaccgtctcatgaatcctctgattgaccagtacctgtactacttgtctaagactatcaacggatccggccagaatcagcagactctgaagttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 83 - DNA, 1605 bp, synthetic construct
  misc_feature 1..1605: note = AAV214-AB VP3
  source 1..1605: mol_type = other DNA
atggcttcaggcggtggcgcaccaatggcggacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagcagcacatctggaggatcttcaaatgacaacgcctacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtcctcggctctgcgcaccagggctgcctccctccgttcccggcggacgtgttcatgattccgcagtacggctacctaacgctcaacgacggcagccaggcagtgggacggtcatccttttactgcctggaatatttcccatcgcagatgctgagaacgggcaacaactttaccttcagctacacctttgaggacgttcctttccacagcagctacgctcacagccagagtctggaccgtctcatgaatcctctgattgaccagtacctgtactacttgtctaagactatcaacggatccggccagaatcagcagactctgaagttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 84 - protein, 736 aa, synthetic construct
  REGION 1..736: note = AAV214AB VP1
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 85 - protein, 599 aa, synthetic construct
  REGION 1..599: note = AAV214AB VP2
MAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 86 - protein, 534 aa, synthetic construct
  REGION 1..534: note = AAV214AB VP3
MASGGGAPMADNNEGADGVGNASGNWHCDSTWLGDRVITTSTRTWALPTYNNHLYKQISSSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 87 - protein, 8 aa, synthetic construct
  REGION 1..8: note = AAV214AB VR-1 amino acid
SSTSGGSS

SEQ ID NO: 88 - DNA, 6719 bp, synthetic construct
  misc_feature 1..6719: note = pA-CF1
  source 1..6719: mol_type = other DNA
tcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcatggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcataccggtagtactcgccaccatgcagagaagccccctggagaaggcctctgtggtgagcaagctgttcttcagctggaccagacccatcctgagaaagggctacagacagagactggagctgtctgacatctaccagatcccctctgtggactctgctgacaacctgtctgagaagctggagagagagtgggacagagagctggccagcaagaagaaccccaagctgatcaatgccctgagaagatgcttcttctggagattcatgttctatggcatcttcctgtacctgggggaggtgaccaaggctgtgcagcccctgctgctgggcagaatcattgccagctatgaccctgacaacaaggaggagagaagcattgccatctacctgggcattggcctgtgcctgctgttcattgtgagaaccctgctgctgcaccctgccatctttggcctgcaccacattggcatgcagatgagaattgccatgttcagcctgatctacaagaagaccctgaagctgagcagcagagtgctggacaagatcagcattggccagctggtgagcctgctgagcaacaacctgaacaagtttgatgagggcctggccctggcccactttgtgtggattgcccccctgcaggtggccctgctgatgggcctgatctgggagctgctgcaggcctctgccttctgtggcctgggcttcctgattgtgctggccctgttccaggctggcctgggcagaatgatgatgaagtacagagaccagagagctggcaagatctctgagagactggtgatcacctctgagatgattgagaacatccagtctgtgaaggcctactgctgggaggaggccatggagaagatgattgagaacctgagacagacagagctgaagctgaccagaaaggctgcctatgtgagatacttcaacagctctgccttcttcttctctggcttctttgtggtgttcctgtctgtgctgccctatgccctgatcaagggcatcatcctgagaaagatcttcaccaccatcagcttctgcattgtgctgagaatggctgtgaccagacagttcccctgggctgtgcagacctggtatgacagcctgggggccatcaacaagatccaggacttcctgcagaagcaggagtacaagaccctggagtacaacctgaccaccacagaggtggtgatggagaatgtgacagccttctgggaggagggctttggggagctgtttgagaaggccaagcagaacaacaacaacagaaagaccagcaatggggatgacagcctgttcttcagcaacttcagcctgctgggcacccctgtgctgaaggacatcaacttcaagattgagagaggccagctgctggctgtggctggcagcacaggggctggcaagaccagcctgctgatgatgatcatgggggagctggagccctctgagggcaagatcaagcactctggcagaatcagcttctgcagccagttcagctggatcatgcctggcaccatcaaggagaacatcatctttggggtgagctatgatgagtacagatacagatctgtgatcaaggcctgccagctggaggaggacatcagcaagtttgctgagaaggacaacattgtgctgggggaggggggcatcaccctgtctgggggccagagagccagaatcagcctggccagagctgtgtacaaggatgctgacctgtacctgctggacagcccctttggctacctggatgtgctgacagagaaggagatctttgagagctgtgtgtgcaagctgatggccaacaagaccagaatcctggtgaccagcaagatggagcacctgaagaaggctgacaagatcctgatcctgcatgagggcagcagctacttctatggcaccttctctgagctgcagaacctgcagcctgacttcagcagcaagctgatgggctgtgacagctttgaccagttctctgctgagagaagaaacagcatcctgacagagaccctgcacagattcagcctggagggggatgcccctgtgagctggacagagaccaagaagcagagcttcaagcagacaggggagtttggggagaagagaaagaacagcatcctgaaccccatcaacagcaccctgcaggccagaagaagacagtctgtgctgaacctgatgacccactctgtgaaccagggccagaacatccacagaaagaccacagccagcaccagaaaggtgagcctggccccccaggccaacctgacagagctggacatctacagcagaagactgagccaggagacaggcctggagatctctgaggagatcaatgaggaggacctgaaggagtgcttctttgatgacatggagagcatccctgctgtgaccacctggaacacctacctgagatacatcacagtgcacaagagcctgatctttgtgctgatctggtgcctggtgatcttcctggctgaggtggctgccagcctggtggtgctgtggctgctgggcaacacccccctgcaggacaagggcaacagcacccacagcagaaacaacagctatgctgtgatcatcaccagcaccagcagctactatgtgttctacatctatgtgggggtggctgacaccctgctggccatgggcttcttcagaggcctgcccctggtgcacaccctgatcacagtgagcaagatcctgcaccacaagatgctgcactctgtgctgcaggcccccatgagcaccctgaacaccctgaaggctgggggcatcctgaacagattcagcaaggacattgccatcctggatgacctgctgcccctgaccatctttgacttcatccagctgctgctgattgtgattggggccattgctgtggtggctgtgctgcagccctacatctttgtggccacagtgcctgtgattgtggccttcatcatgctgagagcctacttcctgcagaccagccagcagctgaagcagctggagtctgagggcagaagccccatcttcacccacctggtgaccagcctgaagggcctgtggaccctgagagcctttggcagacagccctactttgagaccctgttccacaaggccctgaacctgcacacagccaactggttcctgtacctgagcaccctgagatggttccagatgagaattgagatgatctttgtgatcttcttcattgctgtgaccttcatcagcatcctgaccacaggggagggggagggcagagtgggcatcatcctgaccctggccatgaacatcatgagcaccctgcagtgggctgtgaacagcagcattgatgtggacagcctgatgagatctgtgagcagagtgttcaagttcattgacatgcccacagagggcaagcccaccaagagcaccaagccctacaagaatggccagctgagcaaggtgatgatcattgagaacagccatgtgaagaaggatgacatctggccctctgggggccagatgacagtgaaggacctgacagccaagtacacagaggggggcaatgccatcctggagaacatcagcttcagcatcagccctggccagagagtgggcctgctgggcagaacaggctctggcaagagcaccctgctgtctgccttcctgagactgctgaacacagagggggagatccagattgatggggtgagctgggacagcatcaccctgcagcagtggagaaaggcctttggggtgatcccccagaaggtgttcatcttctctggcaccttcagaaagaacctggacccctatgagcagtggtctgaccaggagatctggaaggtggctgatgaggtgggcctgagatctgtgattgagcagttccctggcaagctggactttgtgctggtggatgggggctgtgtgctgagccatggccacaagcagctgatgtgcctggccagatctgtgctgagcaaggccaagatcctgctgctggatgagccctctgcccacctggaccctgtgacctaccagatcatcagaagaaccctgaagcaggcctttgctgactgcacagtgatcctgtgtgagcacagaattgaggccatgctggagtgccagcagttcctggtgattgaggagaacaaggtgagacagtatgacagcatccagaagctgctgaatgagagaagcctgttcagacaggccatcagcccctctgacagagtgaagctgttcccccacagaaacagcagcaagtgcaagagcaagccccagattgctgccctgaaggaggagaccgaggaggaggtgcaggacaccagactgtaaataaaatacgaaatggatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaattaattaaggcgatgaacggtaatcgtaaaactagcatgtcaatcatatgtaccccggttgataatcagaaaagccccaaaaacaggaagattgtataagcattaattaatttaaatacatggacatgtcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaaagcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtttaaaccatg

SEQ ID NO: 89 - DNA, 6751 bp, synthetic construct
  misc_feature 1..6751: note = pA-CF3
  source 1..6751: mol_type = other DNA
tcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcatggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcataccggtagtactcgccaccatgcagagaagccccctggagaaggcctctgtggtgagcaagctgttcttcagctggaccagacccatcctgagaaagggctacagacagagactggagctgtctgacatctaccagatcccctctgtggactctgctgacaacctgtctgagaagctggagagagagtgggacagagagctggccagcaagaagaaccccaagctgatcaatgccctgagaagatgcttcttctggagattcatgttctatggcatcttcctgtacctgggggaggtgaccaaggctgtgcagcccctgctgctgggcagaatcattgccagctatgaccctgacaacaaggaggagagaagcattgccatctacctgggcattggcctgtgcctgctgttcattgtgagaaccctgctgctgcaccctgccatctttggcctgcaccacattggcatgcagatgagaattgccatgttcagcctgatctacaagaagaccctgaagctgagcagcagagtgctggacaagatcagcattggccagctggtgagcctgctgagcaacaacctgaacaagtttgatgagggcctggccctggcccactttgtgtggattgcccccctgcaggtggccctgctgatgggcctgatctgggagctgctgcaggcctctgccttctgtggcctgggcttcctgattgtgctggccctgttccaggctggcctgggcagaatgatgatgaagtacagagaccagagagctggcaagatctctgagagactggtgatcacctctgagatgattgagaacatccagtctgtgaaggcctactgctgggaggaggccatggagaagatgattgagaacctgagacagacagagctgaagctgaccagaaaggctgcctatgtgagatacttcaacagctctgccttcttcttctctggcttctttgtggtgttcctgtctgtgctgccctatgccctgatcaagggcatcatcctgagaaagatcttcaccaccatcagcttctgcattgtgctgagaatggctgtgaccagacagttcccctgggctgtgcagacctggtatgacagcctgggggccatcaacaagatccaggacttcctgcagaagcaggagtacaagaccctggagtacaacctgaccaccacagaggtggtgatggagaatgtgacagccttctgggaggagggctttggggagctgtttgagaaggccaagcagaacaacaacaacagaaagaccagcaatggggatgacagcctgttcttcagcaacttcagcctgctgggcacccctgtgctgaaggacatcaacttcaagattgagagaggccagctgctggctgtggctggcagcacaggggctggcaagaccagcctgctgatgatgatcatgggggagctggagccctctgagggcaagatcaagcactctggcagaatcagcttctgcagccagttcagctggatcatgcctggcaccatcaaggagaacatcatctttggggtgagctatgatgagtacagatacagatctgtgatcaaggcctgccagctggaggaggacatcagcaagtttgctgagaaggacaacattgtgctgggggaggggggcatcaccctgtctgggggccagagagccagaatcagcctggccagagctgtgtacaaggatgctgacctgtacctgctggacagcccctttggctacctggatgtgctgacagagaaggagatctttgagagctgtgtgtgcaagctgatggccaacaagaccagaatcctggtgaccagcaagatggagcacctgaagaaggctgacaagatcctgatcctgcatgagggcagcagctacttctatggcaccttctctgagctgcagaacctgcagcctgacttcagcagcaagctgatgggctgtgacagctttgaccagttctctgctgagagaagaaacagcatcctgacagagaccctgcacagattcagcctggagggggatgcccctgtgagctggacagagaccaagaagcagagcttcaagcagacaggggagtttggggagaagagaaagaacagcatcctgaaccccatcaacagcaccctgcaggccagaagaagacagtctgtgctgaacctgatgacccactctgtgaaccagggccagaacatccacagaaagaccacagccagcaccagaaaggtgagcctggccccccaggccaacctgacagagctggacatctacagcagaagactgagccaggagacaggcctggagatctctgaggagatcaatgaggaggacctgaaggagtgcttctttgatgacatggagagcatccctgctgtgaccacctggaacacctacctgagatacatcacagtgcacaagagcctgatctttgtgctgatctggtgcctggtgatcttcctggctgaggtggctgccagcctggtggtgctgtggctgctgggcaacacccccctgcaggacaagggcaacagcacccacagcagaaacaacagctatgctgtgatcatcaccagcaccagcagctactatgtgttctacatctatgtgggggtggctgacaccctgctggccatgggcttcttcagaggcctgcccctggtgcacaccctgatcacagtgagcaagatcctgcaccacaagatgctgcactctgtgctgcaggcccccatgagcaccctgaacaccctgaaggctgggggcatcctgaacagattcagcaaggacattgccatcctggatgacctgctgcccctgaccatctttgacttcatccagctgctgctgattgtgattggggccattgctgtggtggctgtgctgcagccctacatctttgtggccacagtgcctgtgattgtggccttcatcatgctgagagcctacttcctgcagaccagccagcagctgaagcagctggagtctgagggcagaagccccatcttcacccacctggtgaccagcctgaagggcctgtggaccctgagagcctttggcagacagccctactttgagaccctgttccacaaggccctgaacctgcacacagccaactggttcctgtacctgagcaccctgagatggttccagatgagaattgagatgatctttgtgatcttcttcattgctgtgaccttcatcagcatcctgaccacaggggagggggagggcagagtgggcatcatcctgaccctggccatgaacatcatgagcaccctgcagtgggctgtgaacagcagcattgatgtggacagcctgatgagatctgtgagcagagtgttcaagttcattgacatgcccacagagggcaagcccaccaagagcaccaagccctacaagaatggccagctgagcaaggtgatgatcattgagaacagccatgtgaagaaggatgacatctggccctctgggggccagatgacagtgaaggacctgacagccaagtacacagaggggggcaatgccatcctggagaacatcagcttcagcatcagccctggccagagagtgggcctgctgggcagaacaggctctggcaagagcaccctgctgtctgccttcctgagactgctgaacacagagggggagatccagattgatggggtgagctgggacagcatcaccctgcagcagtggagaaaggcctttggggtgatcccccagaaggtgttcatcttctctggcaccttcagaaagaacctggacccctatgagcagtggtctgaccaggagatctggaaggtggctgatgaggtgggcctgagatctgtgattgagcagttccctggcaagctggactttgtgctggtggatgggggctgtgtgctgagccatggccacaagcagctgatgtgcctggccagatctgtgctgagcaaggccaagatcctgctgctggatgagccctctgcccacctggaccctgtgacctaccagatcatcagaagaaccctgaagcaggcctttgctgactgcacagtgatcctgtgtgagcacagaattgaggccatgctggagtgccagcagttcctggtgattgaggagaacaaggtgagacagtatgacagcatccagaagctgctgaatgagagaagcctgttcagacaggccatcagcccctctgacagagtgaagctgttcccccacagaaacagcagcaagtgcaagagcaagccccagattgctgccctgaaggaggagaccgaggaggaggtgcaggacaccagactgtaaataaatatctttattttcattacatctgtgtgttggttttttgtgtggatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaattaattaaggcgatgaacggtaatcgtaaaactagcatgtcaatcatatgtaccccggttgataatcagaaaagccccaaaaacaggaagattgtataagcattaattaatttaaatacatggacatgtcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaaagcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtttaaaccatg

SEQ ID NO: 90 - DNA, 6603 bp, synthetic construct
  misc_feature 1..6603: note = pA-CF5
  source 1..6603: mol_type = other DNA
tcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcaatatttgcatgtcgctatgtgttctgggaaatcaccataaacgtgaaatgtctttggatttgggaatcttcgaagttctgtatgagaccacagatctccaccggtagtactcgccaccatgcagagaagccccctggagaaggcctctgtggtgagcaagctgttcttcagctggaccagacccatcctgagaaagggctacagacagagactggagctgtctgacatctaccagatcccctctgtggactctgctgacaacctgtctgagaagctggagagagagtgggacagagagctggccagcaagaagaaccccaagctgatcaatgccctgagaagatgcttcttctggagattcatgttctatggcatcttcctgtacctgggggaggtgaccaaggctgtgcagcccctgctgctgggcagaatcattgccagctatgaccctgacaacaaggaggagagaagcattgccatctacctgggcattggcctgtgcctgctgttcattgtgagaaccctgctgctgcaccctgccatctttggcctgcaccacattggcatgcagatgagaattgccatgttcagcctgatctacaagaagaccctgaagctgagcagcagagtgctggacaagatcagcattggccagctggtgagcctgctgagcaacaacctgaacaagtttgatgagggcctggccctggcccactttgtgtggattgcccccctgcaggtggccctgctgatgggcctgatctgggagctgctgcaggcctctgccttctgtggcctgggcttcctgattgtgctggccctgttccaggctggcctgggcagaatgatgatgaagtacagagaccagagagctggcaagatctctgagagactggtgatcacctctgagatgattgagaacatccagtctgtgaaggcctactgctgggaggaggccatggagaagatgattgagaacctgagacagacagagctgaagctgaccagaaaggctgcctatgtgagatacttcaacagctctgccttcttcttctctggcttctttgtggtgttcctgtctgtgctgccctatgccctgatcaagggcatcatcctgagaaagatcttcaccaccatcagcttctgcattgtgctgagaatggctgtgaccagacagttcccctgggctgtgcagacctggtatgacagcctgggggccatcaacaagatccaggacttcctgcagaagcaggagtacaagaccctggagtacaacctgaccaccacagaggtggtgatggagaatgtgacagccttctgggaggagggctttggggagctgtttgagaaggccaagcagaacaacaacaacagaaagaccagcaatggggatgacagcctgttcttcagcaacttcagcctgctgggcacccctgtgctgaaggacatcaacttcaagattgagagaggccagctgctggctgtggctggcagcacaggggctggcaagaccagcctgctgatgatgatcatgggggagctggagccctctgagggcaagatcaagcactctggcagaatcagcttctgcagccagttcagctggatcatgcctggcaccatcaaggagaacatcatctttggggtgagctatgatgagtacagatacagatctgtgatcaaggcctgccagctggaggaggacatcagcaagtttgctgagaaggacaacattgtgctgggggaggggggcatcaccctgtctgggggccagagagccagaatcagcctggccagagctgtgtacaaggatgctgacctgtacctgctggacagcccctttggctacctggatgtgctgacagagaaggagatctttgagagctgtgtgtgcaagctgatggccaacaagaccagaatcctggtgaccagcaagatggagcacctgaagaaggctgacaagatcctgatcctgcatgagggcagcagctacttctatggcaccttctctgagctgcagaacctgcagcctgacttcagcagcaagctgatgggctgtgacagctttgaccagttctctgctgagagaagaaacagcatcctgacagagaccctgcacagattcagcctggagggggatgcccctgtgagctggacagagaccaagaagcagagcttcaagcagacaggggagtttggggagaagagaaagaacagcatcctgaaccccatcaacagcaccctgcaggccagaagaagacagtctgtgctgaacctgatgacccactctgtgaaccagggccagaacatccacagaaagaccacagccagcaccagaaaggtgagcctggccccccaggccaacctgacagagctggacatctacagcagaagactgagccaggagacaggcctggagatctctgaggagatcaatgaggaggacctgaaggagtgcttctttgatgacatggagagcatccctgctgtgaccacctggaacacctacctgagatacatcacagtgcacaagagcctgatctttgtgctgatctggtgcctggtgatcttcctggctgaggtggctgccagcctggtggtgctgtggctgctgggcaacacccccctgcaggacaagggcaacagcacccacagcagaaacaacagctatgctgtgatcatcaccagcaccagcagctactatgtgttctacatctatgtgggggtggctgacaccctgctggccatgggcttcttcagaggcctgcccctggtgcacaccctgatcacagtgagcaagatcctgcaccacaagatgctgcactctgtgctgcaggcccccatgagcaccctgaacaccctgaaggctgggggcatcctgaacagattcagcaaggacattgccatcctggatgacctgctgcccctgaccatctttgacttcatccagctgctgctgattgtgattggggccattgctgtggtggctgtgctgcagccctacatctttgtggccacagtgcctgtgattgtggccttcatcatgctgagagcctacttcctgcagaccagccagcagctgaagcagctggagtctgagggcagaagccccatcttcacccacctggtgaccagcctgaagggcctgtggaccctgagagcctttggcagacagccctactttgagaccctgttccacaaggccctgaacctgcacacagccaactggttcctgtacctgagcaccctgagatggttccagatgagaattgagatgatctttgtgatcttcttcattgctgtgaccttcatcagcatcctgaccacaggggagggggagggcagagtgggcatcatcctgaccctggccatgaacatcatgagcaccctgcagtgggctgtgaacagcagcattgatgtggacagcctgatgagatctgtgagcagagtgttcaagttcattgacatgcccacagagggcaagcccaccaagagcaccaagccctacaagaatggccagctgagcaaggtgatgatcattgagaacagccatgtgaagaaggatgacatctggccctctgggggccagatgacagtgaaggacctgacagccaagtacacagaggggggcaatgccatcctggagaacatcagcttcagcatcagccctggccagagagtgggcctgctgggcagaacaggctctggcaagagcaccctgctgtctgccttcctgagactgctgaacacagagggggagatccagattgatggggtgagctgggacagcatcaccctgcagcagtggagaaaggcctttggggtgatcccccagaaggtgttcatcttctctggcaccttcagaaagaacctggacccctatgagcagtggtctgaccaggagatctggaaggtggctgatgaggtgggcctgagatctgtgattgagcagttccctggcaagctggactttgtgctggtggatgggggctgtgtgctgagccatggccacaagcagctgatgtgcctggccagatctgtgctgagcaaggccaagatcctgctgctggatgagccctctgcccacctggaccctgtgacctaccagatcatcagaagaaccctgaagcaggcctttgctgactgcacagtgatcctgtgtgagcacagaattgaggccatgctggagtgccagcagttcctggtgattgaggagaacaaggtgagacagtatgacagcatccagaagctgctgaatgagagaagcctgttcagacaggccatcagcccctctgacagagtgaagctgttcccccacagaaacagcagcaagtgcaagagcaagccccagattgctgccctgaaggaggagaccgaggaggaggtgcaggacaccagactgtaaataaatatctttattttcattacatctgtgtgttggttttttgtgtggatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaattaattaaggcgatgaacggtaatcgtaaaactagcatgtcaatcatatgtaccccggttgataatcagaaaagccccaaaaacaggaagattgtataagcattaattaatttaaatacatggacatgtcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaaagcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtttaaaccatg

SEQ ID NO: 91 - DNA, 7519 bp, synthetic construct
  misc_feature 1..7519: note = pA-CF7
  source 1..7519: mol_type = other DNA
tcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcaatatttgcatgtcgctatgtgttctgggaaatcaccataaacgtgaaatgtctttggatttgggaatcttcgaagttctgtatgagaccacagatctccaccggtagtactcgccaccatgcagagaagccccctggagaaggcctctgtggtgagcaagctgttcttcccccctggagaaggcctctgtggtgagcaagctgttcttcagctggaccagacccatcctgagaaagggctacagacagagactggagctgtctgacatctaccagatcccctctgtggactctgctgacaacctgtctgagaagctggagagagagtgggacagagagctggccagcaagaagaaccccaagctgatcaatgccctgagaagatgcttcttctggagattcatgttctatggcatcttcctgtacctgggggaggtgaccaaggctgtgcagcccctgctgctgggcagaatcattgccagctatgacccagcccctgctgctgggcagaatcattgccagctatgaccctgacaacaaggaggagagaagcattgccatctacctgggcattggcctgtgcctgctgttcattgtgagaaccctgctgctgcaccctgccatctttggcctgcaccacattggcatgcagatgagaattgccatgttcagcctgatctacaagaagaccctgaagctgagcagcagagtgctggacaagatcagcattggccagctggtgagcctgctgagcaacaacctgaacaagtttgatgagggcctggccctggcccactttgtgtggattgcttgatgagggcctggccctggcccactttgtgtggattgcccccctgcaggtggccctgctgatgggcctgatctgggagctgctgcaggcctctgccttctgtggcctgggcttcctgattgtgctggccctgttccaggctggcctgggcagaatgatgatgaagtacagagaccagagagctggcaagatctctgagagactggtgatcacctctgagatgattgagaacatccagtctgtgaaggcctactgctgggaggaggccatggagaagatgattgagaacctgagacagacagagctgaagctgaccagaaaggctgcctatgtgagatacttcaacagctctgccttcttcttctctggcttctttgtggtgttcctgtctgtgctgcctctgccttcttcttctctggcttctttgtggtgttcctgtctgtgctgccctatgccctgatcaagggcatcatcctgagaaagatcttcaccaccatcagcttctgcattgtgctgagaatggctgtgaccagacagttcccctgggctgtgcagacctggtatgacagcctgggggccatcaacaagatccaggacttcctgcagaagcaggagtacaagaccctggagtacaacctgaccaccacagaggtggtgatggagaatgtgacagccttctgggaggagggctttggggagctgtttgagaaggccaagcagaacaacaacaacagaaagaccagcaatggggatgacagcctgttcttcagcaacttcagcctgctgggcacccctgtgcggatgacagcctgttcttcagcaacttcagcctgctgggcacccctgtgctgaaggacatcaacttcaagattgagagaggccagctgctggctgtggctggcagcacaggggctggcaagaccagcctgctgatgatgatcatgggggagctggagccctctgagggcaagatcaagcactctggcagaatcagcttctgcagccagttcagctggatcatgcctggcaccatcaaggagaacatcatctttggggtgagctatgatgagtacagatacagatctgtgatcaaggcctgccagctggaggaggacatcaagatctgtgatcaaggcctgccagctggaggaggacatcagcaagtttgctgagaaggacaacattgtgctgggggaggggggcatcaccctgtctgggggccagagagccagaatcagcctggccagagctgtgtacaaggatgctgacctgtacctgctggacagcccctttggctacctggatgtgctgacagagaaggagatctttgagagctgtgtgtgcaagctgatggccaacaagaccagaatcctggtgaccagcaagatggagcacctgaagaaggctgacaagatcctgatcctgcatgagggcagcagagaaggctgacaagatcctgatcctgcatgagggcagcagctacttctatggcaccttctctgagctgcagaacctgcagcctgacttcagcagcaagctgatgggctgtgacagctttgaccagttctctgctgagagaagaaacagcatcctgacagagaccctgcacagattcagcctggagggggatgcccctgtgagctggacagagaccaagaagcagagcttcaagcagacaggggagtttggggagaagagaaagaacagcatcctgaaccccatcaacagcatcagaaagttcagcattgtgcagaagacccatcaacagcatcagaaagttcagcattgtgcagaagacccccctgcagatgaatggcattgaggaggactctgatgagcccctggagagaagactgagcctggtgcctgactctgagcagggggaggccatcctgcccagaatctctgtgatcagcacaggccccaccctgcaggccagaagaagacagtctgtgctgaacctgatgacccactctgtgaaccagggccagaacatccaccactctgtgaaccagggccagaacatccacagaaagaccacagccagcaccagaaaggtgagcctggccccccaggccaacctgacagagctggacatctacagcagaagactgagccaggagacaggcctggagatctctgaggagatcaatgaggaggacctgaaggagtgcttctttgatgacatggagagcatccctgctgtgaccacctggaacacctacctgagatacatcacagtgcacaagagcctgatctttgtgctgatctggtgcctggtgatcttcctggctgaggtggctgccagcctggtggtgcgtgatcttcctggctgaggtggctgccagcctggtggtgctgtggctgctgggcaacacccccctgcaggacaagggcaacagcacccacagcagaaacaacagctatgctgtgatcatcaccagcaccagcagctactatgtgttctacatctatgtgggggtggctgacaccctgctggccatgggcttcttcagaggcctgcccctggtgcacaccctgatcacagtgagcaagatcctgcaccacaagatgctgcactctgtgctgcaggcccccatgagcaccctgaacaccctgaaggctgggggcatcctgaatgagcaccctgaacaccctgaaggctgggggcatcctgaacagattcagcaaggacattgccatcctggatgacctgctgcccctgaccatctttgacttcatccagctgctgctgattgtgattggggccattgctgtggtggctgtgctgcagccctacatctttgtggccacagtgcctgtgattgtggccttcatcatgctgagagcctacttcctgcagaccagccagcagctgaagcagctggagtctgagggcagaagccccatcttcacccacctggtgaccagcctgaagggcctgtggaccctgagagcccctggtgaccagcctgaagggcctgtggaccctgagagcctttggcagacagccctactttgagaccctgttccacaaggccctgaacctgcacacagccaactggttcctgtacctgagcaccctgagatggttccagatgagaattgagatgatctttgtgatcttcttcattgctgtgaccttcatcagcatcctgaccacaggggagggggagggcagagtgggcatcatcctgaccctggccatgaacatcatgagcaccctgcagtgggctgtgaacagcagcattgatgtggacagcctgatgagatctgtgagcagagtgttcaagttcattgacatgcccacagagggcaagcccaccaagagcaccaagccctacaagaatggccagctgcagagggcaagcccaccaagagcaccaagccctacaagaatggccagctgagcaaggtgatgatcattgagaacagccatgtgaagaaggatgacatctggccctctgggggccagatgacagtgaaggacctgacagccaagtacacagaggggggcaatgccatcctggagaacatcagcttcagcatcagccctggccagagagtgggcctgctgggcagaacaggctctggcaagagcaccctgctgtctgccttcctgagactgctgaacacagagggggagatccagattgatggggtgagctgggacagcatcaccctgcagcagtggagaaaggcctttggggtgatcccccagaaggtgttcatcttctctggcaccttcagaaagaacctgtgatcccccagaaggtgttcatcttctctggcaccttcagaaagaacctggacccctatgagcagtggtctgaccaggagatctggaaggtggctgatgaggtgggcctgagatctgtgattgagcagttccctggcaagctggactttgtgctggtggatgggggctgtgtgctgagccatggccacaagcagctgatgtgcctggccagatctgtgctgagcaaggccaagatcctgctgctggatgagccctctgcccacctggaccctgtgacctaccagatcatcagaagaaccctgaagcaggcctttgctgaaccagatcatcagaagaaccctgaagcaggcctttgctgactgcacagtgatcctgtgtgagcacagaattgaggccatgctggagtgccagcagttcctggtgattgaggagaacaaggtgagacagtatgacagcatccagaagctgctgaatgagagaagcctgttcagacaggccatcagcccctctgacagagtgaagctgttcccccacagaaacagcagcaagtgcaagagcaagccccagattgctgccctgaaggaggagaccgaggaggaggtgcaggacaccagactgtaaataaatatctttattttcattacatctgtgtgttggttttttgtgtggatctgaggaacccctagtgatggagttggccactccctctctgcgcgctcatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaattaattaaggcgatgaacggtaatcgtaaaactagcatgtcaatcatatgtaccccggttgataatcagaaaagccccaaaaacaggaagattgtataagcattaattaatttaaatacatggacatgtcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaaagcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggaccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtttaaaccatg

SEQ ID NO: 92 - DNA, 11577 bp, synthetic construct
  misc_feature 1..11577: note = pHELPK plasmid DNA
  source 1..11577: mol_type = other DNA
ggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacgtcgacgtttaaaccatatgatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaacaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgatggatcc

SEQ ID NO: 93 - DNA, 4443 bp, synthetic construct
  misc_feature 1..4443: note = CFTR
  source 1..4443: mol_type = other DNA
atgcagagaagccccctggagaaggcctctgtggtgagcaagctgttcttcagctggaccagacccatcctgagaaagggctacagacagagactggagctgtctgacatctaccagatcccctctgtggactctgctgacaacctgtctgagaagctggagagagagtgggacagagagctggccagcaagaagaaccccaagctgatcaatgccctgagaagatgcttcttctggagattcatgttctatggcatcttcctgtacctgggggaggtgaccaaggctgtgcagcccctgctgctgggcagaatcattgccagctatgaccctgacaacaaggaggagagaagcattgccatctacctgggcattggcctgtgcctgctgttcattgtgagaaccctgctgctgcaccctgccatctttggcctgcaccacattggcatgcagatgagaattgccatgttcagcctgatctacaagaagaccctgaagctgagcagcagagtgctggacaagatcagcattggccagctggtgagcctgctgagcaacaacctgaacaagtttgatgagggcctggccctggcccactttgtgtggattgcccccctgcaggtggccctgctgatgggcctgatctgggagctgctgcaggcctctgccttctgtggcctgggcttcctgattgtgctggccctgttccaggctggcctgggcagaatgatgatgaagtacagagaccagagagctggcaagatctctgagagactggtgatcacctctgagatgattgagaacatccagtctgtgaaggcctactgctgggaggaggccatggagaagatgattgagaacctgagacagacagagctgaagctgaccagaaaggctgcctatgtgagatacttcaacagctctgccttcttcttctctggcttctttgtggtgttcctgtctgtgctgccctatgccctgatcaagggcatcatcctgagaaagatcttcaccaccatcagcttctgcattgtgctgagaatggctgtgaccagacagttcccctgggctgtgcagacctggtatgacagcctgggggccatcaacaagatccaggacttcctgcagaagcaggagtacaagaccctggagtacaacctgaccaccacagaggtggtgatggagaatgtgacagccttctgggaggagggctttggggagctgtttgagaaggccaagcagaacaacaacaacagaaagaccagcaatggggatgacagcctgttcttcagcaacttcagcctgctgggcacccctgtgctgaaggacatcaacttcaagattgagagaggccagctgctggctgtggctggcagcacaggggctggcaagaccagcctgctgatgatgatcatgggggagctggagccctctgagggcaagatcaagcactctggcagaatcagcttctgcagccagttcagctggatcatgcctggcaccatcaaggagaacatcatctttggggtgagctatgatgagtacagatacagatctgtgatcaaggcctgccagctggaggaggacatcagcaagtttgctgagaaggacaacattgtgctgggggaggggggcatcaccctgtctgggggccagagagccagaatcagcctggccagagctgtgtacaaggatgctgacctgtacctgctggacagcccctttggctacctggatgtgctgacagagaaggagatctttgagagctgtgtgtgcaagctgatggccaacaagaccagaatcctggtgaccagcaagatggagcacctgaagaaggctgacaagatcctgatcctgcatgagggcagcagctacttctatggcaccttctctgagctgcagaacctgcagcctgacttcagcagcaagctgatgggctgtgacagctttgaccagttctctgctgagagaagaaacagcatcctgacagagaccctgcacagattcagcctggagggggatgcccctgtgagctggacagagaccaagaagcagagcttcaagcagacaggggagtttggggagaagagaaagaacagcatcctgaaccccatcaacagcatcagaaagttcagcattgtgcagaagacccccctgcagatgaatggcattgaggaggactctgatgagcccctggagagaagactgagcctggtgcctgactctgagcagggggaggccatcctgcccagaatctctgtgatcagcacaggccccaccctgcaggccagaagaagacagtctgtgctgaacctgatgacccactctgtgaaccagggccagaacatccacagaaagaccacagccagcaccagaaaggtgagcctggccccccaggccaacctgacagagctggacatctacagcagaagactgagccaggagacaggcctggagatctctgaggagatcaatgaggaggacctgaaggagtgcttctttgatgacatggagagcatccctgctgtgaccacctggaacacctacctgagatacatcacagtgcacaagagcctgatctttgtgctgatctggtgcctggtgatcttcctggctgaggtggctgccagcctggtggtgctgtggctgctgggcaacacccccctgcaggacaagggcaacagcacccacagcagaaacaacagctatgctgtgatcatcaccagcaccagcagctactatgtgttctacatctatgtgggggtggctgacaccctgctggccatgggcttcttcagaggcctgcccctggtgcacaccctgatcacagtgagcaagatcctgcaccacaagatgctgcactctgtgctgcaggcccccatgagcaccctgaacaccctgaaggctgggggcatcctgaacagattcagcaaggacattgccatcctggatgacctgctgcccctgaccatctttgacttcatccagctgctgctgattgtgattggggccattgctgtggtggctgtgctgcagccctacatctttgtggccacagtgcctgtgattgtggccttcatcatgctgagagcctacttcctgcagaccagccagcagctgaagcagctggagtctgagggcagaagccccatcttcacccacctggtgaccagcctgaagggcctgtggaccctgagagcctttggcagacagccctactttgagaccctgttccacaaggccctgaacctgcacacagccaactggttcctgtacctgagcaccctgagatggttccagatgagaattgagatgatctttgtgatcttcttcattgctgtgaccttcatcagcatcctgaccacaggggagggggagggcagagtgggcatcatcctgaccctggccatgaacatcatgagcaccctgcagtgggctgtgaacagcagcattgatgtggacagcctgatgagatctgtgagcagagtgttcaagttcattgacatgcccacagagggcaagcccaccaagagcaccaagccctacaagaatggccagctgagcaaggtgatgatcattgagaacagccatgtgaagaaggatgacatctggccctctgggggccagatgacagtgaaggacctgacagccaagtacacagaggggggcaatgccatcctggagaacatcagcttcagcatcagccctggccagagagtgggcctgctgggcagaacaggctctggcaagagcaccctgctgtctgccttcctgagactgctgaacacagagggggagatccagattgatggggtgagctgggacagcatcaccctgcagcagtggagaaaggcctttggggtgatcccccagaaggtgttcatcttctctggcaccttcagaaagaacctggacccctatgagcagtggtctgaccaggagatctggaaggtggctgatgaggtgggcctgagatctgtgattgagcagttccctggcaagctggactttgtgctggtggatgggggctgtgtgctgagccatggccacaagcagctgatgtgcctggccagatctgtgctgagcaaggccaagatcctgctgctggatgagccctctgcccacctggaccctgtgacctaccagatcatcagaagaaccctgaagcaggcctttgctgactgcacagtgatcctgtgtgagcacagaattgaggccatgctggagtgccagcagttcctggtgattgaggagaacaaggtgagacagtatgacagcatccagaagctgctgaatgagagaagcctgttcagacaggccatcagcccctctgacagagtgaagctgttcccccacagaaacagcagcaagtgcaagagcaagccccagattgctgccctgaaggaggagaccgaggaggaggtgcaggacaccagactgtaa

SEQ ID NO: 94 - protein, 1480 aa, synthetic construct
  REGION 1..1480: note = CFTR protein
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMMIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 95 - protein, 1428 aa, synthetic construct
  REGION 1..1428: note = CFTRdeltaR protein
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMMIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 96 - DNA, 250 bp, synthetic construct
  misc_feature 1..250: note = Mouse U1a promoter sequence
  source 1..250: mol_type = other DNA
atggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatac

SEQ ID NO: 97 - DNA, 101 bp, synthetic construct
  misc_feature 1..101: note = Polymerase III H1 mutant promoter sequence
  source 1..101: mol_type = other DNA
aatatttgcatgtcgctatgtgttctgggaaatcaccataaacgtgaaatgtctttggatttgggaatcttcgaagttctgtatgagaccacagatctcca

SEQ ID NO: 98 - DNA, 2214 bp, synthetic construct
  misc_feature 1..2214: note = AAV110 DNA
  source 1..2214: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 99 - DNA, 1509 bp, Homo sapiens
  misc_feature 1..1509: note = Sulfoglucosamine sulfohydrolase (SGSH)
  source 1..1509: mol_type = other DNA
atgagctgccccgtgcccgcctgctgcgcgctgctgctagtcctggggctctgccgggcgcgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccagcctttcttcctctacgtcgccttccacgacccccaccgctgtgggcactcccagccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtcggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcaccacagctggtcagcccacgggctggtacaaggacctccgtcattactactaccgggcgcgctgggagctctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgtga

SEQ ID NO: 100 - DNA, 1509 bp, synthetic construct
  misc_feature 1..1509: note = Codon optimized CO1-SGSH
  source 1..1509: mol_type = other DNA
atgagctgtcctgttccagcctgttgtgccctgctgctggttctgggactgtgcagagccagacctaggaacgctctgctgctgctcgctgacgatggcggatttgagagcggcgcctacaacaacagcgccattgccacacctcacctggatgccctggccagaagaagcctgctgttcagaaacgccttcaccagcgtgtccagctgcagcccttctagagctagcctgctgacaggactgccccagcaccagaatgggatgtatggcctgcaccaggacgtgcaccacttcaacagcttcgacaaagtgcggagcctgcctctgcttctgtctcaagccggcgtcagaacaggcatcatcggcaagaaacacgtgggccccgagacagtgtaccccttcgatttcgcctacaccgaagagaacggcagcgtgctgcaagtgggcagaaacatcacccggatcaagctgctcgtgcggaagttcctgcagacccaggacgaccagcctttcttcctgtacgtggccttccacgatcctcacagatgcggccatagccagcctcagtacggcaccttctgcgagaagtttggcaacggcgagagcggcatgggcagaatccctgattggacccctcaggcctacgatcccctggatgtgctggtgccttacttcgtgcctaacacaccagccgccagagccgatctggccgctcagtatacaaccgtgggaagaatggaccaaggcgtcggcctggttctgcaagagcttagagatgccggcgtgctgaacgacaccctggtcatctttaccagcgacaacggcatcccctttccatctggccggaccaatctgtactggcctggaacagctgagcccctgctggtgtctagccctgagcaccctaagagatggggccaagtgtctgaggcctacgtgtccctgctggatctgacccctaccatcctggactggttcagcatcccctatcctagctacgccatcttcggcagcaagaccatccacctgaccggcagatctctgctgccagctctggaagctgaacctctgtgggccacagtgtttggcagccagtctcaccacgaagtgacaatgagctaccccatgcggagcgtgcagcacagacacttcagactggtgcacaacctgaacttcaagatgccctttccaatcgaccaggacttctatgtgtccccaaccttccaggacctgctgaacagaaccacagccggccaacctaccggctggtacaaggacctgcggcactactactatagagccagatgggagctgtacgaccggtccagagatccccacgagacacagaacctggccaccgatcctagattcgcccagctgctggaaatgctgagagatcagctggccaagtggcagtgggagacacacgatccttgggtctgcgctcctgatggcgtgctggaagagaagctgtcccctcagtgtcagcccctgcacaacgagctttaa

SEQ ID NO: 101 - DNA, 1596 bp, synthetic construct
  misc_feature 1..1596: note = Codon optimized + GET CO1-SGSH-GET
  source 1..1596: mol_type = other DNA
atgagctgtcctgttccagcctgttgtgccctgctgctggttctgggactgtgcagagccagacctaggaacgctctgctgctgctcgctgacgatggcggatttgagagcggcgcctacaacaacagcgccattgccacacctcacctggatgccctggccagaagaagcctgctgttcagaaacgccttcaccagcgtgtccagctgcagcccttctagagctagcctgctgacaggactgccccagcaccagaatgggatgtatggcctgcaccaggacgtgcaccacttcaacagcttcgacaaagtgcggagcctgcctctgcttctgtctcaagccggcgtcagaacaggcatcatcggcaagaaacacgtgggccccgagacagtgtaccccttcgatttcgcctacaccgaagagaacggcagcgtgctgcaagtgggcagaaacatcacccggatcaagctgctcgtgcggaagttcctgcagacccaggacgaccagcctttcttcctgtacgtggccttccacgatcctcacagatgcggccatagccagcctcagtacggcaccttctgcgagaagtttggcaacggcgagagcggcatgggcagaatccctgattggacccctcaggcctacgatcccctggatgtgctggtgccttacttcgtgcctaacacaccagccgccagagccgatctggccgctcagtatacaaccgtgggaagaatggaccaaggcgtcggcctggttctgcaagagcttagagatgccggcgtgctgaacgacaccctggtcatctttaccagcgacaacggcatcccctttccatctggccggaccaatctgtactggcctggaacagctgagcccctgctggtgtctagccctgagcaccctaagagatggggccaagtgtctgaggcctacgtgtccctgctggatctgacccctaccatcctggactggttcagcatcccctatcctagctacgccatcttcggcagcaagaccatccacctgaccggcagatctctgctgccagctctggaagctgaacctctgtgggccacagtgtttggcagccagtctcaccacgaagtgacaatgagctaccccatgcggagcgtgcagcacagacacttcagactggtgcacaacctgaacttcaagatgccctttccaatcgaccaggacttctatgtgtccccaaccttccaggacctgctgaacagaaccacagccggccaacctaccggctggtacaaggacctgcggcactactactatagagccagatgggagctgtacgaccggtccagagatccccacgagacacagaacctggccaccgatcctagattcgcccagctgctggaaatgctgagagatcagctggccaagtggcagtgggagacacacgatccttgggtctgcgctcctgatggcgtgctggaagagaagctgtcccctcagtgtcagcccctgcacaacgagctgcggcgtcgtcggcgaagaagaagaaagcgcaagaaaaaaggcaaaggcctgggcaagaagcgggacccctgtctgagaaagtacaaataa

SEQ ID NO: 102 - DNA, 1509 bp, synthetic construct
  misc_feature 1..1509: note = Codon optimized CO2-SGSH
  source 1..1509: mol_type = other DNA
atgagctgccctgtgcctgcctgctgtgccctgctgctggtgctgggcctgtgcagagccagacctaggaatgccctgctgctgctggctgatgatgggggctttgagagtggggcctacaacaacagtgccattgccaccccccacctggatgccctggccagaagaagcctgctgttcagaaatgccttcaccagtgtgagcagctgcagccccagcagagccagcctgctgacaggcctgccccagcaccagaatggcatgtatggcctgcaccaggatgtgcaccacttcaacagctttgacaaggtgagaagcctgcccctgctgctgagccaggctggggtgagaacaggcatcattggcaagaagcatgtgggccctgagacagtgtacccctttgactttgcctacacagaggagaatggcagtgtgctgcaggtgggcagaaacatcaccagaatcaagctgctggtgagaaagttcctgcagacccaggatgaccagcccttcttcctgtatgtggccttccatgacccccacagatgtggccacagccagccccagtatggcaccttctgtgagaagtttggcaatggggagagtggcatgggcagaatccctgactggaccccccaggcctatgaccccctggatgtgctggtgccctactttgtgcccaacacccctgctgccagagctgacctggctgcccagtacaccacagtgggcagaatggaccagggggtgggcctggtgctgcaggagctgagagatgctggggtgctgaatgacaccctggtgatcttcaccagtgacaatggcatccccttccccagtggcagaaccaacctgtactggcctggcacagctgagcccctgctggtgagcagccctgagcaccccaagagatggggccaggtgagtgaggcctatgtgagcctgctggacctgacccccaccatcctggactggttcagcatcccctaccccagctatgccatctttggcagcaagaccatccacctgacaggcagaagcctgctgcctgccctggaggctgagcccctgtgggccacagtgtttggcagccagagccaccatgaggtgaccatgagctaccccatgagaagtgtgcagcacagacacttcagactggtgcacaacctgaacttcaagatgcccttccccattgaccaggacttctatgtgagccccaccttccaggacctgctgaacagaaccacagctggccagcccacaggctggtacaaggacctgagacactactactacagagccagatgggagctgtatgacagaagcagagacccccatgagacccagaacctggccacagaccccagatttgcccagctgctggagatgctgagagaccagctggccaagtggcagtgggagacccatgacccctgggtgtgtgcccctgatggggtgctggaggagaagctgagcccccagtgccagcccctgcacaatgagctgtga

SEQ ID NO: 103 - DNA, 921 bp, synthetic construct
  misc_feature 1..921: note = Codon optimized Ceroid Lipofuscinosis, Neuronal, 1 (CLN1)
  source 1..921: mol_type = other DNA
atggcttctccggggtgtctgtggctgctggcagtggcactccttccctggacttgcgccagccgggctctgcagcacctcgaccctccagcccctcttccactggtgatttggcacggaatgggtgattcctgctgtaatcccctgtcaatgggagccatcaagaagatggtggagaagaagatccctggaatctacgtgctgtcactggagattggaaagaccctgatggaggacgtcgagaactccttcttcctcaatgtcaactctcaagtgaccaccgtctgccaggccctggccaaggacccgaagctgcagcaggggtataatgctatggggttcagccagggaggacagttccttcgggctgtggcccaacgctgccctagcccacccatgatcaacctgatctcagtgggtggccagcatcagggcgtgttcggacttccccggtgtcccggggaatcctctcatatctgcgacttcatccgcaaaactctcaatgcaggcgcttattcaaaggtcgtccaagagaggctggtgcaagccgagtactggcacgatcccattaaggaggacgtgtacagaaatcactcaatctttctggccgacattaaccaggagaggggaattaacgaatcatataagaagaatctcatggccctcaaaaagttcgtcatggtgaagttccttaacgatagcattgtggacccagtggacagcgaatggttcggattttaccgctcaggccaggcaaaagaaaccatccctctccaagagacttctctttacacccaagacagacttgggcttaaggaaatggataacgctggtcagctggtgttcctcgccaccgaaggtgaccatctgcagctcagcgaagagtggttctacgctcatatcatcccgtttcttggttga

SEQ ID NO: 104 - DNA, 885 bp, Homo sapiens
  misc_feature 1..885: note = Survival Motor Neuron 1 (SMN1)
  source 1..885: mol_type = other DNA
atggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacagaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggttttagacaaaatcaaaaagaaggaaggtgctcacattccttaaattaa

SEQ ID NO: 105 - DNA, 885 bp, synthetic construct
  misc_feature 1..885: note = Codon optimized CO1-SMN1
  source 1..885: mol_type = other DNA
atggcgatgtctagtggtggatctggtggcggcgtgcccgagcaagaagatagcgtcctgttcagaagaggcaccggccagagcgacgacagcgacatctgggatgatacagccctgatcaaggcctacgacaaggccgtggccagctttaagcacgccctgaagaacggcgatatctgcgagacaagcggcaagcccaagaccacacctaagagaaagcccgccaagaagaacaagagccagaagaagaataccgccgccagcctgcagcagtggaaagtgggcgataagtgcagcgccatttggagcgaggacggctgtatctaccctgccacaatcgccagcatcgacttcaagcgggaaacctgcgtggtggtgtacacaggctacggcaacagagaggaacagaacctgagcgacctgctgtccccaatttgcgaggtggccaacaacatcgagcagaacgcccaagagaacgagaacgagtcccaggtgtccaccgacgagagcgagaatagcagaagccccggcaacaagagcgacaacatcaagcctaagagcgccccttggaacagcttcctgcctcctcctccaccaatgcctggacctagactcggacctggaaagcccggcctgaagttcaatggacctccaccaccgccaccacctccgcctccacatcttctgtcttgttggctgcctccatttcctagcggccctccaatcatcccgccacctccacctatctgccccgacagtctggatgatgctgatgccctgggctccatgctgatctcttggtacatgagcggctaccacaccggctactacatgggcttcagacagaaccagaaagagggccgttgcagccacagcctgaactga

SEQ ID NO: 106 - DNA, 885 bp, synthetic construct
  misc_feature 1..885: note = Codon optimized CO2-SMN1
  source 1..885: mol_type = other DNA
atggccatgagcagtgggggcagtggaggaggggtgcctgagcaggaggacagtgtgctgttcagaagaggcacaggccagagtgatgacagtgacatctgggatgacacagccctgatcaaggcctatgacaaggctgtggccagcttcaagcatgccctgaagaatggggacatctgtgagaccagtggcaagcccaagaccacccccaagagaaagcctgccaagaagaacaagagccagaagaagaacacagctgccagcctgcagcagtggaaggtgggagacaagtgcagtgccatctggagtgaggatggctgcatctaccctgccaccattgccagcattgacttcaagagagagacctgtgtggtggtgtacacaggctatggcaacagagaggagcagaacctgagtgacctgctgagccccatctgtgaggtggccaacaacattgagcagaatgcccaggagaatgagaatgagagccaggtgagcacagatgagagtgagaacagcagaagccctggcaacaagagtgacaacatcaagcccaagagtgccccttggaacagcttcctgccacccccaccacccatgcctggccccagactgggccctggcaagcctggcctgaagttcaatggcccaccaccccctcctccaccaccccctccccacctgctgagctgctggctgccccccttccccagtggcccacccatcatcccacctcccccacccatctgccctgacagcctggatgatgctgatgccctgggcagcatgctgatcagctggtacatgagtggctaccacacaggctactacatgggcttcagacagaaccagaaggagggcagatgcagccacagcctgaactga

SEQ ID NO: 107 - DNA, 1548 bp, Homo sapiens
  misc_feature 1..1548: note = Tissue Non-specific Alkaline Phosphatase (TNALP)
  source 1..1548: mol_type = other DNA
atgatttcaccattcttagtactggccattggcacctgccttactaactcactagtgccagagaaagagaaagaccccaagtactggcgagaccaagcgcaagagacactgaaatatgccctggagcttcagaagctcaacaccaacgtggctaagaatgtcatcatgttcctgggagatgggatgggtgtctccacagtgacggctgcccgcatcctcaagggtcagctccaccacaaccctggggaggagaccaggctggagatggacaagttccccttcgtggccctctccaagacgtacaacaccaatgcccaggtccctgacagcgccggcaccgccaccgcctacctgtgtggggtgaaggccaatgagggcaccgtgggggtaagcgcagccactgagcgttcccggtgcaacaccacccaggggaacgaggtcacctccatcctgcgctgggccaaggacgctgggaaatctgtgggcattgtgaccaccacgagagtgaaccatgccacccccagcgccgcctacgcccactcggctgaccgggactggtactcagacaacgagatgccccctgaggccttgagccagggctgtaaggacatcgcctaccagctcatgcataacatcagggacattgacgtgatcatggggggtggccggaaatacatgtaccccaagaataaaactgatgtggagtatgagagtgacgagaaagccaggggcacgaggctggacggcctggacctcgttgacacctggaagagcttcaaaccgagatacaagcactcccacttcatctggaaccgcacggaactcctgacccttgacccccacaatgtggactacctattgggtctcttcgagccaggggacatgcagtacgagctgaacaggaacaacgtgacggacccgtcactctccgagatggtggtggtggccatccagatcctgcggaagaaccccaaaggcttcttcttgctggtggaaggaggcagaattgaccacgggcaccatgaaggaaaagccaagcaggccctgcatgaggcggtggagatggaccgggccatcgggcaggcaggcagcttgacctcctcggaagacactctgaccgtggtcactgcggaccattcccacgtcttcacatttggtggatacaccccccgtggcaactctatctttggtctggcccccatgctgagtgacacagacaagaagcccttcactgccatcctgtatggcaatgggcctggctacaaggtggtgggcggtgaacgagagaatgtctccatggtggactatgctcacaacaactaccaggcgcagtctgctgtgcccctgcgccacgagacccacggcggggaggacgtggccgtcttctccaagggccccatggcgcacctgctgcacggcgtccacgagcagaactacgtcccccacgtgatggcgtatgcagcctgcatcggggccaacctcggccactgtgctcctgccagctcggcaggatccgatgatgacgacgacgatgacgatgatgattga

SEQ ID NO: 108 - DNA, 1548 bp, synthetic construct
  misc_feature 1..1548: note = Codon optimized, CO1-TNALP contains D10 tag at C end
  source 1..1548: mol_type = other DNA
atgatctctccatttctggtgctggccatcggcacctgtctgaccaactcactagtgcccgagaaagagaaggaccccaagtactggcgcgatcaggcccaagagacactgaagtacgccctggaactgcagaaactgaacaccaacgtggccaagaacgtgatcatgttcctcggcgacggcatgggcgtgtccacagttacagccgccagaatcctgaagggccagctgcaccataatcctggcgaagagacacggctggaaatggacaagttcccattcgtggccctgagcaagacctacaacaccaatgctcaggtgcccgattctgccggaacagccacagcttatctgtgcggcgtgaaggccaatgagggcaccgttggagtgtctgccgccaccgaaagatcccggtgcaataccacacagggcaacgaagtgaccagcatcctgagatgggccaaagacgccggcaagtctgtgggcatcgtgaccaccaccagagtgaaccacgccacacctagcgccgcctatgctcactctgccgacagagactggtacagcgacaacgagatgcctcctgaggctctgtctcagggctgcaaggatatcgcctaccagctgatgcacaacatccgggacattgatgtgatcatgggcggaggccggaagtacatgtatcccaagaacaagaccgacgtcgagtacgagagcgacgagaaggccagaggcacaagactggatggcctggacctggtggatacctggaagtccttcaagccccggtacaagcacagccacttcatctggaaccggaccgagctgctgacactggaccctcacaatgtggactacctgctgggcctgttcgagcccggcgatatgcagtacgagctgaaccggaacaacgtgacagaccccagcctgagcgagatggtggttgtggccattcagatcctgcggaagaaccccaagggattcttcctgctggtggaaggcggcaggatcgatcacggacaccatgagggaaaagccaagcaggccctgcacgaggccgtcgaaatggatagagccattggccaggccggcagcctgacaagctctgaggatacactgaccgtggtcaccgccgatcacagccacgtgttcacattcggcggctacacccctagaggcaacagcatctttggactggcccctatgctgagcgacaccgacaagaagcctttcaccgccatcctgtacggcaacggccctggctataaggttgtcggaggcgagagggaaaacgtgtccatggtggattacgcccacaacaactaccaggctcagagcgccgtgcctctgagacacgaaacacacggcggagaagatgtggccgtgttcagcaagggccccatggctcatctgctgcatggcgtgcacgagcagaattacgtgccacacgtgatggcctacgccgcctgtattggagccaatctgggacattgtgcccctgccagtagcgccggatccgacgatgatgacgacgacgatgacgatgactga

SEQ ID NO: 109 - DNA, 1548 bp, synthetic construct
  misc_feature 1..1548: note = Codon optimized, CO2-TNALP contains D10 tag at C end
  source 1..1548: mol_type = other DNA
atgatcagccccttcctggtgctggccattggcacctgcctgaccaacagcctggtgcctgagaaggagaaggaccccaagtactggagagaccaggcccaggagaccctgaagtatgccctggagctgcagaagctgaacaccaatgtggccaagaatgtgatcatgttcctgggggatggcatgggggtgagcacagtgacagctgccagaatcctgaagggccagctgcaccacaaccctggggaggagaccagactggagatggacaagttcccctttgtggccctgagcaagacctacaacaccaatgcccaggtgcctgacagtgctggcacagccacagcctacctgtgtggggtgaaggccaatgagggcacagtgggggtgagtgctgccacagagagaagcagatgcaacaccacccagggcaatgaggtgaccagcatcctgagatgggccaaggatgctggcaagagtgtgggcattgtgaccaccaccagagtgaaccatgccacccccagtgctgcctatgcccacagtgctgacagagactggtacagtgacaatgagatgccccctgaggccctgagccagggctgcaaggacattgcctaccagctgatgcacaacatcagagacattgatgtgatcatgggggggggcagaaagtacatgtaccccaagaacaagacagatgtggagtatgagagtgatgagaaggccagaggcaccagactggatggcctggacctggtggacacctggaagagcttcaagcccagatacaagcacagccacttcatctggaacagaacagagctgctgaccctggacccccacaatgtggactacctgctgggcctgtttgagcctggggacatgcagtatgagctgaacagaaacaatgtgacagaccccagcctgagtgagatggtggtggtggccatccagatcctgagaaagaaccccaagggcttcttcctgctggtggaggggggcagaattgaccatggccaccatgagggcaaggccaagcaggccctgcatgaggctgtggagatggacagagccattggccaggctggcagcctgaccagcagtgaggacaccctgacagtggtgacagctgaccacagccatgtgttcacctttgggggctacacccccagaggcaacagcatctttggcctggcccccatgctgagtgacacagacaagaagcccttcacagccatcctgtatggcaatggccctggctacaaggtggtggggggggagagagagaatgtgagcatggtggactatgcccacaacaactaccaggcccagagtgctgtgcccctgagacatgagacccatgggggggaggatgtggctgtgttcagcaagggccccatggcccacctgctgcatggggtgcatgagcagaactatgtgccccatgtgatggcctatgctgcctgcattggggccaacctgggccactgtgcccctgccagcagtgctggatccgatgatgatgatgatgatgatgatgatgactga

SEQ ID NO: 110 - DNA, 636 bp, Homo sapiens
  misc_feature 1..636: note = Glial Cell Derived Neurotrophic Factor (GDNF)
  source 1..636: mol_type = other DNA
atgaagttatgggatgtcgtggctgtctgcctggtgctgctccacaccgcgtccgccttcccgctgcccgccggcaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctaa

SEQ ID NO: 111 - DNA, 1611 bp, Homo sapiens
  misc_feature 1..1611: note = Tissue Glucosyl Ceramidase beta (GBA1)
  source 1..1611: mol_type = other DNA
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtaccaatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtga

SEQ ID NO: 112 - DNA, 1611 bp, synthetic construct
  misc_feature 1..1611: note = Codon optimized CO1-GBA1
  source 1..1611: mol_type = other DNA
atggagttcagcagccccagcagagaggagtgccccaagcccctgagcagagtgagcatcatggctggcagcctgacaggcctgctgctgctgcaggctgtgagctgggccagtggggccagaccctgcatccccaagagctttggctacagcagtgtggtgtgtgtgtgcaatgccacctactgtgacagctttgacccccccaccttccctgccctgggcaccttcagcagatatgagagcaccagaagtggcagaagaatggagctgagcatgggccccatccaggccaaccacacaggcacaggcctgctgctgaccctgcagcctgagcagaagttccagaaggtgaagggctttgggggggccatgacagatgctgctgccctgaacatcctggccctgagcccccctgcccagaacctgctgctgaagagctacttcagtgaggagggcattggctacaacatcatcagagtgccaatggccagctgtgacttcagcatcagaacctacacctatgctgacacccctgatgacttccagctgcacaacttcagcctgcctgaggaggacaccaagctgaagatccccctgatccacagagccctgcagctggcccagagacctgtgagcctgctggccagcccctggaccagccccacctggctgaagaccaatggggctgtgaatggcaagggcagcctgaagggccagcctggggacatctaccaccagacctgggccagatactttgtgaagttcctggatgcctatgctgagcacaagctgcagttctgggctgtgacagctgagaatgagcccagtgctggcctgctgagtggctaccccttccagtgcctgggcttcacccctgagcaccagagagacttcattgccagagacctgggccccaccctggccaacagcacccaccacaatgtgagactgctgatgctggatgaccagagactgctgctgccccactgggccaaggtggtgctgacagaccctgaggctgccaagtatgtgcatggcattgctgtgcactggtacctggacttcctggcccctgccaaggccaccctgggggagacccacagactgttccccaacaccatgctgtttgccagtgaggcctgtgtgggcagcaagttctgggagcagagtgtgagactgggcagctgggacagaggcatgcagtacagccacagcatcatcaccaacctgctgtaccatgtggtgggctggacagactggaacctggccctgaaccctgaggggggccccaactgggtgagaaactttgtggacagccccatcattgtggacatcaccaaggacaccttctacaagcagcccatgttctaccacctgggccacttcagcaagttcatccctgagggcagccagagagtgggcctggtggccagccagaagaatgacctggatgctgtggccctgatgcaccctgatggcagtgctgtggtggtggtgctgaacagaagcagcaaggatgtgcccctgaccatcaaggaccctgctgtgggcttcctggagaccatcagccctggctacagcatccacacctacctgtggagaagacagtga

SEQ ID NO: 113 - DNA, 1611 bp, synthetic construct
  misc_feature 1..1611: note = Codon optimized CO2-GBA1
  source 1..1611: mol_type = other DNA
atggagtttagcagccctagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatccgggtgccaatggccagctgcgacttcagcatccggacctacacctacgccgacacacccgacgatttccagctgcacaacttcagcctgcctgaagaggacaccaagctgaagatccctctgatccacagagccctgcagctggcacaaagacccgtttctctgctggctagcccctggacatctcccacctggctgaaaacaaatggcgccgtgaatggcaagggcagcctgaaaggccaacctggcgatatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgccagagatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggccagccagaagaatgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcggagacagtga

SEQ ID NO: 114 - DNA, 1962 bp, Homo sapiens
  misc_feature 1..1962: note = Iduronidase alpha-L- (IDUA)
  source 1..1962: mol_type = other DNA
atgcgtcccctgcgcccccgcgccgcgctgctggcgctcctggcctcgctcctggccgcgcccccggtggccccggccgaggccccgcacctggtgcatgtggacgcggcccgcgcgctgtggcccctgcggcgcttctggaggagcacaggcttctgccccccgctgccacacagccaggctgaccagtacgtcctcagctgggaccagcagctcaacctcgcctatgtgggcgccgtccctcaccgcggcatcaagcaggtccggacccactggctgctggagcttgtcaccaccagggggtccactggacggggcctgagctacaacttcacccacctggacgggtacctggaccttctcagggagaaccagctcctcccagggtttgagctgatgggcagcgcctcgggccacttcactgactttgaggacaagcagcaggtgtttgagtggaaggacttggtctccagcctggccaggagatacatcggtaggtacggactggcgcatgtttccaagtggaacttcgagacgtggaatgagccagaccaccacgactttgacaacgtctccatgaccatgcaaggcttcctgaactactacgatgcctgctcggagggtctgcgcgccgccagccccgccctgcggctgggaggccccggcgactccttccacaccccaccgcgatccccgctgagctggggcctcctgcgccactgccacgacggtaccaacttcttcactggggaggcgggcgtgcggctggactacatctccctccacaggaagggtgcgcgcagctccatctccatcctggagcaggagaaggtcgtcgcgcagcagatccggcagctcttccccaagttcgcggacacccccatttacaacgacgaggcggacccgctggtgggctggtccctgccacagccgtggagggcggacgtgacctacgcggccatggtggtgaaggtcatcgcgcagcatcagaacctgctactggccaacaccacctccgccttcccctacgcgctcctgagcaacgacaatgccttcctgagctaccacccgcaccccttcgcgcagcgcacgctcaccgcgcgcttccaggtcaacaacacccgcccgccgcacgtgcagctgttgcgcaagccggtgctcacggccatggggctgctggcgctgctggatgaggagcagctctgggccgaagtgtcgcaggccgggaccgtcctggacagcaaccacacggtgggcgtcctggccagcgcccaccgcccccagggcccggccgacgcctggcgcgccgcggtgctgatctacgcgagcgacgacacccgcgcccaccccaaccgcagcgtcgcggtgaccctgcggctgcgcggggtgccccccggcccgggcctggtctacgtcacgcgctacctggacaacgggctctgcagccccgacggcgagtggcggcgcctgggccggcccgtcttccccacggcagagcagttccggcgcatgcgcgcggctgaggacccggtggccgcggcgccccgccccttacccgccggcggccgcctgaccctgcgccccgcgctgcggctgccgtcgcttttgctggtgcacgtgtgtgcgcgccccgagaagccgcccgggcaggtcacgcggctccgcgccctgcccctgacccaagggcagctggttctggtctggtcggatgaacacgtgggctccaagtgcctgtggacatacgagatccagttctctcaggacggtaaggcgtacaccccggtcagcaggaagccatcgaccttcaacctctttgtgttcagcccagacacaggtgctgtctctggctcctaccgagttcgagccctggactactgggcccgaccaggccccttctcggaccctgtgccgtacctggaggtccctgtgccaagagggcccccatccccgggcaatccatga

SEQ ID NO: 115 - DNA, 1962 bp, synthetic construct
  misc_feature 1..1962: note = Codon optimized CO1-IDUA
  source 1..1962: mol_type = other DNA
atgagacccctgagacccagagctgccctgctggccctgctggccagcctgctggctgccccccctgtggcccctgctgaggccccccaccttgtacatgtggatgctgccagagccctgtggcccctgagaagattctggagaagcacaggcttctgcccccccctgccccacagccaggctgaccagtatgtgctgagctgggaccagcagctgaacctggcctatgtgggggctgtgccccacagaggcatcaagcaggtgagaacccactggctgctggagctggtgaccaccagaggcagcacaggcagaggcctgagctacaacttcacccacctggatggctacctggacctgctgagagagaaccagctgctgcctggctttgagctgatgggcagtgccagtggccacttcacagactttgaggacaagcagcaggtgtttgagtggaaggacctggtgagcagcctggccagaagatacattggcagatatggcctggcccatgtgagcaagtggaactttgagacctggaatgagcctgaccaccatgactttgacaatgtgagcatgaccatgcagggcttcctgaactactatgatgcctgcagtgagggcctgagagctgccagccctgccctgagactggggggccctggggacagcttccacaccccccccagaagccccctgagctggggcctgctgagacactgccatgatggcaccaacttcttcacaggggaggctggggtgagactggactacatcagcctgcacagaaagggggccagaagcagcatcagcatcctggagcaggagaaggtggtggcccagcagatcagacagctgttccccaagtttgctgacacccccatctacaatgatgaggctgaccccctggtgggctggagcctgccccagccctggagagctgatgtgacctatgctgccatggtggtgaaggtgattgcccagcaccagaacctgctgctggccaacaccaccagtgccttcccctatgccctgctgagcaatgacaatgccttcctgagctaccacccccacccctttgcccagagaaccctgacagccagattccaggtgaacaacaccagacccccccatgtgcagctgctgagaaagcctgtgctgacagccatgggcctgctggccctgctggatgaggagcagctgtgggctgaggtgagccaggctggcacagtgctggacagcaaccacacagtgggggtgctggccagtgcccacagaccccagggccctgctgatgcctggagagctgctgtgctgatctatgccagtgatgacaccagagcccaccccaacagaagtgtggctgtgaccctgagactgagaggggtgccccctggccctggcctggtgtatgtgaccagatacctggacaatggcctgtgcagccctgatggggagtggagaagactgggcagacctgtgttccccacagctgagcagttcagaagaatgagagctgctgaggaccctgtggctgctgcccccagacccctgcctgctgggggcagactgaccctgagacctgccctgagactgcccagcctgctgctggtgcatgtgtgtgccagacctgagaagccccctggccaggtgaccagactgagagccctgcccctgacccagggccagctggtgctggtgtggagtgatgagcatgtgggcagcaagtgcctgtggacctatgagatccagttcagccaggatggcaaggcctacacccctgtgagcagaaagcccagcaccttcaacctgtttgtgttcagccctgacacaggggctgtgagtggcagctacagagtgagagccctggactactgggccagacctggccccttcagtgaccctgtgccctacctggaggtgcctgtgcccagaggcccccccagccctggcaacccctga

SEQ ID NO: 116 - DNA, 1578 bp, Homo sapiens
  misc_feature 1..1578: note = Cytochrome P450 family 4 subfamily V member 2 (CYP4V2)
  source 1..1578: mol_type = other DNA
atggcggggctctggctggggctcgtgtggcagaagctgctgctgtggggcgcggcgagtgccctttccctggccggcgccagtctggtcctgagcctgctgcagagggtggcgagctacgcgcggaaatggcagcagatgcggcccatccccacggtggcccgcgcctacccactggtgggccacgcgctgctgatgaagccggacgggcgagaattttttcagcagatcattgagtacacagaggaataccgccacatgccgctgctgaagctctgggtcgggccagtgcccatggtggccctttataatgcagaaaatgtggaggtaattttaactagttcaaagcaaattgacaaatcctctatgtacaagtttttagaaccatggcttggcctaggacttcttacaagtactggaaacaaatggcgctccaggagaaagatgttaacacccactttccattttaccattctggaagatttcttagatatcatgaatgaacaagcaaatatattggttaagaaacttgaaaaacacattaaccaagaagcatttaactgctttttttacatcactctttgtgccttagatatcatctgtgaaacagctatggggaagaatattggtgctcaaagtaatgatgattccgagtatgtccgtgcagtttatagaatgagtgagatgatatttcgaagaataaagatgccctggctttggcttgatctctggtatcttatgtttaaagaaggatgggaacacaaaaagagccttcagatcctacatacttttaccaacagtgtcatcgctgaacgggccaatgaaatgaacgccaatgaagactgtagaggtgatggcaggggctctgccccctccaaaaataaacgcagggcctttcttgacttgcttttaagtgtgactgatgacgaagggaacaggctaagtcatgaagatattcgagaagaagttgacaccttcatgtttgaggggcacgatacaactgcagctgcaataaactggtccttatacctgttgggttctaacccagaagtccagaaaaaagtggatcatgaattggatgacgtgtttgggaagtctgaccgtcccgctacagtagaagacctgaagaaacttcggtatctggaatgtgttattaaggagacccttcgcctttttccttctgttcctttatttgcccgtagtgttagtgaagattgtgaagtggcaggttacagagttctaaaaggcactgaagccgtcatcattccctatgcattgcacagagatccgagatacttccccaaccccgaggagttccagcctgagcggttcttccccgagaatgcacaagggcgccatccatatgcctacgtgcccttctctgctggccccaggaactgtataggtcaaaagtttgctgtgatggaagaaaagaccattctttcgtgcatcctgaggcacttttggatagaatccaaccagaaaagagaagagcttggtctagaaggacagttgattcttcgtccaagtaatggcatctggatcaagttgaagaggagaaatgcagatgaacgctaa

SEQ ID NO: 117 - DNA, 711 bp, Homo sapiens
  misc_feature 1..711: note = Retinoschisin 1 (RS1)
  source 1..711: mol_type = other DNA
atgagccgcaagatagaaggctttttgttattacttctctttggctatgaagccacattgggattatcgtctaccgaggatgaaggcgaggacccctggtatcaaaaagcatgcgatgaaggcgaggacccctggtatcaaaaagcatgcaagtgcgattgccaaggaggacccaatgctctgtggtctgcaggtgccacctccttggactgtataccagaatgcccatatcacaagcctctgggtttcgagtcaggggaggtcacaccggaccagatcacctgctctaacccggagcagtatgtgggctggtattcttcgtggactgcaaacaaggcccggctcaacagtcaaggctttgggtgtgcctggctctccaagttccaggacagtagccagtggttacagatagatctgaaggagatcaaagtgatttcagggatcctcacccaggggcgctgtgacatcgatgagtggatgaccaagtacagcgtgcagtacaggaccgatgagcgcctgaactggatttactacaaggaccagactggaaacaaccgggtcttctatggcaactcggaccgcacctccacggttcagaacctgctgcggccccccatcatctcccgcttcatccgcctcatcccgctgggctggcacgtccgcattgccatccggatggagctgctggagtgcgtcagcaagtgtgcctga

SEQ ID NO: 118 - DNA, 2565 bp, Homo sapiens
  misc_feature 1..2565: note = Phosphodiesterase 6B (PDE6B)
  source 1..2565: mol_type = other DNA
atgagcctcagtgaggagcaggcccggagctttctggaccagaaccccgattttgcccgccagtactttgggaagaaactgagccctgagaatgtggccgcggcctgcgaggacgggtgcccgccggactgcgacagcctccgggacctctgccaggtggaggagagcacggcgctgctggagctggtgcaggatatgcaggagagcatcaacatggagcgcgtggtcttcaaggtcctgcggcgcctctgcaccctgctgcaggccgaccgctgcagcctcttcatgtaccgccagcgcaacggcgtggccgagctggccaccaggcttttcagcgtgcagccggacagcgtcctggaggactgcctggtgccccccgactccgagatcgtcttcccactggacatcggggtcgtgggccacgtggctcagaccaaaaagatggtgaacgtcgaggacgtggccgagtgccctcacttcagctcatttgctgacgagctcactgactacaagacaaagaatatgctggccacacccatcatgaatggcaaagacgtcgtggcggtgatcatggcagtgaacaagctcaacggcccattcttcaccagcgaagacgaagatgtgttcttgaagtacctgaattttgccacgttgtacctgaaaatctatcacctgagctacctccacaactgcgagacgcgccgcggccaggtgctgctgtggtcggccaacaaggtgtttgaggagctgacggacatcgagaggcagttccacaaggccttctacacggtgcgggcctacctcaactgcgagcggtactccgtgggcctcctggacatgaccaaggagaaggaattttttgacgtgtggtctgtgctgatgggagagtcccagccgtactcgggcccacgcacgcctgatggccgggaaattgtcttctacaaagtgatcgactacatcctccacggcaaggaggagatcaaggtcattcccacaccctcagccgatcactgggccctggccagcggccttccaagctacgtggcagaaagcggctttatttgtaacatcatgaatgcttccgctgacgaaatgttcaaatttcaggaaggggccctggacgactccgggtggctcatcaagaatgtgctgtccatgcccatcgtcaacaagaaggaggagattgtgggagtcgccacattttacaacaggaaagacgggaagccctttgacgaacaggacgaggttctcatggagtccctgacacagttcctgggctggtcagtgatgaacaccgacacctacgacaagatgaacaagctggagaaccgcaaggacatcgcacaggacatggtcctttaccacgtgaagtgcgacagggacgagatccagctcatcctgccaaccagagcgcgcctggggaaggagcctgctgactgcgatgaggacgagctgggcgaaatcctgaaggaggagctgccagggcccaccacatttgacatctacgaattccacttctctgacctggagtgcaccgaactggacctggtcaaatgtggcatccagatgtactacgagctgggcgtggtccgaaagttccagatcccccaggaggtcctggtgcggttcctgttctccatcagcaaaggctaccggagaatcacctaccacaactggcgccacggcttcaacgtggcccagacgatgttcacgctgctcatgaccggcaaactgaagagctactacacggacctggaggccttcgccatggtgacagccggcctgtgccatgacatcgaccaccgcggcaccaacaacctgtaccagatgaagtcccagaaccccttggctaaactccacggctcctcgattttggagcggcaccacctggagtttgggaagttcctgctctcggaggagaccctgaacatctaccagaacctgaaccggcggcagcacgagcacgtgatccacctgatggacatcgccatcatcgccacggacctggccctgtacttcaagaagagagcgatgtttcagaagatcgtggatgagtccaagaactaccaggacaagaagagctgggtggagtacctgtccctggagacgacccggaaggagatcgtcatggccatgatgatgacagcctgcgacctgtctgccatcaccaagccctgggaagtccagagcaaggtcgcacttctcgtggctgctgagttctgggagcaaggtgacttggaaaggacagtcttggatcagcagcccattcctatgatggaccggaacaaggcggccgagctccccaagctgcaagtgggcttcatcgacttcgtgtgcacattcgtgtacaaggagttctctcgtttccacgaagagatcctgcccatgttcgaccgactgcagaacaataggaaagagtggaaggcgctggctgatgagtatgaggccaaagtgaaggctctggaggagaaggaggaggaggagagggtggcagccaagaaagtaggcacagaaatttgcaatggcggcccagcacccaagtcttcaacctgctgtatcctgtga

SEQ ID NO: 119 - DNA, 1497 bp, Homo sapiens
  misc_feature 1..1497: note = Methyl-CpG Binding Protein (MeCP2)
  source 1..1497: mol_type = other DNA
atggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctag

SEQ ID NO: 120 - DNA, 2232 bp, Homo sapiens
  misc_feature 1..2232: note = N-acetyl-alpha-glucosaminidase (NAGLU)
  source 1..2232: mol_type = other DNA
atggaggcggtggcggtggccgcggcggtgggggtccttctcctggccggggccgggggcgcggcaggcgacgaggcccgggaggcggcggccgtgcgggcgctcgtggcccggctgctggggccaggccccgcggccgacttctccgtgtcggtggagcgcgctctggctgccaagccgggcttggacacctacagcctgggcggcggcggcgcggcgcgcgtgcgggtgcgcggctccacgggcgtggcggccgccgcggggctgcaccgctacctgcgcgacttctgtggctgccacgtggcctggtccggctctcagctgcgcctgccgcggccactgccagccgtgccgggggagctgaccgaggccacgcccaacaggtaccgctattaccagaatgtgtgcacgcaaagctactccttcgtgtggtgggactgggcccgctgggagcgagagatagactggatggcgctgaatggcatcaacctggcactggcctggagcggccaggaggccatctggcagcgggtgtacctggccttgggcctgacccaggcagagatcaatgagttctttactggtcctgccttcctggcctgggggcgaatgggcaacctgcacacctgggatggccccctgcccccctcctggcacatcaagcagctttacctgcagcaccgggtcctggaccagatgcgctccttcggcatgaccccagtgctgcctgcattcgcggggcatgttcccgaggctgtcaccagggtgttccctcaggtcaatgtcacgaagatgggcagttggggccactttaactgttcctactcctgctccttccttctggctccggaagaccccatattccccatcatcgggagcctcttcctgcgagagctgatcaaagagtttggcacagaccacatctatggggccgacactttcaatgagatgcagccaccttcctcagagccctcctaccttgccgcagccaccactgccgtctatgaggccatgactgcagtggatactgaggctgtgtggctgctccaaggctggctcttccagcaccagccgcagttctgggggcccgcccagatcagggctgtgctgggagctgtgccccgtggccgcctcctggttctggacctgtttgctgagagccagcctgtgtatacccgcactgcctccttccagggccagcccttcatctggtgcatgctgcacaactttgggggaaaccatggtctttttggagccctagaggctgtgaacggaggcccagaagctgcccgcctcttccccaactccaccatggtaggcacgggcatggcccccgagggcatcagccagaacgaagtggtctattccctcatggctgagctgggctggcgaaaggacccagtgccagatttggcagcctgggtgaccagctttgccgcccggcggtatggggtctcccacccggacgcaggggcagcgtggaggctactgctccggagtgtgtacaactgctccggggaggcctgcaggggccacaatcgtagcccgctggtcaggcggccgtccctacagatgaataccagcatctggtacaaccgatctgatgtgtttgaggcctggcggctgctgctcacatctgctccctccctggccaccagccccgccttccgctacgacctgctggacctcactcggcaggcagtgcaggagctggtcagcttgtactatgaggaggcaagaagcgcctacctgagcaaggagctggcctccctgttgagggctggaggcgtcctggcctatgagctgctgccggcactggacgaggtgctggctagtgacagccgcttcttgctgggcagctggctagagcaggcccgagcagcggcagtcagtgaggccgaggccgatttctacgagcagaacagccgctaccagctgaccttgtgggggccagaaggcaacatcctggactatgccaacaagcagctggcggggttggtggccaactactacacccctcgctggcggcttttcctggaggcgctggttgacagtgtggcccagggcatccctttccaacagcaccagtttgacaaaaatgtcttccaactggagcaggccttcgttctcagcaagcagaggtaccccagccagccgcgaggagacactgtggacctggccaagaagatcttcctcaaatattaccccggctgggtggccggctcttggtga

SEQ ID NO: 121 - DNA, 1317 bp, Homo sapiens; identical to SEQ ID NO: 12
  misc_feature 1..1317: note = Ceroid Lipofuscinosis, Neuronal 3 (CLN3)
  source 1..1317: mol_type = other DNA

SEQ ID NO: 122 - DNA, 1317 bp, synthetic construct; identical to SEQ ID NO: 13
  misc_feature 1..1317: note = Codon optimized CO1-CLN3
  source 1..1317: mol_type = other DNA

SEQ ID NO: 123 - DNA, 2859 bp, Homo sapiens
  misc_feature 1..2859: note = Acid Alpha-Glucosidase (GAA)
  source 1..2859: mol_type = other DNA
atgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 124 - DNA, 2859 bp, synthetic construct; identical to SEQ ID NO: 5
  misc_feature 1..2859: note = Codon optimized CO1-GAA
  source 1..2859: mol_type = other DNA

SEQ ID NO: 125 - DNA, 2859 bp, synthetic construct; identical to SEQ ID NO: 7
  misc_feature 1..2859: note = Codon optimized CO2-GAA
  source 1..2859: mol_type = other DNA

SEQ ID NO: 126 - DNA, 2859 bp, synthetic construct
  misc_feature 1..2859: note = Codon optimized CO3-GAA
  source 1..2859: mol_type = other DNA
atgggggtgagacaccccccctgcagccacagactgctggctgtgtgtgccctggtgagcctggccacagctgccctgctgggccacatcctgctgcatgacttcctactagtgcccagagagctgagtggcagcagccctgtgctggaggagacccaccctgcccaccagcagggggccagcagacctggccccagagatgcccaggcccaccctggcagacccagagctgtgcccacccagtgtgatgtgccccccaacagcagatttgactgtgcccctgacaaggccatcacccaggagcagtgtgaggccagaggctgctgctacatccctgccaagcagggcctgcagggggcccagatgggccagccctggtgcttcttcccccccagctaccccagctacaagctggagaacctgagcagcagtgagatgggctacacagccaccctgaccagaaccacccccaccttcttccccaaggacatcctgaccctgagactggatgtgatgatggagacagagaacagactgcacttcaccatcaaggaccctgccaacagaagatatgaggtgcccctggagaccccccatgtgcacagcagagcccccagccccctgtacagtgtggagttcagtgaggagccctttggggtgattgtgagaagacagctggatggcagagtgctgctgaacaccacagtggcccccctgttctttgctgaccagttcctgcagctgagcaccagcctgcccagccagtacatcacaggcctggctgagcacctgagccccctgatgctgagcaccagctggaccagaatcaccctgtggaacagagacctggcccccacccctggggccaacctgtatggcagccaccccttctacctggccctggaggatgggggcagtgcccatggggtgttcctgctgaacagcaatgccatggatgtggtgctgcagcccagccctgccctgagctggagaagcacagggggcatcctggatgtgtacatcttcctgggccctgagcccaagagtgtggtgcagcagtacctggatgtggtgggctaccccttcatgcccccctactggggcctgggcttccacctgtgcagatggggctacagcagcacagccatcaccagacaggtggtggagaacatgaccagagcccacttccccctggatgtgcagtggaatgacctggactacatggacagcagaagagacttcaccttcaacaaggatggcttcagagacttccctgccatggtgcaggagctgcaccaggggggcagaagatacatgatgattgtggaccctgccatcagcagcagtggccctgctggcagctacagaccctatgatgagggcctgagaagaggggtgttcatcaccaatgagacaggccagcccctgattggcaaggtgtggcctggcagcacagccttccctgacttcaccaaccccacagccctggcctggtgggaggacatggtggctgagttccatgaccaggtgccctttgatggcatgtggattgacatgaatgagcccagcaacttcatcagaggcagtgaggatggctgccccaacaatgagctggagaaccccccctatgtgcctggggtggtggggggcaccctgcaggctgccaccatctgtgccagcagccaccagttcctgagcacccactacaacctgcacaacctgtatggcctgacagaggccattgccagccacagagccctggtgaaggccagaggcaccagaccctttgtgatcagcagaagcacctttgctggccatggcagatatgctggccactggacaggggatgtgtggagcagctgggagcagctggccagcagtgtgcctgagatcctgcagttcaacctgctgggggtgcccctggtgggggctgatgtgtgtggcttcctgggcaacaccagtgaggagctgtgtgtgagatggacccagctgggggccttctaccccttcatgagaaaccacaacagcctgctgagcctgccccaggagccctacagcttcagtgagcctgcccagcaggccatgagaaaggccctgaccctgagatatgccctgctgccccacctgtacaccctgttccaccaggcccatgtggctggggagacagtggccagacccctgttcctggagttccccaaggacagcagcacctggacagtggaccaccagctgctgtggggggaggccctgctgatcacccctgtgctgcaggctggcaaggctgaggtgacaggctacttccccctgggcacctggtatgacctgcagacagtgcctgtggaggccctgggcagcctgccccccccccctgctgcccccagagagcctgccatccacagtgagggccagtgggtgaccctgcctgcccccctggacaccatcaatgtgcacctgagagctggctacatcatccccctgcagggccctggcctgaccaccacagagagcagacagcagcccatggccctggctgtggccctgaccaaggggggggaggccagaggggagctgttctgggatgatggggagagcctggaggtgctggagagaggggcctacacccaggtgatcttcctggccagaaacaacaccattgtgaatgagctggtgagagtgaccagtgagggggctggcctgcagctgcagaaggtgacagtgctgggggtggccacagccccccagcaggtgctgagcaatggggtgcctgtgagcaacttcacctacagccctgacaccaaggtgctggacatctgtgtgagcctgctgatgggggagcagttcctggtgagctggtgctga

SEQ ID NO: 127 - DNA, 1290 bp, Homo sapiens; identical to SEQ ID NO: 9
  misc_feature 1..1290: note = Alpha-Galactosidase A (GLA)
  source 1..1290: mol_type = other DNA

SEQ ID NO: 128 - DNA, 1290 bp, synthetic construct; identical to SEQ ID NO: 10
  misc_feature 1..1290: note = Codon optimized CO1-GLA
  source 1..1290: mol_type = other DNA

SEQ ID NO: 129 - DNA, 1377 bp, synthetic construct
  misc_feature 1..1377: note = Codon optimized + GET, CO1-GLA-GET
  source 1..1377: mol_type = other DNA
atgcagctgagaaatcctgaactgcacctgggctgtgccctggctctgagatttctggctctggtgtcctgggacattcctggcgctagagccctggataatggcctggccagaacacctacaatgggctggctgcactgggagagattcatgtgcaacctggactgccaagaggaacccgacagctgcatcagcgagaagctgttcatggaaatggccgagctgatggtgtccgaaggctggaaggatgccggctacgagtacctgtgcatcgacgattgctggatggcccctcagagagattctgagggcagactgcaggccgatcctcagagatttcctcacggaatccggcagctggccaactacgtgcactctaagggactgaagctgggcatctacgccgacgtgggcaacaagacatgtgccggctttccaggcagcttcggctactacgatatcgacgcccagacctttgccgattggggcgtcgacctgctgaagttcgatggctgctactgcgacagcctggaaaacctggccgacggctacaaacacatgtctctggccctgaaccggaccggcagatctatcgtgtactcttgcgagtggcccctgtacatgtggcccttccagaagcctaactacaccgagatcagacagtactgcaaccactggcggaacttcgccgacatcgatgacagctggaagtccatcaagagcatcctggactggaccagcttcaatcaagagcggatcgtggatgtggctggcccaggcggatggaacgatcctgatatgctggtcatcggcaacttcggcctgagctggaatcagcaagtgacccagatggccctgtgggccattatggccgctcctctgttcatgagcaacgacctgagacacatcagccctcaggccaaggctctgctgcaggataaggacgtgatcgccatcaaccaggatcctctgggcaagcagggctatcagctgagacagggcgacaatttcgaagtgtgggaaagacctctgagcggcctggcttgggccgtcgccatgatcaatagacaagagatcggcggaccccggtcctatacaattgccgtggcttctctcggaaaaggcgtggcctgcaatcctgcctgctttatcacacagctgctccccgtgaagagaaagctgggcttttacgagtggaccagcagactgagatcccacatcaaccccacaggcactgttctgctgcaactggaaaacacaatgcagatgagcctgaaggacctgctgcggagaagaagaaggcgcagacgcaagcgcaagaagaaaggcaaaggcctcggcaagaagcgggacccctgtctgagaaagtacaagtaa

SEQ ID NO: 130 - DNA, 1290 bp, synthetic construct
  misc_feature 1..1290: note = Codon optimized CO2-GLA
  source 1..1290: mol_type = other DNA
atgcagctgagaaaccctgagctgcacctgggctgtgccctggccctgagattcctggccctggtgagctgggacatccctggggccagagccctggacaatgggctagccagaacccccaccatgggctggctgcactgggagagattcatgtgcaacctggactgccaggaggagcctgacagctgcatcagtgagaagctgttcatggagatggctgagctgatggtgagtgagggctggaaggatgctggctatgagtacctgtgcattgatgactgctggatggccccccagagagacagtgagggcagactgcaggctgacccccagagattcccccatggcatcagacagctggccaactatgtgcacagcaagggcctgaagctgggcatctatgctgatgtgggcaacaagacctgtgctggcttccctggcagctttggctactatgacattgatgcccagacctttgctgactggggggtggacctgctgaagtttgatggctgctactgtgacagcctggagaacctggctgatggctacaagcacatgagcctggccctgaacagaacaggcagaagcattgtgtacagctgtgagtggcccctgtacatgtggcccttccagaagcccaactacacagagatcagacagtactgcaaccactggagaaactttgctgacattgatgacagctggaagagcatcaagagcatcctggactggaccagcttcaaccaggagagaattgtggatgtggctggccctgggggctggaatgaccctgacatgctggtgattggcaactttggcctgagctggaaccagcaggtgacccagatggccctgtgggccatcatggctgcccccctgttcatgagcaatgacctgagacacatcagcccccaggccaaggccctgctgcaggacaaggatgtgattgccatcaaccaggaccccctgggcaagcagggctaccagctgagacagggggacaactttgaggtgtgggagagacccctgagtggcctggcctgggctgtggccatgatcaacagacaggagattgggggccccagaagctacaccattgctgtggccagcctgggcaagggggtggcctgcaaccctgcctgcttcatcacccagctgctgcctgtgaagagaaagctgggcttctatgagtggaccagcagactgagaagccacatcaaccccacaggcacagtgctgctgcagctggagaacaccatgcagatgagcctgaaggacctgctgtga

SEQ ID NO: 131 - DNA, 1290 bp, synthetic construct
  misc_feature 1..1290: note = Codon optimized CO3-GLA
  source 1..1290: mol_type = other DNA
atgcagctgagaaaccctgagctgcacctgggctgtgccctggccctgagattcctggccctggtgagctgggacatccctggggccagagccctggacaatgggctagccagaacccccaccatgggctggctgcactgggagagattcatgtgcaacctggactgccaggaggagcctgacagctgcatcagtgagaagctgttcatggagatggctgagctgatggtgagtgagggctggaaggatgctggctatgagtacctgtgcattgatgactgctggatggccccccagagagacagtgagggcagactgcaggctgacccccagagattcccccatggcatcagacagctggccaactatgtgcacagcaagggcctgaagctgggcatctatgctgatgtgggcaacaagacctgtgctggcttccctggcagctttggctactatgacattgatgcccagacctttgctgactggggggtggacctgctgaagtttgatggctgctactgtgacagcctggagaacctggctgatggctacaagcacatgagcctggccctgaacagaacaggcagaagcattgtgtacagctgtgagtggcccctgtacatgtggcccttccagaagcccaactacacagagatcagacagtactgcaaccactggagaaactttgctgacattgatgacagctggaagagcatcaagagcatcctggactggaccagcttcaaccaggagagaattgtggatgtggctggccctgggggctggaatgaccctgacatgctggtgattggcaactttggcctgagctggaaccagcaggtgacccagatggccctgtgggccatcatggctgcccccctgttcatgagcaatgacctgagacacatcagcccccaggccaaggccctgctgcaggacaaggatgtgattgccatcaaccaggaccccctgggcaagcagggctaccagctgagacagggggacaactttgaggtgtgggagagacccctgagtggcctggcctgggctgtggccatgatcaacagacaggagattgggggccccagaagctacaccattgctgtggcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 132 - DNA, 4287 bp, synthetic construct; identical to SEQ ID NO: 4
  misc_feature 1..4287: note = Codon optimized, Cystic Fibrosis Transmembrane Regulator deltaR (CFTRdeltaR) contains R domain deletion
  source 1..4287: mol_type = other DNA

SEQ ID NO: 133 - DNA, 4443 bp, synthetic construct; identical to SEQ ID NO: 93
  misc_feature 1..4443: note = Codon optimized, full length Cystic Fibrosis Transmembrane Regulator (CFTR)
  source 1..4443: mol_type = other DNA

SEQ ID NO: 134 - protein, 502 aa, Homo sapiens
  REGION 1..502: note = MISC_FEATURE - Sulfoglucosamine sulfohydrolase (SGSH)
MSCPVPACCALLLVLGLCRARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDQPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 135 - protein, 531 aa, synthetic construct
  REGION 1..531: note = Codon optimized + GET CO1-SGSH-GET
MSCPVPACCALLLVLGLCRARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDQPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELRRRRRRRRKRKKKGKGLGKKRDPCLRKYK

SEQ ID NO: 136 - protein, 306 aa, synthetic construct
  REGION 1..306: note = Codon optimized Ceroid Lipofuscinosis, Neuronal, 1 (CLN1)
MASPGCLWLLAVALLPWTCASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 137 - protein, 294 aa, Homo sapiens
  REGION 1..294: note = MISC_FEATURE - Survival Motor Neuron 1 (SMN1)
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKPGLKFNGPPPPPPPPPPHLLSCWLPPFPSGPPIIPPPPPICPDSLDDADALGSMLISWYMSGYHTGYYMGFRQNQKEGRCSHSLN

SEQ ID NO: 138 - protein, 515 aa, Homo sapiens
  REGION 1..515: note = MISC_FEATURE - Tissue Non-specific Alkaline Phosphatase (TNALP)
MISPFLVLAIGTCLTNSLVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSDDDDDDDDDD

SEQ ID NO: 139 - protein, 211 aa, Homo sapiens
  REGION 1..211: note = MISC_FEATURE - Glial Cell Derived Neurotrophic Factor (GDNF)
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 140 - protein, 536 aa, Homo sapiens
  REGION 1..536: note = MISC_FEATURE - Tissue Glucosyl Ceramidase beta (GBA1)
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 141 - protein, 653 aa, Homo sapiens
  REGION 1..653: note = MISC_FEATURE - Iduronidase alpha-L- (IDUA)
MRPLRPRAALLALLASLLAAPPVAPAEAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 142 - protein, 525 aa, Homo sapiens
  REGION 1..525: note = MISC_FEATURE - Cytochrome P450 family 4 subfamily V member 2 (CYP4V2)
MAGLWLGLVWQKLLLWGAASALSLAGASLVLSLLQRVASYARKWQQMRPIPTVARAYPLVGHALLMKPDGREFFQQIIEYTEEYRHMPLLKLWVGPVPMVALYNAENVEVILTSSKQIDKSSMYKFLEPWLGLGLLTSTGNKWRSRRKMLTPTFHFTILEDFLDIMNEQANILVKKLEKHINQEAFNCFFYITLCALDIICETAMGKNIGAQSNDDSEYVRAVYRMSEMIFRRIKMPWLWLDLWYLMFKEGWEHKKSLQILHTFTNSVIAERANEMNANEDCRGDGRGSAPSKNKRRAFLDLLLSVTDDEGNRLSHEDIREEVDTFMFEGHDTTAAAINWSLYLLGSNPEVQKKVDHELDDVFGKSDRPATVEDLKKLRYLECVIKETLRLFPSVPLFARSVSEDCEVAGYRVLKGTEAVIIPYALHRDPRYFPNPEEFQPERFFPENAQGRHPYAYVPFSAGPRNCIGQKFAVMEEKTILSCILRHFWIESNQKREELGLEGQLILRPSNGIWIKLKRRNADER

SEQ ID NO: 143 - protein, 236 aa, Homo sapiens
  REGION 1..236: note = MISC_FEATURE - Retinoschisin 1 (RS1)
MSRKIEGFLLLLLFGYEATLGLSSTEDEGEDPWYQKACDEGEDPWYQKACKCDCQGGPNALWSAGATSLDCIPECPYHKPLGFESGEVTPDQITCSNPEQYVGWYSSWTANKARLNSQGFGCAWLSKFQDSSQWLQIDLKEIKVISGILTQGRCDIDEWMTKYSVQYRTDERLNWIYYKDQTGNNRVFYGNSDRTSTVQNLLRPPIISRFIRLIPLGWHVRIAIRMELLECVSKCA

SEQ ID NO: 144 - protein, 854 aa, Homo sapiens
  REGION 1..854: note = MISC_FEATURE - Phosphodiesterase 6B (PDE6B)
MSLSEEQARSFLDQNPDFARQYFGKKLSPENVAAACEDGCPPDCDSLRDLCQVEESTALLELVQDMQESINMERVVFKVLRRLCTLLQADRCSLFMYRQRNGVAELATRLFSVQPDSVLEDCLVPPDSEIVFPLDIGVVGHVAQTKKMVNVEDVAECPHFSSFADELTDYKTKNMLATPIMNGKDVVAVIMAVNKLNGPFFTSEDEDVFLKYLNFATLYLKIYHLSYLHNCETRRGQVLLWSANKVFEELTDIERQFHKAFYTVRAYLNCERYSVGLLDMTKEKEFFDVWSVLMGESQPYSGPRTPDGREIVFYKVIDYILHGKEEIKVIPTPSADHWALASGLPSYVAESGFICNIMNASADEMFKFQEGALDDSGWLIKNVLSMPIVNKKEEIVGVATFYNRKDGKPFDEQDEVLMESLTQFLGWSVMNTDTYDKMNKLENRKDIAQDMVLYHVKCDRDEIQLILPTRARLGKEPADCDEDELGEILKEELPGPTTFDIYEFHFSDLECTELDLVKCGIQMYYELGVVRKFQIPQEVLVRFLFSISKGYRRITYHNWRHGFNVAQTMFTLLMTGKLKSYYTDLEAFAMVTAGLCHDIDHRGTNNLYQMKSQNPLAKLHGSSILERHHLEFGKFLLSEETLNIYQNLNRRQHEHVIHLMDIAIIATDLALYFKKRAMFQKIVDESKNYQDKKSWVEYLSLETTRKEIVMAMMMTACDLSAITKPWEVQSKVALLVAAEFWEQGDLERTVLDQQPIPMMDRNKAAELPKLQVGFIDFVCTFVYKEFSRFHEEILPMFDRLQNNRKEWKALADEYEAKVKALEEKEEEERVAAKKVGTEICNGGPAPKSSTCCIL

SEQ ID NO: 145 - protein, 498 aa, Homo sapiens [sequence omitted]

SEQ ID NO: 146 - protein, 743 aa, Homo sapiens
  REGION 1..743: note = MISC_FEATURE - N-acetyl-alpha-glucosaminidase (NAGLU)
MEAVAVAAAVGVLLLAGAGGAAGDEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPGWVAGSW

SEQ ID NO: 147 - skipped

SEQ ID NO: 148 - protein, 429 aa, Homo sapiens; identical to SEQ ID NO: 11
  REGION 1..429: note = MISC_FEATURE - Alpha-Galactosidase A (GLA)

SEQ ID NO: 149 - protein, 458 aa, synthetic construct
  REGION 1..458: note = Codon optimized + GET, CO1-GLA-GET
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLLRRRRRRRRKRKKKGKGLGKKRDPCLRKYK

SEQ ID NO: 150 - protein, 1428 aa, synthetic construct; identical to SEQ ID NO: 95
  REGION 1..1428: note = Codon optimized, Cystic Fibrosis Transmembrane Regulator deltaR (CFTRdeltaR) contains R domain deletion

SEQ ID NO: 151 - protein, 1480 aa, synthetic construct; identical to SEQ ID NO: 94
  REGION 1..1480: note = Codon optimized, full length Cystic Fibrosis Transmembrane Regulator (CFTR)

SEQ ID NO: 152 - DNA, 250 bp, synthetic construct; identical to SEQ ID NO: 96
  misc_feature 1..250: note = Mouse U1a promoter
  source 1..250: mol_type = other DNA

SEQ ID NO: 153 - DNA, 101 bp, synthetic construct; identical to SEQ ID NO: 97
  misc_feature 1..101: note = Polymerase III H1 mutant promoter
  source 1..101: mol_type = other DNA

SEQ ID NO: 154 - DNA, 701 bp, synthetic construct
  misc_feature 1..701: note = Chicken beta-actin hybrid promoter CBh (CBh promoter consists of CMV enhancer, CBA promoter, first CBA exon and partial intron)
  source 1..701: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagc

SEQ ID NO: 155 - DNA, 229 bp, synthetic construct
  misc_feature 1..229: note = MeCP2 min promoter sequence
  source 1..229: mol_type = other DNA
agctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgc

SEQ ID NO: 156 - DNA, 737 bp, synthetic construct
  misc_feature 1..737: note = MeCP2 promoter sequence
  source 1..737: mol_type = other DNA
tcaaaccatctgattcaacaatgcacgaccgatctcttatgggcttggcacacaccatctgcccattataaacgtctgcaaagaccaaggtttgatatgttgattttactgtcagccttaagagtgcgacatctgctaatttagtgtaataatacaatcagtagaccctttaaaacaagtcccttggcttggaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaa

SEQ ID NO: 157 - DNA, 418 bp, synthetic construct
  misc_feature 1..418: note = MeCP418 promoter sequence
  source 1..418: mol_type = other DNA
ataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccg

SEQ ID NO: 158 - DNA, 426 bp, synthetic construct
  misc_feature 1..426: note = MeCP426 promoter sequence
  source 1..426: mol_type = other DNA
ataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaa

SEQ ID NO: 159 - DNA, 400 bp, synthetic construct
  misc_feature 1..400: note = VMD2 promoter
  source 1..400: mol_type = other DNA
aattctgtcattttactagggtgatgaaattcccaagcaacaccatccttttcagataagggcactgaggctgagagaggagctgaaacctacccggggtcaccacacacaggtggcaaggctgggaccagaaaccaggactgttgactctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccac

SEQ ID NO: 160 - DNA, 136 bp, synthetic construct
  misc_feature 1..136: note = PDE6b promoter
  source 1..136: mol_type = other DNA
cccatttgtaggagtgagtcagctgacccgcccccggggttcctaatctcactaagaaagactttgctgatgacagggtttcctgggagtccatgcgtgcctggagcagcagcgtctccagggacaggcagccacc

SEQ ID NO: 161 - DNA, 2035 bp, synthetic construct
  misc_feature 1..2035: note = mRho promoter
  source 1..2035: mol_type = other DNA
gcgccaatcagccgatgacttctaacaatactcttaactcacacagagcttgtctcactgagccaacaccctgtaccctcagctcagtgacggctttcaacctgtggggctgcctctgttacccaagtgagagagggccagtgctcccagaggtgaccttgtttgcccattctctccctgggtcagccagtgtttatctgttgtatacccagtccaccctgcaggctcacatcagagcctaggagatggctagtgtccccgcggagaccacgatgaagcttcccagctgtctcaagcacaagctggctgcagaggctgctgaggcactgctagctggggatgggggcagggtagatctggggctgaccaccagggtcagaatcagaacctccaccttgacctcattaacgctggtcttaatcaccaagccaagctccttaaactgctagtggccaactcccaggccctgacacacatacctgccctgtgttcccaaacaagacacctgcatggaaggaagggggttgcttttctaagcaaacatctaggaatcccgggtgcagtgtgaggagactaggcgagggagtactttaagggcctcaaggctcagagaggaatacttcttccctggttagcctcgtgcctaggctccagggtctttgtcctgcctggatacctatgtggcaaggggcatagcatttcccccaccatcagctcttagctcaaccttatcttctcggaaagactgcgcagtgtaacaacacagcagagacttttcttttgtcccctgtctacccctgtaactgctactcagaagcatctttctcacagggtactggcttcttgcatccagagttttttgtctccctcgggcccccagaatcaaattcttcctctgggactcagtggatgtttcacacacgtatcggcctgacagtcatcctggagcatcctacacaggggccatcacagctgcatgtcagaaatgctggcctcacatcctcagacaccaggcctagtgctggtcttcctcagactggcgtccccagcaggccagtaggatcatcttttagcctacagagttctgaagcctcagagccccaggtccctggtcatcttctctgcccctgagatttttccaagttgtatgccttctaggtaaggcaaaacttcttacgcccctcctcgtggcctccaggccccacatgctcacctgaataacctggcagcctgctccctcatgcagggaccacgtcctgctgcacccagcaggccatcccgtctccatagcccatggtcatccctccctggacaggaatgtgtctcctccccgggctgagtcttgctcaagctagaagcactccgaacagggttatgggcgcctcctccatctcccaagtggctggcttatgaatgtttaatgtacatgtgagtgaacaaattccaattgaacgcaacaaatagttatcgagccgctgagccggggggcggggggtgtgagactggaggcgatggacggagctgacggcacacacagctcagatctgtcaagtgagccattgtcagggcttggggactggataagtcagggggtctcctgggaagagatgggataggtgagttcaggaggagacattgtcaactggagccatgtggagaagtgaatttagggcccaaaggttccagtcgcagcctgaggccaccagactgacatggggaggaattcccagaggactctggggcagacaagatgagacaccctttcctttctttacctaagggcctccacccgatgtcaccttggcccctctgcaagccaattaggccccggtggcagcagtgggattagcgttagtatgatatctcgcggatgctgaatcagcctctggcttagggagagaaggtcactttataagggtctggggggggtcagtgcctggagttgcgctgtgggagccgtcagtggctgagctcgccaagcagccttggtctctgtctacgaagagcccgtggggcagcctcgagagccgcagcc

SEQ ID NO: 162 - DNA, 511 bp, synthetic construct
  misc_feature 1..511: note = CMV promoter
  source 1..511: mol_type = other DNA
ccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagat

SEQ ID NO: 163 - DNA, 334 bp, synthetic construct
  misc_feature 1..334: note = UbC promoter
  source 1..334: mol_type = other DNA
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagct

SEQ ID NO: 164 - protein, 342 aa, synthetic construct
  REGION 1..342: note = Cre recombinase
SNLLTVHQNLPALPVDATSDEVRKNLMDMFRDRQAFSEHTWKMLQSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 165 - protein, 7 aa, synthetic construct
  REGION 1..7: note = NLS
PKKKRKV

SEQ ID NO: 166 - protein, 349 aa, synthetic construct
  REGION 1..349: note = Cre with NLS
PKKKRKVSNLLTVHQNLPALPVDATSDEVRKNLMDMFRDRQAFSEHTWKMLQSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 167 - DNA, 1050 bp, synthetic construct
  misc_feature 1..1050: note = Cre with NLS
  source 1..1050: mol_type = other DNA
cccaagaagaagaggaaggtgtccaatttactgaccgtacaccaaaatttgcctgcactgccggtcgatgcaacgagtgatgaggttcgcaagaacctgatggacatgttcagggatcgccaggcgttttctgagcatacctggaaaatgcttcagtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctaggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctcgaggatggcgattaa

SEQ ID NO: 168 - DNA, 34 bp, synthetic construct
  misc_feature 1..34: note = LoxP wildtype
  source 1..34: mol_type = other DNA
ataacttcgtataatgtatgctatacgaagttat

SEQ ID NO: 169 - DNA, 34 bp, synthetic construct
  misc_feature 1..34: note = Lox 511
  source 1..34: mol_type = other DNA
ataacttcgtataatgtatactatacgaagttat

SEQ ID NO: 170 - DNA, 34 bp, synthetic construct
  misc_feature 1..34: note = Lox 5171
  source 1..34: mol_type = other DNA
ataacttcgtataatgtgtactatacgaagttat

SEQ ID NO: 171 - DNA, 34 bp, synthetic construct
  misc_feature 1..34: note = Lox 2272
  source 1..34: mol_type = other DNA
ataacttcgtataaagtatcctatacgaagttat

SEQ ID NO: 172 - DNA, 34 bp, synthetic construct
  misc_feature 1..34: note = M2
  source 1..34: mol_type = other DNA
ataacttcgtataagaaaccatatacgaagttat

SEQ ID NO: 173 - DNA, 34 bp, synthetic construct
  misc_feature 1..34: note = M3
  source 1..34: mol_type = other DNA
ataacttcgtatataataccatatacgaagttat

SEQ ID NO: 174 - DNA, 34 bp, synthetic construct
  misc_feature 1..34: note = M7
  source 1..34: mol_type = other DNA
ataacttcgtataagatagaatatacgaagttat

SEQ ID NO: 175 - DNA, 34 bp, synthetic construct
  misc_feature 1..34: note = M11
  source 1..34: mol_type = other DNA
ataacttcgtatacgataccatatacgaagttat

SEQ ID NO: 176 - DNA, 34 bp, synthetic construct
  misc_feature 1..34: note = Lox 71
  misc_feature 14..16: note = n is a, c, g, or t
  misc_feature 19..21: note = n is a, c, g, or t
  source 1..34: mol_type = other DNA
taccgttcgtatannntannntatacgaagttat

SEQ ID NO: 177 - DNA, 34 bp, synthetic construct
  misc_feature 1..34: note = Lox 66
  misc_feature 14..16: note = n is a, c, g, or t
  misc_feature 19..21: note = n is a, c, g, or t
  source 1..34: mol_type = other DNA
ataacttcgtatannntannntatacgaacggta

SEQ ID NO: 178 - DNA, 34 bp, synthetic construct
  misc_feature 1..34: note = Lox 71
  source 1..34: mol_type = other DNA
taccgttcgtataatgtatgctatacgaagttat

SEQ ID NO: 179 - DNA, 34 bp, synthetic construct
  misc_feature 1..34: note = Lox 66
  source 1..34: mol_type = other DNA
ataacttcgtataatgtatgctatacgaacggta

SEQ ID NO: 180 - protein, 18 aa, synthetic construct
  REGION 1..18: note = T2A
EGRGSLLTCGDVEENPGP

SEQ ID NO: 181 - protein, 19 aa, synthetic construct
  REGION 1..19: note = P2A
ATNFSLLKQAGDVEENPGP

SEQ ID NO: 182 - protein, 20 aa, synthetic construct
  REGION 1..20: note = E2A
QCTNYALLKLAGDVESNPGP

SEQ ID NO: 183 - protein, 22 aa, synthetic construct
  REGION 1..22: note = F2A
VKQTLNFDLLKLAGDVESNPGP

SEQ ID NO: 184 - protein, 22 aa, synthetic construct
  REGION 1..22: note = P2A
GSGATNFSLLKQAGDVEENPGP

SEQ ID NO: 185 - DNA, 66 bp, synthetic construct
  misc_feature 1..66: note = P2A
  source 1..66: mol_type = other DNA
ggaagcggagctactaacttcagcctgctgaagcaggctggagacgtggaggagaaccctggaccc

SEQ ID NO: 186 - DNA, 82 bp, synthetic construct
  misc_feature 1..82: note = Splice Donor
  source 1..82: mol_type = other DNA
gtaagtatcaaggttacaagacaggtttaaggagaccaatagaaactgggcttgtcgagacagagaagactcttgcgtttct

SEQ ID NO: 187 - DNA, 51 bp, synthetic construct
  misc_feature 1..51: note = Splice Acceptor
  source 1..51: mol_type = other DNA
gataggcacctattggtcttactgacatccactttgcctttctctccacag

SEQ ID NO: 188 - DNA, 295 bp, Homo sapiens
  source 1..295: mol_type = other DNA
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggctcccaggggcttcccagtggtccccaggaaccctcgacagggccagggcgtctctctcgtccagcaagggcagggacgggccacaggccaagggc

SEQ ID NO: 189 - DNA, 49 bp, Homo sapiens
  source 1..49: mol_type = other DNA
aaataaatatctttattttcattacatctgtgtgttggttttttgtgtg

SEQ ID NO: 190 - protein, 2273 aa, Homo sapiens [sequence omitted]

SEQ ID NO: 191 - DNA, 6822 bp, Homo sapiens
  source 1..6822: mol_type = other DNA
atgggcttcgtgagacagatacagcttttgctctggaagaactggaccctgcggaaaaggcaaaagattcgctttgtggtggaactcgtgtggcctttatctttatttctggtcttgatctggttaaggaatgccaacccactctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaaggttgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagccatgacctccctctgcaccctcgagctgctggagccagtcgacaagcccaggactaa

SEQ ID NO: 192 - DNA, 2653 bp, Homo sapiens
  source 1..2653: mol_type = other DNA
atgggcttcgtgagacagatacagcttttgctctggaagaactggaccctgcggaaaaggcaaaagattcgctttgtggtggaactcgtgtggcctttatctttatttctggtcttgatctggttaaggaatgccaacccactctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaag

SEQ ID NO: 193 - DNA, 4169 bp, Homo sapiens
  source 1..4169: mol_type = other DNA
gttgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagccatgacctccctctgcaccctcgagctgctggagccagtcgacaagcccaggactaa

SEQ ID NO: 194 - protein, 2317 aa, synthetic construct
  REGION 1..2317: note = ABCA4 with FLAG tag
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDDYKDHDGDYKDHDIDYKDDDDKSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCSGTPLFLKNCFGTGLYLTLVRKMKNIQSQRKGSEGTCSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQDDYKDHDGDYKDHDIDYKDDDDK

SEQ ID NO: 195 - DNA, 6954 bp, synthetic construct
  misc_feature 1..6954: note = ABCA4 with FLAG tag
  source 1..6954: mol_type = other DNA
atgggcttcgtgagacagatacagcttttgctctggaagaactggaccctgcggaaaaggcaaaagattcgctttgtggtggaactcgtgtggcctttatctttatttctggtcttgatctggttaaggaatgccaacccactctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggatgactacaaagaccatgacggtgattataaagatcatgacatcgattacaaggatgacgatgacaagtctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggcaagcttggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtatcttgatcaggtgtttccaggagactatggaaccccacttccttggtactttcttctacaagagtcgtattggcttggcggtgaaggttgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagccatgacctccctctgcaccctcgagctgctggagccagtcgacaagcccaggacgactacaaagaccatgacggtgattataaagatcatgacatcgattacaaggatgacgatgacaagtaa

SEQ ID NO: 196 - protein, 735 aa, unidentified [sequence omitted]

SEQ ID NO: 197 - DNA, 2208 bp, unidentified
  source 1..2208: mol_type = other DNA; note = Adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 198 - DNA, 2818 bp, Homo sapiens
  source 1..2818: mol_type = other DNA
gctccttcctgtactgcccagctccgcttgctccctgaccatccctgcagcagccctgatgtgtcattgtccccctcttaacctgcgctgcagtgctgcagggctgggctctggagctgggtctggtcatttctccttagatatgtagaggcccaggaaaggtttggagcctaagaagccctaggactccaggtctccagggcagccccagcctcttggaatgactttccctaataccacaggggtgttctaatcccaggcagacccaagctgcccctcaccaactcctacgtcctcaacttcctttcataacttctaggatggaaacacctaatcctccagcaatactgaggcttttctccttattctgttttcccttttgaagaagccaaggctcagagcagtcgagtcacctaatcatggtctcatgtcgcctgatcaaggtctcatgtcaccttatcaagatctcacccactcacctattcagttctcaccagttcagttcaggatggcttctaagctaccctgcacagctctgcccacaggacatttgtataagtgagggggtgcaggccttccagccccctccaactccaaaactcagcccccaagatcaagtggactctctgaacccaccctggccctacagttgtcagggtctggatgggaagatgtagagctctcggctttcactctggggacttacccagaacatattctcctcatgagctaaggaggctggctgccatcttcctacatccccccacggcctgggggcaaggacaccctggccccctggagtctggagaactctgaggacagaacttgctcttccacctgcttgggccttacccacaggagaagcactgcttctctacccatgccccatccaactcaggcaccccagggacttgcaacagtctgattttttctcacgtccttcttaaggctctgggctagccacacaaatcaaatcccagtgataggtccagacaatcctatcctgaaactacatcttagtaagactccagggaatcctttccccaaagacagtcttactcctgttctccccccaagcctttctgggccagaagctttgcctggactcaagcaatggcagacaagtgccctctgaggacacggaagtgcatgctcagaactgtgattctccaagtggaggcagaggagaaggcccaggcttcccagcagggctaaggatatgcaaggagtgcattcatccggaggtgttggcagcatcccagccccaccccattctcatcgtaaatcaggctcacttccattggctgcatacggtggagtgatgtgaccatatgtcacttgagcattacacaaatcctaatgagctaaaaatatgtttgttttagctaattgacctctttggccttcataaagcagttggtaaacatcctcagataatgatttccaaagagcagattgtgggtctcagctgtgcagagaaagcccacgtccctgagaccaccttctccagctgcctactgaggcacacaggggcgcctgcctgctgcccgctcagccaaggcggtgttgctggagccagcttgggacagctctcccaacgctctgccctggccttgcgaccactctctgggccgtagttgtctgtctgttaagtgaggaaagtgcccatctccagaggcattcagcggcaaagcagggcttccaggttccgaccccatagcaggacttcttggatttctacagccagtcagttgcaagcagcacccatattatttctataagaagtggcaggagctgggatctgaagagttcagcagtctacctttccctgtttcttgtgctttatgcagtcaggaggaatgatctggattccatgtgaagcctgggaccacggagacccaagacttcctgcttgattctccctgcgaactgcaggctgtgggctgagccttcaagaagcaggagtcccctctagccattaactctcagagctaacctcatttgaatgggaacactagtcctgtgatgtctggaaggtgggcgcctctacactccacaccctacatggtggtccagacacatcattcccagcattagaaagctgtagggggacccgttctgttccctggaggcattaaagggacatagaaataaatctcaagctctgaggctgatgccagcctcagactcagcctctgcactgtatgggccaattgtagccccaaggacttcttcttgctgcaccccctatctgtccacacctaaaacgatgggcttctatttagttacagaactctctggcctgttttgttttgctttgctttgttttgttttgtttttttgtttttttgttttttagctatgaaacagaggtaatatctaatacagataacttaccagtaatgagtgcttcctacttactgggtactgggaagaagtgctttacacatattttctcatttaatctacacaataagtaattaagacatttccctgaggccacgggagagacagtggcagaacagttctccaaggaggacttgcaagttaataactggactttgcaaggctctggtggaaactgtcagcttgtaaaggatggagcacagtgtctggcatgtagcaggaactaaaataatggcagtgattaatgttatgatatgcagacacaacacagcaagataagatgcaatgtaccttctgggtcaaaccaccctggccactcctccccgatacccagggttgatgtgcttgaattagacaggattaaaggcttactggagctggaagccttgccccaactcaggagtttagcccc

SEQ ID NO: 199 - DNA, 2100 bp, Homo sapiens
  source 1..2100: mol_type = other DNA
ccctgctaatttttgtattttttgtagagacagggtctcactatgttgcccaggctagtctcgaactcctgggctcaagtgatcctctcacctcggcctcccaaagaactgggattacaggcatgagctaccatgtccagcccaattgcccattgatgggcacgggttgcttccatgtttcagctgttgtgaatcacgctgctgtgaacatgcgtgtgcaaacagcccttccagaccctgccttccattcctctgggcctatacccagcagtgcggttgctgggtcctatgggaattctacgtttaacttttggaggagctgccaaactgttttccacagtggctgcgccatcacaatccaattttaggacatttttatcacccataaagcactccctgtacccattaagaagtcatcctccatttccctccctcccctgtcctggcacccattcctctgctttgtgtgtctctggattgccctatctaagcatttcacagagatggagccatgcgctccgtggtcttttgtgtctggcttcgctcactgagcatgctgttctccaggtccatccacgttgtagcgtaggtcagcccttcattccctgttatggccaaatgatactccattgtacagacaggccactttttacccactcatctgcttctggacttttgggttgcttctaccatgtggcctgttgggaacagtgctctgtttgtattcatgtacgggtttttgtgtggacacacattttcaagtctcttgggtacacaggtgtagaagtgccagagttggggaaaaagctcacctttctaggctgtgaatgggccctggcaagtctgtggccaggactcgtcttctcttccacatggggcccctagcttggcacctagcacgtggcaggcagcgacagatgttaaaagccattcttgctatgggtagccaggctggggctccatgcagccctggccttcagcttggcagccagggcccccttgtgcctgcagcagaagccatgctgccaggagtgtaagtgtgagccaggaatgctggagaatcgtggctctgagaacagggacaagaggccacaagctcacgccttggctttcctaagcttaaggaataaacccaaaaggaggtacctggaaggagctggatttggggactgaggagctgggagctgatggaagccgtgaaaggggatgtgctcctggggaggcgctggggcgggtgggccgtggaggggacagggcccgttggttggaaactgaggcgaggctacggagttgggcactaacaggtcatccgtgcccctgcgaagcgtggggacacagggacagcagagatggcctgtctggacactctgtcgacggggggcctgtggttggtgaagcccaaggcaaggctgtgaactcagggcaagggagacgtgagcaggcgctgccgtgggctgatgtgggcactgcatgtgcaccctggcggccaaaggacctacagctcatggggggcaagggggaggagggaagccaacagcaggatgtgcgcagtcagtctgccccccctacactggaggaggagccccccggcacaaatctcgcccgtttgggcccacggacatggctggcctcgcaaggaggatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggc

SEQ ID NO: 200 - DNA, 136 bp, Homo sapiens; identical to SEQ ID NO: 160
  source 1..136: mol_type = other DNA

SEQ ID NO: 201 - protein, 2215 aa, Homo sapiens
MVILQQGDHVWMDLRLGQEFDVPIGAVVKLCDSGQVQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNSRDQCCIISGESGAGKTESTKLILQFLAAISGQHSWIEQQVLEATPILEAFGNAKTIRNDNSSRFGKYIDIHFNKRGAIEGAKIEQYLLEKSRVCRQALDERNYHVFYCMLEGMSEDQKKKLGLGQASDYNYLAMGNCITCEGRVDSQEYANIRSAMKVLMFTDTENWEISKLLAAILHLGNLQYEARTFENLDACEVLFSPSLATAASLLEVNPPDLMSCLTSRTLITRGETVSTPLSREQALDVRDAFVKGIYGRLFVWIVDKINAAIYKPPSQDVKNSRRSIGLLDIFGFENFAVNSFEQLCINFANEHLQQFFVRHVFKLEQEEYDLESIDWLHIEFTDNQDALDMIANKPMNIISLIDEESKFPKGTDTTMLHKLNSQHKLNANYIPPKNNHETQFGINHFAGIVYYETQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRRAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQKKSSVRHKLVHLTLKKKSKLTEEVTKRLHDGESTVQGNSMLEDRPTSNLEKLHFIIGNGILRPALRDEIYCQISKQLTHNPSKSSYARGWILVSLCVGCFAPSEKFVKYLRNFIHGGPPGYAPYCEERLRRTFVNGTRTQPPSWLELQATKSKKPIMLPVTFMDGTTKTLLTDSATTAKELCNALADKISLKDRFGFSLYIALFDKVSSLGSGSDHVMDAISQCEQYAKEQGAQERNAPWRLFFRKEVFTPWHSPSEDNVATNLIYQQVVRGVKFGEYRCEKEDDLAELASQQYFVDYGSEMILERLLNLVPTYIPDREITPLKTLEKWAQLAIAAHKKGIYAQRRTDAQKVKEDVVSYARFKWPLLFSRFYEAYKFSGPSLPKNDVIVAVNWTGVYFVDEQEQVLLELSFPEIMAVSSSRECRVWLSLGCSDLGCAAPHSGWAGLTPAGPCSPCWSCRGAKTTAPSFTLATIKGDEYTFTSSNAEDIRDLVVTFLEGLRKRSKYVVALQDNPNPAGEESGFLSFAKGDLIILDHDTGEQVMNSGWANGINERTKQRGDFPTDSVYVMPTVTMPPREIVALVTMTPDQRQDVVRLLQLRTAEPEVRAKPYTLEEFSYDYFRPPPKHTLSRVMVSKARGKDRLWSHTREPLKQALLKKLLGSEELSQEACLAFIAVLKYMGDYPSKRTRSVNELTDQIFEGPLKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIATRLLLKSSEGFSLFVKIADKVLSVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEVKQTTEPNFPEILLIAINKYGVSLIDPKTKDILTTHPFTKISNWSSGNTYFHITIGNLVRGSKLLCETSLGYKMDDLLTSYISQMLTAMSKQRGSRSGK

SEQ ID NO: 202 - DNA, 6648 bp, Homo sapiens
  source 1..6648: mol_type = other DNA
atggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggtatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcagggagtgccgtgtctggctctcactgggctgctctgatcttggctgtgctgcgcctcactcaggctgggcaggactgaccccggcggggccctgttctccgtgttggtcctgcaggggagcgaaaacgacggcccccagcttcacgctggccaccatcaagggggacgaatacaccttcacctccagcaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgacagtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaagtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcctcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaagtga

SEQ ID NO: 203 - protein, 2479 aa, Homo sapiens
MPPNINWKEIMKVDPDDLPRQEELADNLLISLSKVEVNELKSEKQENVIHLFRITQSLMKMKAQEVELALEEVEKAGEEQAKFENQLKTKVMKLENELEMAQQSAGGRDTRFLRNEICQLEKQLEQKDRELEDMEKELEKEKKVNEQLALRNEEAENENSKLRRENKRLKKKNEQLCQDIIDYQKQIDSQKETLLSRRGEDSDYRSQLSKKNYELIQYLDEIQTLTEANEKIEVQNQEMRKNLEESVQEMEKMTDEYNRMKAIVHQTDNVIDQLKKENDHYQLQVQELTDLLKSKNEEDDPIMVAVNAKVEEWKLILSSKDDEIIEYQQMLHNLREKLKNAQLDADKSNVMALQQGIQERDSQIKMLTEQVEQYTKEMEKNTCIIEDLKNELQRNKGASTLSQQTHMKIQSTLDILKEKTKEAERTAELAEADAREKDKELVEALKRLKDYESGVYGLEDAVVEIKNCKNQIKIRDREIEILTKEINKLELKISDFLDENEALRERVGLEPKTMIDLTEFRNSKHLKQQQYRAENQILLKEIESLEEERLDLKKKIRQMAQERGKRSATSGLTTEDLNLTENISQGDRISERKLDLLSLKNMSEAQSKNEFLSRELIEKERDLERSRTVIAKFQNKLKELVEENKQLEEGMKEILQAIKEMQKDPDVKGGETSLIIPSLERLVNAIESKNAEGIFDASLHLKAQVDQLTGRNEELRQELRESRKEAINYSQQLAKANLKIDHLEKETSLLRQSEGSNVVFKGIDLPDGIAPSSASIINSQNEYLIHLLQELENKEKKLKNLEDSLEDYNRKFAVIRHQQSLLYKEYLSEKETWKTESKTIKEEKRKLEDQVQQDAIKVKEYNNLLNALQMDSDEMKKILAENSRKITVLQVNEKSLIRQYTTLVELERQLRKENEKQKNELLSMEAEVCEKIGCLQRFKEMAIFKIAALQKVVDNSVSLSELELANKQYNELTAKYRDILQKDNMLVQRTSNLEHLECENISLKEQVESINKELEITKEKLHTIEQAWEQETKLGNESSMDKAKKSITNSDIVSISKKITMLEMKELNERQRAEHCQKMYEHLRTSLKQMEERNFELETKFAELTKINLDAQKVEQMLRDELADSVSKAVSDADRQRILELEKNEMELKVEVSKLREISDIARRQVEILNAQQQSRDKEVESLRMQLLDYQAQSDEKSLIAKLHQHNVSLQLSEATALGKLESITSKLQKMEAYNLRLEQKLDEKEQALYYARLEGRNRAKHLRQTIQSLRRQFSGALPLAQQEKFSKTMIQLQNDKLKIMQEMKNSQQEHRNMENKTLEMELKLKGLEELISTLKDTKGAQKVINWHMKIEELRLQELKLNRELVKDKEEIKYLNNIISEYERTISSLEEEIVQQNKFHEERQMAWDQREVDLERQLDIFDRQQNEILNAAQKFEEATGSIPDPSLPLPNQLEIALRKIKENIRIILETRATCKSLEEKLKEKESALRLAEQNILSRDKVINELRLRLPATAEREKLIAELGRKEMEPKSHHTLKIAHQTIANMQARLNQKEEVLKKYQRLLEKAREEQREIVKKHEEDLHILHHRLELQADSSLNKFKQTAWDLMKQSPTPVPTNKHFIRLAEMEQTVAEQDDSLSSLLVKLKKVSQDLERQREITELKVKEFENIKLQLQENHEDEVKKVKAEVEDLKYLLDQSQKESQCLKSELQAQKEANSRAPTTTMRNLVERLKSQLALKEKQQKALSRALLELRAEMTAAAEERIISATSQKEAHLNVQQIVDRHTRELKTQVEDLNENLLKLKEALKTSKNRENSLTDNLNDLNNELQKKQKAYNKILREKEEIDQENDELKRQIKRLTSGLQGKPLTDNKQSLIEELQRKVKKLENQLEGKVEEVDLKPMKEKNAKEELIRWEEGKKWQAKIEGIRNKLKEKEGEVFTLTKQLNTLKDLFAKADKEKLTLQRKLKTTGMTVDQVLGIRALESEKELEELKKRNLDLENDILYMRAHQALPRDSVVEDLHLQNRYLQEKLHALEKQFSKDTYSKPSISGIESDDHCQREQELQKENLKLSSENIELKFQLEQANKDLPRLKNQVRDLKEMCEFLKKEKAEVQRKLGHVRGSGRSGKTIPELEKTIGLMKKVVEKVQRENEQLKKASGILTSEKMANIEQENEKLKAELEKLKAHLGHQLSMHYESKTKGTEKIIAENERLRKELKKETDAAEKLRIAKNNLEILNEKMTVQLEETGKRLQFAESRGPQLEGADSKSWKSIVVTRMYETKLKELETDIAKKNQSITDLKQLVKEATEREQKVNKYNEDLEQQIKILKHVPEGAETEQGLKRELQVLRLANHQLDKEKAELIHQIEANKDQSGAESTIPDADQLKEKIKDLETQLKMSDLEKQHLKEEIKKLKKELENFDPSFFEEIEDLKYNYKEEVKKNILLEEKVKKLSEQLGVELTSPVAASEEFEDEEESPVNFPIY

SEQ ID NO: 204 - DNA, 7440 bp, Homo sapiens
  source 1..7440: mol_type = other DNA
atgccacctaatataaactggaaagaaataatgaaagttgacccagatgacctgccccgtcaagaagaactggcagataatttattgatttccttatccaaggtggaagtaaatgagctaaaaagtgaaaagcaagaaaatgtgatacaccttttcagaattactcagtcactaatgaagatgaaagctcaagaagtggagctggctttggaagaagtagaaaaagctggagaagaacaagcaaaatttgaaaatcaattaaaaactaaagtaatgaaactggaaaatgaactggagatggctcagcagtctgcaggtggacgagatactcggtttttacgtaatgaaatttgccaacttgaaaaacaattagaacaaaaagatagagaattggaggacatggaaaaggagttggagaaagagaagaaagttaatgagcaattggctcttcgaaatgaggaggcagaaaatgaaaacagcaaattaagaagagagaacaaacgtctaaagaaaaagaatgaacaactttgtcaggatattattgactaccagaaacaaatagattcacagaaagaaacacttttatcaagaagaggggaagacagtgactaccgatcacagttgtctaaaaaaaactatgagcttatccaatatcttgatgaaattcagactttaacagaagctaatgagaaaattgaagttcagaatcaagaaatgagaaaaaatttagaagagtctgtacaggaaatggagaagatgactgatgaatataatagaatgaaagctattgtgcatcagacagataatgtaatagatcagttaaaaaaagaaaacgatcattatcaacttcaagtgcaggagcttacagatcttctgaaatcaaaaaatgaagaagatgatccaattatggtagctgtcaatgcaaaagtagaagaatggaagctaattttgtcttctaaagatgatgaaattattgagtatcagcaaatgttacataacctaagggagaaacttaagaatgctcagcttgatgctgataaaagtaatgttatggctctacagcagggtatacaggaacgagacagtcaaattaagatgctcaccgaacaagtagaacaatatacaaaagaaatggaaaagaatacttgtattattgaagatttgaaaaatgagctccaaagaaacaaaggtgcttcaaccctttctcaacagactcatatgaaaattcagtcaacgttagacattttaaaagagaaaactaaagaggctgagagaacagctgaactggctgaggctgatgctagggaaaaggataaagaattagttgaggctctgaagaggttaaaagattatgaatcgggagtatatggtttagaagatgctgtcgttgaaataaagaattgtaaaaaccaaattaaaataagagatcgagagattgaaatattaacaaaggaaatcaataaacttgaattgaagatcagtgatttccttgatgaaaatgaggcacttagagagcgtgtgggccttgaaccaaagacaatgattgatttaactgaatttagaaatagcaaacacttaaaacagcagcagtacagagctgaaaaccagattcttttgaaagagattgaaagtctagaggaagaacgacttgatctgaaaaaaaaaattcgtcaaatggctcaagaaagaggaaaaagaagtgcaacttcaggattaaccactgaggacctgaacctaactgaaaacatttctcaaggagatagaataagtgaaagaaaattggatttattgagcctcaaaaatatgagtgaagcacaatcaaagaatgaatttctttcaagagaactaattgaaaaagaaagagatttagaaaggagtaggacagtgatagccaaatttcagaataaattaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgctatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaagatagaccatcttgaaaaagaaactagtcttttacgacaatcagaaggatcaaatgttgtttttaaaggaattgacttacctgatgggatagcaccatctagtgccagtatcattaattctcagaatgaatatttaatacatttgttacaggaactagaaaataaagaaaaaaagttaaagaatttagaagattctcttgaagattacaacagaaaatttgctgtaattcgtcatcaacaaagtttgttgtataaagaatacctaagtgaaaaggagacctggaaaacagaatctaaaacaataaaagaggaaaagagaaaacttgaggatcaagtccaacaagatgctataaaagtaaaagaatataataatttgctcaatgctcttcagatggattcggatgaaatgaaaaaaatacttgcagaaaatagtaggaaaattactgttttgcaagtgaatgaaaaatcacttataaggcaatatacaaccttagtagaattggagcgacaacttagaaaagaaaatgagaagcaaaagaatgaattgttgtcaatggaggctgaagtttgtgaaaaaattgggtgtttgcaaagatttaaggaaatggccattttcaagattgcagctctccaaaaagttgtagataatagtgtttctttgtctgaactagaactggctaataaacagtacaatgaactgactgctaagtacagggacatcttgcaaaaagataatatgcttgttcaaagaacaagtaacttggaacacctggagtgtgaaaacatctccttaaaagaacaagtggagtctataaataaagaactggagattaccaaggaaaaacttcacactattgaacaagcctgggaacaggaaactaaattaggtaatgaatctagcatggataaggcaaagaaatcaataaccaacagtgacattgtttccatttcaaaaaaaataactatgctggaaatgaaggaattaaatgaaaggcagcgggctgaacattgtcaaaaaatgtatgaacacttacggacttcgttaaagcaaatggaggaacgtaattttgaattggaaaccaaatttgctgagcttaccaaaatcaatttggatgcacagaaggtggaacagatgttaagagatgaattagctgatagtgtgagcaaggcagtaagtgatgctgataggcaacggattctagaattagagaagaatgaaatggaactaaaagttgaagtgtcaaaactgagagagatttctgatattgccagaagacaagttgaaattttgaatgcacaacaacaatctagggacaaggaagtagagtccctcagaatgcaactgctagactatcaggcacagtctgatgaaaagtcgctcattgccaagttgcaccaacataatgtctctcttcaactgagtgaggctactgctcttggtaagttggagtcaattacatctaaactgcagaagatggaggcctacaacttgcgcttagagcagaaacttgatgaaaaagaacaggctctctattatgctcgtttggagggaagaaacagagcaaaacatctgcgccaaacaattcagtctctacgacgacagtttagtggagctttacccttggcacaacaggaaaagttctccaaaacaatgattcaactacaaaatgacaaacttaagataatgcaagaaatgaaaaattctcaacaagaacatagaaatatggagaacaaaacattggagatggaattaaaattaaagggcctggaagagttaataagcactttaaaggataccaaaggagcccaaaaggtaatcaactggcatatgaaaatagaagaacttcgtcttcaagaacttaaactaaatcgggaattagtcaaggataaagaagaaataaaatatttgaataacataatttctgaatatgaacgtacaatcagcagtcttgaagaagaaattgtgcaacagaacaagtttcatgaagaaagacaaatggcctgggatcaaagagaagttgacctggaacgccaactagacatttttgaccgtcagcaaaatgaaatactaaatgcggcacaaaagtttgaagaagctacaggatcaatccctgaccctagtttgccccttccaaatcaacttgagatcgctctaaggaaaattaaggagaacattcgaataattctagaaacacgggcaacttgcaaatcactagaagagaaactaaaagagaaagaatctgctttaaggttagcagaacaaaatatactgtcaagagacaaagtaatcaatgaactgaggcttcgattgcctgccactgcagaaagagaaaagctcatagctgagctaggcagaaaagagatggaaccaaaatctcaccacacattgaaaattgctcatcaaaccattgcaaacatgcaagcaaggttaaatcaaaaagaagaagtattaaagaagtatcaacgtcttctagaaaaagccagagaggagcaaagagaaattgtgaagaaacatgaggaagaccttcatattcttcatcacagattagaactacaggctgatagttcactaaataaattcaaacaaacggcttgggatttaatgaaacagtctcccactccagttcctaccaacaagcattttattcgtctggctgagatggaacagacagtagcagaacaagatgactctctttcctcactcttggtcaaactaaagaaagtatcacaagatttggagagacaaagagaaatcactgaattaaaagtaaaagaatttgaaaatatcaaattacagcttcaagaaaaccatgaagatgaagtgaaaaaagtaaaagcggaagtagaggatttaaagtatcttctggaccagtcacaaaaggagtcacagtgtttaaaatctgaacttcaggctcaaaaagaagcaaattcaagagctccaacaactacaatgagaaatctagtagaacggctaaagagccaattagccttgaaggagaaacaacagaaagcacttagtcgggcacttttagaactccgggcagaaatgacagcagctgctgaagaacgtattatttctgcaacttctcaaaaagaggcccatctcaatgttcaacaaatcgttgatcgacatactagagagctaaagacacaagttgaagatttaaatgaaaatcttttaaaattgaaagaagcacttaaaacaagtaaaaacagagaaaactcactaactgataatttgaatgacttaaataatgaactgcaaaagaaacaaaaagcctataataaaatacttagagagaaagaggaaattgatcaagagaatgatgaactgaaaaggcaaattaaaagactaaccagtggattacagggcaaacccctgacagataataaacaaagtctaattgaagaactccaaaggaaagttaaaaaactagagaaccaattagagggaaaggtggaggaagtagacctaaaacctatgaaagaaaagaatgctaaagaagaattaattaggtgggaagaaggtaaaaagtggcaagccaaaatagaaggaattcgaaacaagttaaaagagaaagagggggaagtctttactttaacaaagcagttgaatactttgaaggatctttttgccaaagccgataaagagaaacttactttgcagaggaaactaaaaacaactggcatgactgttgatcaggttttgggaatacgagctttggagtcagaaaaagaattggaagaattaaaaaagagaaatcttgacttagaaaatgatatattgtatatgagggcccaccaagctcttcctcgagattctgttgtagaagatttacatttacaaaatagatacctccaagaaaaacttcatgctttagaaaaacagttttcaaaggatacatattctaagccttcaatttcaggaatagagtcagatgatcattgtcagagagaacaggagcttcagaaggaaaacttgaagttgtcatctgaaaatattgaactgaaatttcagcttgaacaagcaaataaagatttgccaagattaaagaatcaagtcagagatttgaaggaaatgtgtgaatttcttaagaaagaaaaagcagaagttcagcggaaacttggccatgttagagggtctggtagaagtggaaagacaatcccagaactggaaaaaaccattggtttaatgaaaaaagtagttgaaaaagtccagagagaaaatgaacagttgaaaaaagcatcaggaatattgactagtgaaaaaatggctaatattgagcaggaaaatgaaaaattgaaggctgaattagaaaaacttaaagctcatcttgggcatcagttgagcatgcactatgaatccaagaccaaaggcacagaaaaaattattgctgaaaatgaaaggcttcgtaaagaacttaaaaaagaaactgatgctgcagagaaattacggatagcaaagaataatttagagatattaaatgagaagatgacagttcaactagaagagactggtaagagattgcagtttgcagaaagcagaggtccacagcttgaaggtgctgacagtaagagctggaaatccattgtggttacaagaatgtatgaaaccaagttaaaagaattggaaactgatattgccaaaaaaaatcaaagcattactgaccttaaacagcttgtaaaagaagcaacagagagagaacaaaaagttaacaaatacaatgaagaccttgaacaacagattaagattcttaaacatgttcctgaaggtgctgagacagagcaaggccttaaacgggagcttcaagttcttagattagctaatcatcagctggataaagagaaagcagaattaatccatcagatagaagctaacaaggaccaaagtggagctgaaagcaccatacctgatgctgatcaactaaaggaaaaaataaaagatctagagacacagctcaaaatgtcagatctagaaaagcagcatttgaaggaggaaataaagaagctgaaaaaagaactggaaaattttgatccttcattttttgaagaaattgaagatcttaagtataattacaaggaagaagtgaagaagaatattctcttagaagagaaggtaaaaaaactttcagaacaattgggagttgaattaactagccctgttgctgcttctgaagagtttgaagatgaagaagaaagtcctgttaatttccccatttactaa

SEQ ID NO: 205 - protein, 724 aa, unidentified
  source 1..724: note = Adeno-associated virus
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 206 - protein, 737 aa, unidentified
  source 1..737: note = Adeno-associated virus
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 207 - DNA, 34 bp, synthetic construct
  misc_feature 1..34: note = LoxN
  source 1..34: mol_type = other DNA
ataacttcgtatagtataccttatacgaagttat

SEQ ID NO: 208 - DNA, 240 bp, synthetic construct
  source 1..240: mol_type = other DNA
tggaggcagaggagaaggcccaggcttcccagcagggctaaggatatgcaaggagtgcattcatccggaggtgttggcagcatcccagccccaccccattctcatcgtaaatcaggctcacttccattggctgcatacggtggagtgatgtgaccatatgtcacttgagcattacacaaatcctaatgagctaaaaatatgtttgttttagctaattgacctctttggccttcataaagc

SEQ ID NO: 209 - DNA, 319 bp, synthetic construct
  source 1..319: mol_type = other DNA
agttaggggaccttctcctcccttttcctggatcctgagtacctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatccagctggagccctgagtggctgagctcaggccttcgcagcattcttgggtgggagcagccacgggtcagccacaagggccac

SEQ ID NO: 210 - DNA, 126 bp, synthetic construct
  source 1..126: mol_type = other DNA
gccccggctctgactgaccgcgttactcccacaggtgagcgggcggtttaagggaaggttggttggtggggtattaatctttaattacctggagcacctgcctgaaatcactttttttcaggttgg

SEQ ID NO: 211 - DNA, 642 bp, synthetic construct
  source 1..642: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactga

SEQ ID NO: 212 - DNA, 2653 bp, synthetic construct
  source 1..2653: mol_type = other DNA
atgggcttcgtgagacagatacagcttttgctctggaagaactggaccctgcggaaaaggcaaaagattcgctttgtggtggaactcgtgtggcctttatctttatttctggtcttgatctggttaaggaatgccaacccactctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgaggtcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccaaatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggcaagcttggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtatcttgatcaggtgtttccaggagactatggaaccccacttccttggtactttcttctacaagagtcgtattggcttggcggtgaag

SEQ ID NO: 213 - DNA, 1162 bp, synthetic construct
  source 1..1162: mol_type = other DNA
tccaatttactgaccgtacaccaaaatttgcctgcactgccggtcgatgcaacgagtgatgaggttcgcaagaacctgatggacatgttcagggatcgccaggcgttttctgagcatacctggaaaatgcttcagtccgtttgccggtcgtgggcggcatggtgcaagttgaataaccggaaatggtttcccgcagaacctgaagatgttcgagattatctgctatatctgcaggtaagtatcaaggttacaagacaggtttaaggagaccaatagaaactgggcttgtcgagacagagaagactcttgcgtttctgataggcacctattggtcttactgacatccactttgcctttctctccacaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctaggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctcgaggatggcgattaa

SEQ ID NO: 214 - DNA, 133 bp, synthetic construct
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 215 - DNA, 130 bp, synthetic construct
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 216 - DNA, 143 bp, synthetic construct
  source 1..143: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 217 - DNA, 34 bp, synthetic construct
  source 1..34: mol_type = other DNA
ataacttcgtataatgtatgctatagcaatttat

SEQ ID NO: 218 - DNA, 286 bp, synthetic construct
  source 1..286: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatg

SEQ ID NO: 219 - DNA, 278 bp, synthetic construct
  source 1..278: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 220 - DNA, 6517 bp, synthetic construct
  source 1..6517: mol_type = other DNA
tcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacacgtgcacggagagtcaagaataaacctaggataacttcgtataatgtatgctatagcaatttatctagtgataggcacctattggtcttactgacatccactttgcctttctctccacaggttgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagccatgacctccctctgcaccctcgagctgctggagccagtcgacaagcccaggactacccatacgatgttccagattacgcttaaataaatatctttattttcattacatctgtgtgttggttttttgtgtgagatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaattaattaaggcgatgaacggtaatcgtaaaactagcatgtcaatcatatgtaccccggttgataatcagaaaagccccaaaaacaggaagattgtataagcattaattaatttaaatacatggacatgtcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaaagcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtttaaaccatg

SEQ ID NO: 221 - DNA, 6931 bp, synthetic construct
  source 1..6931: mol_type = other DNA
tcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgctggaggcagaggagaaggcccaggcttcccagcagggctaaggatatgcaaggagtgcattcatccggaggtgttggcagcatcccagccccaccccattctcatcgtaaatcaggctcacttccattggctgcatacggtggagtgatgtgaccatatgtcacttgagcattacacaaatcctaatgagctaaaaatatgtttgttttagctaattgacctctttggccttcataaagcctgcaggcgtaccagttaggggaccttctcctcccttttcctggatcctgagtacctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatccagctggagccctgagtggctgagctcaggccttcgcagcattcttgggtgggagcagccacgggtcagccacaagggccactccggagccccggctctgactgaccgcgttactcccacaggtgagcgggcggtttaagggaaggttggttggtggggtattaatctttaattacctggagcacctgcctgaaatcactttttttcaggttggaccggtcgccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccactctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgaggtcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccaaatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggcaagcttggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtatcttgatcaggtgtttccaggagactatggaaccccacttccttggtactttcttctacaagagtcgtattggcttggcggtgaaggtaagtatcaaggttacaagacaggtttaaggagaccaatagaaactgggcttgtcgagacagagaagactcttgcgtttctaactagttaccgttcgtataatgtatgctatacgaagttatctagaggaagcggagctactaacttcagcctgctgaagcaggctggagacgtggaggagaaccctggacccaagaagaagaggaaggtgtccaatttactgaccgtacaccaaaatttgcctgcactgccggtcgatgcaacgagtgatgaggttcgcaagaacctgatggacatgttcagggatcgccaggcgttttctgagcatacctggaaaatgcttcagtccgtttgccggtcgtgggcggcatggtgcaagttgaataaccggaaatggtttcccgcagaacctgaagatgttcgagattatctgctatatctgcaggtaagtatcaaggttacaagacaggtttaaggagaccaatagaaactgggcttgtcgagacagagaagactcttgcgtttctgataggcacctattggtcttactgacatccactttgcctttctctccacaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctaggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctcgaggatggcgattaaataaatatctttattttcattacatctgtgtgttggttttttgtgtgagatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaattaattaaggcgatgaacggtaatcgtaaaactagcatgtcaatcatatgtaccccggttgataatcagaaaagccccaaaaacaggaagattgtataagcattaattaatttaaatacatggacatgtcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaaagcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtttaaaccatg

SEQ ID NO: 222 - DNA, 6742 bp, synthetic construct
  source 1..6742: mol_type = other DNA
tcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgctggaggcagaggagaaggcccaggcttcccagcagggctaaggatatgcaaggagtgcattcatccggaggtgttggcagcatcccagccccaccccattctcatcgtaaatcaggctcacttccattggctgcatacggtggagtgatgtgaccatatgtcacttgagcattacacaaatcctaatgagctaaaaatatgtttgttttagctaattgacctctttggccttcataaagcctgcaggcccatttgtaggagtgagtcagctgacccgcccccggggttcctaatctcactaagaaagactttgctgatgacagggtttcctgggagtccatgcgtgcctggagcagcagcgtctccagggacaggcagccaccaccggagccccggctctgactgaccgcgttactcccacaggtgagcgggcggtttaagggaaggttggttggtggggtattaatctttaattacctggagcacctgcctgaaatcactttttttcaggttggaccggtcgccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccactctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgaggtcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccaaatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggcaagcttggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtatcttgatcaggtgtttccaggagactatggaaccccacttccttggtactttcttctacaagagtcgtattggcttggcggtgaaggtaagtatcaaggttacaagacaggtttaaggagaccaatagaaactgggcttgtcgagacagagaagactcttgcgtttctaactagttaccgttcgtataatgtatgctatacgaagttatctagaggaagcggagctactaacttcagcctgctgaagcaggctggagacgtggaggagaaccctggacccaagaagaagaggaaggtgtccaatttactgaccgtacaccaaaatttgcctgcactgccggtcgatgcaacgagtgatgaggttcgcaagaacctgatggacatgttcagggatcgccaggcgttttctgagcatacctggaaaatgcttcagtccgtttgccggtcgtgggcggcatggtgcaagttgaataaccggaaatggtttcccgcagaacctgaagatgttcgagattatctgctatatctgcaggtaagtatcaaggttacaagacaggtttaaggagaccaatagaaactgggcttgtcgagacagagaagactcttgcgtttctgataggcacctattggtcttactgacatccactttgcctttctctccacaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctaggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctcgaggatggcgattaaataaatatctttattttcattacatctgtgtgttggttttttgtgtgagatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaattaattaaggcgatgaacggtaatcgtaaaactagcatgtcaatcatatgtaccccggttgataatcagaaaagccccaaaaacaggaagattgtataagcattaattaatttaaatacatggacatgtcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaaagcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtttaaaccatg

SEQ ID NO: 223 - DNA, 6867 bp, synthetic construct
  source 1..6867: mol_type = other DNA
tcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaaccggtccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccactctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgaggtcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccaaatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggcaagcttggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtatcttgatcaggtgtttccaggagactatggaaccccacttccttggtactttcttctacaagagtcgtattggcttggcggtgaaggtaagtatcaaggttacaagacaggtttaaggagaccaatagaaactgggcttgtcgagacagagaagactcttgcgtttctaactagttaccgttcgtataatgtatgctatacgaagttatctagaggaagcggagctactaacttcagcctgctgaagcaggctggagacgtggaggagaaccctggacccaagaagaagaggaaggtgtccaatttactgaccgtacaccaaaatttgcctgcactgccggtcgatgcaacgagtgatgaggttcgcaagaacctgatggacatgttcagggatcgccaggcgttttctgagcatacctggaaaatgcttcagtccgtttgccggtcgtgggcggcatggtgcaagttgaataaccggaaatggtttcccgcagaacctgaagatgttcgagattatctgctatatctgcaggtaagtatcaaggttacaagacaggtttaaggagaccaatagaaactgggcttgtcgagacagagaagactcttgcgtttctgataggcacctattggtcttactgacatccactttgcctttctctccacaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctaggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctcgaggatggcgattaaataaatatctttattttcattacatctgtgtgttggttttttgtgtgagatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaattaattaaggcgatgaacggtaatcgtaaaactagcatgtcaatcatatgtaccccggttgataatcagaaaagccccaaaaacaggaagattgtataagcattaattaatttaaatacatggacatgtcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaaagcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtttaaaccatg

SEQ ID NO: 224 - DNA, 6517 bp, synthetic construct; identical to SEQ ID NO: 220
  source 1..6517: mol_type = other DNA